SEQUENCE LISTING (118 sequences)

SEQ ID NO: 1 - protein, 39 aa, Homo sapiens
KPLDGEYFTLQIRGRERFEMFRELNEALELKDAQAGKEP

SEQ ID NO: 2 - DNA, 117 bp, Homo sapiens
  source 1..117: mol_type = unassigned DNA
aaacctctggatggcgagtactttaccctgcagattagaggccgcgaacgattcgagatgtttcgcgaactgaatgaggccctggaactgaaggatgctcaggcaggcaaggagcca

SEQ ID NO: 3 - protein, 57 aa, Homo sapiens
RSPDDELLYLPVRGRETYEMLLKIKESLELMQYLPQHTIETYRQQQQQQHQHLLQKQ

SEQ ID NO: 4 - DNA, 171 bp, Homo sapiens
  source 1..171: mol_type = unassigned DNA
agatcccccgacgatgagctgctgtacctgcctgtgaggggccgggagacctatgaaatgctgctgaagatcaaagagagcctggaactgatgcagtacctgccacagcacaccattgaaacatataggcaacaacagcagcagcagcatcagcatctgctgcagaagcag

SEQ ID NO: 5 - protein, 52 aa, Homo sapiens
RHGDEDTYYLQVRGRENFEILMKLKESLELMELVPQPLVDSYRQQQQLLQRP

SEQ ID NO: 6 - DNA, 156 bp, Homo sapiens
  source 1..156: mol_type = unassigned DNA
aggcacggcgacgaagatacctactatctgcaggtgaggggacgggagaacttcgaaatcctgatgaagctgaaagagtccctggaactgatggagctggtgccccagcctctggtcgacagctacagacagcagcagcagctgctgcagaggcca

SEQ ID NO: 7 - protein, 27 aa, Homo sapiens
QAIKKELTQIKQKVDSLLENLEKIEKE

SEQ ID NO: 8 - DNA, 81 bp, Homo sapiens
  source 1..81: mol_type = unassigned DNA
caagctataaagaaggaactcacccagattaagcaaaaggttgactcactgttggaaaatcttgagaaaatagaaaaggaa

SEQ ID NO: 9 - protein, 54 aa, Homo sapiens
STRRILGLAIESQDAGIKTITMLDEQKEQLNRIEEGLDQINKDMRETEKTLTEL

SEQ ID NO: 10 - DNA, 162 bp, Homo sapiens
  source 1..162: mol_type = unassigned DNA
tctacccgcaggatcttgggacttgctatagagtcacaggacgccggaataaaaactatcactatgcttgatgaacagaaggaacaactgaatcggattgaggaaggactggaccagattaacaaggacatgcgagagaccgaaaaaacactcactgagttg

SEQ ID NO: 11 - protein, 97 aa, Homo sapiens [sequence omitted]

SEQ ID NO: 12 - DNA, 291 bp, Homo sapiens
  source 1..291: mol_type = unassigned DNA
atgtgcggggcgccctccgccacgcagccggccaccgccgagacccagcacatcgccgaccaggtgaggtcccagcttgaagagaaagaaaacaagaagttccctgtgtttaaggccgtgtcattcaagagccaggtggtcgcggggacaaactacttcatcaaggtgcacgtcggcgacgaggacttcgtacacctgcgagtgttccaatctctccctcatgaaaacaagcccttgaccttatctaactaccagaccaacaaagccaagcatgatgagctgacctatttc

SEQ ID NO: 13 - protein, 30 aa, Homo sapiens
DEISMMGRVVKVEKQVQSIEHKLDLLLGFY

SEQ ID NO: 14 - DNA, 90 bp, Homo sapiens
  source 1..90: mol_type = unassigned DNA
gatgaaatcagcatgatgggacgcgtggtcaaggtggagaagcaggtgcagtccatcgagcacaagctggacctgctgttgggcttctat

SEQ ID NO: 15 - protein, 60 aa, Homo sapiens
TVAEAKRQAAEDALAVINQQEDSSESCWNCGRKASETCSGCNTARYCGSFCQHKDWEKHH

SEQ ID NO: 16 - DNA, 180 bp, Homo sapiens
  source 1..180: mol_type = unassigned DNA
acggtcgccgaggccaaacggcaggcggcggaggacgcactggcagttatcaatcagcaggaggattcaagcgagagttgctggaattgtggccgtaaagcgagtgaaacctgcagtggctgtaacacagcccgatactgtggctcattttgccagcacaaagactgggagaagcaccat

SEQ ID NO: 17 - protein, 559 aa, synthetic construct
  REGION 1..559: note = Description of Artificial Sequence: Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGA

SEQ ID NO: 18 - DNA, 1677 bp, synthetic construct
  misc_feature 1..1677: note = Description of Artificial Sequence: Synthetic polynucleotide
  source 1..1677: mol_type = other DNA
gaaatcgtcatgactcagactcccgcaaccctgtcagtgtccgctggggaacgtgtcactattacctgcaaggcatctcagagcgtgagcaacgacgtgacctggtatcagcagaagcctggccaggctccacgactgctgatctattccgcaagcaatcgctactccggagtgcccgcacgattctctggaagtgggtacggtaccgagttcacttttaccatttccagcgtgcagagcgaagacttcgctgtctatttttgccagcaggattactctagttttggctgtggaacaaagctggagatcaaaaggggaggaggaggttctggcggaggaggtagtggcggagggggttcacaggtgcagctggtcgaatctgggccaggcgtggtccagccaggacgttccctgaggattagctgcgccgtgagcgggttctctgtcacaaactacggagtgcactgggtccgtcagccacctggcaaatgtctggagtggctgggagtgatctgggcaggaggaatcactaactacaactctgcttttatgagtcgcctgaccatctcaaaggacaactccaaaaatacagtgtacctgcagatgaattcactgcgggcagaagataccgccatgtactattgcgcctccagggggggtcattacggctatgccctggactattggggccagggaacactggtgactgtctcatccggaggaggaggatccggaggaggaggtagcggcggagggggttctggcggagggggtagtcacgtgcagctggtcgagtccggaggagggctggtgcagcctggtggcagcctgcgactgtcttgtgccgctagtggcttctcactgacagattacggcgtgcattgggtccgacaggctccagggaagggtctggaatggctgggagtgatttggtctggagggggtacagcttataacactgcactgatcagtcggttcactatcagtagagacaactcaaagaacaccctgtacctgcagatgaactctctgcgggccgaggataccgctgtgtactattgcgctaggcggggcagttacccttataattactttgacgcatggggctgtggaaccctggtgacagtcagctctggcggagggggttcaggcggcggcggttccggcggaggaggtagccaggccgtggtcactcaggagccttccctgaccgtgagcccaggaggaacagtcactctgacctgcgggagttcaaccggtgccgtgacagcctccaactacgctaattgggtccagcagaagcccgggcagtgtcctagaggtctgatcgggggtcacaacaatcgtccacccggagtgccagccaggttctcaggctccctgctgggcggaaaagcagcactgactctgctgggcgctcagccagaggacgaagcagagtactattgcgccctgtggtattctgatcactgggtcatcgggggtggcactaagctgaccgtgctgggcacacccctgggagacaccacacatactagtggcaaacctctggatggagagtactttaccctgcagattagaggccgcgaacgattcgagatgtttcgcgaactgaatgaggccctggaactgaaggatgctcaggcaggcaaggaaccaggcggtagcggcggcgca

SEQ ID NO: 19 - protein, 581 aa, synthetic construct
  REGION 1..581: note = Description of Artificial Sequence: Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGAPHHHHHH

SEQ ID NO: 20 - DNA, 1743 bp, synthetic construct
  misc_feature 1..1743: note = Description of Artificial Sequence: Synthetic polynucleotide
  source 1..1743: mol_type = other DNA
gagatcgtgatgacccagacacccgcaacactgagcgtgtctgccggcgaaagggtcactattacctgcaaggccagtcagtcagtgtccaacgacgtgacttggtaccagcagaaaccaggccaggctccccggctgctgatctacagcgcatctaatagatatagcggagtgcctgctcgcttcagtggttcaggctatggaactgagttcaccttcaccatttccagcgtgcagtccgaagacttcgcagtgtacttttgccagcaggattattctagttttgggtgtggtacaaagctggagatcaaaaggggaggaggaggtagtggcggaggaggttcaggcggagggggtagcggcggagggggttctggcggcggcggtagtggcggcggaggtagccaggtgcagctggtcgaatccggccctggagtggtccagccaggcaggtctctgcggatcagttgcgccgtgtccggattcagcgtcaccaactacggagtgcactgggtcagacagccacctggcaagtgtctggagtggctgggagtgatctgggcaggaggaatcacaaactacaactcagcttttatgtcccgcctgactattagcaaggacaactctaaaaataccgtgtatctgcagatgaattctctgcgagccgaagataccgctatgtactattgtgcatcccgtgggggtcattacggctatgccctggattattgggggcagggtaccctggtgacagtctcatccggcggagggggatccggaggaggaggtagcggcggagggggttctggcggagggggtagtcacgtgcagctggtcgagtccggaggagggctggtgcagcctggtggcagcctgcgactgtcttgtgccgctagtggcttctcactgacagattacggcgtgcattgggtccgacaggctccagggaagggtctggaatggctgggagtgatttggtctggagggggtacagcttataacactgcactgatcagtcggttcactatcagtagagacaactcaaagaacaccctgtacctgcagatgaactctctgcgggccgaggataccgctgtgtactattgcgctaggcggggcagttacccttataattactttgacgcatggggctgtggaaccctggtgacagtcagctctggcggagggggttcaggcggcggcggttccggcggaggaggtagccaggccgtggtcactcaggagccttccctgaccgtgagcccaggaggaacagtcactctgacctgcgggagttcaaccggtgccgtgacagcctccaactacgctaattgggtccagcagaagcccgggcagtgtcctagaggtctgatcgggggtcacaacaatcgtccacccggagtgccagccaggttctcaggctccctgctgggcggaaaagcagcactgactctgctgggcgctcagccagaggacgaagcagagtactattgcgccctgtggtattctgatcactgggtcatcgggggtggcactaagctgaccgtgctgggcacacccctgggagacaccacacatactagtgggaaacctctggatggcgagtactttaccctgcagattagaggccgcgaacgattcgagatgtttcgcgaactgaatgaggccctggaactgaaggatgctcaggcaggcaaggagccaggagggtcaggaggagcaccgcaccatcatcatcaccat

SEQ ID NO: 21 - protein, 599 aa, synthetic construct
  REGION 1..599: note = Description of Artificial Sequence: Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGRSPDDELLYLPVRGRETYEMLLKIKESLELMQYLPQHTIETYRQQQQQQHQHLLQKQGGSGGAPHHHHHH

SEQ ID NO: 22 - DNA, 1797 bp, synthetic construct
  misc_feature 1..1797: note = Description of Artificial Sequence: Synthetic polynucleotide
  source 1..1797: mol_type = other DNA
gagatcgtgatgacccagacacccgcaacactgagcgtgtctgccggcgaaagggtcactattacctgcaaggccagtcagtcagtgtccaacgacgtgacttggtaccagcagaaaccaggccaggctccccggctgctgatctacagcgcatctaatagatatagcggagtgcctgctcgcttcagtggttcaggctatggaactgagttcaccttcaccatttccagcgtgcagtccgaagacttcgcagtgtacttttgccagcaggattattctagttttgggtgtggtacaaagctggagatcaaaaggggaggaggaggtagtggcggaggaggttcaggcggagggggtagcggcggagggggttctggcggcggcggtagtggcggcggaggtagccaggtgcagctggtcgaatccggccctggagtggtccagccaggcaggtctctgcggatcagttgcgccgtgtccggattcagcgtcaccaactacggagtgcactgggtcagacagccacctggcaagtgtctggagtggctgggagtgatctgggcaggaggaatcacaaactacaactcagcttttatgtcccgcctgactattagcaaggacaactctaaaaataccgtgtatctgcagatgaattctctgcgagccgaagataccgctatgtactattgtgcatcccgtgggggtcattacggctatgccctggattattgggggcagggtaccctggtgacagtctcatccggaggaggaggatccggaggaggaggtagcggcggagggggttctggcggagggggtagtcacgtgcagctggtcgagtccggaggagggctggtgcagcctggtggcagcctgcgactgtcttgtgccgctagtggcttctcactgacagattacggcgtgcattgggtccgacaggctccagggaagggtctggaatggctgggagtgatttggtctggagggggtacagcttataacactgcactgatcagtcggttcactatcagtagagacaactcaaagaacaccctgtacctgcagatgaactctctgcgggccgaggataccgctgtgtactattgcgctaggcggggcagttacccttataattactttgacgcatggggctgtggaaccctggtgacagtcagctctggcggagggggttcaggcggcggcggttccggcggaggaggtagccaggccgtggtcactcaggagccttccctgaccgtgagcccaggaggaacagtcactctgacctgcgggagttcaaccggtgccgtgacagcctccaactacgctaattgggtccagcagaagcccgggcagtgtcctagaggtctgatcgggggtcacaacaatcgtccacccggagtgccagccaggttctcaggctccctgctgggcggaaaagcagcactgactctgctgggcgctcagccagaggacgaagcagagtactattgcgccctgtggtattctgatcactgggtcatcgggggtggcactaagctgaccgtgctgggcacacccctgggagacaccacacatactagtgggagatcccccgacgatgagctgctgtacctgcctgtgaggggccgggagacctatgaaatgctgctgaagatcaaagagagcctggaactgatgcagtacctgccacagcacaccattgaaacatataggcaacaacagcagcagcagcatcagcatctgctgcagaagcagggagggtcaggaggagcaccgcaccatcatcatcaccat

SEQ ID NO: 23 - protein, 594 aa, synthetic construct
  REGION 1..594: note = Description of Artificial Sequence: Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGRHGDEDTYYLQVRGRENFEILMKLKESLELMELVPQPLVDSYRQQQQLLQRPGGSGGAPHHHHHH

SEQ ID NO: 24 - DNA, 1782 bp, synthetic construct
  misc_feature 1..1782: note = Description of Artificial Sequence: Synthetic polynucleotide
  source 1..1782: mol_type = other DNA
gagatcgtgatgacccagacacccgcaacactgagcgtgtctgccggcgaaagggtcactattacctgcaaggccagtcagtcagtgtccaacgacgtgacttggtaccagcagaaaccaggccaggctccccggctgctgatctacagcgcatctaatagatatagcggagtgcctgctcgcttcagtggttcaggctatggaactgagttcaccttcaccatttccagcgtgcagtccgaagacttcgcagtgtacttttgccagcaggattattctagttttgggtgtggtacaaagctggagatcaaaaggggaggaggaggtagtggcggaggaggttcaggcggagggggtagcggcggagggggttctggcggcggcggtagtggcggcggaggtagccaggtgcagctggtcgaatccggccctggagtggtccagccaggcaggtctctgcggatcagttgcgccgtgtccggattcagcgtcaccaactacggagtgcactgggtcagacagccacctggcaagtgtctggagtggctgggagtgatctgggcaggaggaatcacaaactacaactcagcttttatgtcccgcctgactattagcaaggacaactctaaaaataccgtgtatctgcagatgaattctctgcgagccgaagataccgctatgtactattgtgcatcccgtgggggtcattacggctatgccctggattattgggggcagggtaccctggtgacagtctcatccggaggaggaggatccggaggaggaggtagcggcggagggggttctggcggagggggtagtcacgtgcagctggtcgagtccggaggagggctggtgcagcctggtggcagcctgcgactgtcttgtgccgctagtggcttctcactgacagattacggcgtgcattgggtccgacaggctccagggaagggtctggaatggctgggagtgatttggtctggagggggtacagcttataacactgcactgatcagtcggttcactatcagtagagacaactcaaagaacaccctgtacctgcagatgaactctctgcgggccgaggataccgctgtgtactattgcgctaggcggggcagttacccttataattactttgacgcatggggctgtggaaccctggtgacagtcagctctggcggagggggttcaggcggcggcggttccggcggaggaggtagccaggccgtggtcactcaggagccttccctgaccgtgagcccaggaggaacagtcactctgacctgcgggagttcaaccggtgccgtgacagcctccaactacgctaattgggtccagcagaagcccgggcagtgtcctagaggtctgatcgggggtcacaacaatcgtccacccggagtgccagccaggttctcaggctccctgctgggcggaaaagcagcactgactctgctgggcgctcagccagaggacgaagcagagtactattgcgccctgtggtattctgatcactgggtcatcgggggtggcactaagctgaccgtgctgggcacacccctgggagacaccacacatactagtgggaggcacggcgacgaagatacctactatctgcaggtgaggggacgggagaacttcgaaatcctgatgaagctgaaagagtccctggaactgatggagctggtgccccagcctctggtcgacagctacagacagcagcagcagctgctgcagaggccaggagggtcaggaggagcaccgcaccatcatcatcaccat

SEQ ID NO: 25 - protein, 566 aa, synthetic construct
  REGION 1..566: note = Description of Artificial Sequence: Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGAPHHHHHH

SEQ ID NO: 26 - DNA, 1698 bp, synthetic construct
  misc_feature 1..1698: note = Description of Artificial Sequence: Synthetic polynucleotide
  source 1..1698: mol_type = other DNA
gaaatcgtcatgactcagactcccgcaaccctgtcagtgtccgctggggaacgtgtcactattacctgcaaggcatctcagagcgtgagcaacgacgtgacctggtatcagcagaagcctggccaggctccacgactgctgatctattccgcaagcaatcgctactccggagtgcccgcacgattctctggaagtgggtacggtaccgagttcacttttaccatttccagcgtgcagagcgaagacttcgctgtctatttttgccagcaggattactctagttttggctgtggaacaaagctggagatcaaaaggggaggaggaggttctggcggaggaggtagtggcggagggggttcacaggtgcagctggtcgaatctgggccaggcgtggtccagccaggacgttccctgaggattagctgcgccgtgagcgggttctctgtcacaaactacggagtgcactgggtccgtcagccacctggcaaatgtctggagtggctgggagtgatctgggcaggaggaatcactaactacaactctgcttttatgagtcgcctgaccatctcaaaggacaactccaaaaatacagtgtacctgcagatgaattcactgcgggcagaagataccgccatgtactattgcgcctccagggggggtcattacggctatgccctggactattggggccagggaacactggtgactgtctcatccggaggaggaggatccggaggaggaggtagcggcggagggggttctggcggagggggtagtcacgtgcagctggtcgagtccggaggagggctggtgcagcctggtggcagcctgcgactgtcttgtgccgctagtggcttctcactgacagattacggcgtgcattgggtccgacaggctccagggaagggtctggaatggctgggagtgatttggtctggagggggtacagcttataacactgcactgatcagtcggttcactatcagtagagacaactcaaagaacaccctgtacctgcagatgaactctctgcgggccgaggataccgctgtgtactattgcgctaggcggggcagttacccttataattactttgacgcatggggctgtggaaccctggtgacagtcagctctggcggagggggttcaggcggcggcggttccggcggaggaggtagccaggccgtggtcactcaggagccttccctgaccgtgagcccaggaggaacagtcactctgacctgcgggagttcaaccggtgccgtgacagcctccaactacgctaattgggtccagcagaagcccgggcagtgtcctagaggtctgatcgggggtcacaacaatcgtccacccggagtgccagccaggttctcaggctccctgctgggcggaaaagcagcactgactctgctgggcgctcagccagaggacgaagcagagtactattgcgccctgtggtattctgatcactgggtcatcgggggtggcactaagctgaccgtgctgggcacacccctgggagacaccacacatactagtgggaaacctctggatggcgagtactttaccctgcagattagaggccgcgaacgattcgagatgtttcgcgaactgaatgaggccctggaactgaaggatgctcaggcaggcaaggagccaggagggtcaggaggagcaccgcaccatcatcatcaccat

SEQ ID NO: 27 - protein, 584 aa, synthetic construct
  REGION 1..584: note = Description of Artificial Sequence: Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGRSPDDELLYLPVRGRETYEMLLKIKESLELMQYLPQHTIETYRQQQQQQHQHLLQKQGGSGGAPHHHHHH

SEQ ID NO: 28 - DNA, 1753 bp, synthetic construct
  misc_feature 1..1753: note = Description of Artificial Sequence: Synthetic polynucleotide
  source 1..1753: mol_type = other DNA
gaaatcgtcatgactcagactcccgcaaccctgtcagtgtccgctggggaacgtgtcactattacctgcaaggcatctcagagcgtgagcaacgacgtgacctggtatcagcagaagcctggccaggctccacgactgctgatctattccgcaagcaatcgctactccggagtgcccgcacgattctctggaagtgggtacggtaccgagttcacttttaccatttccagcgtgcagagcgaagacttcgctgtctatttttgccagcaggattactctagttttggctgtggaacaaagctggagatcaaaaggggaggaggaggttctggcggaggaggtagtggcggagggggttcacaggtgcagctggtcgaatctgggccaggcgtggtccagccaggacgttccctgaggattagctgcgccgtgagcgggttctctgtcacaaactacggagtgcactgggtccgtcagccacctggcaaatgtctggagtggctgggagtgatctgggcaggaggaatcactaactacaactctgcttttatgagtcgcctgaccatctcaaaggacaactccaaaaatacagtgtacctgcagatgaattcactgcgggcagaagataccgccatgtactattgcgcctccagggggggtcattacggctatgccctggactattggggccagggaacactggtgactgtctcatccggaggaggaggatccggaggaggaggtagcggcggagggggttctggcggagggggtagtcacgtgcagctggtcgagtccggaggagggctggtgcagcctggtggcagcctgcgactgtcttgtgccgctagtggcttctcactgacagattacggcgtgcattgggtccgacaggctccagggaagggtctggaatggctgggagtgatttggtctggagggggtacagcttataacactgcactgatcagtcggttcactatcagtagagacaactcaaagaacaccctgtacctgcagatgaactctctgcgggccgaggataccgctgtgtactattgcgctaggcggggcagttacccttataattactttgacgcatggggctgtggaaccctggtgacagtcagctctggcggagggggttcaggcggcggcggttccggcggaggaggtagccaggccgtggtcactcaggagccttccctgaccgtgagcccaggaggaacagtcactctgacctgcgggagttcaaccggtgccgtgacagcctccaactacgctaattgggtccagcagaagcccgggcagtgtcctagaggtctgatcgggggtcacaacaatcgtccacccggagtgccagccaggttctcaggctccctgctgggcggaaaagcagcactgactctgctgggcgctcagccagaggacgaagcagagtactattgcgccctgtggtattctgatcactgggtcatcgggggtggcactaagctgaccgtgctgggcacacccctgggagacaccacacatactagtgggagatcccccgacgatgagctgctgtacctgcctgtgaggggccgggagacctatgaaatgctgctgaagatcaaagagagcctggaactgatgcagtacctgccacagcacaccattgaaacatataggcaacaacagcagcagcagcatcagcatctgctgcagaagcagggagggtcaggaggagcaccgcaccatcatcatcaccatt

SEQ ID NO: 29 - protein, 579 aa, synthetic construct
  REGION 1..579: note = Description of Artificial Sequence: Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGRHGDEDTYYLQVRGRENFEILMKLKESLELMELVPQPLVDSYRQQQQLLQRPGGSGGAPHHHHHH

SEQ ID NO: 30 - DNA, 1737 bp, synthetic construct
  misc_feature 1..1737: note = Description of Artificial Sequence: Synthetic polynucleotide
  source 1..1737: mol_type = other DNA
gaaatcgtcatgactcagactcccgcaaccctgtcagtgtccgctggggaacgtgtcactattacctgcaaggcatctcagagcgtgagcaacgacgtgacctggtatcagcagaagcctggccaggctccacgactgctgatctattccgcaagcaatcgctactccggagtgcccgcacgattctctggaagtgggtacggtaccgagttcacttttaccatttccagcgtgcagagcgaagacttcgctgtctatttttgccagcaggattactctagttttggctgtggaacaaagctggagatcaaaaggggaggaggaggttctggcggaggaggtagtggcggagggggttcacaggtgcagctggtcgaatctgggccaggcgtggtccagccaggacgttccctgaggattagctgcgccgtgagcgggttctctgtcacaaactacggagtgcactgggtccgtcagccacctggcaaatgtctggagtggctgggagtgatctgggcaggaggaatcactaactacaactctgcttttatgagtcgcctgaccatctcaaaggacaactccaaaaatacagtgtacctgcagatgaattcactgcgggcagaagataccgccatgtactattgcgcctccagggggggtcattacggctatgccctggactattggggccagggaacactggtgactgtctcatccggaggaggaggatccggaggaggaggtagcggcggagggggttctggcggagggggtagtcacgtgcagctggtcgagtccggaggagggctggtgcagcctggtggcagcctgcgactgtcttgtgccgctagtggcttctcactgacagattacggcgtgcattgggtccgacaggctccagggaagggtctggaatggctgggagtgatttggtctggagggggtacagcttataacactgcactgatcagtcggttcactatcagtagagacaactcaaagaacaccctgtacctgcagatgaactctctgcgggccgaggataccgctgtgtactattgcgctaggcggggcagttacccttataattactttgacgcatggggctgtggaaccctggtgacagtcagctctggcggagggggttcaggcggcggcggttccggcggaggaggtagccaggccgtggtcactcaggagccttccctgaccgtgagcccaggaggaacagtcactctgacctgcgggagttcaaccggtgccgtgacagcctccaactacgctaattgggtccagcagaagcccgggcagtgtcctagaggtctgatcgggggtcacaacaatcgtccacccggagtgccagccaggttctcaggctccctgctgggcggaaaagcagcactgactctgctgggcgctcagccagaggacgaagcagagtactattgcgccctgtggtattctgatcactgggtcatcgggggtggcactaagctgaccgtgctgggcacacccctgggagacaccacacatactagtgggaggcacggcgacgaagatacctactatctgcaggtgaggggacgggagaacttcgaaatcctgatgaagctgaaagagtccctggaactgatggagctggtgccccagcctctggtcgacagctacagacagcagcagcagctgctgcagaggccaggagggtcaggaggagcaccgcaccatcatcatcaccat

SEQ ID NO: 31 - protein, 596 aa, synthetic construct
  REGION 1..596: note = Description of Artificial Sequence: Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGAPHHHHHH

SEQ ID NO: 32 - DNA, 1788 bp, synthetic construct
  misc_feature 1..1788: note = Description of Artificial Sequence: Synthetic polynucleotide
  source 1..1788: mol_type = other DNA
gagatcgtgatgacccagacacccgcaacactgagcgtgtctgccggcgaaagggtcactattacctgcaaggccagtcagtcagtgtccaacgacgtgacttggtaccagcagaaaccaggccaggctccccggctgctgatctacagcgcatctaatagatatagcggagtgcctgctcgcttcagtggttcaggctatggaactgagttcaccttcaccatttccagcgtgcagtccgaagacttcgcagtgtacttttgccagcaggattattctagttttgggtgtggtacaaagctggagatcaaaaggggaggaggaggtagtggcggaggaggttcaggcggagggggtagcggcggagggggttctggcggcggcggtagtggcggcggaggtagccaggtgcagctggtcgaatccggccctggagtggtccagccaggcaggtctctgcggatcagttgcgccgtgtccggattcagcgtcaccaactacggagtgcactgggtcagacagccacctggcaagtgtctggagtggctgggagtgatctgggcaggaggaatcacaaactacaactcagcttttatgtcccgcctgactattagcaaggacaactctaaaaataccgtgtatctgcagatgaattctctgcgagccgaagataccgctatgtactattgtgcatcccgtgggggtcattacggctatgccctggattattgggggcagggtaccctggtgacagtctcatccggcggagggggatccggcggcggaggatctggcggaggtggaagtgggggaggcggatctcatgtgcagctggtggaaagcggaggcggcctggtgcagcctgggggatctctgagactgtcttgtgccgccagcggcttctccctgaccgattatggcgtgcactgggtgcgacaggcccctggcaaaggactggaatggctgggagtgatttggagtggcggaggcaccgcctacaacaccgccctgatctcccggttcaccatcagccgggacaactccaagaacaccctgtacctgcagatgaactccctgcgggccgaggacaccgctgtgtactactgcgccagacggggctcctacccctacaactacttcgacgcttggggctgcggcaccctcgtgacagtgtctagcggagggggaggttctgggggcggaggttcaggtggtggtggttccgggggtggtggctctggtggcggtggttctggcggtggcggatctcaggctgtcgtgacccaggaacccagcctgactgtgtctcctggcggaaccgtgaccctgacctgcggatcttctaccggcgctgtgaccgccagcaactacgccaattgggtgcagcagaaacctggacagtgccctagaggcctgatcggcggccacaacaacagacctccaggcgtgccagcccggttctctggatctctgctgggcggaaaggccgctctgacactgctgggtgctcagcctgaggacgaggccgagtactactgtgccctgtggtactccgaccactgggtcatcggaggcgggaccaagctgaccgtgctgggaacacccctgggagacaccacacatactagtgggaaacctctggatggcgagtactttaccctgcagattagaggccgcgaacgattcgagatgtttcgcgaactgaatgaggccctggaactgaaggatgctcaggcaggcaaggagccaggagggtcaggaggagcaccgcaccatcatcatcaccat

SEQ ID NO: 33 - protein, 614 aa, synthetic construct
  REGION 1..614: note = Description of Artificial Sequence: Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGRSPDDELLYLPVRGRETYEMLLKIKESLELMQYLPQHTIETYRQQQQQQHQHLLQKQGGSGGAPHHHHHH

SEQ ID NO: 34 - DNA, 1842 bp, synthetic construct
  misc_feature 1..1842: note = Description of Artificial Sequence: Synthetic polynucleotide
  source 1..1842: mol_type = other DNA
gagatcgtgatgacccagacacccgcaacactgagcgtgtctgccggcgaaagggtcactattacctgcaaggccagtcagtcagtgtccaacgacgtgacttggtaccagcagaaaccaggccaggctccccggctgctgatctacagcgcatctaatagatatagcggagtgcctgctcgcttcagtggttcaggctatggaactgagttcaccttcaccatttccagcgtgcagtccgaagacttcgcagtgtacttttgccagcaggattattctagttttgggtgtggtacaaagctggagatcaaaaggggaggaggaggtagtggcggaggaggttcaggcggagggggtagcggcggagggggttctggcggcggcggtagtggcggcggaggtagccaggtgcagctggtcgaatccggccctggagtggtccagccaggcaggtctctgcggatcagttgcgccgtgtccggattcagcgtcaccaactacggagtgcactgggtcagacagccacctggcaagtgtctggagtggctgggagtgatctgggcaggaggaatcacaaactacaactcagcttttatgtcccgcctgactattagcaaggacaactctaaaaataccgtgtatctgcagatgaattctctgcgagccgaagataccgctatgtactattgtgcatcccgtgggggtcattacggctatgccctggattattgggggcagggtaccctggtgacagtctcatccggaggaggaggatccggaggaggaggtagcggcggagggggttctggcggagggggtagtcatgtgcagctggtggaaagcggaggcggcctggtgcagcctgggggatctctgagactgtcttgtgccgccagcggcttctccctgaccgattatggcgtgcactgggtgcgacaggcccctggcaaaggactggaatggctgggagtgatttggagtggcggaggcaccgcctacaacaccgccctgatctcccggttcaccatcagccgggacaactccaagaacaccctgtacctgcagatgaactccctgcgggccgaggacaccgctgtgtactactgcgccagacggggctcctacccctacaactacttcgacgcttggggctgcggcaccctcgtgacagtgtctagcggagggggaggttctgggggcggaggttcaggtggtggtggttccgggggtggtggctctggtggcggtggttctggcggtggcggatctcaggctgtcgtgacccaggaacccagcctgactgtgtctcctggcggaaccgtgaccctgacctgcggatcttctaccggcgctgtgaccgccagcaactacgccaattgggtgcagcagaaacctggacagtgccctagaggcctgatcggcggccacaacaacagacctccaggcgtgccagcccggttctctggatctctgctgggcggaaaggccgctctgacactgctgggtgctcagcctgaggacgaggccgagtactactgtgccctgtggtactccgaccactgggtcatcggaggcgggaccaagctgaccgtgctgggaacacccctgggagacaccacacatactagtgggagatcccccgacgatgagctgctgtacctgcctgtgaggggccgggagacctatgaaatgctgctgaagatcaaagagagcctggaactgatgcagtacctgccacagcacaccattgaaacatataggcaacaacagcagcagcagcatcagcatctgctgcagaagcagggagggtcaggaggagcaccgcaccatcatcatcaccat

SEQ ID NO: 35 - protein, 609 aa, synthetic construct
  REGION 1..609: note = Description of Artificial Sequence: Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGRHGDEDTYYLQVRGRENFEILMKLKESLELMELVPQPLVDSYRQQQQLLQRPGGSGGAPHHHHHH

SEQ ID NO: 36 - DNA, 1827 bp, synthetic construct
  misc_feature 1..1827: note = Description of Artificial Sequence: Synthetic polynucleotide
  source 1..1827: mol_type = other DNA
gagatcgtgatgacccagacacccgcaacactgagcgtgtctgccggcgaaagggtcactattacctgcaaggccagtcagtcagtgtccaacgacgtgacttggtaccagcagaaaccaggccaggctccccggctgctgatctacagcgcatctaatagatatagcggagtgcctgctcgcttcagtggttcaggctatggaactgagttcaccttcaccatttccagcgtgcagtccgaagacttcgcagtgtacttttgccagcaggattattctagttttgggtgtggtacaaagctggagatcaaaaggggaggaggaggtagtggcggaggaggttcaggcggagggggtagcggcggagggggttctggcggcggcggtagtggcggcggaggtagccaggtgcagctggtcgaatccggccctggagtggtccagccaggcaggtctctgcggatcagttgcgccgtgtccggattcagcgtcaccaactacggagtgcactgggtcagacagccacctggcaagtgtctggagtggctgggagtgatctgggcaggaggaatcacaaactacaactcagcttttatgtcccgcctgactattagcaaggacaactctaaaaataccgtgtatctgcagatgaattctctgcgagccgaagataccgctatgtactattgtgcatcccgtgggggtcattacggctatgccctggattattgggggcagggtaccctggtgacagtctcatccggaggaggaggatccggaggaggaggtagcggcggagggggttctggcggagggggtagtcatgtgcagctggtggaaagcggaggcggcctggtgcagcctgggggatctctgagactgtcttgtgccgccagcggcttctccctgaccgattatggcgtgcactgggtgcgacaggcccctggcaaaggactggaatggctgggagtgatttggagtggcggaggcaccgcctacaacaccgccctgatctcccggttcaccatcagccgggacaactccaagaacaccctgtacctgcagatgaactccctgcgggccgaggacaccgctgtgtactactgcgccagacggggctcctacccctacaactacttcgacgcttggggctgcggcaccctcgtgacagtgtctagcggagggggaggttctgggggcggaggttcaggtggtggtggttccgggggtggtggctctggtggcggtggttctggcggtggcggatctcaggctgtcgtgacccaggaacccagcctgactgtgtctcctggcggaaccgtgaccctgacctgcggatcttctaccggcgctgtgaccgccagcaactacgccaattgggtgcagcagaaacctggacagtgccctagaggcctgatcggcggccacaacaacagacctccaggcgtgccagcccggttctctggatctctgctgggcggaaaggccgctctgacactgctgggtgctcagcctgaggacgaggccgagtactactgtgccctgtggtactccgaccactgggtcatcggaggcgggaccaagctgaccgtgctgggaacacccctgggagacaccacacatactagtgggaggcacggcgacgaagatacctactatctgcaggtgaggggacgggagaacttcgaaatcctgatgaagctgaaagagtccctggaactgatggagctggtgccccagcctctggtcgacagctacagacagcagcagcagctgctgcagaggccaggagggtcaggaggagcaccgcaccatcatcatcaccat

SEQ ID NO: 37 - protein, 559 aa, synthetic construct
  REGION 1..559: note = Description of Artificial Sequence: Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGCGTTVTVSSGGGGSGGGGSGGGGSQAVVIQESALTTPPGETVTLTCGSSTGAVTASNYANWVQEKPDHCFTGLIGGHNNRPPGVPARFSGSLIGDKAALTIAGTQTEDEAIYFCALWYSDHWVIGGGTRLTVLGTPLGDTTHTSGKPLDGEYFTLQIRGRERFEMFRELNEALELKDAQAGKEPGGSGGA

SEQ ID NO: 38 - DNA, 1677 bp, synthetic construct
  misc_feature 1..1677: note = Description of Artificial Sequence: Synthetic polynucleotide
  source 1..1677: mol_type = other DNA
gaaatcgtcatgactcagactcccgcaaccctgtcagtgtccgctggggaacgtgtcactattacctgcaaggcatctcagagcgtgagcaacgacgtgacctggtatcagcagaagcctggccaggctccacgactgctgatctattccgcaagcaatcgctactccggagtgcccgcacgattctctggaagtgggtacggtaccgagttcacttttaccatttccagcgtgcagagcgaagacttcgctgtctatttttgccagcaggattactctagttttggctgtggaacaaagctggagatcaaaaggggaggaggaggttctggcggaggaggtagtggcggagggggttcacaggtgcagctggtcgaatctgggccaggcgtggtccagccaggacgttccctgaggattagctgcgccgtgagcgggttctctgtcacaaactacggagtgcactgggtccgtcagccacctggcaaatgtctggagtggctgggagtgatctgggcaggaggaatcactaactacaactctgcttttatgagtcgcctgaccatctcaaaggacaactccaaaaatacagtgtacctgcagatgaattcactgcgggcagaagataccgccatgtactattgcgcctccagggggggtcattacggctatgccctggactattggggccagggaacactggtgactgtctcatccggaggaggaggatccggaggaggaggtagcggcggagggggttctggcggagggggtagtcacgtgaagctgcaggaaagcggccctggactggtgcagccttcccagtctctgtccctgacctgcaccgtgtccggcttctccctgaccgattacggcgtgcactgggtgcgacagtctccaggcaagggcctggaatggctgggagtgatttggagcggtggcggaaccgcctacaacaccgccctgatctcccggctgaacatctaccgggacaactccaagaaccaggtgttcctggaaatgaactccctgcaggcagaggacaccgccatgtactactgcgccagacggggctcctacccctacaactacttcgacgcttggggctgcggcaccaccgtgacagtgtctagcggaggtggtggatctgggggcggaggtagcggagggggaggttctcaggctgtcgtgatccaggaatctgccctgaccaccccccctggcgagacagtgacactgacctgcggatcttccaccggcgctgtgaccgcctccaactacgccaactgggtgcaggaaaagcccgaccactgcttcaccggcctgatcggcggccacaacaacagacctccaggcgtgccagcccggttctccggctctctgatcggagataaggccgccctgacaatcgccggcacccagacagaggacgaggctatctacttctgcgccctgtggtacagcgaccactgggtcatcggcggaggcaccagactgaccgtgctgggaacacccctgggagacaccacacatactagtggcaaacctctggatggagagtactttaccctgcagattagaggccgcgaacgattcgagatgtttcgcgaactgaatgaggccctggaactgaaggatgctcaggcaggcaaggaaccaggcggtagcggcggcgca

SEQ ID NO: 39 - protein, 581 aa, synthetic construct
  REGION 1..581: note = Description of Artificial Sequence: Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGCGTTVTVSSGGGGSGGGGSGGGGSQAVVIQESALTTPPGETVTLTCGSSTGAVTASNYANWVQEKPDHCFTGLIGGHNNRPPGVPARFSGSLIGDKAALTIAGTQTEDEAIYFCALWYSDHWVIGGGTRLTVLGTPLGDTTHTSGKPLDGEYFTLQIRGRERFEMFRELNEALELKDAQAGKEPGGSGGAPHHHHHH

SEQ ID NO: 40 - DNA, 1743 bp, synthetic construct
  misc_feature 1..1743: note = Description of Artificial Sequence: Synthetic polynucleotide
  source 1..1743: mol_type = other DNA
gagatcgtgatgacccagacacccgcaacactgagcgtgtctgccggcgaaagggtcactattacctgcaaggccagtcagtcagtgtccaacgacgtgacttggtaccagcagaaaccaggccaggctccccggctgctgatctacagcgcatctaatagatatagcggagtgcctgctcgcttcagtggttcaggctatggaactgagttcaccttcaccatttccagcgtgcagtccgaagacttcgcagtgtacttttgccagcaggattattctagttttgggtgtggtacaaagctggagatcaaaaggggaggaggaggtagtggcggaggaggttcaggcggagggggtagcggcggagggggttctggcggcggcggtagtggcggcggaggtagccaggtgcagctggtcgaatccggccctggagtggtccagccaggcaggtctctgcggatcagttgcgccgtgtccggattcagcgtcaccaactacggagtgcactgggtcagacagccacctggcaagtgtctggagtggctgggagtgatctgggcaggaggaatcacaaactacaactcagcttttatgtcccgcctgactattagcaaggacaactctaaaaataccgtgtatctgcagatgaattctctgcgagccgaagataccgctatgtactattgtgcatcccgtgggggtcattacggctatgccctggattattgggggcagggtaccctggtgacagtctcatccggcggagggggatccggaggaggaggtagcggcggagggggttctggcggagggggtagtcacgtgaagctgcaggaaagcggccctggactggtgcagccttcccagtctctgtccctgacctgcaccgtgtccggcttctccctgaccgattacggcgtgcactgggtgcgacagtctccaggcaagggcctggaatggctgggagtgatttggagcggtggcggaaccgcctacaacaccgccctgatctcccggctgaacatctaccgggacaactccaagaaccaggtgttcctggaaatgaactccctgcaggcagaggacaccgccatgtactactgcgccagacggggctcctacccctacaactacttcgacgcttggggctgcggcaccaccgtgacagtgtctagcggaggtggtggatctgggggcggaggtagcggagggggaggttctcaggctgtcgtgatccaggaatctgccctgaccaccccccctggcgagacagtgacactgacctgcggatcttccaccggcgctgtgaccgcctccaactacgccaactgggtgcaggaaaagcccgaccactgcttcaccggcctgatcggcggccacaacaacagacctccaggcgtgccagcccggttctccggctctctgatcggagataaggccgccctgacaatcgccggcacccagacagaggacgaggctatctacttctgcgccctgtggtacagcgaccactgggtcatcggcggaggcaccagactgaccgtgctgggaacacccctgggagacaccacacatactagtgggaaacctctggatggcgagtactttaccctgcagattagaggccgcgaacgattcgagatgtttcgcgaactgaatgaggccctggaactgaaggatgctcaggcaggcaaggagccaggagggtcaggaggagcaccgcaccatcatcatcaccat

SEQ ID NO: 41 - protein, 599 aa, synthetic construct
  REGION 1..599: note = Description of Artificial Sequence: Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGCGTTVTVSSGGGGSGGGGSGGGGSQAVVIQESALTTPPGETVTLTCGSSTGAVTASNYANWVQEKPDHCFTGLIGGHNNRPPGVPARFSGSLIGDKAALTIAGTQTEDEAIYFCALWYSDHWVIGGGTRLTVLGTPLGDTTHTSGRSPDDELLYLPVRGRETYEMLLKIKESLELMQYLPQHTIETYRQQQQQQHQHLLQKQGGSGGAPHHHHHH

SEQ ID NO: 42 - DNA, 1798 bp, synthetic construct
  misc_feature 1..1798: note = Description of Artificial Sequence: Synthetic polynucleotide
  source 1..1798: mol_type = other DNA
gagatcgtgatgacccagacacccgcaacactgagcgtgtctgccggcgaaagggtcactattacctgcaaggccagtcagtcagtgtccaacgacgtgacttggtaccagcagaaaccaggccaggctccccggctgctgatctacagcgcatctaatagatatagcggagtgcctgctcgcttcagtggttcaggctatggaactgagttcaccttcaccatttccagcgtgcagtccgaagacttcgcagtgtacttttgccagcaggattattctagttttgggtgtggtacaaagctggagatcaaaaggggaggaggaggtagtggcggaggaggttcaggcggagggggtagcggcggagggggttctggcggcggcggtagtggcggcggaggtagccaggtgcagctggtcgaatccggccctggagtggtccagccaggcaggtctctgcggatcagttgcgccgtgtccggattcagcgtcaccaactacggagtgcactgggtcagacagccacctggcaagtgtctggagtggctgggagtgatctgggcaggaggaatcacaaactacaactcagcttttatgtcccgcctgactattagcaaggacaactctaaaaataccgtgtatctgcagatgaattctctgcgagccgaagataccgctatgtactattgtgcatcccgtgggggtcattacggctatgccctggattattgggggcagggtaccctggtgacagtctcatccggaggaggaggatccggaggaggaggtagcggcggagggggttctggcggagggggtagtcacgtgaagctgcaggaaagcggccctggactggtgcagccttcccagtctctgtccctgacctgcaccgtgtccggcttctccctgaccgattacggcgtgcactgggtgcgacagtctccaggcaagggcctggaatggctgggagtgatttggagcggtggcggaaccgcctacaacaccgccctgatctcccggctgaacatctaccgggacaactccaagaaccaggtgttcctggaaatgaactccctgcaggcagaggacaccgccatgtactactgcgccagacggggctcctacccctacaactacttcgacgcttggggctgcggcaccaccgtgacagtgtctagcggaggtggtggatctgggggcggaggtagcggagggggaggttctcaggctgtcgtgatccaggaatctgccctgaccaccccccctggcgagacagtgacactgacctgcggatcttccaccggcgctgtgaccgcctccaactacgccaactgggtgcaggaaaagcccgaccactgcttcaccggcctgatcggcggccacaacaacagacctccaggcgtgccagcccggttctccggctctctgatcggagataaggccgccctgacaatcgccggcacccagacagaggacgaggctatctacttctgcgccctgtggtacagcgaccactgggtcatcggcggaggcaccagactgaccgtgctgggaacacccctgggagacaccacacatactagtgggagatcccccgacgatgagctgctgtacctgcctgtgaggggccgggagacctatgaaatgctgctgaagatcaaagagagcctggaactgatgcagtacctgccacagcacaccattgaaacatataggcaacaacagcagcagcagcatcagcatctgctgcagaagcagggagggtcaggaggagcaccgcaccatcatcatcaccatt

SEQ ID NO: 43 - protein, 594 aa, synthetic construct
  REGION 1..594: note = Description of Artificial Sequence: Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGCGTTVTVSSGGGGSGGGGSGGGGSQAVVIQESALTTPPGETVTLTCGSSTGAVTASNYANWVQEKPDHCFTGLIGGHNNRPPGVPARFSGSLIGDKAALTIAGTQTEDEAIYFCALWYSDHWVIGGGTRLTVLGTPLGDTTHTSGRHGDEDTYYLQVRGRENFEILMKLKESLELMELVPQPLVDSYRQQQQLLQRPGGSGGAPHHHHHH

SEQ ID NO: 44 - DNA, 1782 bp, synthetic construct
  misc_feature 1..1782: note = Description of Artificial Sequence: Synthetic polynucleotide
  source 1..1782: mol_type = other DNA
gagatcgtgatgacccagacacccgcaacactgagcgtgtctgccggcgaaagggtcactattacctgcaaggccagtcagtcagtgtccaacgacgtgacttggtaccagcagaaaccaggccaggctccccggctgctgatctacagcgcatctaatagatatagcggagtgcctgctcgcttcagtggttcaggctatggaactgagttcaccttcaccatttccagcgtgcagtccgaagacttcgcagtgtacttttgccagcaggattattctagttttgggtgtggtacaaagctggagatcaaaaggggaggaggaggtagtggcggaggaggttcaggcggagggggtagcggcggagggggttctggcggcggcggtagtggcggcggaggtagccaggtgcagctggtcgaatccggccctggagtggtccagccaggcaggtctctgcggatcagttgcgccgtgtccggattcagcgtcaccaactacggagtgcactgggtcagacagccacctggcaagtgtctggagtggctgggagtgatctgggcaggaggaatcacaaactacaactcagcttttatgtcccgcctgactattagcaaggacaactctaaaaataccgtgtatctgcagatgaattctctgcgagccgaagataccgctatgtactattgtgcatcccgtgggggtcattacggctatgccctggattattgggggcagggtaccctggtgacagtctcatccggaggaggaggatccggaggaggaggtagcggcggagggggttctggcggagggggtagtcacgtgaagctgcaggaaagcggccctggactggtgcagccttcccagtctctgtccctgacctgcaccgtgtccggcttctccctgaccgattacggcgtgcactgggtgcgacagtctccaggcaagggcctggaatggctgggagtgatttggagcggtggcggaaccgcctacaacaccgccctgatctcccggctgaacatctaccgggacaactccaagaaccaggtgttcctggaaatgaactccctgcaggcagaggacaccgccatgtactactgcgccagacggggctcctacccctacaactacttcgacgcttggggctgcggcaccaccgtgacagtgtctagcggaggtggtggatctgggggcggaggtagcggagggggaggttctcaggctgtcgtgatccaggaatctgccctgaccaccccccctggcgagacagtgacactgacctgcggatcttccaccggcgctgtgaccgcctccaactacgccaactgggtgcaggaaaagcccgaccactgcttcaccggcctgatcggcggccacaacaacagacctccaggcgtgccagcccggttctccggctctctgatcggagataaggccgccctgacaatcgccggcacccagacagaggacgaggctatctacttctgcgccctgtggtacagcgaccactgggtcatcggcggaggcaccagactgaccgtgctgggaacacccctgggagacaccacacatactagtgggaggcacggcgacgaagatacctactatctgcaggtgaggggacgggagaacttcgaaatcctgatgaagctgaaagagtccctggaactgatggagctggtgccccagcctctggtcgacagctacagacagcagcagcagctgctgcagaggccaggagggtcaggaggagcaccgcaccatcatcatcaccat

SEQ ID NO: 45 - protein, 566 aa, synthetic construct
  REGION 1..566: note = Description of Artificial Sequence: Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGCGTTVTVSSGGGGSGGGGSGGGGSQAVVIQESALTTPPGETVTLTCGSSTGAVTASNYANWVQEKPDHCFTGLIGGHNNRPPGVPARFSGSLIGDKAALTIAGTQTEDEAIYFCALWYSDHWVIGGGTRLTVLGTPLGDTTHTSGKPLDGEYFTLQIRGRERFEMFRELNEALELKDAQAGKEPGGSGGAPHHHHHH

SEQ ID NO: 46 - DNA, 1698 bp, synthetic construct
  misc_feature 1..1698: note = Description of Artificial Sequence: Synthetic polynucleotide
  source 1..1698: mol_type = other DNA
gaaatcgtcatgactcagactcccgcaaccctgtcagtgtccgctggggaacgtgtcactattacctgcaaggcatctcagagcgtgagcaacgacgtgacctggtatcagcagaagcctggccaggctccacgactgctgatctattccgcaagcaatcgctactccggagtgcccgcacgattctctggaagtgggtacggtaccgagttcacttttaccatttccagcgtgcagagcgaagacttcgctgtctatttttgccagcaggattactctagttttggctgtggaacaaagctggagatcaaaaggggaggaggaggttctggcggaggaggtagtggcggagggggttcacaggtgcagctggtcgaatctgggccaggcgtggtccagccaggacgttccctgaggattagctgcgccgtgagcgggttctctgtcacaaactacggagtgcactgggtccgtcagccacctggcaaatgtctggagtggctgggagtgatctgggcaggaggaatcactaactacaactctgcttttatgagtcgcctgaccatctcaaaggacaactccaaaaatacagtgtacctgcagatgaattcactgcgggcagaagataccgccatgtactattgcgcctccagggggggtcattacggctatgccctggactattggggccagggaacactggtgactgtctcatccggaggaggaggatccggaggaggaggtagcggcggagggggttctggcggagggggtagtcacgtgaagctgcaggaaagcggccctggactggtgcagccttcccagtctctgtccctgacctgcaccgtgtccggcttctccctgaccgattacggcgtgcactgggtgcgacagtctccaggcaagggcctggaatggctgggagtgatttggagcggtggcggaaccgcctacaacaccgccctgatctcccggctgaacatctaccgggacaactccaagaaccaggtgttcctggaaatgaactccctgcaggcagaggacaccgccatgtactactgcgccagacggggctcctacccctacaactacttcgacgcttggggctgcggcaccaccgtgacagtgtctagcggaggtggtggatctgggggcggaggtagcggagggggaggttctcaggctgtcgtgatccaggaatctgccctgaccaccccccctggcgagacagtgacactgacctgcggatcttccaccggcgctgtgaccgcctccaactacgccaactgggtgcaggaaaagcccgaccactgcttcaccggcctgatcggcggccacaacaacagacctccaggcgtgccagcccggttctccggctctctgatcggagataaggccgccctgacaatcgccggcacccagacagaggacgaggctatctacttctgcgccctgtggtacagcgaccactgggtcatcggcggaggcaccagactgaccgtgctgggaacacccctgggagacaccacacatactagtgggaaacctctggatggcgagtactttaccctgcagattagaggccgcgaacgattcgagatgtttcgcgaactgaatgaggccctggaactgaaggatgctcaggcaggcaaggagccaggagggtcaggaggagcaccgcaccatcatcatcaccat

SEQ ID NO: 47 - protein, 584 aa, synthetic construct
  REGION 1..584: note = Description of Artificial Sequence: Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGCGTTVTVSSGGGGSGGGGSGGGGSQAVVIQESALTTPPGETVTLTCGSSTGAVTASNYANWVQEKPDHCFTGLIGGHNNRPPGVPARFSGSLIGDKAALTIAGTQTEDEAIYFCALWYSDHWVIGGGTRLTVLGTPLGDTTHTSGRSPDDELLYLPVRGRETYEMLLKIKESLELMQYLPQHTIETYRQQQQQQHQHLLQKQGGSGGAPHHHHHH

SEQ ID NO: 48 - DNA, 1753 bp, synthetic construct
  misc_feature 1..1753: note = Description of Artificial Sequence: Synthetic polynucleotide
  source 1..1753: mol_type = other DNA
gaaatcgtcatgactcagactcccgcaaccctgtcagtgtccgctggggaacgtgtcactattacctgcaaggcatctcagagcgtgagcaacgacgtgacctggtatcagcagaagcctggccaggctccacgactgctgatctattccgcaagcaatcgctactccggagtgcccgcacgattctctggaagtgggtacggtaccgagttcacttttaccatttccagcgtgcagagcgaagacttcgctgtctatttttgccagcaggattactctagttttggctgtggaacaaagctggagatcaaaaggggaggaggaggttctggcggaggaggtagtggcggagggggttcacaggtgcagctggtcgaatctgggccaggcgtggtccagccaggacgttccctgaggattagctgcgccgtgagcgggttctctgtcacaaactacggagtgcactgggtccgtcagccacctggcaaatgtctggagtggctgggagtgatctgggcaggaggaatcactaactacaactctgcttttatgagtcgcctgaccatctcaaaggacaactccaaaaatacagtgtacctgcagatgaattcactgcgggcagaagataccgccatgtactattgcgcctccagggggggtcattacggctatgccctggactattggggccagggaacactggtgactgtctcatccggaggaggaggatccggaggaggaggtagcggcggagggggttctggcggagggggtagtcacgtgaagctgcaggaaagcggccctggactggtgcagccttcccagtctctgtccctgacctgcaccgtgtccggcttctccctgaccgattacggcgtgcactgggtgcgacagtctccaggcaagggcctggaatggctgggagtgatttggagcggtggcggaaccgcctacaacaccgccctgatctcccggctgaacatctaccgggacaactccaagaaccaggtgttcctggaaatgaactccctgcaggcagaggacaccgccatgtactactgcgccagacggggctcctacccctacaactacttcgacgcttggggctgcggcaccaccgtgacagtgtctagcggaggtggtggatctgggggcggaggtagcggagggggaggttctcaggctgtcgtgatccaggaatctgccctgaccaccccccctggcgagacagtgacactgacctgcggatcttccaccggcgctgtgaccgcctccaactacgccaactgggtgcaggaaaagcccgaccactgcttcaccggcctgatcggcggccacaacaacagacctccaggcgtgccagcccggttctccggctctctgatcggagataaggccgccctgacaatcgccggcacccagacagaggacgaggctatctacttctgcgccctgtggtacagcgaccactgggtcatcggcggaggcaccagactgaccgtgctgggaacacccctgggagacaccacacatactagtgggagatcccccgacgatgagctgctgtacctgcctgtgaggggccgggagacctatgaaatgctgctgaagatcaaagagagcctggaactgatgcagtacctgccacagcacaccattgaaacatataggcaacaacagcagcagcagcatcagcatctgctgcagaagcagggagggtcaggaggagcaccgcaccatcatcatcaccatt

SEQ ID NO: 49 - protein, 579 aa, synthetic construct
  REGION 1..579: note = Description of Artificial Sequence: Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGCGTTVTVSSGGGGSGGGGSGGGGSQAVVIQESALTTPPGETVTLTCGSSTGAVTASNYANWVQEKPDHCFTGLIGGHNNRPPGVPARFSGSLIGDKAALTIAGTQTEDEAIYFCALWYSDHWVIGGGTRLTVLGTPLGDTTHTSGRHGDEDTYYLQVRGRENFEILMKLKESLELMELVPQPLVDSYRQQQQLLQRPGGSGGAPHHHHHH

SEQ ID NO: 50 - DNA, 1737 bp, synthetic construct
  misc_feature 1..1737: note = Description of Artificial Sequence: Synthetic polynucleotide
  source 1..1737: mol_type = other DNA
gaaatcgtcatgactcagactcccgcaaccctgtcagtgtccgctggggaacgtgtcactattacctgcaaggcatctcagagcgtgagcaacgacgtgacctggtatcagcagaagcctggccaggctccacgactgctgatctattccgcaagcaatcgctactccggagtgcccgcacgattctctggaagtgggtacggtaccgagttcacttttaccatttccagcgtgcagagcgaagacttcgctgtctatttttgccagcaggattactctagttttggctgtggaacaaagctggagatcaaaaggggaggaggaggttctggcggaggaggtagtggcggagggggttcacaggtgcagctggtcgaatctgggccaggcgtggtccagccaggacgttccctgaggattagctgcgccgtgagcgggttctctgtcacaaactacggagtgcactgggtccgtcagccacctggcaaatgtctggagtggctgggagtgatctgggcaggaggaatcactaactacaactctgcttttatgagtcgcctgaccatctcaaaggacaactccaaaaatacagtgtacctgcagatgaattcactgcgggcagaagataccgccatgtactattgcgcctccagggggggtcattacggctatgccctggactattggggccagggaacactggtgactgtctcatccggaggaggaggatccggaggaggaggtagcggcggagggggttctggcggagggggtagtcacgtgaagctgcaggaaagcggccctggactggtgcagccttcccagtctctgtccctgacctgcaccgtgtccggcttctccctgaccgattacggcgtgcactgggtgcgacagtctccaggcaagggcctggaatggctgggagtgatttggagcggtggcggaaccgcctacaacaccgccctgatctcccggctgaacatctaccgggacaactccaagaaccaggtgttcctggaaatgaactccctgcaggcagaggacaccgccatgtactactgcgccagacggggctcctacccctacaactacttcgacgcttggggctgcggcaccaccgtgacagtgtctagcggaggtggtggatctgggggcggaggtagcggagggggaggttctcaggctgtcgtgatccaggaatctgccctgaccaccccccctggcgagacagtgacactgacctgcggatcttccaccggcgctgtgaccgcctccaactacgccaactgggtgcaggaaaagcccgaccactgcttcaccggcctgatcggcggccacaacaacagacctccaggcgtgccagcccggttctccggctctctgatcggagataaggccgccctgacaatcgccggcacccagacagaggacgaggctatctacttctgcgccctgtggtacagcgaccactgggtcatcggcggaggcaccagactgaccgtgctgggaacacccctgggagacaccacacatactagtgggaggcacggcgacgaagatacctactatctgcaggtgaggggacgggagaacttcgaaatcctgatgaagctgaaagagtccctggaactgatggagctggtgccccagcctctggtcgacagctacagacagcagcagcagctgctgcagaggccaggagggtcaggaggagcaccgcaccatcatcatcaccat

SEQ ID NO: 51 - protein, 596 aa, synthetic construct
  REGION 1..596: note = Description of Artificial Sequence: Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGCGTTVTVSSGGGGSGGGGSGGGGSGGGGSGGGGSGGGGSQAVVIQESALTTPPGETVTLTCGSSTGAVTASNYANWVQEKPDHCFTGLIGGHNNRPPGVPARFSGSLIGDKAALTIAGTQTEDEAIYFCALWYSDHWVIGGGTRLTVLGTPLGDTTHTSGKPLDGEYFTLQIRGRERFEMFRELNEALELKDAQAGKEPGGSGGAPHHHHHH

SEQ ID NO: 52 - DNA, 1788 bp, synthetic construct
  misc_feature 1..1788: note = Description of Artificial Sequence: Synthetic polynucleotide
  source 1..1788: mol_type = other DNA
gagatcgtgatgacccagacacccgcaacactgagcgtgtctgccggcgaaagggtcactattacctgcaaggccagtcagtcagtgtccaacgacgtgacttggtaccagcagaaaccaggccaggctccccggctgctgatctacagcgcatctaatagatatagcggagtgcctgctcgcttcagtggttcaggctatggaactgagttcaccttcaccatttccagcgtgcagtccgaagacttcgcagtgtacttttgccagcaggattattctagttttgggtgtggtacaaagctggagatcaaaaggggaggaggaggtagtggcggaggaggttcaggcggagggggtagcggcggagggggttctggcggcggcggtagtggcggcggaggtagccaggtgcagctggtcgaatccggccctggagtggtccagccaggcaggtctctgcggatcagttgcgccgtgtccggattcagcgtcaccaactacggagtgcactgggtcagacagccacctggcaagtgtctggagtggctgggagtgatctgggcaggaggaatcacaaactacaactcagcttttatgtcccgcctgactattagcaaggacaactctaaaaataccgtgtatctgcagatgaattctctgcgagccgaagataccgctatgtactattgtgcatcccgtgggggtcattacggctatgccctggattattgggggcagggtaccctggtgacagtctcatccggcggagggggatccggcggcggaggatctggcggaggtggaagtgggggaggcggatctcacgtgaagctgcaggaaagcggccctggactggtgcagccttcccagtctctgtccctgacctgcaccgtgtccggcttctccctgaccgattacggcgtgcactgggtgcgacagtctccaggcaagggcctggaatggctgggagtgatttggagcggtggcggaaccgcctacaacaccgccctgatctcccggctgaacatctaccgggacaactccaagaaccaggtgttcctggaaatgaactccctgcaggcagaggacaccgccatgtactactgcgccagacggggctcctacccctacaactacttcgacgcttggggctgcggcaccaccgtgacagtgtctagcggaggtggtggatctgggggcggaggtagcggagggggaggttctggaggtggtggatctgggggcggaggtagcggagggggaggttctcaggctgtcgtgatccaggaatctgccctgaccaccccccctggcgagacagtgacactgacctgcggatcttccaccggcgctgtgaccgcctccaactacgccaactgggtgcaggaaaagcccgaccactgcttcaccggcctgatcggcggccacaacaacagacctccaggcgtgccagcccggttctccggctctctgatcggagataaggccgccctgacaatcgccggcacccagacagaggacgaggctatctacttctgcgccctgtggtacagcgaccactgggtcatcggcggaggcaccagactgaccgtgctgggaacacccctgggagacaccacacatactagtgggaaacctctggatggcgagtactttaccctgcagattagaggccgcgaacgattcgagatgtttcgcgaactgaatgaggccctggaactgaaggatgctcaggcaggcaaggagccaggagggtcaggaggagcaccgcaccatcatcatcaccat

SEQ ID NO: 53 - protein, 614 aa, synthetic construct
  REGION 1..614: note = Description of Artificial Sequence: Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGCGTTVTVSSGGGGSGGGGSGGGGSGGGGSGGGGSGGGGSQAVVIQESALTTPPGETVTLTCGSSTGAVTASNYANWVQEKPDHCFTGLIGGHNNRPPGVPARFSGSLIGDKAALTIAGTQTEDEAIYFCALWYSDHWVIGGGTRLTVLGTPLGDTTHTSGRSPDDELLYLPVRGRETYEMLLKIKESLELMQYLPQHTIETYRQQQQQQHQHLLQKQGGSGGAPHHHHHH

SEQ ID NO: 54 - DNA, 1842 bp, synthetic construct
  misc_feature 1..1842: note = Description of Artificial Sequence: Synthetic polynucleotide
  source 1..1842: mol_type = other DNA
gagatcgtgatgacccagacacccgcaacactgagcgtgtctgccggcgaaagggtcactattacctgcaaggccagtcagtcagtgtccaacgacgtgacttggtaccagcagaaaccaggccaggctccccggctgctgatctacagcgcatctaatagatatagcggagtgcctgctcgcttcagtggttcaggctatggaactgagttcaccttcaccatttccagcgtgcagtccgaagacttcgcagtgtacttttgccagcaggattattctagttttgggtgtggtacaaagctggagatcaaaaggggaggaggaggtagtggcggaggaggttcaggcggagggggtagcggcggagggggttctggcggcggcggtagtggcggcggaggtagccaggtgcagctggtcgaatccggccctggagtggtccagccaggcaggtctctgcggatcagttgcgccgtgtccggattcagcgtcaccaactacggagtgcactgggtcagacagccacctggcaagtgtctggagtggctgggagtgatctgggcaggaggaatcacaaactacaactcagcttttatgtcccgcctgactattagcaaggacaactctaaaaataccgtgtatctgcagatgaattctctgcgagccgaagataccgctatgtactattgtgcatcccgtgggggtcattacggctatgccctggattattgggggcagggtaccctggtgacagtctcatccggaggaggaggatccggaggaggaggtagcggcggagggggttctggcggagggggtagtcacgtgaagctgcaggaaagcggccctggactggtgcagccttcccagtctctgtccctgacctgcaccgtgtccggcttctccctgaccgattacggcgtgcactgggtgcgacagtctccaggcaagggcctggaatggctgggagtgatttggagcggtggcggaaccgcctacaacaccgccctgatctcccggctgaacatctaccgggacaactccaagaaccaggtgttcctggaaatgaactccctgcaggcagaggacaccgccatgtactactgcgccagacggggctcctacccctacaactacttcgacgcttggggctgcggcaccaccgtgacagtgtctagcggaggtggtggatctgggggcggaggtagcggagggggaggttctggaggtggtggatctgggggcggaggtagcggagggggaggttctcaggctgtcgtgatccaggaatctgccctgaccaccccccctggcgagacagtgacactgacctgcggatcttccaccggcgctgtgaccgcctccaactacgccaactgggtgcaggaaaagcccgaccactgcttcaccggcctgatcggcggccacaacaacagacctccaggcgtgccagcccggttctccggctctctgatcggagataaggccgccctgacaatcgccggcacccagacagaggacgaggctatctacttctgcgccctgtggtacagcgaccactgggtcatcggcggaggcaccagactgaccgtgctgggaacacccctgggagacaccacacatactagtgggagatcccccgacgatgagctgctgtacctgcctgtgaggggccgggagacctatgaaatgctgctgaagatcaaagagagcctggaactgatgcagtacctgccacagcacaccattgaaacatataggcaacaacagcagcagcagcatcagcatctgctgcagaagcagggagggtcaggaggagcaccgcaccatcatcatcaccat

SEQ ID NO: 55 - protein, 609 aa, synthetic construct
  REGION 1..609: note = Description of Artificial Sequence: Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGCGTTVTVSSGGGGSGGGGSGGGGSGGGGSGGGGSGGGGSQAVVIQESALTTPPGETVTLTCGSSTGAVTASNYANWVQEKPDHCFTGLIGGHNNRPPGVPARFSGSLIGDKAALTIAGTQTEDEAIYFCALWYSDHWVIGGGTRLTVLGTPLGDTTHTSGRHGDEDTYYLQVRGRENFEILMKLKESLELMELVPQPLVDSYRQQQQLLQRPGGSGGAPHHHHHH

SEQ ID NO: 56 - DNA, 1827 bp, synthetic construct
  misc_feature 1..1827: note = Description of Artificial Sequence: Synthetic polynucleotide
  source 1..1827: mol_type = other DNA
gagatcgtgatgacccagacacccgcaacactgagcgtgtctgccggcgaaagggtcactattacctgcaaggccagtcagtcagtgtccaacgacgtgacttggtaccagcagaaaccaggccaggctccccggctgctgatctacagcgcatctaatagatatagcggagtgcctgctcgcttcagtggttcaggctatggaactgagttcaccttcaccatttccagcgtgcagtccgaagacttcgcagtgtacttttgccagcaggattattctagttttgggtgtggtacaaagctggagatcaaaaggggaggaggaggtagtggcggaggaggttcaggcggagggggtagcggcggagggggttctggcggcggcggtagtggcggcggaggtagccaggtgcagctggtcgaatccggccctggagtggtccagccaggcaggtctctgcggatcagttgcgccgtgtccggattcagcgtcaccaactacggagtgcactgggtcagacagccacctggcaagtgtctggagtggctgggagtgatctgggcaggaggaatcacaaactacaactcagcttttatgtcccgcctgactattagcaaggacaactctaaaaataccgtgtatctgcagatgaattctctgcgagccgaagataccgctatgtactattgtgcatcccgtgggggtcattacggctatgccctggattattgggggcagggtaccctggtgacagtctcatccggaggaggaggatccggaggaggaggtagcggcggagggggttctggcggagggggtagtcacgtgaagctgcaggaaagcggccctggactggtgcagccttcccagtctctgtccctgacctgcaccgtgtccggcttctccctgaccgattacggcgtgcactgggtgcgacagtctccaggcaagggcctggaatggctgggagtgatttggagcggtggcggaaccgcctacaacaccgccctgatctcccggctgaacatctaccgggacaactccaagaaccaggtgttcctggaaatgaactccctgcaggcagaggacaccgccatgtactactgcgccagacggggctcctacccctacaactacttcgacgcttggggctgcggcaccaccgtgacagtgtctagcggaggtggtggatctgggggcggaggtagcggagggggaggttctggaggtggtggatctgggggcggaggtagcggagggggaggttctcaggctgtcgtgatccaggaatctgccctgaccaccccccctggcgagacagtgacactgacctgcggatcttccaccggcgctgtgaccgcctccaactacgccaactgggtgcaggaaaagcccgaccactgcttcaccggcctgatcggcggccacaacaacagacctccaggcgtgccagcccggttctccggctctctgatcggagataaggccgccctgacaatcgccggcacccagacagaggacgaggctatctacttctgcgccctgtggtacagcgaccactgggtcatcggcggaggcaccagactgaccgtgctgggaacacccctgggagacaccacacatactagtgggaggcacggcgacgaagatacctactatctgcaggtgaggggacgggagaacttcgaaatcctgatgaagctgaaagagtccctggaactgatggagctggtgccccagcctctggtcgacagctacagacagcagcagcagctgctgcagaggccaggagggtcaggaggagcaccgcaccatcatcatcaccat

SEQ ID NO: 57 - protein, 128 aa, synthetic construct
  REGION 1..128: note = Description of Artificial Sequence: Synthetic polypeptide
ITCPPPMSVEHADIWVKSYSLYSRERYICNSGFKRKAGTSSLTECVLNKATNVAHWTTPSLKCIRTPLGDTTHTSGKPLDGEYFTLQIRGRERFEMFRELNEALELKDAQAGKEPGGSGGAPHHHHHH

SEQ ID NO: 58 - DNA, 384 bp, synthetic construct
  misc_feature 1..384: note = Description of Artificial Sequence: Synthetic polynucleotide
  source 1..384: mol_type = other DNA
atcacctgtcctccacccatgtctgtggaacacgccgacatctgggtcaagtcctactccctgtactccagagagcggtacatctgcaactccggcttcaagcggaaggccggcacctctagcctgaccgagtgcgtgctgaacaaggccaccaacgtggcccactggaccaccccatccctgaagtgcatcagaacacccctgggtgacaccacacatactagtgggaaacctctggatggcgagtactttaccctgcagattagaggccgcgaacgattcgagatgtttcgcgaactgaatgaggccctggaactgaaggatgctcaggcaggcaaggagccaggagggtcaggaggagcaccgcaccatcatcatcaccat

SEQ ID NO: 59 - protein, 146 aa, synthetic construct
  REGION 1..146: note = Description of Artificial Sequence: Synthetic polypeptide
ITCPPPMSVEHADIWVKSYSLYSRERYICNSGFKRKAGTSSLTECVLNKATNVAHWTTPSLKCIRTPLGDTTHTSGRSPDDELLYLPVRGRETYEMLLKIKESLELMQYLPQHTIETYRQQQQQQHQHLLQKQGGSGGAPHHHHHH

SEQ ID NO: 60 - DNA, 438 bp, synthetic construct
  misc_feature 1..438: note = Description of Artificial Sequence: Synthetic polynucleotide
  source 1..438: mol_type = other DNA
atcacctgtcctccacccatgtctgtggaacacgccgacatctgggtcaagtcctactccctgtactccagagagcggtacatctgcaactccggcttcaagcggaaggccggcacctctagcctgaccgagtgcgtgctgaacaaggccaccaacgtggcccactggaccaccccatccctgaagtgcatcagaacacccctgggtgacaccacacatactagtgggagatcccccgacgatgagctgctgtacctgcctgtgaggggccgggagacctatgaaatgctgctgaagatcaaagagagcctggaactgatgcagtacctgccacagcacaccattgaaacatataggcaacaacagcagcagcagcatcagcatctgctgcagaagcagggagggtcaggaggagcaccgcaccatcatcatcaccat

SEQ ID NO: 61 - protein, 141 aa, synthetic construct
  REGION 1..141: note = Description of Artificial Sequence: Synthetic polypeptide
ITCPPPMSVEHADIWVKSYSLYSRERYICNSGFKRKAGTSSLTECVLNKATNVAHWTTPSLKCIRTPLGDTTHTSGRHGDEDTYYLQVRGRENFEILMKLKESLELMELVPQPLVDSYRQQQQLLQRPGGSGGAPHHHHHH

SEQ ID NO: 62 - DNA, 423 bp, synthetic construct
  misc_feature 1..423: note = Description of Artificial Sequence: Synthetic polynucleotide
  source 1..423: mol_type = other DNA
atcacctgtcctccacccatgtctgtggaacacgccgacatctgggtcaagtcctactccctgtactccagagagcggtacatctgcaactccggcttcaagcggaaggccggcacctctagcctgaccgagtgcgtgctgaacaaggccaccaacgtggcccactggaccaccccatccctgaagtgcatcagaacacccctgggtgacaccacacatactagtgggaggcacggcgacgaagatacctactatctgcaggtgaggggacgggagaacttcgaaatcctgatgaagctgaaagagtccctggaactgatggagctggtgccccagcctctggtcgacagctacagacagcagcagcagctgctgcagaggccaggagggtcaggaggagcaccgcaccatcatcatcaccat

SEQ ID NO: 63 - protein, 114 aa, synthetic construct
  REGION 1..114: note = Description of Artificial Sequence: Synthetic polypeptide
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 64 - DNA, 390 bp, synthetic construct
  misc_feature 1..390: note = Description of Artificial Sequence: Synthetic polynucleotide
  source 1..390: mol_type = other DNA
atgggctggtcctgcatcatcctgtttctggtggctaccgccaccggcaactgggtcaacgtgatctccgacctgaagaagatcgaggacctgatccagtccatgcacatcgacgccaccctgtacaccgagtccgacgtgcacccctcctgcaaagtgaccgccatgaagtgctttctgctggaactgcaagtgatctccctggaatccggcgacgcctccatccacgacaccgtggaaaatctgatcatcctggccaacaactccctgtcctccaacggcaacgtgaccgagagcggctgcaaagagtgcgaggaactggaagagaagaacatcaaagagtttctgcagtccttcgtgcacatcgtgcagatgttcatcaacaccagc

SEQ ID NO: 65 - protein, 600 aa, synthetic construct
  REGION 1..600: note = Description of Artificial Sequence: Synthetic polypeptide
EVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQCTKVEIKRGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGAPHHHHHH

SEQ ID NO: 66 - DNA, 1800 bp, synthetic construct
  misc_feature 1..1800: note = Description of Artificial Sequence: Synthetic polynucleotide
  source 1..1800: mol_type = other DNA
gaagtgcagctggtcgaatccggggggggcctggtgcagcctggagggtcactgagactgtcctgtgccgcatctgggttcaatatcaaggacacctacatccactgggtgcggcaggcacctggcaagtgtctggagtgggtggcaaggatctatccaaccaacggctacacacggtatgccgactccgtgaagggccggttcaccatctccgccgatacctctaagaacacagcctacctgcagatgaattctctgagggccgaggatacagccgtgtactattgcagccgctggggaggcgacggcttctacgctatggactattggggccagggcaccctggtgacagtgagctctggcggcggcggatccggaggaggaggcagcggcggaggaggctccggaggaggcggctctggcggcggcggcagcggcggcggcggctccgacatccagatgacccagtccccatctagcctgagcgcctccgtgggcgacagggtgaccatcacatgccgcgccagccaggatgtgaatacagccgtggcctggtaccagcagaagccaggcaaggcccccaagctgctgatctactctgccagcttcctgtatagcggagtgccatcccggttttccggcagccggagcggcaccgacttcaccctgacaatctcctctctgcagcctgaggattttgccacatactattgtcagcagcactataccacaccccctacattcggacagtgtacaaaggtcgagatcaaacgcggcggagggggatccggcggcggaggatctggcggaggtggaagtgggggaggcggatctcatgtgcagctggtggaaagcggaggcggcctggtgcagcctgggggatctctgagactgtcttgtgccgccagcggcttctccctgaccgattatggcgtgcactgggtgcgacaggcccctggcaaaggactggaatggctgggagtgatttggagtggcggaggcaccgcctacaacaccgccctgatctcccggttcaccatcagccgggacaactccaagaacaccctgtacctgcagatgaactccctgcgggccgaggacaccgctgtgtactactgcgccagacggggctcctacccctacaactacttcgacgcttggggctgcggcaccctcgtgacagtgtctagcggagggggaggttctgggggcggaggttcaggtggtggtggttccgggggtggtggctctggtggcggtggttctggcggtggcggatctcaggctgtcgtgacccaggaacccagcctgactgtgtctcctggcggaaccgtgaccctgacctgcggatcttctaccggcgctgtgaccgccagcaactacgccaattgggtgcagcagaaacctggacagtgccctagaggcctgatcggcggccacaacaacagacctccaggcgtgccagcccggttctctggatctctgctgggcggaaaggccgctctgacactgctgggtgctcagcctgaggacgaggccgagtactactgtgccctgtggtactccgaccactgggtcatcggaggcgggaccaagctgaccgtgctgggaacacccctgggagacaccacacatactagtgggaaacctctggatggcgagtactttaccctgcagattagaggccgcgaacgattcgagatgtttcgcgaactgaatgaggccctggaactgaaggatgctcaggcaggcaaggagccaggagggtcaggaggagcaccgcaccatcatcatcaccat

SEQ ID NO: 67 - protein, 600 aa, synthetic construct
  REGION 1..600: note = Description of Artificial Sequence: Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGAPHHHHHH

SEQ ID NO: 68 - DNA, 1800 bp, synthetic construct
  misc_feature 1..1800: note = Description of Artificial Sequence: Synthetic polynucleotide
  source 1..1800: mol_type = other DNA
gaagtgcagctggtcgaatccggggggggcctggtgcagcctggagggtcactgagactgtcctgtgccgcatctgggttcaatatcaaggacacctacatccactgggtgcggcaggcacctggcaagggactggagtgggtggcaaggatctatccaaccaacggctacacacggtatgccgactccgtgaagggccggttcaccatctccgccgatacctctaagaacacagcctacctgcagatgaattctctgagggccgaggatacagccgtgtactattgcagccgctggggaggcgacggcttctacgctatggactattggggccagggcaccctggtgacagtgagctctggcggcggcggatccggaggaggaggcagcggcggaggaggctccggaggaggcggctctggcggcggcggcagcggcggcggcggctccgacatccagatgacccagtccccatctagcctgagcgcctccgtgggcgacagggtgaccatcacatgccgcgccagccaggatgtgaatacagccgtggcctggtaccagcagaagccaggcaaggcccccaagctgctgatctactctgccagcttcctgtatagcggagtgccatcccggttttccggcagccggagcggcaccgacttcaccctgacaatctcctctctgcagcctgaggattttgccacatactattgtcagcagcactataccacaccccctacattcggacaggggacaaaggtcgagatcaaacgcggcggagggggatccggcggcggaggatctggcggaggtggaagtgggggaggcggatctcatgtgcagctggtggaaagcggaggcggcctggtgcagcctgggggatctctgagactgtcttgtgccgccagcggcttctccctgaccgattatggcgtgcactgggtgcgacaggcccctggcaaaggactggaatggctgggagtgatttggagtggcggaggcaccgcctacaacaccgccctgatctcccggttcaccatcagccgggacaactccaagaacaccctgtacctgcagatgaactccctgcgggccgaggacaccgctgtgtactactgcgccagacggggctcctacccctacaactacttcgacgcttggggctgcggcaccctcgtgacagtgtctagcggagggggaggttctgggggcggaggttcaggtggtggtggttccgggggtggtggctctggtggcggtggttctggcggtggcggatctcaggctgtcgtgacccaggaacccagcctgactgtgtctcctggcggaaccgtgaccctgacctgcggatcttctaccggcgctgtgaccgccagcaactacgccaattgggtgcagcagaaacctggacagtgccctagaggcctgatcggcggccacaacaacagacctccaggcgtgccagcccggttctctggatctctgctgggcggaaaggccgctctgacactgctgggtgctcagcctgaggacgaggccgagtactactgtgccctgtggtactccgaccactgggtcatcggaggcgggaccaagctgaccgtgctgggaacacccctgggagacaccacacatactagtgggaaacctctggatggcgagtactttaccctgcagattagaggccgcgaacgattcgagatgtttcgcgaactgaatgaggccctggaactgaaggatgctcaggcaggcaaggagccaggagggtcaggaggagcaccgcaccatcatcatcaccat

SEQ ID NO: 69 - protein, 600 aa, synthetic construct
  REGION 1..600: note = Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQCTKVEIKRGGGGSGGGGSGGGGSGGGGSGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGAPHHHHHH

SEQ ID NO: 70 - DNA, 1800 bp, synthetic construct
  misc_feature 1..1800: note = Description of Artificial Sequence: Synthetic polynucleotide
  source 1..1800: mol_type = other DNA
gatattcagatgactcagtcccctagttcactgtctgcctcagtcggagatcgggtcactatcacttgtcgggcttctcaggatgtgaacaccgccgtggcctggtaccagcagaagccaggcaaggcccccaagctgctgatctactctgccagcttcctgtattccggagtgccatctcggttttccggcagccggagcggcaccgacttcaccctgacaatcagctccctgcagcctgaggattttgccacatactattgccagcagcactataccacaccccctaccttcggccagtgcacaaaggtggagatcaagaggggaggaggaggatccggaggaggaggcagcggaggcggcggctccggcggcggcggctctggcggcggcggcagcggaggaggcggctccgaggtgcagctggtggagtccggcggcggcctggtgcagcccggcggcagcctgcggctgtcctgtgccgcctctggctttaacatcaaggacacctacatccactgggtgaggcaggcacctggcaagtgcctggagtgggtggcaaggatctatccaaccaatggctacacaagatatgccgactccgtgaagggccgctttaccatcagcgccgatacctccaagaacacagcctacctgcagatgaattctctgcgggccgaggatacagccgtgtactattgctccagatggggcggcgacggcttctatgctatggactattgggggcagggaactctggtcactgtctcctctggcggagggggatccggcggcggaggatctggcggaggtggaagtgggggaggcggatctcatgtgcagctggtggaaagcggaggcggcctggtgcagcctgggggatctctgagactgtcttgtgccgccagcggcttctccctgaccgattatggcgtgcactgggtgcgacaggcccctggcaaaggactggaatggctgggagtgatttggagtggcggaggcaccgcctacaacaccgccctgatctcccggttcaccatcagccgggacaactccaagaacaccctgtacctgcagatgaactccctgcgggccgaggacaccgctgtgtactactgcgccagacggggctcctacccctacaactacttcgacgcttggggctgcggcaccctcgtgacagtgtctagcggagggggaggttctgggggcggaggttcaggtggtggtggttccgggggtggtggctctggtggcggtggttctggcggtggcggatctcaggctgtcgtgacccaggaacccagcctgactgtgtctcctggcggaaccgtgaccctgacctgcggatcttctaccggcgctgtgaccgccagcaactacgccaattgggtgcagcagaaacctggacagtgccctagaggcctgatcggcggccacaacaacagacctccaggcgtgccagcccggttctctggatctctgctgggcggaaaggccgctctgacactgctgggtgctcagcctgaggacgaggccgagtactactgtgccctgtggtactccgaccactgggtcatcggaggcgggaccaagctgaccgtgctgggaacacccctgggagacaccacacatactagtgggaaacctctggatggcgagtactttaccctgcagattagaggccgcgaacgattcgagatgtttcgcgaactgaatgaggccctggaactgaaggatgctcaggcaggcaaggagccaggagggtcaggaggagcaccgcaccatcatcatcaccat

SEQ ID NO: 71 - protein, 600 aa, synthetic construct
  REGION 1..600: note = Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRGGGGSGGGGSGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGAPHHHHHH

SEQ ID NO: 72 - DNA, 1800 bp, synthetic construct
  misc_feature 1..1800: note = Description of Artificial Sequence: Synthetic polynucleotide
  source 1..1800: mol_type = other DNA
gatattcagatgactcagtcccctagttcactgtctgcctcagtcggagatcgggtcactatcacttgtcgggcttctcaggatgtgaacaccgccgtggcctggtaccagcagaagccaggcaaggcccccaagctgctgatctactctgccagcttcctgtattccggagtgccatctcggttttccggcagccggagcggcaccgacttcaccctgacaatcagctccctgcagcctgaggattttgccacatactattgccagcagcactataccacaccccctaccttcggccagggcacaaaggtggagatcaagaggggaggaggaggatccggaggaggaggcagcggaggcggcggctccggcggcggcggctctggcggcggcggcagcggaggaggcggctccgaggtgcagctggtggagtccggcggcggcctggtgcagcccggcggcagcctgcggctgtcctgtgccgcctctggctttaacatcaaggacacctacatccactgggtgaggcaggcacctggcaagggcctggagtgggtggcaaggatctatccaaccaatggctacacaagatatgccgactccgtgaagggccgctttaccatcagcgccgatacctccaagaacacagcctacctgcagatgaattctctgcgggccgaggatacagccgtgtactattgctccagatggggcggcgacggcttctatgctatggactattgggggcagggaactctggtcactgtctcctctggcggagggggatccggcggcggaggatctggcggaggtggaagtgggggaggcggatctcatgtgcagctggtggaaagcggaggcggcctggtgcagcctgggggatctctgagactgtcttgtgccgccagcggcttctccctgaccgattatggcgtgcactgggtgcgacaggcccctggcaaaggactggaatggctgggagtgatttggagtggcggaggcaccgcctacaacaccgccctgatctcccggttcaccatcagccgggacaactccaagaacaccctgtacctgcagatgaactccctgcgggccgaggacaccgctgtgtactactgcgccagacggggctcctacccctacaactacttcgacgcttggggctgcggcaccctcgtgacagtgtctagcggagggggaggttctgggggcggaggttcaggtggtggtggttccgggggtggtggctctggtggcggtggttctggcggtggcggatctcaggctgtcgtgacccaggaacccagcctgactgtgtctcctggcggaaccgtgaccctgacctgcggatcttctaccggcgctgtgaccgccagcaactacgccaattgggtgcagcagaaacctggacagtgccctagaggcctgatcggcggccacaacaacagacctccaggcgtgccagcccggttctctggatctctgctgggcggaaaggccgctctgacactgctgggtgctcagcctgaggacgaggccgagtactactgtgccctgtggtactccgaccactgggtcatcggaggcgggaccaagctgaccgtgctgggaacacccctgggagacaccacacatactagtgggaaacctctggatggcgagtactttaccctgcagattagaggccgcgaacgattcgagatgtttcgcgaactgaatgaggccctggaactgaaggatgctcaggcaggcaaggagccaggagggtcaggaggagcaccgcaccatcatcatcaccat

SEQ ID NO: 73 - protein, 618 aa, synthetic construct
  REGION 1..618: note = Description of Artificial Sequence: Synthetic polypeptide
EVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQCTKVEIKRGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGRSPDDELLYLPVRGRETYEMLLKIKESLELMQYLPQHTIETYRQQQQQQHQHLLQKQGGSGGAPHHHHHH

SEQ ID NO: 74 - DNA, 1854 bp, synthetic construct
  misc_feature 1..1854: note = Description of Artificial Sequence: Synthetic polynucleotide
  source 1..1854: mol_type = other DNA
gaagtgcagctggtcgaatccggggggggcctggtgcagcctggagggtcactgagactgtcctgtgccgcatctgggttcaatatcaaggacacctacatccactgggtgcggcaggcacctggcaagtgtctggagtgggtggcaaggatctatccaaccaacggctacacacggtatgccgactccgtgaagggccggttcaccatctccgccgatacctctaagaacacagcctacctgcagatgaattctctgagggccgaggatacagccgtgtactattgcagccgctggggaggcgacggcttctacgctatggactattggggccagggcaccctggtgacagtgagctctggcggcggcggatccggaggaggaggcagcggcggaggaggctccggaggaggcggctctggcggcggcggcagcggcggcggcggctccgacatccagatgacccagtccccatctagcctgagcgcctccgtgggcgacagggtgaccatcacatgccgcgccagccaggatgtgaatacagccgtggcctggtaccagcagaagccaggcaaggcccccaagctgctgatctactctgccagcttcctgtatagcggagtgccatcccggttttccggcagccggagcggcaccgacttcaccctgacaatctcctctctgcagcctgaggattttgccacatactattgtcagcagcactataccacaccccctacattcggacagtgtacaaaggtcgagatcaaacgcggcggagggggatccggcggcggaggatctggcggaggtggaagtgggggaggcggatctcatgtgcagctggtggaaagcggaggcggcctggtgcagcctgggggatctctgagactgtcttgtgccgccagcggcttctccctgaccgattatggcgtgcactgggtgcgacaggcccctggcaaaggactggaatggctgggagtgatttggagtggcggaggcaccgcctacaacaccgccctgatctcccggttcaccatcagccgggacaactccaagaacaccctgtacctgcagatgaactccctgcgggccgaggacaccgctgtgtactactgcgccagacggggctcctacccctacaactacttcgacgcttggggctgcggcaccctcgtgacagtgtctagcggagggggaggttctgggggcggaggttcaggtggtggtggttccgggggtggtggctctggtggcggtggttctggcggtggcggatctcaggctgtcgtgacccaggaacccagcctgactgtgtctcctggcggaaccgtgaccctgacctgcggatcttctaccggcgctgtgaccgccagcaactacgccaattgggtgcagcagaaacctggacagtgccctagaggcctgatcggcggccacaacaacagacctccaggcgtgccagcccggttctctggatctctgctgggcggaaaggccgctctgacactgctgggtgctcagcctgaggacgaggccgagtactactgtgccctgtggtactccgaccactgggtcatcggaggcgggaccaagctgaccgtgctgggaacacccctgggagacaccacacatactagtgggagatcccccgacgatgagctgctgtacctgcctgtgaggggccgggagacctatgaaatgctgctgaagatcaaagagagcctggaactgatgcagtacctgccacagcacaccattgaaacatataggcaacaacagcagcagcagcatcagcatctgctgcagaagcagggagggtcaggaggagcaccgcaccatcatcatcaccat

SEQ ID NO: 75 - protein, 618 aa, synthetic construct
  REGION 1..618: note = Description of Artificial Sequence: Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGRSPDDELLYLPVRGRETYEMLLKIKESLELMQYLPQHTIETYRQQQQQQHQHLLQKQGGSGGAPHHHHHH

SEQ ID NO: 76 - DNA, 1854 bp, synthetic construct
  misc_feature 1..1854: note = Description of Artificial Sequence: Synthetic polynucleotide
  source 1..1854: mol_type = other DNA
gaagtgcagctggtcgaatccggggggggcctggtgcagcctggagggtcactgagactgtcctgtgccgcatctgggttcaatatcaaggacacctacatccactgggtgcggcaggcacctggcaagggactggagtgggtggcaaggatctatccaaccaacggctacacacggtatgccgactccgtgaagggccggttcaccatctccgccgatacctctaagaacacagcctacctgcagatgaattctctgagggccgaggatacagccgtgtactattgcagccgctggggaggcgacggcttctacgctatggactattggggccagggcaccctggtgacagtgagctctggcggcggcggatccggaggaggaggcagcggcggaggaggctccggaggaggcggctctggcggcggcggcagcggcggcggcggctccgacatccagatgacccagtccccatctagcctgagcgcctccgtgggcgacagggtgaccatcacatgccgcgccagccaggatgtgaatacagccgtggcctggtaccagcagaagccaggcaaggcccccaagctgctgatctactctgccagcttcctgtatagcggagtgccatcccggttttccggcagccggagcggcaccgacttcaccctgacaatctcctctctgcagcctgaggattttgccacatactattgtcagcagcactataccacaccccctacattcggacaggggacaaaggtcgagatcaaacgcggcggagggggatccggcggcggaggatctggcggaggtggaagtgggggaggcggatctcatgtgcagctggtggaaagcggaggcggcctggtgcagcctgggggatctctgagactgtcttgtgccgccagcggcttctccctgaccgattatggcgtgcactgggtgcgacaggcccctggcaaaggactggaatggctgggagtgatttggagtggcggaggcaccgcctacaacaccgccctgatctcccggttcaccatcagccgggacaactccaagaacaccctgtacctgcagatgaactccctgcgggccgaggacaccgctgtgtactactgcgccagacggggctcctacccctacaactacttcgacgcttggggctgcggcaccctcgtgacagtgtctagcggagggggaggttctgggggcggaggttcaggtggtggtggttccgggggtggtggctctggtggcggtggttctggcggtggcggatctcaggctgtcgtgacccaggaacccagcctgactgtgtctcctggcggaaccgtgaccctgacctgcggatcttctaccggcgctgtgaccgccagcaactacgccaattgggtgcagcagaaacctggacagtgccctagaggcctgatcggcggccacaacaacagacctccaggcgtgccagcccggttctctggatctctgctgggcggaaaggccgctctgacactgctgggtgctcagcctgaggacgaggccgagtactactgtgccctgtggtactccgaccactgggtcatcggaggcgggaccaagctgaccgtgctgggaacacccctgggagacaccacacatactagtgggagatcccccgacgatgagctgctgtacctgcctgtgaggggccgggagacctatgaaatgctgctgaagatcaaagagagcctggaactgatgcagtacctgccacagcacaccattgaaacatataggcaacaacagcagcagcagcatcagcatctgctgcagaagcagggagggtcaggaggagcaccgcaccatcatcatcaccat

SEQ ID NO: 77 - protein, 618 aa, synthetic construct
  REGION 1..618: note = Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQCTKVEIKRGGGGSGGGGSGGGGSGGGGSGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGRSPDDELLYLPVRGRETYEMLLKIKESLELMQYLPQHTIETYRQQQQQQHQHLLQKQGGSGGAPHHHHHH

SEQ ID NO: 78 - DNA, 1854 bp, synthetic construct
  misc_feature 1..1854: note = Description of Artificial Sequence: Synthetic polynucleotide
  source 1..1854: mol_type = other DNA
gatattcagatgactcagtcccctagttcactgtctgcctcagtcggagatcgggtcactatcacttgtcgggcttctcaggatgtgaacaccgccgtggcctggtaccagcagaagccaggcaaggcccccaagctgctgatctactctgccagcttcctgtattccggagtgccatctcggttttccggcagccggagcggcaccgacttcaccctgacaatcagctccctgcagcctgaggattttgccacatactattgccagcagcactataccacaccccctaccttcggccagtgcacaaaggtggagatcaagaggggaggaggaggatccggaggaggaggcagcggaggcggcggctccggcggcggcggctctggcggcggcggcagcggaggaggcggctccgaggtgcagctggtggagtccggcggcggcctggtgcagcccggcggcagcctgcggctgtcctgtgccgcctctggctttaacatcaaggacacctacatccactgggtgaggcaggcacctggcaagtgcctggagtgggtggcaaggatctatccaaccaatggctacacaagatatgccgactccgtgaagggccgctttaccatcagcgccgatacctccaagaacacagcctacctgcagatgaattctctgcgggccgaggatacagccgtgtactattgctccagatggggcggcgacggcttctatgctatggactattgggggcagggaactctggtcactgtctcctctggcggagggggatccggcggcggaggatctggcggaggtggaagtgggggaggcggatctcatgtgcagctggtggaaagcggaggcggcctggtgcagcctgggggatctctgagactgtcttgtgccgccagcggcttctccctgaccgattatggcgtgcactgggtgcgacaggcccctggcaaaggactggaatggctgggagtgatttggagtggcggaggcaccgcctacaacaccgccctgatctcccggttcaccatcagccgggacaactccaagaacaccctgtacctgcagatgaactccctgcgggccgaggacaccgctgtgtactactgcgccagacggggctcctacccctacaactacttcgacgcttggggctgcggcaccctcgtgacagtgtctagcggagggggaggttctgggggcggaggttcaggtggtggtggttccgggggtggtggctctggtggcggtggttctggcggtggcggatctcaggctgtcgtgacccaggaacccagcctgactgtgtctcctggcggaaccgtgaccctgacctgcggatcttctaccggcgctgtgaccgccagcaactacgccaattgggtgcagcagaaacctggacagtgccctagaggcctgatcggcggccacaacaacagacctccaggcgtgccagcccggttctctggatctctgctgggcggaaaggccgctctgacactgctgggtgctcagcctgaggacgaggccgagtactactgtgccctgtggtactccgaccactgggtcatcggaggcgggaccaagctgaccgtgctgggaacacccctgggagacaccacacatactagtgggagatcccccgacgatgagctgctgtacctgcctgtgaggggccgggagacctatgaaatgctgctgaagatcaaagagagcctggaactgatgcagtacctgccacagcacaccattgaaacatataggcaacaacagcagcagcagcatcagcatctgctgcagaagcagggagggtcaggaggagcaccgcaccatcatcatcaccat

SEQ ID NO: 79 - protein, 618 aa, synthetic construct
  REGION 1..618: note = Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRGGGGSGGGGSGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGRSPDDELLYLPVRGRETYEMLLKIKESLELMQYLPQHTIETYRQQQQQQHQHLLQKQGGSGGAPHHHHHH

SEQ ID NO: 80 - DNA, 1854 bp, synthetic construct
  misc_feature 1..1854: note = Description of Artificial Sequence: Synthetic polynucleotide
  source 1..1854: mol_type = other DNA
gatattcagatgactcagtcccctagttcactgtctgcctcagtcggagatcgggtcactatcacttgtcgggcttctcaggatgtgaacaccgccgtggcctggtaccagcagaagccaggcaaggcccccaagctgctgatctactctgccagcttcctgtattccggagtgccatctcggttttccggcagccggagcggcaccgacttcaccctgacaatcagctccctgcagcctgaggattttgccacatactattgccagcagcactataccacaccccctaccttcggccagggcacaaaggtggagatcaagaggggaggaggaggatccggaggaggaggcagcggaggcggcggctccggcggcggcggctctggcggcggcggcagcggaggaggcggctccgaggtgcagctggtggagtccggcggcggcctggtgcagcccggcggcagcctgcggctgtcctgtgccgcctctggctttaacatcaaggacacctacatccactgggtgaggcaggcacctggcaagggcctggagtgggtggcaaggatctatccaaccaatggctacacaagatatgccgactccgtgaagggccgctttaccatcagcgccgatacctccaagaacacagcctacctgcagatgaattctctgcgggccgaggatacagccgtgtactattgctccagatggggcggcgacggcttctatgctatggactattgggggcagggaactctggtcactgtctcctctggcggagggggatccggcggcggaggatctggcggaggtggaagtgggggaggcggatctcatgtgcagctggtggaaagcggaggcggcctggtgcagcctgggggatctctgagactgtcttgtgccgccagcggcttctccctgaccgattatggcgtgcactgggtgcgacaggcccctggcaaaggactggaatggctgggagtgatttggagtggcggaggcaccgcctacaacaccgccctgatctcccggttcaccatcagccgggacaactccaagaacaccctgtacctgcagatgaactccctgcgggccgaggacaccgctgtgtactactgcgccagacggggctcctacccctacaactacttcgacgcttggggctgcggcaccctcgtgacagtgtctagcggagggggaggttctgggggcggaggttcaggtggtggtggttccgggggtggtggctctggtggcggtggttctggcggtggcggatctcaggctgtcgtgacccaggaacccagcctgactgtgtctcctggcggaaccgtgaccctgacctgcggatcttctaccggcgctgtgaccgccagcaactacgccaattgggtgcagcagaaacctggacagtgccctagaggcctgatcggcggccacaacaacagacctccaggcgtgccagcccggttctctggatctctgctgggcggaaaggccgctctgacactgctgggtgctcagcctgaggacgaggccgagtactactgtgccctgtggtactccgaccactgggtcatcggaggcgggaccaagctgaccgtgctgggaacacccctgggagacaccacacatactagtgggagatcccccgacgatgagctgctgtacctgcctgtgaggggccgggagacctatgaaatgctgctgaagatcaaagagagcctggaactgatgcagtacctgccacagcacaccattgaaacatataggcaacaacagcagcagcagcatcagcatctgctgcagaagcagggagggtcaggaggagcaccgcaccatcatcatcaccat

SEQ ID NO: 81 - protein, 613 aa, synthetic construct
  REGION 1..613: note = Description of Artificial Sequence: Synthetic polypeptide
EVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQCTKVEIKRGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGRHGDEDTYYLQVRGRENFEILMKLKESLELMELVPQPLVDSYRQQQQLLQRPGGSGGAPHHHHHH

SEQ ID NO: 82 - DNA, 1839 bp, synthetic construct
  misc_feature 1..1839: note = Description of Artificial Sequence: Synthetic polynucleotide
  source 1..1839: mol_type = other DNA
gaagtgcagctggtcgaatccggggggggcctggtgcagcctggagggtcactgagactgtcctgtgccgcatctgggttcaatatcaaggacacctacatccactgggtgcggcaggcacctggcaagtgtctggagtgggtggcaaggatctatccaaccaacggctacacacggtatgccgactccgtgaagggccggttcaccatctccgccgatacctctaagaacacagcctacctgcagatgaattctctgagggccgaggatacagccgtgtactattgcagccgctggggaggcgacggcttctacgctatggactattggggccagggcaccctggtgacagtgagctctggcggcggcggatccggaggaggaggcagcggcggaggaggctccggaggaggcggctctggcggcggcggcagcggcggcggcggctccgacatccagatgacccagtccccatctagcctgagcgcctccgtgggcgacagggtgaccatcacatgccgcgccagccaggatgtgaatacagccgtggcctggtaccagcagaagccaggcaaggcccccaagctgctgatctactctgccagcttcctgtatagcggagtgccatcccggttttccggcagccggagcggcaccgacttcaccctgacaatctcctctctgcagcctgaggattttgccacatactattgtcagcagcactataccacaccccctacattcggacagtgtacaaaggtcgagatcaaacgcggcggagggggatccggcggcggaggatctggcggaggtggaagtgggggaggcggatctcatgtgcagctggtggaaagcggaggcggcctggtgcagcctgggggatctctgagactgtcttgtgccgccagcggcttctccctgaccgattatggcgtgcactgggtgcgacaggcccctggcaaaggactggaatggctgggagtgatttggagtggcggaggcaccgcctacaacaccgccctgatctcccggttcaccatcagccgggacaactccaagaacaccctgtacctgcagatgaactccctgcgggccgaggacaccgctgtgtactactgcgccagacggggctcctacccctacaactacttcgacgcttggggctgcggcaccctcgtgacagtgtctagcggagggggaggttctgggggcggaggttcaggtggtggtggttccgggggtggtggctctggtggcggtggttctggcggtggcggatctcaggctgtcgtgacccaggaacccagcctgactgtgtctcctggcggaaccgtgaccctgacctgcggatcttctaccggcgctgtgaccgccagcaactacgccaattgggtgcagcagaaacctggacagtgccctagaggcctgatcggcggccacaacaacagacctccaggcgtgccagcccggttctctggatctctgctgggcggaaaggccgctctgacactgctgggtgctcagcctgaggacgaggccgagtactactgtgccctgtggtactccgaccactgggtcatcggaggcgggaccaagctgaccgtgctgggaacacccctgggagacaccacacatactagtgggaggcacggcgacgaagatacctactatctgcaggtgaggggacgggagaacttcgaaatcctgatgaagctgaaagagtccctggaactgatggagctggtgccccagcctctggtcgacagctacagacagcagcagcagctgctgcagaggccaggagggtcaggaggagcaccgcaccatcatcatcaccat

SEQ ID NO: 83 - protein, 613 aa, synthetic construct
  REGION 1..613: note = Description of Artificial Sequence: Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGRHGDEDTYYLQVRGRENFEILMKLKESLELMELVPQPLVDSYRQQQQLLQRPGGSGGAPHHHHHH

SEQ ID NO: 84 - DNA, 1839 bp, synthetic construct
  misc_feature 1..1839: note = Description of Artificial Sequence: Synthetic polynucleotide
  source 1..1839: mol_type = other DNA
gaagtgcagctggtcgaatccggggggggcctggtgcagcctggagggtcactgagactgtcctgtgccgcatctgggttcaatatcaaggacacctacatccactgggtgcggcaggcacctggcaagggactggagtgggtggcaaggatctatccaaccaacggctacacacggtatgccgactccgtgaagggccggttcaccatctccgccgatacctctaagaacacagcctacctgcagatgaattctctgagggccgaggatacagccgtgtactattgcagccgctggggaggcgacggcttctacgctatggactattggggccagggcaccctggtgacagtgagctctggcggcggcggatccggaggaggaggcagcggcggaggaggctccggaggaggcggctctggcggcggcggcagcggcggcggcggctccgacatccagatgacccagtccccatctagcctgagcgcctccgtgggcgacagggtgaccatcacatgccgcgccagccaggatgtgaatacagccgtggcctggtaccagcagaagccaggcaaggcccccaagctgctgatctactctgccagcttcctgtatagcggagtgccatcccggttttccggcagccggagcggcaccgacttcaccctgacaatctcctctctgcagcctgaggattttgccacatactattgtcagcagcactataccacaccccctacattcggacaggggacaaaggtcgagatcaaacgcggcggagggggatccggcggcggaggatctggcggaggtggaagtgggggaggcggatctcatgtgcagctggtggaaagcggaggcggcctggtgcagcctgggggatctctgagactgtcttgtgccgccagcggcttctccctgaccgattatggcgtgcactgggtgcgacaggcccctggcaaaggactggaatggctgggagtgatttggagtggcggaggcaccgcctacaacaccgccctgatctcccggttcaccatcagccgggacaactccaagaacaccctgtacctgcagatgaactccctgcgggccgaggacaccgctgtgtactactgcgccagacggggctcctacccctacaactacttcgacgcttggggctgcggcaccctcgtgacagtgtctagcggagggggaggttctgggggcggaggttcaggtggtggtggttccgggggtggtggctctggtggcggtggttctggcggtggcggatctcaggctgtcgtgacccaggaacccagcctgactgtgtctcctggcggaaccgtgaccctgacctgcggatcttctaccggcgctgtgaccgccagcaactacgccaattgggtgcagcagaaacctggacagtgccctagaggcctgatcggcggccacaacaacagacctccaggcgtgccagcccggttctctggatctctgctgggcggaaaggccgctctgacactgctgggtgctcagcctgaggacgaggccgagtactactgtgccctgtggtactccgaccactgggtcatcggaggcgggaccaagctgaccgtgctgggaacacccctgggagacaccacacatactagtgggaggcacggcgacgaagatacctactatctgcaggtgaggggacgggagaacttcgaaatcctgatgaagctgaaagagtccctggaactgatggagctggtgccccagcctctggtcgacagctacagacagcagcagcagctgctgcagaggccaggagggtcaggaggagcaccgcaccatcatcatcaccat

SEQ ID NO: 85 - protein, 613 aa, synthetic construct
  REGION 1..613: note = Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQCTKVEIKRGGGGSGGGGSGGGGSGGGGSGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGRHGDEDTYYLQVRGRENFEILMKLKESLELMELVPQPLVDSYRQQQQLLQRPGGSGGAPHHHHHH

SEQ ID NO: 86 - DNA, 1839 bp, synthetic construct
  misc_feature 1..1839: note = Description of Artificial Sequence: Synthetic polynucleotide
  source 1..1839: mol_type = other DNA
gatattcagatgactcagtcccctagttcactgtctgcctcagtcggagatcgggtcactatcacttgtcgggcttctcaggatgtgaacaccgccgtggcctggtaccagcagaagccaggcaaggcccccaagctgctgatctactctgccagcttcctgtattccggagtgccatctcggttttccggcagccggagcggcaccgacttcaccctgacaatcagctccctgcagcctgaggattttgccacatactattgccagcagcactataccacaccccctaccttcggccagtgcacaaaggtggagatcaagaggggaggaggaggatccggaggaggaggcagcggaggcggcggctccggcggcggcggctctggcggcggcggcagcggaggaggcggctccgaggtgcagctggtggagtccggcggcggcctggtgcagcccggcggcagcctgcggctgtcctgtgccgcctctggctttaacatcaaggacacctacatccactgggtgaggcaggcacctggcaagtgcctggagtgggtggcaaggatctatccaaccaatggctacacaagatatgccgactccgtgaagggccgctttaccatcagcgccgatacctccaagaacacagcctacctgcagatgaattctctgcgggccgaggatacagccgtgtactattgctccagatggggcggcgacggcttctatgctatggactattgggggcagggaactctggtcactgtctcctctggcggagggggatccggcggcggaggatctggcggaggtggaagtgggggaggcggatctcatgtgcagctggtggaaagcggaggcggcctggtgcagcctgggggatctctgagactgtcttgtgccgccagcggcttctccctgaccgattatggcgtgcactgggtgcgacaggcccctggcaaaggactggaatggctgggagtgatttggagtggcggaggcaccgcctacaacaccgccctgatctcccggttcaccatcagccgggacaactccaagaacaccctgtacctgcagatgaactccctgcgggccgaggacaccgctgtgtactactgcgccagacggggctcctacccctacaactacttcgacgcttggggctgcggcaccctcgtgacagtgtctagcggagggggaggttctgggggcggaggttcaggtggtggtggttccgggggtggtggctctggtggcggtggttctggcggtggcggatctcaggctgtcgtgacccaggaacccagcctgactgtgtctcctggcggaaccgtgaccctgacctgcggatcttctaccggcgctgtgaccgccagcaactacgccaattgggtgcagcagaaacctggacagtgccctagaggcctgatcggcggccacaacaacagacctccaggcgtgccagcccggttctctggatctctgctgggcggaaaggccgctctgacactgctgggtgctcagcctgaggacgaggccgagtactactgtgccctgtggtactccgaccactgggtcatcggaggcgggaccaagctgaccgtgctgggaacacccctgggagacaccacacatactagtgggaggcacggcgacgaagatacctactatctgcaggtgaggggacgggagaacttcgaaatcctgatgaagctgaaagagtccctggaactgatggagctggtgccccagcctctggtcgacagctacagacagcagcagcagctgctgcagaggccaggagggtcaggaggagcaccgcaccatcatcatcaccat

SEQ ID NO: 87 - protein, 613 aa, synthetic construct
  REGION 1..613: note = Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRGGGGSGGGGSGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGRHGDEDTYYLQVRGRENFEILMKLKESLELMELVPQPLVDSYRQQQQLLQRPGGSGGAPHHHHHH

SEQ ID NO: 88 - DNA, 1839 bp, synthetic construct
  misc_feature 1..1839: note = Description of Artificial Sequence: Synthetic polynucleotide
  source 1..1839: mol_type = other DNA
gatattcagatgactcagtcccctagttcactgtctgcctcagtcggagatcgggtcactatcacttgtcgggcttctcaggatgtgaacaccgccgtggcctggtaccagcagaagccaggcaaggcccccaagctgctgatctactctgccagcttcctgtattccggagtgccatctcggttttccggcagccggagcggcaccgacttcaccctgacaatcagctccctgcagcctgaggattttgccacatactattgccagcagcactataccacaccccctaccttcggccagggcacaaaggtggagatcaagaggggaggaggaggatccggaggaggaggcagcggaggcggcggctccggcggcggcggctctggcggcggcggcagcggaggaggcggctccgaggtgcagctggtggagtccggcggcggcctggtgcagcccggcggcagcctgcggctgtcctgtgccgcctctggctttaacatcaaggacacctacatccactgggtgaggcaggcacctggcaagggcctggagtgggtggcaaggatctatccaaccaatggctacacaagatatgccgactccgtgaagggccgctttaccatcagcgccgatacctccaagaacacagcctacctgcagatgaattctctgcgggccgaggatacagccgtgtactattgctccagatggggcggcgacggcttctatgctatggactattgggggcagggaactctggtcactgtctcctctggcggagggggatccggcggcggaggatctggcggaggtggaagtgggggaggcggatctcatgtgcagctggtggaaagcggaggcggcctggtgcagcctgggggatctctgagactgtcttgtgccgccagcggcttctccctgaccgattatggcgtgcactgggtgcgacaggcccctggcaaaggactggaatggctgggagtgatttggagtggcggaggcaccgcctacaacaccgccctgatctcccggttcaccatcagccgggacaactccaagaacaccctgtacctgcagatgaactccctgcgggccgaggacaccgctgtgtactactgcgccagacggggctcctacccctacaactacttcgacgcttggggctgcggcaccctcgtgacagtgtctagcggagggggaggttctgggggcggaggttcaggtggtggtggttccgggggtggtggctctggtggcggtggttctggcggtggcggatctcaggctgtcgtgacccaggaacccagcctgactgtgtctcctggcggaaccgtgaccctgacctgcggatcttctaccggcgctgtgaccgccagcaactacgccaattgggtgcagcagaaacctggacagtgccctagaggcctgatcggcggccacaacaacagacctccaggcgtgccagcccggttctctggatctctgctgggcggaaaggccgctctgacactgctgggtgctcagcctgaggacgaggccgagtactactgtgccctgtggtactccgaccactgggtcatcggaggcgggaccaagctgaccgtgctgggaacacccctgggagacaccacacatactagtgggaggcacggcgacgaagatacctactatctgcaggtgaggggacgggagaacttcgaaatcctgatgaagctgaaagagtccctggaactgatggagctggtgccccagcctctggtcgacagctacagacagcagcagcagctgctgcagaggccaggagggtcaggaggagcaccgcaccatcatcatcaccat

SEQ ID NO: 89 - protein, 588 aa, synthetic construct
  REGION 1..588: note = Description of Artificial Sequence: Synthetic polypeptide
EVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQCTKVEIKRGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGQAIKKELTQIKQKVDSLLENLEKIEKEGGSGGAPHHHHHH

SEQ ID NO: 90 - DNA, 1764 bp, synthetic construct
  misc_feature 1..1764: note = Description of Artificial Sequence: Synthetic polynucleotide
  source 1..1764: mol_type = other DNA
gaagtgcagctggtcgaatccggggggggcctggtgcagcctggagggtcactgagactgtcctgtgccgcatctgggttcaatatcaaggacacctacatccactgggtgcggcaggcacctggcaagtgtctggagtgggtggcaaggatctatccaaccaacggctacacacggtatgccgactccgtgaagggccggttcaccatctccgccgatacctctaagaacacagcctacctgcagatgaattctctgagggccgaggatacagccgtgtactattgcagccgctggggaggcgacggcttctacgctatggactattggggccagggcaccctggtgacagtgagctctggcggcggcggatccggaggaggaggcagcggcggaggaggctccggaggaggcggctctggcggcggcggcagcggcggcggcggctccgacatccagatgacccagtccccatctagcctgagcgcctccgtgggcgacagggtgaccatcacatgccgcgccagccaggatgtgaatacagccgtggcctggtaccagcagaagccaggcaaggcccccaagctgctgatctactctgccagcttcctgtatagcggagtgccatcccggttttccggcagccggagcggcaccgacttcaccctgacaatctcctctctgcagcctgaggattttgccacatactattgtcagcagcactataccacaccccctacattcggacagtgtacaaaggtcgagatcaaacgcggcggagggggatccggcggcggaggatctggcggaggtggaagtgggggaggcggatctcatgtgcagctggtggaaagcggaggcggcctggtgcagcctgggggatctctgagactgtcttgtgccgccagcggcttctccctgaccgattatggcgtgcactgggtgcgacaggcccctggcaaaggactggaatggctgggagtgatttggagtggcggaggcaccgcctacaacaccgccctgatctcccggttcaccatcagccgggacaactccaagaacaccctgtacctgcagatgaactccctgcgggccgaggacaccgctgtgtactactgcgccagacggggctcctacccctacaactacttcgacgcttggggctgcggcaccctcgtgacagtgtctagcggagggggaggttctgggggcggaggttcaggtggtggtggttccgggggtggtggctctggtggcggtggttctggcggtggcggatctcaggctgtcgtgacccaggaacccagcctgactgtgtctcctggcggaaccgtgaccctgacctgcggatcttctaccggcgctgtgaccgccagcaactacgccaattgggtgcagcagaaacctggacagtgccctagaggcctgatcggcggccacaacaacagacctccaggcgtgccagcccggttctctggatctctgctgggcggaaaggccgctctgacactgctgggtgctcagcctgaggacgaggccgagtactactgtgccctgtggtactccgaccactgggtcatcggaggcgggaccaagctgaccgtgctgggaacacccctgggagacaccacacatactagtgggcaggccatcaagaaggagctgacccagatcaagcagaaggtggacagcctgctggagaacctggagaagatcgagaaggagggagggtcaggaggagcaccgcaccatcatcatcaccat

SEQ ID NO: 91 - protein, 588 aa, synthetic construct
  REGION 1..588: note = Description of Artificial Sequence: Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGQAIKKELTQIKQKVDSLLENLEKIEKEGGSGGAPHHHHHH

SEQ ID NO: 92 - DNA, 1764 bp, synthetic construct
  misc_feature 1..1764: note = Description of Artificial Sequence: Synthetic polynucleotide
  source 1..1764: mol_type = other DNA
gaagtgcagctggtcgaatccggggggggcctggtgcagcctggagggtcactgagactgtcctgtgccgcatctgggttcaatatcaaggacacctacatccactgggtgcggcaggcacctggcaagggactggagtgggtggcaaggatctatccaaccaacggctacacacggtatgccgactccgtgaagggccggttcaccatctccgccgatacctctaagaacacagcctacctgcagatgaattctctgagggccgaggatacagccgtgtactattgcagccgctggggaggcgacggcttctacgctatggactattggggccagggcaccctggtgacagtgagctctggcggcggcggatccggaggaggaggcagcggcggaggaggctccggaggaggcggctctggcggcggcggcagcggcggcggcggctccgacatccagatgacccagtccccatctagcctgagcgcctccgtgggcgacagggtgaccatcacatgccgcgccagccaggatgtgaatacagccgtggcctggtaccagcagaagccaggcaaggcccccaagctgctgatctactctgccagcttcctgtatagcggagtgccatcccggttttccggcagccggagcggcaccgacttcaccctgacaatctcctctctgcagcctgaggattttgccacatactattgtcagcagcactataccacaccccctacattcggacaggggacaaaggtcgagatcaaacgcggcggagggggatccggcggcggaggatctggcggaggtggaagtgggggaggcggatctcatgtgcagctggtggaaagcggaggcggcctggtgcagcctgggggatctctgagactgtcttgtgccgccagcggcttctccctgaccgattatggcgtgcactgggtgcgacaggcccctggcaaaggactggaatggctgggagtgatttggagtggcggaggcaccgcctacaacaccgccctgatctcccggttcaccatcagccgggacaactccaagaacaccctgtacctgcagatgaactccctgcgggccgaggacaccgctgtgtactactgcgccagacggggctcctacccctacaactacttcgacgcttggggctgcggcaccctcgtgacagtgtctagcggagggggaggttctgggggcggaggttcaggtggtggtggttccgggggtggtggctctggtggcggtggttctggcggtggcggatctcaggctgtcgtgacccaggaacccagcctgactgtgtctcctggcggaaccgtgaccctgacctgcggatcttctaccggcgctgtgaccgccagcaactacgccaattgggtgcagcagaaacctggacagtgccctagaggcctgatcggcggccacaacaacagacctccaggcgtgccagcccggttctctggatctctgctgggcggaaaggccgctctgacactgctgggtgctcagcctgaggacgaggccgagtactactgtgccctgtggtactccgaccactgggtcatcggaggcgggaccaagctgaccgtgctgggaacacccctgggagacaccacacatactagtgggcaggccatcaagaaggagctgacccagatcaagcagaaggtggacagcctgctggagaacctggagaagatcgagaaggagggagggtcaggaggagcaccgcaccatcatcatcaccat

SEQ ID NO: 93 - protein, 588 aa, synthetic construct
  REGION 1..588: note = Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQCTKVEIKRGGGGSGGGGSGGGGSGGGGSGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGQAIKKELTQIKQKVDSLLENLEKIEKEGGSGGAPHHHHHH

SEQ ID NO: 94 - DNA, 1764 bp, synthetic construct
  misc_feature 1..1764: note = Description of Artificial Sequence: Synthetic polynucleotide
  source 1..1764: mol_type = other DNA
gatattcagatgactcagtcccctagttcactgtctgcctcagtcggagatcgggtcactatcacttgtcgggcttctcaggatgtgaacaccgccgtggcctggtaccagcagaagccaggcaaggcccccaagctgctgatctactctgccagcttcctgtattccggagtgccatctcggttttccggcagccggagcggcaccgacttcaccctgacaatcagctccctgcagcctgaggattttgccacatactattgccagcagcactataccacaccccctaccttcggccagtgcacaaaggtggagatcaagaggggaggaggaggatccggaggaggaggcagcggaggcggcggctccggcggcggcggctctggcggcggcggcagcggaggaggcggctccgaggtgcagctggtggagtccggcggcggcctggtgcagcccggcggcagcctgcggctgtcctgtgccgcctctggctttaacatcaaggacacctacatccactgggtgaggcaggcacctggcaagtgcctggagtgggtggcaaggatctatccaaccaatggctacacaagatatgccgactccgtgaagggccgctttaccatcagcgccgatacctccaagaacacagcctacctgcagatgaattctctgcgggccgaggatacagccgtgtactattgctccagatggggcggcgacggcttctatgctatggactattgggggcagggaactctggtcactgtctcctctggcggagggggatccggcggcggaggatctggcggaggtggaagtgggggaggcggatctcatgtgcagctggtggaaagcggaggcggcctggtgcagcctgggggatctctgagactgtcttgtgccgccagcggcttctccctgaccgattatggcgtgcactgggtgcgacaggcccctggcaaaggactggaatggctgggagtgatttggagtggcggaggcaccgcctacaacaccgccctgatctcccggttcaccatcagccgggacaactccaagaacaccctgtacctgcagatgaactccctgcgggccgaggacaccgctgtgtactactgcgccagacggggctcctacccctacaactacttcgacgcttggggctgcggcaccctcgtgacagtgtctagcggagggggaggttctgggggcggaggttcaggtggtggtggttccgggggtggtggctctggtggcggtggttctggcggtggcggatctcaggctgtcgtgacccaggaacccagcctgactgtgtctcctggcggaaccgtgaccctgacctgcggatcttctaccggcgctgtgaccgccagcaactacgccaattgggtgcagcagaaacctggacagtgccctagaggcctgatcggcggccacaacaacagacctccaggcgtgccagcccggttctctggatctctgctgggcggaaaggccgctctgacactgctgggtgctcagcctgaggacgaggccgagtactactgtgccctgtggtactccgaccactgggtcatcggaggcgggaccaagctgaccgtgctgggaacacccctgggagacaccacacatactagtgggcaggccatcaagaaggagctgacccagatcaagcagaaggtggacagcctgctggagaacctggagaagatcgagaaggagggagggtcaggaggagcaccgcaccatcatcatcaccat

SEQ ID NO: 95 - protein, 588 aa, synthetic construct
  REGION 1..588: note = Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRGGGGSGGGGSGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGQAIKKELTQIKQKVDSLLENLEKIEKEGGSGGAPHHHHHH

SEQ ID NO: 96 - DNA, 1764 bp, synthetic construct
  misc_feature 1..1764: note = Description of Artificial Sequence: Synthetic polynucleotide
  source 1..1764: mol_type = other DNA
gatattcagatgactcagtcccctagttcactgtctgcctcagtcggagatcgggtcactatcacttgtcgggcttctcaggatgtgaacaccgccgtggcctggtaccagcagaagccaggcaaggcccccaagctgctgatctactctgccagcttcctgtattccggagtgccatctcggttttccggcagccggagcggcaccgacttcaccctgacaatcagctccctgcagcctgaggattttgccacatactattgccagcagcactataccacaccccctaccttcggccagggcacaaaggtggagatcaagaggggaggaggaggatccggaggaggaggcagcggaggcggcggctccggcggcggcggctctggcggcggcggcagcggaggaggcggctccgaggtgcagctggtggagtccggcggcggcctggtgcagcccggcggcagcctgcggctgtcctgtgccgcctctggctttaacatcaaggacacctacatccactgggtgaggcaggcacctggcaagggcctggagtgggtggcaaggatctatccaaccaatggctacacaagatatgccgactccgtgaagggccgctttaccatcagcgccgatacctccaagaacacagcctacctgcagatgaattctctgcgggccgaggatacagccgtgtactattgctccagatggggcggcgacggcttctatgctatggactattgggggcagggaactctggtcactgtctcctctggcggagggggatccggcggcggaggatctggcggaggtggaagtgggggaggcggatctcatgtgcagctggtggaaagcggaggcggcctggtgcagcctgggggatctctgagactgtcttgtgccgccagcggcttctccctgaccgattatggcgtgcactgggtgcgacaggcccctggcaaaggactggaatggctgggagtgatttggagtggcggaggcaccgcctacaacaccgccctgatctcccggttcaccatcagccgggacaactccaagaacaccctgtacctgcagatgaactccctgcgggccgaggacaccgctgtgtactactgcgccagacggggctcctacccctacaactacttcgacgcttggggctgcggcaccctcgtgacagtgtctagcggagggggaggttctgggggcggaggttcaggtggtggtggttccgggggtggtggctctggtggcggtggttctggcggtggcggatctcaggctgtcgtgacccaggaacccagcctgactgtgtctcctggcggaaccgtgaccctgacctgcggatcttctaccggcgctgtgaccgccagcaactacgccaattgggtgcagcagaaacctggacagtgccctagaggcctgatcggcggccacaacaacagacctccaggcgtgccagcccggttctctggatctctgctgggcggaaaggccgctctgacactgctgggtgctcagcctgaggacgaggccgagtactactgtgccctgtggtactccgaccactgggtcatcggaggcgggaccaagctgaccgtgctgggaacacccctgggagacaccacacatactagtgggcaggccatcaagaaggagctgacccagatcaagcagaaggtggacagcctgctggagaacctggagaagatcgagaaggagggagggtcaggaggagcaccgcaccatcatcatcaccat

SEQ ID NO: 97 - protein, 584 aa, synthetic construct
  REGION 1..584: note = Description of Artificial Sequence: Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGQAIKKELTQIKQKVDSLLENLEKIEKEGGSGGAPHHHHHH

SEQ ID NO: 98 - DNA, 1752 bp, synthetic construct
  misc_feature 1..1752: note = Description of Artificial Sequence: Synthetic polynucleotide
  source 1..1752: mol_type = other DNA
gagatcgtgatgacccagacacccgcaacactgagcgtgtctgccggcgaaagggtcactattacctgcaaggccagtcagtcagtgtccaacgacgtgacttggtaccagcagaaaccaggccaggctccccggctgctgatctacagcgcatctaatagatatagcggagtgcctgctcgcttcagtggttcaggctatggaactgagttcaccttcaccatttccagcgtgcagtccgaagacttcgcagtgtacttttgccagcaggattattctagttttgggtgtggtacaaagctggagatcaaaaggggaggaggaggtagtggcggaggaggttcaggcggagggggtagcggcggagggggttctggcggcggcggtagtggcggcggaggtagccaggtgcagctggtcgaatccggccctggagtggtccagccaggcaggtctctgcggatcagttgcgccgtgtccggattcagcgtcaccaactacggagtgcactgggtcagacagccacctggcaagtgtctggagtggctgggagtgatctgggcaggaggaatcacaaactacaactcagcttttatgtcccgcctgactattagcaaggacaactctaaaaataccgtgtatctgcagatgaattctctgcgagccgaagataccgctatgtactattgtgcatcccgtgggggtcattacggctatgccctggattattgggggcagggtaccctggtgacagtctcatccggcggagggggatccggcggcggaggatctggcggaggtggaagtgggggaggcggatctcatgtgcagctggtggaaagcggaggcggcctggtgcagcctgggggatctctgagactgtcttgtgccgccagcggcttctccctgaccgattatggcgtgcactgggtgcgacaggcccctggcaaaggactggaatggctgggagtgatttggagtggcggaggcaccgcctacaacaccgccctgatctcccggttcaccatcagccgggacaactccaagaacaccctgtacctgcagatgaactccctgcgggccgaggacaccgctgtgtactactgcgccagacggggctcctacccctacaactacttcgacgcttggggctgcggcaccctcgtgacagtgtctagcggagggggaggttctgggggcggaggttcaggtggtggtggttccgggggtggtggctctggtggcggtggttctggcggtggcggatctcaggctgtcgtgacccaggaacccagcctgactgtgtctcctggcggaaccgtgaccctgacctgcggatcttctaccggcgctgtgaccgccagcaactacgccaattgggtgcagcagaaacctggacagtgccctagaggcctgatcggcggccacaacaacagacctccaggcgtgccagcccggttctctggatctctgctgggcggaaaggccgctctgacactgctgggtgctcagcctgaggacgaggccgagtactactgtgccctgtggtactccgaccactgggtcatcggaggcgggaccaagctgaccgtgctgggaacacccctgggagacaccacacatactagtgggcaggccatcaagaaggagctgacccagatcaagcagaaggtggacagcctgctggagaacctggagaagatcgagaaggagggagggtcaggaggagcaccgcaccatcatcatcaccat

SEQ ID NO: 99 - protein, 15 aa, synthetic construct
  REGION 1..15: note = Description of Artificial Sequence: Synthetic peptide
GGGGSGGGGSGGGGS

SEQ ID NO: 100 - protein, 30 aa, synthetic construct
  REGION 1..30: note = Description of Artificial Sequence: Synthetic polypeptide
GGGGSGGGGSGGGGSGGGGSGGGGSGGGGS

SEQ ID NO: 101 - protein, 5 aa, synthetic construct
  REGION 1..5: note = Description of Artificial Sequence: Synthetic peptide
GGGGS

SEQ ID NO: 102 - protein, 150 aa, synthetic construct
  REGION 1..150: note = Description of Artificial Sequence: Synthetic polypeptide
  VARIANT 1..150: note = MISC_FEATURE - This sequence may encompass 1-30 GGGGS repeating units
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 103 - protein, 5 aa, synthetic construct
  REGION 1..5: note = VH-CDR1
DYGVH

SEQ ID NO: 104 - protein, 16 aa, synthetic construct
  REGION 1..16: note = VH-CDR2
VIWSGGGTAYNTALIS

SEQ ID NO: 105 - protein, 11 aa, synthetic construct
  REGION 1..11: note = VH-CDR3
RGSYPYNYFDA

SEQ ID NO: 106 - protein, 14 aa, synthetic construct
  REGION 1..14: note = VL-CDR1
GSSTGAVTASNYAN

SEQ ID NO: 107 - protein, 7 aa, synthetic construct
  REGION 1..7: note = VL-CDR2
GHNNRPP

SEQ ID NO: 108 - protein, 9 aa, synthetic construct
  REGION 1..9: note = VH-CDR3
ALWYSDHWV

SEQ ID NO: 109 - protein, 8 aa, synthetic construct
  REGION 1..8: note = VH-CDR1
GFSVTNYG

SEQ ID NO: 110 - protein, 7 aa, synthetic construct
  REGION 1..7: note = VH-CDR2
IWAGGIT

SEQ ID NO: 111 - protein, 13 aa, synthetic construct
  REGION 1..13: note = VH-CDR3
ASRGGHYGYALDY

SEQ ID NO: 112 - protein, 6 aa, synthetic construct
  REGION 1..6: note = VL-CDR1
QSVSND

SEQ ID NO: 113 - protein, 6 aa, synthetic construct
  REGION 1..6: note = VH-CDR3
QQDYSS

SEQ ID NO: 114 - protein, 8 aa, synthetic construct
  REGION 1..8: note = VH-CDR1
GFNIKDTY

SEQ ID NO: 115 - protein, 8 aa, synthetic construct
  REGION 1..8: note = VH-CDR2
IYPTNGYT

SEQ ID NO: 116 - protein, 13 aa, synthetic construct
  REGION 1..13: note = VH-CDR3
SRWGGDGFYAMDY

SEQ ID NO: 117 - protein, 6 aa, synthetic construct
  REGION 1..6: note = VL-CDR1
QDVNTA

SEQ ID NO: 118 - protein, 9 aa, synthetic construct
  REGION 1..9: note = VH-CDR3
QQHYTTPPT

(19) United States
(12) Patent Application Publication
(10) Pub. No.: US 2025/0295794 A1
(43) Pub. Date: Sep. 25, 2025

(54) MODULAR SELF ASSEMBLY DISASSEMBLY (SADA) TECHNOLOGIES

(71) Applicant: Memorial Sloan Kettering Cancer Center, New York, NY (US)

(72) Inventors: Brian H. Santich, New York, NY (US); Mahiuddin Ahmed, New York, NY (US); Nai-Kong V. Cheung, New York, NY (US)

(73) Assignee: Memorial Sloan Kettering Cancer Center, New York, NY (US)

(21) Appl. No.: 19/231,378

(22) Filed: Jun. 6, 2025

Related U.S. Application Data
(62) Division of application No. 18/153,228, filed on Jan. 11, 2023, now Pat. No. 12,343,401.
Application No. 18/153,228 is a division of application No. 16/609,401, filed on Oct. 29, 2019, now Pat. No. 11,583,588.
(60) Provisional application No. 62/502,151, filed on May 5, 2017.

(51) Int. Cl. A61K 47/64; A61K 47/54; A61K 51/04; A61K 51/10; C07K 14/47; C07K 14/715; C07K 16/30; C07K 16/44

(52) U.S. Cl. CPC A61K 47/641; A61K 47/547; A61K 51/0495; A61K 51/10; A61K 51/1096; C07K 14/4746; C07K 14/7155; C07K 16/3084; C07K 16/44; C07K 2317/622; C07K 2317/92; C07K 2317/94; C07K 2319/70

(57) ABSTRACT

The present invention relates to compositions and methods employing conjugates that include a self-assembly and disassembly (SADA) polypeptide and a binding domain. The present invention encompasses the recognition that conjugates with a SADA polypeptide have certain improved biological properties. SADA-conjugates are described, along with uses thereof (e.g., as therapeutic or diagnostic agents) and methods of manufacture.

### CROSS-REFERENCE TO RELATED APPLICATIONS

This application is a divisional of U.S. application Ser. No. 18/153,228, filed Jan. 11, 2023, which is a divisional of U.S. application Ser. No. 16/609,401, filed Oct. 29, 2019, which is a National Stage Application of PCT/US2018/031235, filed May 4, 2018, which claims the benefit of and priority to U.S. Provisional Application No. 62/502,151, filed May 5, 2017, each of which is incorporated herein by reference in its entirety.

### STATEMENT OF GOVERNMENT SUPPORT

This invention was made with government support under CA008748 awarded by the National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on Dec. 13, 2024, is named 115872-2681_SL.xml and is 237,295 bytes in size.

### BACKGROUND

Effective delivery of therapeutic and diagnostic agents to human and animal subjects can present significant challenges.

### SUMMARY

The present disclosure provides, among other things, a novel platform technology using modular domains for self-assembly and disassembly (SADA). The present disclosure encompasses a recognition that SADA domains can impart certain desirable functional characteristics to a conjugate. For example, the present disclosure provides an insight that SADA domains can be designed and/or tailored to achieve environmentally-dependent multimerization with beneficial kinetic, thermodynamic, and/or pharmacologic properties. For example, it is recognized that SADA domains may be part of a conjugate that permit effective delivery of a payload to a target site of interest while minimizing risk of off-target interactions.

Among other things, the present disclosure provides various conjugates comprising a SADA domain linked to one or more binding domains. In some embodiments, such conjugates are characterized in that they multimerize to form a complex of a desired size under relevant conditions (e.g., in a solution in which the conjugate is present above a threshold concentration or pH and/or when present at a target site characterized by a relevant level or density of receptors for the payload), and disassemble to a smaller form under other conditions (e.g., absent the relevant environmental multimerization trigger).

The present disclosure provides an appreciation that assembly/disassembly through a SADA domain enables, at least in part, transition between a first multimeric state (e.g., monomeric or dimeric) and higher order multimeric states (e.g., tetrameric, pentameric, etc.) to occur with predictable kinetics. In some embodiments, a SADA conjugate is characterized in that it forms a higher order multimeric complex that is highly stable in solution at relevant conditions (e.g., sufficiently high concentration or relevant pH). In some embodiments, a SADA conjugate is characterized in that a higher order multimeric complex dissociates to smaller states (e.g., dimers, monomers) with predictable kinetics under conditions that do not meet a multimerization threshold (e.g., below a threshold concentration). In some embodiments, a SADA domain is selected and/or engineered for tunable delivery of a conjugate in vivo (e.g., selected for particular association and/or dissociation kinetics of a SADA domain).

The present disclosure provides, among other things, an appreciation that a SADA conjugate may have improved characteristics compared to a conjugate without a SADA domain. In some embodiments, a SADA conjugate includes a binding domain. In some embodiments, improved characteristics include that a multimeric conjugate has increased avidity/binding to a target, increased specificity for target cells or tissues, and/or extended initial serum half-life. In some embodiments, improved characteristics include that SADA conjugates exhibit reduced non-specific binding, decreased toxicity, and/or improved renal clearance, which may be due, at least in part, through dissociation to smaller states (e.g., dimeric or monomeric).

In some embodiments, a SADA conjugate further comprises a payload. In some embodiments, a SADA conjugate has improved characteristics when compared with a payload not conjugated to a SADA domain or with a payload conjugated to an alternative domain (e.g., an immunoglobulin domain).

In some embodiments, a multimeric SADA conjugate is highly stable in a solution in which the conjugate is present above a threshold concentration. In some embodiments a threshold concentration is 1 nM, 5 nM, 10 nM, 50 nM, 100 nM, 500 nM, 1 mM, 5 mM, 10 mM, 50 mM, 100 mM, 500 mM, 1 μM, 10 μM, 50 μM, 100 μM, 200 μM, 300 μM, 400 μM, 500 μM, 1 mM, etc. In some embodiments, a multimeric SADA conjugate is highly stable in a solution in which the conjugate is present above or below a threshold pH. In some embodiments, a multimeric SADA conjugate under relevant conditions is stable for at least a day, at least a week, at least two weeks, at least a month, at least two months, at least 3 months, at least 6 months, etc., when stored at −80° C., −20° C., 0° C., 20° C., 25° C. or 37° C. In some embodiments, a multimeric SADA conjugate is highly stable under in vivo conditions where the local environment (e.g., a target cell and/or a target tissue) meets multimerization threshold conditions (e.g., local concentration is above a threshold concentration, target density is above a threshold, or at a threshold pH).

In some embodiments, a multimeric SADA conjugate dissociates at a predictable rate under conditions that do not meet the multimerization threshold (e.g., below a threshold concentration). In some embodiments, a SADA conjugate multimer dissociates rapidly under conditions that do not meet the multimerization threshold (e.g., below a threshold concentration or an a pH above/below the relevant pH). In some embodiments, a SADA conjugate multimer dissociates at a relatively slow rate under conditions that do not meet the multimerization threshold. In some embodiments, a SADA conjugate multimer dissociates under conditions that do not meet the multimerization threshold with a k_{off} rate in a range of about 1×10⁻⁷ sec⁻¹ to 1×10⁻³ sec⁻¹. In some embodiments, a SADA conjugate multimer dissociates under conditions that do not meet the multimerization threshold with a k_{off} rate in a range of about 1×10⁻⁶ sec⁻¹ to 5×10⁻⁴ sec⁻¹. In some embodiments, a SADA conjugate multimer dissociates under conditions that do not meet the multimerization threshold with a half life of about 10 min, 20 min, 30 min, 40 min, 50 min, 60 min, 70 min, 80 min, 90 min, 100 min, 125 min, 150 min, 175 min, 200 min, 225 min, 250 min, 275 min, 300 min, 325 min, 350 min, 375 min, or 400 min.

In some embodiments, a SADA conjugate has predictable kinetics in vivo. In some embodiments, a multimerized SADA conjugate has an extended initial serum half-life. In some embodiments, such conjugates are characterized in that they multimerize to form a complex with a molecular weight greater than the threshold for renal clearance (i.e., greater than ^{˜}70 kDa). In some embodiments, a SADA conjugate multimer dissociates under in vivo conditions that do not meet a multimerization threshold (e.g., the do not meet a threshold concentration, such as at an off-target site). In some embodiments, dissociation of a multimerized SADA conjugate into a small units facilitates rapid clearance in vivo (e.g., through the renal clearance system). In some embodiments, a SADA conjugate monomer has a molecular weight less than the threshold for renal clearance (i.e., less than ^{˜}70 kDa). In some embodiments, a SADA conjugate dimer has a molecular weight less than the threshold for renal clearance (i.e., less than ^{˜}70 kDa).

In some embodiments, a multimerized SADA conjugate has a molecular weight greater than 150 kDa and rapidly dissociates to a smaller state (e.g., dimer or monomer of less than ^{˜}70 kDa) under in vivo conditions that do not meet the multimerization threshold (e.g., at off target sites in vivo). In some embodiments, a multimerized SADA conjugate has a molecular weight greater than 150 kDa and dissociates to a smaller state (e.g., dimer or monomer of less than ^{˜}70 kDa) under in vivo conditions that do not meet the multimerization threshold (e.g., at off target sites in vivo) over a discrete period.

In some embodiments, a SADA conjugate comprises (i) a self-assembly disassembly (SADA) polypeptide having an amino acid sequence that shows at least 75% identity (e.g., 75%, 80%, 85%, 90%, 95%, 96%, 97%, 98%, 99%, or 100% identity) with that of a human homo-multimerizing polypeptide and is characterized by one or more multimerization dissociation constants (K_{D}); and (ii) at least a first binding domain that binds to a first target and is covalently linked to the SADA polypeptide. In some embodiments, a SADA conjugate is constructed and arranged so that it adopts a first multimerization state and one or more higher-order multimerization states. In some embodiments, a first multimerization state is less than about ^{˜}70 kDa in size. In some embodiments, a first multimerization state is an unmultimerized state (e.g., a monomer or a dimer). In some embodiments, a first multimerization state is a monomer. In some embodiments, a first multimerization state is a dimer. In some embodiments, a first multimerization state is a multimerized state (e.g., a trimer or a tetramer). In some embodiments, a higher-order multimerization state is a homo-tetramer or higher-order homo-multimer greater than 150 kDa in size. In some embodiments, a higher-order homo-multimerized conjugate is stable in aqueous solution when the conjugate is present at a concentration above the SADA polypeptide K_{D}. In some embodiments, a SADA conjugate transitions from a higher-order multimerization state(s) to a first multimerization state under physiological conditions when the concentration of the conjugate is below the SADA polypeptide K_{D}.

In some embodiments, a higher-order homo-multimerized conjugate is stable for a period of at least 24 hours at a temperature from 25° C. to 37° C. in an aqueous buffer with a pH of about 6.8-7.2. In some embodiments, a higher-order homo-multimerized conjugate is stable for a period of at least 48 hours, 72 hours, 1 week, 2 weeks, 1 month, 2 months, 3 months, or more. In some embodiments, a higher-order homo-multimerized conjugate is stable over 3, 4, 5, or more freeze-thaw cycles.

In some embodiments, a conjugate transitions from a higher order multimerization state to a first multimerization state, and this transition is characterized by a K_{off} within a range of 1×10⁻⁶ to 1×10⁻⁴ (s⁻¹).

In some embodiments, a SADA polypeptide has a total buried surface area of 900 Å2 to 4000 Å2. In some embodiments, a SADA polypeptide lacks unpaired cysteine residues. In some embodiments, a SADA polypeptide comprises a tetramerization, pentamerization or hexamerization domain.

In some embodiments, a SADA polypeptide is or comprises a tetramerization domain of p53, p63, p73, hnRNPC, SNAP-23, Stefin B, KCNQ4, or CBFA2T1. In some embodiments, a SADA polypeptide is or comprises a sequence that is at least 75%, 80%, 85%, 90%, 91%, 92%, 93%, 94%, 95%, 96%, 97%, 98%, 99% or 100% identical to a sequence as set forth in any one of SEQ ID NOs: 1, 3, 5, 7, 9, 11, 13, and 15.

In some certain embodiments, a conjugate comprising a SADA polypeptide is or comprises a sequence that is at least 75%, 80%, 85%, 90%, 91%, 92%, 93%, 94%, 95%, 96%, 97%, 98%, 99% or 100% identical to a sequence as set forth in any one of SEQ ID NOs: 17, 19, 21, 23, 25, 27, 29, 31, 33, 35, 37, 39, 41, 43, 45, 47, 49, 51, 53, 55, 57, 59, 61 and 63. In some certain embodiments, a conjugate comprising a SADA polypeptide is or comprises a sequence that is at least 75%, 80%, 85%, 90%, 91%, 92%, 93%, 94%, 95%, 96%, 97%, 98%, 99% or 100% identical to a sequence as set forth in any one of SEQ ID NOs: 17, 19, 21, 23, 25, 27, 29, 31, 33, 35, 37, 39, 41, 43, 45, 47, 49, 51, 53, 55, 57, 59, 61, 63, 67, 69, 71, 73, 75, 77, 79, 81, 83, 85, 87, 89, 91, 93, 95, and 97.

In some embodiments, a conjugate comprises a first binding domain that binds to a first target selected from the group consisting of an in situ target and a payload target. In some embodiments, a first target is an in situ target that is or comprises an entity selected from the group consisting of: a cell-surface moiety, a cytokine, a receptor ligand, a peptide, a hormone, a metabolite, and a hapten. In some embodiments, a first target is a therapeutic payload. In some embodiments, a first target is a diagnostic payload.

In some embodiments, a conjugate further comprises a second binding domain that binds to a second target, which is different from the first target. In some embodiments, a conjugate comprises at least two binding domains and wherein the conjugate in the second multimerization state is at least octavalent. In some embodiments, a second target is selected from the group consisting of an in situ target and a payload target. In some embodiments, a second target is an in situ target that is or comprises an entity selected from the group consisting of: a cell-surface moiety, a cytokine, a receptor ligand, a peptide, a hormone, a metabolite, and a hapten. In some embodiments, a second target is a therapeutic payload. In some embodiments, a second target is a diagnostic payload.

In some embodiments, a payload target is a drug, a polypeptide (such as a toxin, enzyme, cytokine, chemokine, receptor, or biologic), a chemical probe (such as a fluorescent dye or biotin tag), a radioactive isotope, or a nanoparticle. In some embodiments, a second target is a cell surface moiety. In some embodiments, a cell surface moiety is specifically expressed or enriched on a subset of cells in an organism. In some embodiments, a cell surface moiety is specifically expressed or enriched on tumor cells. In some embodiments, a cell surface moiety is a cell surface receptor. In some embodiments, a first and/or second binding domain is or comprises a ligand for a cell surface receptor. In some embodiments, a first and/or second binding domain is or comprises a cytokine receptor binding domain. In some embodiments, a conjugate is further complexed with a soluble cytokine polypeptide. In some embodiments, a cytokine receptor is IL15Rα and the soluble cytokine polypeptide is IL15.

In some embodiments, a first and/or second binding is or comprises an antibody component specific for a cell surface target. In some embodiments, a first and/or second binding domain may be any polypeptide whose amino acid sequence includes elements characteristic of an antibody-binding region. In some embodiments, a first and/or second binding domain is a VHH. In some embodiments, a first and/or second binding domain is a scFv. In some embodiments, a first and/or second binding domain is an anti-GD2, anti-Globo H, anti-GPA33, anti-PSMA, anti-polysialic acid, anti-Lew, anti-L1CAM, anti-HER2, anti-B7H3, anti-CD33, anti-peptide/MHC, anti-glypican3, or anti-GD3 antibody component.

In some embodiments, a SADA conjugate is characterized in that it comprises a binding domain that binds a target at an in vivo site. In some embodiments, a target at an in vivo site is present at sufficient density such that a conjugate is substantially in the higher-order multimerization state at the target site. In some embodiments, a SADA conjugate is characterized in that it comprises a binding domain that binds a target, wherein the target is present at sufficient concentration such that higher order multimerization state of the SADA polypeptide is stabilized in vivo.

In some embodiments, a SADA conjugate further comprises a second multimerization domain (e.g., a dimerization domain, a trimerization domain, a tetramerization domain, or a second SADA domain). In some embodiments, a SADA conjugate can exist in one or more additional multimeric states.

In some embodiments, a SADA conjugate is substantially not immunogenic in a human subject.

In some embodiments, a payload is a therapeutic payload. In some embodiments, a payload is a diagnostic payload. In some embodiments a payload is or comprises a radioisotope, an antibody agent, a cytokine, a cytotoxic agent, a polypeptide, a protein toxin, a ligand binding domain, a peptide and/or a nanoparticle.

In some embodiments, a SADA conjugate comprises a first binding domain that is an antibody component (e.g., an antibody, a scFv, a VHH, etc.). In some embodiments, a SADA conjugate further comprises a second binding domain, wherein the second binding domain is an antibody component (e.g., an antibody, a scFv, a VHH, etc.). In some embodiments, a first and/or second binding domains are part of a bispecific antibody agent. In some embodiments, a bispecific antibody agent is a tandem scFv comprising a first binding domain that binds a tumor target and a second binding domain that binds a metal-Bn-DOTA. In some embodiments, a bispecific antibody agent is a tandem scFv comprising a first binding domain that binds a tumor target and a second binding domain that binds an immune-cell activating receptor. In some embodiments, a first binding domain that binds a tumor target is an anti-GD2, anti-Globo H, anti-GPA33, anti-PSMA, anti-polysialic acid, anti-Lew, anti-L1CAM, anti-HER2, anti-B7H3, anti-CD33, anti-peptide/MHC, anti-glypican3, or anti-GD3 binding domain (e.g., an antibody component). In some embodiments, a first binding domain that binds a tumor target is an antibody component. In some embodiments, an antibody component is an scFv. In some embodiments, an antibody component is a VHH.

Also provided are nucleic acid sequences encoding SADA domains and SADA-domain containing conjugates, as well as vectors comprising such nucleic acid sequences. In some embodiments, a nucleotide sequence encoding a SADA polypeptide is or comprises a sequence that is at least 75%, 80%, 85%, 90%, 91%, 92%, 93%, 94%, 95%, 96%, 97%, 98%, 99% or 100% identical to a sequence as set forth in any one of SEQ ID NOs: 2, 4, 6, 8, 10, 12, 14 and 16. In some certain embodiments, a nucleotide sequence encoding a conjugate comprising a SADA polypeptide is or comprises a sequence that is at least 75%, 80%, 85%, 90%, 91%, 92%, 93%, 94%, 95%, 96%, 97%, 98%, 99% or 100% identical to a sequence as set forth in any one of SEQ ID NOs: 18, 20, 22, 24, 26, 28, 30, 32, 34, 36, 38, 40, 42, 44, 46, 48, 50, 52, 54, 56, 58, 60, 62 and 64. In some certain embodiments, a nucleotide sequence encoding a conjugate comprising a SADA polypeptide is or comprises a sequence that is at least 75%, 80%, 85%, 90%, 91%, 92%, 93%, 94%, 95%, 96%, 97%, 98%, 99% or 100% identical to a sequence as set forth in any one of SEQ ID NOs: 18, 20, 22, 24, 26, 28, 30, 32, 34, 36, 38, 40, 42, 44, 46, 48, 50, 52, 54, 56, 58, 60, 62, 64, 68, 70, 72, 74, 76, 78, 80, 82, 84, 86, 88, 90, 92, 94, 96, and 98.

Also provided are cells (e.g., host cells) comprising nucleic acids and/or vectors encoding SADA domains or SADA conjugates. In some embodiments, a host cell comprises a vector that comprises a nucleotide sequence encoding a SADA domain or a SADA conjugate. In some embodiments, a host cell is selected from the group consisting of a bacterial, yeast, insect or mammalian cell. In some embodiments, a host cell is selected from the group consisting of E. coli, Pichia pastoris, Sf9, COS, HEK293 and a CHO cell.

Also provided are compositions comprising one or more SADA conjugates. In some embodiments, a composition comprising a SADA conjugate is formulated for injection. In some embodiments, a SADA conjugate is formulated for injection so that stable binding between the conjugate and its target is detectable at its target tissue for a period of time at least 24 hours long, and wherein the conjugate is substantially undetectable in at least one non-target tissue within 72 hours post-injection without any extraneous drug or clearing agent. In some embodiments, a non-target tissue may be or include blood, gastrointestinal tissue, lymphoid tissue, nervous system tissue, renal tissue, hepatic tissue, muscle tissue, or any combinations thereof. In some embodiments, a non-target tissue is or comprises blood. In some certain embodiments, a target tissue is or comprises a tumor tissue. In some embodiments, a SADA conjugate is cleared from the blood serum of a subject within 30 minutes, within 1 hour, within 2 hours, within 3 hours, within 4 hours, within 5 hours, within 6 hours, within 12 hours, within 24 hours, within 36 hours, within 48 hours, within 72 hours, etc.

In some embodiments, a method is provided, said method comprising steps of (i) providing a liquid composition comprising a SADA conjugate in the higher-order multimeric state; and (ii) administering the composition to a subject. In some embodiments, a step of administering comprises delivering so that conjugate that is not bound to the target tissue disassembles into the first multimerization state or a monomeric state, whereas conjugate that is bound to the target is substantially in the higher-order multimeric state. In some embodiments, extent of a conjugate in a higher-order multimeric state may be or is assessed by measuring the retention of a conjugate at a target site. In some embodiments, extent of conjugate in a first multimerization state or monomeric state may be or is assessed by measuring an amount of conjugate in the blood of a subject. In some embodiments, extent of conjugate in a first multimerization state or monomeric state may be or is assessed by direct radiolabeling. In some embodiments, extent of conjugate in a first multimerization state or monomeric state may be or is assessed by measuring a rate of clearance of a conjugate into the urine of a subject. In some embodiments, a step of administering is to a subject suffering from or susceptible to cancer. In some embodiments, a SADA conjugate is cleared from the blood serum of a subject within 30 minutes, within 1 hour, within 2 hours, within 3 hours, within 4 hours, within 5 hours, within 6 hours, within 12 hours, within 24 hours, within 36 hours, within 48 hours, within 72 hours, etc.

In some embodiments, a method is provided, said method comprising steps of (i) providing a liquid composition comprising a SADA conjugate; and (ii) administering the composition to a subject that is suffering from cancer.

In some embodiments, a method of treating or diagnosing cancer in a subject is provided, said method comprising steps of (i) providing a liquid composition comprising a SADA conjugate in a concentration sufficient that greater than 90% of the conjugate is in the higher-order multimerization state; and (ii) administering the composition to a subject that is suffering from or susceptible to cancer. In some embodiments, a composition comprises a conjugate at a concentration within a range of about 100 nM to 10 mM.

In some embodiments, a method of pre-targeted radio immunotherapy is provided, said method comprising steps of (i) providing a liquid composition comprising a SADA conjugate in a higher order multimeric form; (ii) administering the composition to a subject that is suffering from or susceptible to cancer; and (ii) subsequently administering a radiolabeled Bn-DOTA to the subject. In some embodiments, such a method does not include administration of a clearing agent. In some embodiments, a SADA conjugate is cleared from the blood serum of a subject within 30 minutes, within 1 hour, within 2 hours, within 3 hours, within 4 hours, within 5 hours, within 6 hours, within 12 hours, within 24 hours, within 36 hours, within 48 hours, within 72 hours, etc.

In some certain embodiments, the present disclosure provides the insight that SADA-conjugate platform as described herein may be particularly useful, for example, in context of a pre-targeted therapy. In some embodiments, a method of pre-targeted radio immunotherapy is provided, said method comprising steps of (i) providing a liquid composition comprising a SADA conjugate in a concentration of at least 50 nM, 100 nM, 500 nM, 1 μM, 10 μM, 50 μM, 100 μM, 200 μM, 300 μM, 400 μM, 500 μM, or 1 mM; and (ii) administering the composition to a subject that is suffering from or susceptible to cancer. In some embodiments, a liquid composition comprises a conjugate, where at least 90% of the conjugate is in a higher order multimeric form (e.g., a tetramer, pentamer, hexamer, septamer, octamer, nonamer, decamer, etc.). In some embodiments, the conjugate is a SADA-Bispecific DOTA-engaging (SADA-BiDE) conjugate. In some embodiments, the conjugate further comprises a payload, such as Bn-DOTA. In some embodiments, a payload is or comprises Bn-DOTA or a variant thereof. In some embodiments, a Bn-DOTA variant may also comprise a biotin tag, a fluorescent tag, another DOTA tag, or a peptide tag, etc. In some embodiments, a Bn-DOTA or variant thereof is covalently attached to the conjugate. In some embodiments, a Bn-DOTA or variant thereof is non-covalently complexed with the conjugate. In some embodiments, a Bn-DOTA is radiolabeled. In some embodiments, a radiolabeled Bn-DOTA is covalently attached to the conjugate. In some embodiments, a radiolabeled Bn-DOTA is non-covalently complexed with the conjugate. In some embodiments, such a method does not include administration of a clearing agent. In some embodiments, a SADA conjugate is cleared from the blood serum of a subject within 30 minutes, within 1 hour, within 2 hours, within 3 hours, within 4 hours, within 5 hours, within 6 hours, within 12 hours, within 24 hours, within 36 hours, within 48 hours, within 72 hours, etc.

In some embodiments, a method is provided, said method comprising steps of (i) providing a liquid composition comprising a SADA conjugate, wherein at least 90% of the conjugate in the composition is in ae higher order multimeric form; and (ii) administering the composition to a subject from whom a target entity is to be removed, wherein the conjugate is capable of binding the target entity.

The present disclosure provides various technologies for identifying and/or characterizing such conjugates, compositions containing them, and/or useful components thereof. The present disclosure provides, among other things, a recognition of certain characteristics that may be used to select a polypeptide for use as SADA domain. In some embodiments, a SADA domain is a human polypeptide or a fragment and/or derivative thereof. In some embodiments, a SADA domain is substantially non-immunogenic in a human. In some embodiments, a SADA polypeptide is stable as a multimer. In some embodiments, a SADA polypeptide lacks unpaired cysteine residues. In some embodiments, a SADA polypeptide does not have large exposed hydrophobic surfaces. In some embodiments, a SADA domain has or is predicted to have a structure comprising helical bundles that can associate in a parallel or anti-parallel orientation. In some embodiments, a SADA polypeptide is capable of reversible multimerization. In some embodiments, a SADA domain is a tetramerization domain, a heptamerization domain, a hexamerization domain or an octamerization domain. In certain embodiments, a SADA domain is a tetramerization domain. In some embodiments, a SADA polypeptide comprises a multimerization domains from one of following human proteins: p53, p63, p73, heterogeneous nuclear Ribonucleoprotein C (hnRNPC), N-terminal domain of Synaptosomal-associated protein 23 (SNAP-23), Stefin B (Cystatin B), Potassium voltage-gated channel subfamily KQT member 4 (KCNQ4), or Cyclin-D-related protein (CBFA2T1).

In some embodiments, a SADA-conjugate may be identified or characterized by a method comprising steps of (i) providing a conjugate comprising a self-assembly disassembly (SADA) polypeptide and a binding domain, (ii) administering the composition to a subject and (iii) determining the affinity of the conjugate for a target. Any methods known in the art for determining the affinity of a conjugate for a target may be used. In some embodiments, affinity may be assessed as binding affinity. In some embodiments, affinity may be assessed by localization, using any techniques known in the art to visualize localization.

In some embodiments, a SADA-conjugate may be identified or characterized by a method that includes analysis of one or more conjugates in a plurality of conjugates. In some embodiments, a SADA-conjugate may be identified or characterized by a method comprising steps of (i) providing composition comprising a plurality of conjugates, each comprising a SADA polypeptide and a binding domain, (ii) administering the composition to a subject and (iii) determining the affinity of one or more of the conjugates for a target. In some embodiments, a step of determining comprises determining the affinity for a target for each of the conjugates. In some embodiments, a method includes a step of determining the rate of clearance of one or more conjugates from blood. In some embodiments, a method includes a step of determining the rate of clearance of a conjugate from blood for each of a plurality of conjugates. In some embodiments, a plurality of conjugates includes SADA conjugates that comprise the same binding domain but differ in the SADA polypeptide.

In some embodiments, a SADA-conjugate may be identified or characterized as preferred relative to another conjugate in a plurality of conjugates when the preferred conjugate shows increased avidity for a target and/or when the preferred conjugate is more rapidly cleared from the blood.

In some embodiments, a SADA-conjugate may be identified or characterized by a method that includes steps of (i) providing a composition comprising a SADA conjugate, and (ii) formulating the conjugate with a pharmaceutically acceptable carrier or excipient to produce a composition in which the conjugate is present at a concentration sufficient for at least 90% of the conjugate to adopt the higher-order multimerized state. In some embodiments, a conjugate in the composition is at a concentration of about 50 nM, 100 nM, 500 nM, 1 μM, 10 μM, 50 μM, 100 μM, 200 μM, 300 μM, 400 μM, 500 μM, 1 mM, or more.

The present disclosure provides various technologies related to SADA-containing conjugates including, for example, technologies for making such conjugates and/or compositions containing them, technologies for using such conjugates and/or compositions containing them, and/or technologies related to the manufacture of preparations comprising such conjugates.

### BRIEF DESCRIPTION OF THE DRAWING

The Drawing included herein, which is composed of the following Figures, is for illustration purposes only and not for limitation.

FIG. 1A to FIG. 1C illustrate different treatment strategies and exemplifies some unique properties of a SADA domain. FIG. 1A depicts a conventional three-step pretargeting treatment schematic (e.g. radioimmunotherapy, RIT) using an IgG-based targeting agent. Initially (1a) the targeting agent is delivered, followed by a waiting period (1b) where the targeting agent is allowed to bind to its target. After a period of time (e.g., several hours or days), a (2a) clearing agent is administered, which binds and (2b) clears excess targeting agent (e.g., in a matter of hours). Lastly a third step involves the (3a) administration of the payload agent, which is small and can rapidly permeate tissues and bind to a targeting agent. Excess payload agent is (3b) rapidly cleared through the kidneys in a matter of minutes to hours. FIG. 1B depicts a two-step pretargeting treatment strategy using a SADA therapeutic. Initially (1a) the SADA targeting agent is delivered followed by (1b) a waiting period where the SADA targeting agent either binds to its target, or disassembles into monomeric units that are rapidly cleared by the kidneys in a matter of hours to days. The second step involves the administration of (2a) the payload agent that is specific for the SADA targeting agent, which is very small and rapidly permeates the tissues to reach the SADA targeting agent. Excess payload agent is rapidly cleared (2b) through the kidneys (e.g., in a matter of minutes to hours). FIG. 1C depicts a one-step treatment strategy using a SADA therapeutic. Initially (1a) the SADA targeting agent is delivered followed by (1b) a waiting period where the SADA therapeutic agent either binds to its target, or disassembles into monomeric units that are rapidly cleared by the kidneys (e.g., in a matter of hours to days). No other steps are needed and the SADA therapeutic imparts it activity onto its target.

FIG. 2 depicts a schematic of an exemplary conjugate, SADA-Bispecific DOTA-engaging (BiDE), made up of a SADA domain and two binding domains, that may be useful for pre-targeted radioimmunotherapy (PRIT). The diagram illustrates self-assembly and disassembly of a SADA-BiDE into three states: Tetramer (full), Dimer (half), and Monomer (quarter). Black Stars represent bound or unbound payload (i.e. Bn-DOTA). Dark gray boxes represent a SADA domain (shown as the most inner/proximal domain when assembled) (i.e. a human p53-tetramerization domain for P53-BIDE; a human p63 tetramerization domain P63-BiDE and a p73 tetramerization domain for P73-BiDE). Light gray boxes represent first binding domain that binds a payload (i.e., a Bn-DOTA binding domain, such as huC825-scFv). White boxes represent a second binding domain (most distal domain when assembled) that binds a cellular component (e.g., the cell surface tumor cell marker GD2, such as hu3F8-scFv). Black arrows indicate self-assembly of the construct and gray arrows indicate disassembly of the construct.

FIG. 3A to FIG. 3C depict experiments showing the purity and stability of a preparation of SADA-BiDEs. FIG. 3A depicts an HPLC chromatogram that shows the size and purity of a preparation of three SADA-BiDEs after single-step affinity purification. The main peak (^{˜}16 min) denotes the self-assembled tetramer, similar to an IgG-BiDE (Cheal, S. M. et al. (2014) Mol Cancer Ther), matching its calculated molecular weight of ^{˜}200 kDa. The earlier peak (^{˜}14 min) denotes some smaller aggregates of each SADA-BiDE (2-3 complexes). The last peak (^{˜}25 min) is a non-specific peak from the storage buffer (sodium citrate). Plots are normalized to the standard ran that same week. P53-BiDE is depicted in black. P63-BiDE is depicted in dark gray. P73-BiDE is depicted in light gray. The purity (percentage tetramer) of each SADA-BiDE is noted by the main peak. FIG. 3B depicts a summary of HPLC chromatograms of various SADA-BiDEs incubated at 37° C. for a 40 day period. Each line denotes the purity of the SADA-BiDE (fraction that is complete tetramer) over time. P53-BiDE is depicted in black. P63-BiDE is depicted in dark gray. P73-BiDE is depicted in light gray. FIG. 3C depicts a normalized HPLC chromatogram showing the purity of the original SADA-BiDE compared to the purity after the sample is repeatedly frozen and thawed (5 times from −80° C. to 25° C.). The main peak (^{˜}16 min) denotes the self-assembled tetramer. The earlier peak (^{˜}14 min) denotes a higher order aggregate (2-3 complexes). The last peak (^{˜}25 min) is from the storage buffer (sodium citrate). Plots are normalized to a standard ran that same week. P53-BiDE is depicted in black. P63-BiDE is depicted in dark gray. P73-BiDE is depicted in light gray. Solid lines refer to the original purity, dotted lies refer to the purity after the freeze/thaw cycles.

FIG. 4 depicts a summary of fluorescence correlation spectroscopy (FCS) experiment regarding the SADA domains used here. Specifically, P53-BiDE, P63-BiDE and P73-BiDE were labeled with a Cy3-labeled ¹⁷⁵Lu-Bn-DOTA, quickly diluted down to low concentrations, and then fluctuations in fluorescent intensity were measure over the course of 2 hours. Measurements were taken with a Zeiss LSM 880 confocal microscope. Normalized autocorrelations functions G(T) were then plotted to determine the diffusion times for each SADA-BiDE over time. All samples were compared against a monomeric anti-GD2 BiDE. P53-BiDE is depicted in black. P63-BiDE is depicted in dark gray. P73-BiDE is depicted in light gray.

FIG. 5A and FIG. 5B depict target binding affinity and tumor cell binding activity of exemplary SADA constructs. Figure SA depicts normalized SPR curves (Biacore T100) for P53-BiDE (solid black line), P63-BiDE (solid dark gray line) and P73-BiDE (solid light gray line). A corresponding IgG-BiDE (Cheal, S. M. et al. (2014) Mol Cancer Ther) (dotted line) and an anti-GD2 IgG control (dashed line). Each construct was run in a concentration series (400 nM-0 nM) over a GD2-coated CM5 chip. The plotted curves were normalized to both start and end of the binding phases for comparison. Figure SB depicts a histogram overlay of FACS plots of three SADA-BiDE relative to an IgG-BiDE (Cheal, S. M. et al. (2014) Mol Cancer Ther) binding against GD2(+) luciferase-transfected IMR32 and M14 tumor cell lines. 1 μg of either (top to bottom) P53-BIDE, P63-BIDE, P73-BIDE, IgG-BiDE (Cheal, S. M. et al. (2014) Mol Cancer Ther) a control protein was incubated with 1M cells at 4° C. for 30 min. A Cy5-labeled ¹⁷⁵Lu-Bn-DOTA was used to detect and quantify the amount of bound complex.

FIG. 6A to FIG. 6E depict pharmacokinetics of exemplary SADA-BiDE constructs in vivo. FIG. 6A depicts activity over time after P53-BiDE(noHIS) and Bn-DOTA administration. Each line represents one group, with three mice per group. Triangles denote a group that received P53-BiDE(noHIS) followed by clearing agent (CA) 72 hours later. Squares denote a group that received P53-BiDE(noHIS) without any clearing agent before ¹⁷⁷Lu-Bn-DOTA administration. Circles denote a group that only received ¹⁷⁷Lu-Bn-DOTA but not any SADA-BiDE. Dashed lines correspond to the measured blood activity, while solid lines correspond to the activity measured in the tumor. For The Bn-DOTA alone, no tumor activity was detected. FIG. 6B depicts blood activity of radiolabeled ¹³¹I-SADA-BiDE in tumor-free mice. Activity measurements were normalized to the initial measurement for each group. Each line represents one group, with 4-5 mice per group. (+) symbols denote P53-BiDE, (X) symbols denote P63-BiDE and circles denote P73-BiDE. FIG. 6C depicts blood activity in tumor bearing mice treated with either IgG-BiDE (Cheal, S. M. et al. (2014) Mol Cancer Ther) or SADA-BiDE and then injected with ¹⁷⁷Lu-Bn-DOTA. Each line represents one group, with 3-5 mice per group. Circles denote a group that received IgG-BiDE (Cheal, S. M. et al. (2014) Mol Cancer Ther) followed by clearing agent 48 hrs later. Squares denote a group that received P53-BiDE. Diamonds denote a group that received P63-BiDE. Hexagons denote a group that received P73-BiDE. No SADA-BiDE treated mice received any clearing agent. A representative anti-tumor IgG and ¹⁷⁷Lu-Bn-DOTA alone clearance curves were added as a reference. (+) symbols with a dotted line denote the ¹²⁴I-labeled anti-GD2 IgG, and (x) symbols with a dotted line denote ¹⁷⁷Lu-Bn-DOTA alone. FIG. 6D depicts a graph showing tumor activity measurements from mice which received ¹⁷⁷Lu-Bn-DOTA either 24 (black) or 72 (gray) hours after P53-BiDE(noHIS) administration. Measurements were made using SPECT. FIG. 6E depicts a graph showing decay corrected activity at the site of a tumor over a 96 hour time period from mice treated with P53-BiDE. Measurements were made using SPECT.

FIG. 7A and FIG. 7B depict results of biodistribution experiments with exemplary SADA-BiDE conjugates. FIG. 7A depicts a bar graph showing tissue biodistribution from mice treated with SADA-BiDE or IgG-BiDE (Cheal, S. M. et al. (2014) Mol Cancer Ther). Black bars denote measured activity in tissues from mice treated with IgG-BiDE (Cheal, S. M. et al. (2014) Mol Cancer Ther) and clearing agent. Gray bars denote measured activity in tissues from mice treated with P53-BIDE, P63-BiDE, or P73-BiDE (dark to light gray, respectively). Four or five mice were used per group. FIG. 7B depicts a bar graph showing the target to non-target uptake ratio from the biodistribution experimental data shown in FIG. 7A. Each organ had the percent injected dose per gram (% ID/g) calculated and then was divided in reference to the tumor activity. Black bars denote measured activity in tissues from mice treated with IgG-BiDE (Cheal, S. M. et al. (2014) Mol Cancer Ther) and clearing agent. Gray bars denote measured activity in tissues from mice treated with either P53-BIDE, P63-BIDE, or P73-BIDE SADA-BiDEs (dark to light gray, respectively)

FIG. 8A and FIG. 8B depict tumor responses after treatment with a SADA-BiDE construct P53-BIDE(NOHIS) in vivo. FIG. 8A depicts a graph showing the change in tumor volume after administration of 1 (circles) or 4 (squares) doses of P53-BiDE. As a reference other mice were also treated with IgG-BiDE (Cheal, S. M. et al. (2014) Mol Cancer Ther) and clearing agent (triangles). FIG. 8B provides images of an exemplary mouse treated with a single dose of P53-BIDE(NOHIS) from the experimental data shown in FIG. 8A. Images are shown of the mouse on days 1, 8 and 15 with a box around the site of the tumor.

FIG. 9 depicts a schematic of an exemplary conjugate, SADA-Cytokine, made up of a SADA domain and one binding domain (e.g., IL15receptor alpha) which captures a soluble ligand (e.g., soluble IL15) during manufacture, that may be useful for immunotherapy. The circles denote the soluble IL15 (sIL15), which binds to the IL15receptor alpha domain (IL15Rα) (light gray boxes) during manufacture, such that it can be presented to its target as a complex. Dark gray boxes represent a SADA domain (shown as the most inner/proximal domain when assembled) (e.g. a human p53-tetramerization domain for P53-Cytokine; a human p63 tetramerization domain P63-Cytokine and a p73 tetramerization domain for P73-Cytokine). As illustrated, IL15Rα-sIL15 can dimerize, creating apparent octomers when fused with tetrameric SADA domains. Black arrows indicate self-assembly of the construct and gray arrows indicate disassembly of the construct.

FIG. 10A and FIG. 10B depict experiments showing purity and stability of preparations of P53-Cytokine, P63-Cytokine and P73-Cytokine SADA-Cytokines. FIG. 10A depicts an HPLC chromatogram that shows the size and purity of each SADA-Cytokine. All graphs are overlaid and normalized to their peak intensity. The main peak shows over 98% purity for all three versions. The last peak (^{˜}25 min) denotes a non-specific peak from the storage buffer (sodium citrate). P53-Cytokine is shown with a dashed black line, P63-Cytokine is shown with a dark gray line and P73-Cytokine is shown with a light gray line. FIG. 10B depicts a summary of HPLC chromatograms of preparations of P53-Cytokine (circles), P63-Cytokine (triangles) and P73-Cytokine (diamonds) incubated at 37° C. for a 30 day period. Percentage of correctly sized protein (^{˜}16 min) is plotted over each time point for all three versions.

FIG. 11A to FIG. 11D depict in vitro activity of P53-Cytokine, P63-Cytokine and P73-Cytokine SADA-Cytokines. FIG. 11A depicts a graph showing SADA-Cytokine dependent proliferation. The dose dependent proliferative response of TIB214 cells to each of P53-Cytokine (circles), P63-Cytokine (squares) and P73-Cytokine (triangles) is shown. FIG. 11B depicts a graph showing NK Cell cytotoxicity improvement from SADA-Cytokine stimulation. Bar graph summarizes peak cytotoxicity improvement from exposure of human NK cells to each SADA-Cytokine for 3 days. Cytotoxicity was assessed over a 4 hr period using a GD2(+) cell line that is sensitive to NK mediated killing and an anti-GD2 IgG (Ahmed, M. et al. (2015) Oncolmmunology). Control (black bar), P53-Cytokine (medium gray bar), P63-Cytokine (dark gray bar) and P73-Cytokine (light gray bar). FIG. 11C depicts a graph showing T Cell cytotoxicity improvement from SADA-Cytokine stimulation. Bar graph summarizes peak cytotoxicity improvement from exposure of human T cells to each SADA-Cytokine for 3 days. Cytotoxicity was assessed over a 4 hr period using a GD2(+) cell line and a T-cell engaging anti-GD2 IgG-scFv bispecific (Xu, H. et al. (2015) Cancer immunology research). Control (black bar), P53-Cytokine (medium gray bar), P63-Cytokine (dark gray bar) and P73-Cytokine (light gray bar). FIG. 11D depicts a graph showing tumor growth in DKO mice with GD2(+) tumors implanted subcutaneously. Each mouse was treated with PBMCs (gray triangles) and a low dose of an anti-tumor IgG-scFv (Xu, H. et al. (2015) Cancer immunology research) and additional cytokines. Untreated tumors grew out very quickly (black lines). Tumors treated with the IgG-scFv and an Fc-Cytokine (Uu et al. 2016 JBC, http://www.jbc.org/content/291/46/23869) with a mutation to improve binding (light gray line) shrunk tumors slower than mice treated with the IgG-scFv and SADA-Cytokine (dark gray line).

FIG. 12A and FIG. 12B depict ribbon structures of SADA domains and potential SADA domains. FIG. 12A depicts ribbon structures of SADA domains derived from human p53, p63, p73, hnRNPC, SNAP-23, Stefin B, KCNQ4, and CBFA2T1 proteins. FIG. 12B depicts ribbon structures of potential SADA domains derived from human SYCP3, UGP2 and TRIM33 proteins.

FIG. 13A and FIG. 13B depict in vitro analysis of an exemplary anti-HER2 SADA construct. FIG. 13A shows SEC-HPLC chromatograms of two different variants of the anti-HER2 P53-BiDE (anti-HER2 scFv in the HL and LH orientations in upper and lower graphs, respectively). This exemplary anti-HER2 P53-BiDE is exceptionally pure after single-step affinity purification and retains a size of ^{˜}200 kDa (^{˜}16 min). FIG. 13B depicts a FACS analysis of an exemplary anti-HER2 P53-BiDE construct on a HER2(+) cell line HCC1954 (breast cancer) using a fluorescently labeled ¹⁷⁵Lu-Bn-DOTA conjugate for detection. HER2/BnDOTA binding capacity of these anti-HER2 BiDEs (Black solid and dashed, filled) is similar to the comparable to the IgG-BiDE (grey dashed, filled).

FIG. 14A to FIG. 14C depict in vitro analysis of an exemplary HNRNPC-BiDE construct. FIG. 14A depicts an SEC-HPLC chromatogram and stability of an exemplary HNRNPC-BiDE after single-step affinity purification. As shown, an exemplary HNRNPC-BiDE construct forms a stable tetrameric multimer at the expected size of ^{˜}200 kDa (^{˜}16 min, upper graph) and can maintain its purity after five repeated freeze and thaw cycles (^{˜}16 min, lower graph). FIG. 14B shows FACS analysis of an exemplary HNRNPC-BiDE construct with a GD2(+) cell line M14-Luc (Melanoma) using a fluorescently labeled ¹⁷⁵Lu-Bn-DOTA conjugate for detection. The GD2/BnDOTA binding capacity of the HNRNPC-BiDE (Solid Black, filled) is compared against an IgG-BiDE (Cheal, S. M. et al. (2014) Mol Cancer Ther) (Dashed black, filled) a P63-BiDE (dotted grey, filled) or an isotype control (dashed grey, empty). FIG. 14C depicts normalized binding kinetics of an exemplary HNRNPC-BiDE (dotted black) against the tumor antigen GD2 using SPR, compared with the P53—(solid grey), P63—(dashed grey), or IgG-BiDEs (dashed black). Each construct was run as a concentration series across a streptavidin chip coated with biotin-GD2. The highest concentrations of each were then plotted together on a normalized Y-axis to better show the differences in k_{off}. Data was fitted using a two-state reaction model.

### DEFINITIONS

The scope of present invention is defined by the claims appended hereto and is not limited by particular embodiments described herein; those skilled in the art, reading the present disclosure, will be aware of various modifications that may be equivalent to such described embodiments, or otherwise within the scope of the claims.

In general, terminology used herein is in accordance with its understood meaning in the art, unless clearly indicated otherwise. Explicit definitions of certain terms are provided below; meanings of these and other terms in particular instances throughout this specification will be clear to those skilled in the art from context.

References cited within this specification, or relevant portions thereof, are incorporated herein by reference.

In order that the present invention may be more readily understood, certain terms are first defined below. Additional definitions for the following terms and other terms are set forth throughout the specification.

“Affinity”: As is known in the art, “affinity” is a measure of the tightness with a particular ligand binds to its partner. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

“Affinity matured” (or “affinity matured antibody”), as used herein, refers to an antibody with one or more alterations in one or more CDRs thereof which result an improvement in the affinity of the antibody for antigen, compared to a parent antibody which does not possess those alteration(s). In some embodiments, affinity matured antibodies will have nanomolar or even picomolar affinities for a target antigen. Affinity matured antibodies may be produced by any of a variety of procedures known in the art. Marks et al. (1992) BioTechnology 10:779-783 describes affinity maturation by V_{H} and V_{L} domain shuffling. Random mutagenesis of CDR and/or framework residues is described by: Barbas et al. (1994) Proc. Nat. Acad. Sci. U.S.A 91:3809-3813; Schier et al. 1995, Gene 169: 147-155; Yelton et al. (1995) J. Immunol. 155: 1994-2004; Jackson et al. (1995) J. Immunol. 154(7):3310-9; and Hawkins et al. (1992) J. Mol. Biol. 226:889-896.

“Amelioration”, as used herein, refers to the prevention, reduction or palliation of a state, or improvement of the state of a subject. Amelioration includes, but does not require complete recovery or complete prevention of a disease, disorder or condition (e.g., radiation injury).

“Animal”, as used herein refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In certain embodiments, the animal is susceptible to infection by DV. In some embodiments, an animal may be a transgenic animal, genetically engineered animal, and/or a clone.

“Antibody”, as used herein, has its art understood meaning and refers to an immunoglobulin (Ig) that binds specifically to a particular antigen. As is known by those of ordinary skill in the art, antibodies produced in nature are typically comprised of four polypeptide chains, two heavy (H) chains and two light (L) chains. Each heavy and light chain is comprised of a variable region (abbreviated herein as HCVR or V_{H} and LCVR or V_{L}, respectively) and a constant region. The constant region of a heavy chain comprises a C_{H}1, C_{H}2 and C_{H}3 domain (and optionally a C_{H}⁴ domain in the case of IgM and IgE). The constant region of a light chain is comprised of one domain, C_{L}. The V_{H} and V_{L} regions further contain regions of hypervariability, termed complementarity determining regions (CDRs), interspersed with regions that are more conserved, which are termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgM, IgD, IgG, IgA and IgE), class (e.g., IgG1, IgG2, IgG3, IgG4, IgA1 and IgA2) or subclass.

Antibody agent: As used herein, the term “antibody agent” refers to an agent that specifically binds to a particular antigen. In some embodiments, the term encompasses any polypeptide with immunoglobulin structural elements sufficient to confer specific binding. In various embodiments, suitable antibody agents may include, but are not limited to, monoclonal antibodies, polyclonal antibodies, humanized antibodies, primatized antibodies, chimeric antibodies, human antibodies, bi-specific or multi-specific antibodies, single domain antibodies (e.g., shark single domain antibodies (e.g., IgNAR or fragments thereof)), conjugated antibodies (i.e., antibodies conjugated or fused to other proteins, radiolabels, cytotoxins), Small Modular ImmunoPharmaceuticals (“SMIPs™”), single chain antibodies, cameloid antibodies, antibody fragments, etc. In some embodiments, the term can refer to a stapled peptide. In some embodiments, the term can refer to an antibody-like binding peptidomimetic. In some embodiments, the term can refer to an antibody-like binding scaffold protein. In some embodiments, the term can refer to monobodies or adnectins. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments, an included CDR is substantially identical to a reference CDR in that it is either identical in sequence or contains between 1-5 amino acid substitutions as compared with the reference CDR. In some embodiments, an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments, an included CDR is substantially identical to a reference CDR in that it shows at least 96%, 96%, 97%, 98%, 99%, or 100% sequence identity with the reference CDR. In some embodiments, an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an included CDR is substantially identical to a reference CDR in that at least one amino acid within the included CDR is substituted as compared with the reference CDR but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain, which is homologous or largely homologous to an immunoglobulin-binding domain. In some embodiments, an antibody agent is or comprises a polypeptide that includes all CDRs found in a particular reference antibody chain or chains (e.g., heavy chain and/or light chain).

“Antibody component”, as used herein, refers to a polypeptide element (that may be a complete polypeptide, or a portion of a larger polypeptide, such as for example a fusion polypeptide as described herein) that specifically binds to an epitope or antigen and includes one or more immunoglobulin structural features. In general, an antibody component is any polypeptide whose amino acid sequence includes elements characteristic of an antibody-binding region (e.g., an antibody light chain or variable region or one or more complementarity determining regions (“CDRs”) thereof, or an antibody heavy chain or variable region or one more CDRs thereof, optionally in presence of one or more framework regions). In some embodiments, an antibody component is or comprises a full-length antibody. In some embodiments, an antibody component is less than full-length but includes at least one binding site (comprising at least one, and preferably at least two sequences with structure of known antibody “variable regions”). In some embodiments, the term “antibody component” encompasses any protein having a binding domain, which is homologous or largely homologous to an immunoglobulin-binding domain. In particular embodiments, an included “antibody component” encompasses polypeptides having a binding domain that shows at least 99% identity with an immunoglobulin binding domain. In some embodiments, an included “antibody component” is any polypeptide having a binding domain that shows at least 70%, 75%, 80%, 85%, 90%, 95% or 98% identity with an immunoglobulin binding domain, for example a reference immunoglobulin binding domain. An included “antibody component” may have an amino acid sequence identical to that of an antibody (or a portion thereof, e.g., an antigen-binding portion thereof) that is found in a natural source. An antibody component may be monospecific, bi-specific, or multi-specific. An antibody component may include structural elements characteristic of any immunoglobulin class, including any of the human classes: IgG, IgM, IgA, IgD, and IgE. It has been shown that the antigen-binding function of an antibody can be performed by fragments of a full-length antibody. Such antibody embodiments may also be bispecific, dual-specific, or multi-specific formats specifically binding to two or more different antigens. Examples of binding fragments encompassed within the term “antigen-binding portion” of an antibody include (i) a Fab fragment, a monovalent fragment consisting of the V_{H}, V_{L}, C_{H}1 and C_{L} domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{H} and V_{L} domains of a single arm of an antibody, (v) a dAb fragment (Ward et al. (1989) Nature 341:544-546), which comprises a single variable domain; and (vi) an isolated complementarity determining region (CDR). Furthermore, although the two domains of the Fv fragment, V_{H} and V_{L}, are coded for by separate genes, they can be joined, using recombinant methods, by a synthetic linker that enables them to be made as a single protein chain in which the V_{H} and V_{L} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). In some embodiments, an “antibody component”, as described herein, is or comprises such a single chain antibody. In some embodiments, an “antibody component” is or comprises a diabody. Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al., (1993) Proc. Natl. Acad. Sci. USA 90:6444-6448; Poljak, R. J., (1994) Structure 2(12):1121-1123). Such antibody binding portions are known in the art (Kontermann and Dubel eds., Antibody Engineering (2001) Springer-Verlag. New York. 790 pp. (ISBN 3-540-41354-5). In some embodiments, an antibody component is or comprises a single chain “linear antibody” comprising a pair of tandem Fv segments (V_{H}-C_{H}1-V_{H}-C_{H}1) which, together with complementary light chain polypeptides, form a pair of antigen binding regions (Zapata et al., 1995, Protein Eng. 8(10): 1057-1062; and U.S. Pat. No. 5,641,870). In some embodiments, an antibody component may have structural elements characteristic of chimeric or humanized antibodies. In general, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some embodiments, an antibody component may have structural elements characteristic of a human antibody.

“Binding domain”, as used herein, refers to a moiety or entity that specifically binds to a target moiety or entity. Typically, the interaction between a binding domain and its target is non-covalent. In some embodiments, a binding domain may be or comprise a moiety or entity of any chemical class including, for example, a carbohydrate, a lipid, a nucleic acid, a metal, a polypeptide, a small molecule. In some embodiments, a binding domain may be or comprise a polypeptide (or complex thereof). In some embodiments, a binding domain may be or comprise a target-binding portion of an antibody agent, a cytokine, a ligand (e.g., a receptor ligand), a receptor, a toxin, etc. In some embodiments, a binding domain may be or comprise an aptamer. In some embodiments, a binding domain may be or comprise a peptide nucleic acid (PNA).

“Biological activity”, as used herein, refers to an observable biological effect or result achieved by an agent or entity of interest. For example, in some embodiments, a specific binding interaction is a biological activity. In some embodiments, modulation (e.g., induction, enhancement, or inhibition) of a biological pathway or event is a biological activity. In some embodiments, presence or extent of a biological activity is assessed through detection of a direct or indirect product produced by a biological pathway or event of interest.

“Bispecific binding agent”, as used herein, refers a binding agent capable of binding to two antigens, which can be on the same molecule or on different molecules. Bispecific binding agents as described herein are, in some embodiments, engineered to have the two antigen binding sites, and are typically not naturally occurring proteins. Bispecific binding agents as described herein refer to binding agents capable of binding two or more related or unrelated targets. Bispecific binding agents as described herein are, in some embodiments, capable of binding simultaneously to two targets that are of different structure, e.g., two different antigens, two different epitopes on the same antigen, or a hapten and/or an antigen or epitope. In many embodiments, bispecific binding agents of the present invention are proteins engineered to have characteristics of bispecific binding agents as described herein.

“Bispecific antibody”, as used herein, refers to a bispecific binding agent in which at least one, and typically both, of the binding moieties is or comprises an antibody component. A variety of different bi-specific antibody structures are known in the art. In some embodiments, each binding moiety in a bispecific antibody that is or comprises an antibody component includes V_{H} and/or V_{L} regions; in some such embodiments, the V_{H} and/or V_{L} regions are those found in a particular monoclonal antibody. In some embodiments, where the bispecific antibody contains two antibody component-binding moieties, each includes V_{H} and/or V_{L} regions from different monoclonal antibodies. In some embodiments, where the bispecific antibody contains two antibody component binding moieties, wherein one of the two antibody component binding moieties includes an immunoglobulin molecule having V_{H} and/or V_{L} regions that contain CDRs from a first monoclonal antibody, and one of the two antibody component binding moieties includes an antibody fragment (e.g., Fab, F(ab′), F(ab′)₂, Fd, Fv, dAB, scFv, etc.) having V_{H} and/or V_{L} regions that contain CDRs from a second monoclonal antibody.

“Bispecific binding agent”, as used herein, refers to a polypeptide agent with two discrete binding moieties, each of which binds with a distinct target. In some embodiments, a bispecific binding agent is or comprises a single polypeptide; in some embodiments, a bispecific binding agent is or comprises a plurality of peptides which, in some such embodiments may be covalently associated with one another, for example by cross-linking. In some embodiments, the two binding moieties of a bispecific binding agent recognize different sites (e.g., epitopes) the same target (e.g., antigen); in some embodiments, they recognize different targets. In some embodiments, a bispecific binding agent is capable of binding simultaneously to two targets that are of different structure.

“Carrier”, as used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

“CDR”, as used herein, refers to a complementarity determining region within an antibody variable region. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. Certain systems have been established in the art for defining CDR boundaries (e.g., Kabat, Chothia, etc.); those skilled in the art appreciate the differences between and among these systems and are capable of understanding CDR boundaries to the extent required to understand and to practice the claimed invention.

“CDR-grafted antibody”, as used herein, refers to an antibody whose amino acid sequence comprises heavy and light chain variable region sequences from one species but in which the sequences of one or more of the CDR regions of V_{H} and/or V_{L} are replaced with CDR sequences of another species, such as antibodies having murine V_{H} and V_{L} regions in which one or more of the murine CDRs (e.g., CDR3) has been replaced with human CDR sequences. Likewise, a “CDR-grafted antibody” may also refer to antibodies having human V_{H} and V_{L} regions in which one or more of the human CDRs (e.g., CDR3) has been replaced with mouse CDR sequences.

“Combination therapy”: As used herein, the term “combination therapy” refers to those situations in which a subject is simultaneously exposed to two or more therapeutic regimens (e.g., two or more therapeutic agents). In some embodiments, two or more agents or may be administered simultaneously; in some embodiments, such agents may be administered sequentially; in some embodiments, such agents are administered in overlapping dosing regimens.

“Comparable”, as used herein, refers to two or more agents, entities, situations, sets of conditions, etc. that may not be identical to one another but that are sufficiently similar to permit comparison there between so that conclusions may reasonably be drawn based on differences or similarities observed. Those of ordinary skill in the art will understand, in context, what degree of identity is required in any given circumstance for two or more such agents, entities, situations, sets of conditions, etc. to be considered comparable.

“Corresponding to”, as used herein designates the position/identity of an amino acid residue in a polypeptide of interest. Those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids.

“Detection Agents”, as described herein, refer to moieties or agents that are amenable to detection, for example, due to their specific structural and/or chemical characteristics, and/or their functional properties. Non-limiting examples of such agents include enzymes, radiolabels, haptens, fluorescent labels, phosphorescent molecules, chemiluminescent molecules, chromophores, luminescent molecules, photoaffinity molecules, colored particles or ligands, such as biotin. Many detection agents are known in the art, as are systems for their attachment to antibodies (see, for e.g., U.S. Pat. Nos. 5,021,236; 4,938,948; and 4,472,509, each incorporated herein by reference). Particular examples may include paramagnetic ions, radioactive isotopes, fluorochromes, NMR-detectable substances, X-ray imaging agents, among others. In some embodiments of the present invention, the conjugated detection agent is a diagnostic or imaging agent.

“Dosage form” and “unit dosage form”, as used herein, the term “dosage form” refers to physically discrete unit of a therapeutic agent for a subject (e.g., a human patient) to be treated. Each unit contains a predetermined quantity of active material calculated or demonstrated to produce a desired therapeutic effect when administered to a relevant population according to an appropriate dosing regimen. For example, in some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). It will be understood, however, that the total dosage administered to any particular patient will be selected by a medical professional (e.g., a medical doctor) within the scope of sound medical judgment.

“Dosing regimen” (or “therapeutic regimen”), as used herein is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, the therapeutic agent is administered continuously (e.g., by infusion) over a predetermined period. In some embodiments, a therapeutic agent is administered once a day (QD) or twice a day (BID). In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

“Effector function” as used herein refers a biochemical event that results from the interaction of an antibody Fc region with an Fc receptor or ligand. Effector functions include but are not limited to antibody-dependent cell-mediated cytotoxicity (ADCC), antibody-dependent cell-mediated phagocytosis (ADCP), and complement-mediated cytotoxicity (CMC). In some embodiments, an effector function is one that operates after the binding of an antigen, one that operates independent of antigen binding, or both.

“Effector cell” as used herein refers to a cell of the immune system that expresses one or more Fc receptors and mediates one or more effector functions. In some embodiments, effector cells may include, but may not be limited to, one or more of monocytes, macrophages, neutrophils, dendritic cells, eosinophils, mast cells, platelets, large granular lymphocytes, Langerhans' cells, natural killer (NK) cells, T-lymphocytes, B-lymphocytes and may be from any organism including but not limited to humans, mice, rats, rabbits, and monkeys.

“Engineered” as used herein refers, in general, to the aspect of having been manipulated by the hand of man. For example, in some embodiments, a polynucleotide may be considered to be “engineered” when two or more sequences, that are not linked together in that order in nature, are manipulated by the hand of man to be directly linked to one another in the engineered polynucleotide. In some particular such embodiments, an engineered polynucleotide may comprise a regulatory sequence that is found in nature in operative association with a first coding sequence but not in operative association with a second coding sequence, is linked by the hand of man so that it is operatively associated with the second coding sequence. Alternatively or additionally, in some embodiments, first and second nucleic acid sequences that each encode polypeptide elements or domains that in nature are not linked to one another may be linked to one another in a single engineered polynucleotide. Comparably, in some embodiments, a cell or organism may be considered to be “engineered” if it has been manipulated so that its genetic information is altered (e.g., new genetic material not previously present has been introduced, or previously present genetic material has been altered or removed). As is common practice and is understood by those in the art, progeny of an engineered polynucleotide or cell are typically still referred to as “engineered” even though the actual manipulation was performed on a prior entity. Furthermore, as will be appreciated by those skilled in the art, a variety of methodologies are available through which “engineering” as described herein may be achieved. For example, in some embodiments, “engineering” may involve selection or design (e.g., of nucleic acid sequences, polypeptide sequences, cells, tissues, and/or organisms) through use of computer systems programmed to perform analysis or comparison, or otherwise to analyze, recommend, and/or select sequences, alterations, etc. Alternatively or additionally, in some embodiments, “engineering” may involve use of in vitro chemical synthesis methodologies and/or recombinant nucleic acid technologies such as, for example, nucleic acid amplification (e.g., via the polymerase chain reaction), hybridization, mutation, transformation, transfection, etc. As will be appreciated by those skilled in the art, a variety of established such techniques (e.g., for recombinant DNA, oligonucleotide synthesis, and tissue culture and transformation [e.g., electroporation, lipofection, etc.]) are well known in the art and described in various general and more specific references that are cited and/or discussed throughout the present specification. See, e.g., Sambrook et al., Molecular Cloning: A Laboratory Manual (2nd ed., Cold Spring Harbor Laboratory Press, Cold Spring Harbor, N.Y. [1989]), which is incorporated herein by reference for any purpose.

“Epitope”, as used herein, includes any moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

“Excipient”, as used herein, refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

“Fc ligand” as used herein refers to a molecule, preferably a polypeptide, from any organism that binds to the Fc region of an antibody to form an Fc-ligand complex. Fc ligands include but are not limited to FcγRIIA (CD32A), FcγRIIB (CD32B), FcγRIIIA (CD16A), FcγRIIIB (CD16B), FcγRI (CD64), FcεRII (CD23), FcRn, Clq, C3, staphylococcal protein A, streptococcal protein G, and viral FcγR. Fc ligands may include undiscovered molecules that bind Fc.

“Fluorescent Label” as is understood in the art, is a moiety or entity that has fluorescent character and, in some embodiments, may be detectable based on such fluorescence. In some embodiments, a fluorescent label may be or may comprise one or more of Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red, among others.

“Framework” or “framework region”, as used herein, refers to the sequences of a variable region minus the CDRs. Because a CDR sequence can be determined by different systems, likewise a framework sequence is subject to correspondingly different interpretations. The six CDRs divide the framework regions on the heavy and light chains into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, FR1, for example, represents the first framework region closest to the amino terminal end of the variable region and 5′ with respect to CDR1, and FRs represents two or more of the sub-regions constituting a framework region.

“Host cell”, as used herein, refers to a cell into which exogenous DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: CHO (e.g., CHO KI, DXB-11 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, W138, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3 A cell, HT1080 cell, myeloma cell, tumor cell, and a cell line derived from an aforementioned cell. In some embodiments, the cell comprises one or more viral genes, e.g., a retinal cell that expresses a viral gene (e.g., a PER.C6™ cell).

“Human antibody”, as used herein, is intended to include antibodies having variable and constant regions generated (or assembled) from human immunoglobulin sequences. In some embodiments, antibodies (or antibody components) may be considered to be “human” even though their amino acid sequences include residues or elements not encoded by human germline immunoglobulin sequences (e.g., include sequence variations, for example that may (originally) have been introduced by random or site-specific mutagenesis in vitro or by somatic mutation in vivo), for example in one or more CDRs and in particular CDR3.

“Humanized”, as is known in the art, the term “humanized” is commonly used to refer to antibodies (or antibody components) whose amino acid sequence includes V_{H} and V_{L}region sequences from a reference antibody raised in a non-human species (e.g., a mouse), but also includes modifications in those sequences relative to the reference antibody intended to render them more “human-like”, i.e., more similar to human germline variable sequences. In some embodiments, a “humanized” antibody (or antibody component) is one that immunospecifically binds to an antigen of interest and that has a framework (FR) region having substantially the amino acid sequence as that of a human antibody, and a complementary determining region (CDR) having substantially the amino acid sequence as that of a non-human antibody. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor immunoglobulin) and all or substantially all of the framework regions are those of a human immunoglobulin consensus sequence. In some embodiments, a humanized antibody also comprises at least a portion of an immunoglobulin constant region (Fc), typically that of a human immunoglobulin constant region. In some embodiments, a humanized antibody contains both the light chain as well as at least the variable domain of a heavy chain. The antibody also may include a C_{H}1, hinge, C_{H}2, C_{H}3, and, optionally, a C_{H}4region of a heavy chain constant region. In some embodiments, a humanized antibody only contains a humanized V_{L} region. In some embodiments, a humanized antibody only contains a humanized V_{H} region. In some certain embodiments, a humanized antibody contains humanized V_{H} and V_{L} regions.

“Improve,” “increase” or “reduce,” as used herein or grammatical equivalents thereof, indicate values that are relative to a baseline or control measurement. In some embodiments, relative to a baseline or control may refer to a measurement in the same individual prior to initiation of a treatment described herein, or a measurement in a control individual (or multiple control individuals) in the absence of the treatment described herein. A “control individual” is an individual afflicted with the same form of disease or injury as the individual being treated. In some embodiments, values that are relative to a baseline or control may refer to may refer to a measurement in an experiment or animal or individual undergoing analogous treatment with a control or reference agent (e.g., with a therapeutic lacking a SADA domain and/or with a therapeutic with an alternative domain such as an Ig domain, or with no therapeutic agent).

“In vitro”, as used herein refers to events that occur in an artificial environment, e.g., in a test tube or reaction vessel, in cell culture, etc., rather than within a multi-cellular organism.

“In vivo”, as used herein refers to events that occur within a multi-cellular organism, such as a human and a non-human animal. In the context of cell-based systems, the term may be used to refer to events that occur within a living cell (as opposed to, for example, in vitro systems).

“Isolated”, as used herein, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

“K_{D}”, as used herein, refers to the dissociation constant of a binding agent (e.g., a SADA domain, an antibody or binding component thereof) from a complex with its partner (e.g., a corresponding SADA domain or an epitope to which the antibody or binding component thereof binds).

“k_{off}”, as used herein, refers to the off rate constant for dissociation of a binding agent (e.g., a SADA domain, an antibody or binding component thereof) from a complex with its partner (e.g., a corresponding SADA domain or an epitope to which the antibody or binding component thereof binds).

“kₒₙ”, as used herein, refers to the on rate constant for association of a binding agent (e.g., a SADA domain, an antibody or binding component thereof) with its partner (e.g., a corresponding SADA domain or an epitope to which the antibody or binding component thereof binds).

“Linker”, as used herein, typically refers to a portion of a molecule or entity that connects two or more different regions of interest (e.g., particular structural and/or functional domains or moieties of interest). In some embodiments, a linker does not participate significantly in the relevant function of interest (e.g., so that presence or absence of the linker, in association with the relevant domain or moiety of interest does not materially alter the relevant function of the domain or moiety). In some embodiments, a linker in characterized by lack of defined or rigid structure. In some embodiments, particularly when one or more domains or moieties of interest is/are comprised of a polypeptide, a linker is or comprises a polypeptide. In some particular embodiments, a polypeptide (e.g., an engineered polypeptide) as described herein may have general structure S1-L-S2, wherein S1 and S2 are the moieties or domains of interest. In some embodiments, one or both of S1 and S2 may be or comprise a binding element (e.g., an antibody component) as described herein. In some embodiments, a polypeptide linker may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids long. In some embodiments, a polypeptide linker may have an amino acid sequence that is or comprises a sequence as described in Holliger, P., et al. (1993) Proc. Natl. Acad. Sci. USA 90:6444-6448 or Poljak, R. J., et al. (1994) Structure 2: 1121-1123. In some embodiments, a polypeptide linker may have an amino acid sequence that is or comprises GGGGSGGGGSGGGGS (i.e., [G₄S]₃) SEQ ID NO: 99 or GGGGSGGGGSGGGGSGGGGSGGGGSGGGGS (i.e., [G₄S]₆) SEQ ID NO: 100.

“Multimer”, as used herein, refers to a complex of monomeric units. The term “multimer” as used herein excludes dimers, but includes trimers, and multimers of four monomers (tetramers), or of more than four monomers (pentamers, hexamers, septamers, octamers, nonamers, decamers, etc.). A domain that promotes association of monomeric units to form multimeric complexes is referred to herein as a “multimerization domain.”

“Multivalent binding agent”, as used herein, refers to a binding agent capable of binding to two or more targets, which can be on the same molecule or on different molecules. Multivalent binding agents as described herein are, in some embodiments, engineered to have the three or more target binding sites. In some embodiments, a multivalent binding agent is not a naturally occurring polypeptides. Multivalent binding agents as described herein refer to binding agents capable of binding two or more related or unrelated targets. In some embodiments, multivalent binding agents may be composed of multiple copies of a single antibody component or multiple copies of different antibody components. Such binding agents are capable of binding to two or more antigens and are tetravalent or multivalent binding agents. In some embodiments, multivalent binding agents may additionally or alternatively comprise a therapeutic agent, such as, for example, an immunomodulator, toxin or an RNase. Multivalent binding agents as described herein are, in some embodiments, capable of binding simultaneously to at least two targets that are of different structure, e.g., two different antigens, two different epitopes on the same antigen, a hapten, a small molecule, a cytokine, a receptor, or any combination thereof. In some embodiments, multivalent binding agents of the present disclosure are engineered polypeptides and/or fusion proteins. In some embodiments, multivalent binding agents of the present invention may include an antibody agent. In some embodiments, a multivalent binding agent includes an antibody agent that comprises a heavy chain variable domain and a light chain variable domain, which include six CDRs involved in antigen binding per antigen binding site.

“Nucleic acid”, as used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is single stranded; in some embodiments, a nucleic acid is double stranded. In some embodiments a nucleic acid has a nucleotide sequence comprising at least one element that encodes, or is the complement of a sequence that encodes, a polypeptide. In some embodiments, a nucleic acid has enzymatic activity.

“Operably linked”, as used herein, refers to a juxtaposition wherein the components described are in a relationship permitting them to function in their intended manner. A control sequence “operably linked” to a coding sequence is ligated in such a way that expression of the coding sequence is achieved under conditions compatible with the control sequences. “Operably linked” sequences include both expression control sequences that are contiguous with the gene of interest and expression control sequences that act in trans or at a distance to control the gene of interest. The term “expression control sequence” as used herein refers to polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism. For example, in prokaryotes, such control sequences generally include promoter, ribosomal binding site, and transcription termination sequence, while in eukaryotes, typically, such control sequences include promoters and transcription termination sequence. The term “control sequences” is intended to include components whose presence is essential for expression and processing, and can also include additional components whose presence is advantageous, for example, leader sequences and fusion partner sequences.

“Paramagnetic Ion”, as is understood in the art, refers to an ion with paramagnetic character. In some embodiments, a paramagnetic ion is one or more of chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III), erbium (III), lanthanum (III), gold (III), lead (II), and/or bismuth (III).

“Payload”, as used herein, refers to a moiety or entity that is delivered to a site of interest (e.g., to a cell, tissue, tumor, or organism) by association with another entity. In some embodiments, a payload is or comprises a detection agent. In some embodiments, a payload entity is or comprises a therapeutic agent. In some embodiments, a payload entity is or comprises a catalytic agent. Those of ordinary skill in the art will appreciate that a payload entity may be of any chemical class. For example, in some embodiments, a payload entity may be or comprise a carbohydrate, an isotope, a lipid, a nucleic acid, a metal, a nanoparticle (e.g., a ceramic or polymer nanoparticle), polypeptide, a small molecule, etc. To give but a few examples, in some embodiments, a therapeutic agent payload may be or comprise a toxin (e.g., a toxic peptide, small molecule, or isotope [e.g., radioisotope]); in some embodiments, a detection agent payload may be or comprise a fluorescent entity or agent, a radioactive entity or agent, an agent or entity detectable by binding (e.g., a tag, a hapten, a ligand, etc.), a catalytic agent, etc.

“Physiological conditions”, as used herein, has its art-understood meaning referencing conditions under which cells or organisms live and/or reproduce. In some embodiments, the term refers to conditions of the external or internal milieu that may occur in nature for an organism or cell system. In some embodiments, physiological conditions are those conditions present within the body of a human or non-human animal, especially those conditions present at and/or within a surgical site. Physiological conditions typically include, e.g., a temperature range of 20° C. to 40° C., atmospheric pressure of 1, pH of 6 to 8, glucose concentration of 1 mM to 20 mM, oxygen concentration at atmospheric levels, and gravity as it is encountered on earth. In some embodiments, conditions in a laboratory are manipulated and/or maintained at physiologic conditions. In some embodiments, physiological conditions are encountered in an organism.

“Polypeptide”, as used herein, refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modification of or covalent linkage to one or more amino acid side chains, the polypeptide's N-terminus, the polypeptide's C-terminus, or any combination thereof. In some embodiments, such pendant groups or modifications may be selected from acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30%, and is often greater than about 40%, 50%, 60%, 70%, 80%, 90%, 91%, 92%, 93%, 94%, 95%, 96%, 97%, 98%, 99% or more and/or includes at least one region (i.e., a conserved region that may in some embodiments may be or comprise a characteristic sequence element) that shows very high sequence identity, often greater than 90% or even 95%, 96%, 97%, 98%, or 99%. Such a conserved region usually encompasses at least three to four and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice-versa, and/or fragments may be present in a different order in the polypeptide of interest than in the parent), so that the polypeptide of interest is a derivative of its parent polypeptide

“Prevent” or “prevention”, as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

“Pure”: As used herein, an agent or entity is “pure” if it is substantially free of other components. For example, a preparation that contains more than about 80% of a particular agent or entity is typically considered to be a pure preparation. In some embodiments, an agent (or entity, therapeutic, etc.) is at least 85%, at least 90%, at least 91%, at least 92%, at least 93%, at least 94%, at least 95%, at least 96%, at least 97%, at least 98%, or at least 99% pure.

“Radioactive Isotope”: The term “radioactive isotope” as used herein has its art-understood meaning referring to an isotope that undergoes radioactive decay. In some embodiments, a radioactive isotope may be or comprise one or more of actinium-225, astatine-211, bismuth-212, carbon-14, chromium-51, chlorine-36, cobalt-57, cobalt-58, copper-67, Europium-152, gallium-67, hydrogen-3, iodine-123, iodine-124, iodine-125, iodine-131, indium-111, iron-59, lead-212, lutetium-177, phosphorus-32, radium-223, radium-224, rhenium-186, rhenium-188, selenium-75, sulphur-35, technicium-99m, thorium-227, yttrium-90, and zirconium-89.

“Recombinant”, as used herein, is intended to refer to polypeptides (e.g., protein therapeutics with a SADA domain) that are designed, engineered, prepared, expressed, created or isolated by recombinant means, such as polypeptides expressed using a recombinant expression vector transfected into a host cell, polypeptides isolated from a recombinant, combinatorial human polypeptide library (Hoogenboom H. R. (1997) TIB Tech. 15:62-70; Azzazy H., and Highsmith W. E. (2002) Clin. Biochem. 35:425-445; Gavilondo, J. V. and Larrick, J. W. (2002) BioTechniques 29: 128-145; Hoogenboom H., and Chames, P. (2000) Immunology Today 21:371-378), antibodies isolated from an animal (e.g., a mouse) that is transgenic for human immunoglobulin genes (see e.g., Taylor, L. D. et al. (1992) Nucl. Acids Res. 20:6287-6295; Little M. et al. (2000) Immunology Today 21:364-370; Kellermann S-A., and Green L. L. (2002) Current Opinion in Biotechnology 13:593-597; Murphy, A. J. et al. (2014) Proc. Natl. Acad. Sci. U.S.A. 111(14):5153-5158) or polypeptides prepared, expressed, created or isolated by any other means that involves splicing selected sequence elements to one another. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source. For example, in some embodiments, a recombinant antibody polypeptide is comprised of sequences found in the germline of a source organism of interest (e.g., human, mouse, etc.). In some embodiments, a recombinant antibody has an amino acid sequence that resulted from mutagenesis (e.g., in vitro or in vivo, for example in a transgenic animal), so that the amino acid sequences of the V_{H} and V_{L} regions of the recombinant antibodies are sequences that, while originating from and related to germline V_{H} and V_{L} sequences, may not naturally exist within the germline antibody repertoire in vivo.

“Recovering”, as used herein, refers to the process of rendering an agent or entity substantially free of other previously-associated components, for example by isolation, e.g., using purification techniques known in the art. In some embodiments, an agent or entity is recovered from a natural source and/or a source comprising cells.

“Reference”, as used herein describes a standard, control, or other appropriate reference against which a comparison is made as described herein. For example, in some embodiments, a reference is a standard or control agent, animal, individual, population, sample, sequence, series of steps, set of conditions, or value against which an agent, animal, individual, population, sample, sequence, series of steps, set of conditions, or value of interest is compared. In some embodiments, a reference is tested and/or determined substantially simultaneously with the testing or determination of interest. In some embodiments, a reference is a historical reference, optionally embodied in a tangible medium. Typically, as would be understood by those skilled in the art, a reference is determined or characterized under conditions comparable to those utilized in the assessment of interest.

“Risk” as will be understood from context, “risk” of a disease, disorder, and/or condition comprises likelihood that a particular individual will develop a disease, disorder, and/or condition (e.g., a radiation injury). In some embodiments, risk is expressed as a percentage. In some embodiments, risk is from 0, 1, 2, 3, 4, 5, 6, 7, 8, 9, 10, 20, 30, 40, 50, 60, 70, 80, 90 and up to 100%. In some embodiments, risk is expressed as a risk relative to a risk associated with a reference sample or group of reference samples. In some embodiments, a reference sample or group of reference samples have a known risk of a disease, disorder, condition and/or event (e.g., a radiation injury). In some embodiments a reference sample or group of reference samples are from individuals comparable to a particular individual. In some embodiments, relative risk is 0, 1, 2, 3, 4, 5, 6, 7, 8, 9, 10 or more.

“Specific binding”, as used herein, refers to a binding agent's ability to discriminate between possible partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

“Subject”, as used herein, means any mammal, including humans. In certain embodiments of the present invention the subject is an adult, an adolescent or an infant. In some embodiments, terms “individual” or “patient” are used and are intended to be interchangeable with “subject”. Also contemplated by the present invention are the administration of the pharmaceutical compositions and/or performance of the methods of treatment in-utero.

“Substantially”: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

“Substantial sequence homology”, as used herein refers to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues will appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains. Substitution of one amino acid for another of the same type may often be considered a “homologous” substitution. Typical amino acid categorizations are summarized in Table 1 and 2.

TABLE 1

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

TABLE 2

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown | Xaa | X |
| amino acid | | |

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., 1990, J. Mot. Biol., 215(3): 403-410; Altschul et al., 1996, Methods in Enzymology 266:460-80; Altschul et al., 1997, Nucleic Acids Res. 25:3389-3402; Baxevanis et al., 1998, Bioinformatics: A Practical Guide to the Analysis of Genes and Proteins, Wiley; and Misener et al., (eds.), Bioinformatics Methods and Protocols (Methods in Molecular Biology, Vol. 132), Humana Press, 1999; all of the foregoing of which are incorporated herein by reference. In addition to identifying homologous sequences, the programs mentioned above typically provide an indication of the degree of homology. In some embodiments, two sequences are considered to be substantially homologous if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are homologous over a relevant stretch of residues. In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues.

“Substantial identity”, as used herein refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., (1990) J. Mol. Biol., 215(3): 403-410; Altschul et al., (1996) Methods in Enzymology 266:460-80; Altschul et al., (1997) Nucleic Acids Res. 25:3389-3402; Baxevanis et al., (1998) Bioinformatics: A Practical Guide to the Analysis of Genes and Proteins, Wiley; and Misener et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues. In the context of a CDR, reference to “substantial identity” typically refers to a CDR having an amino acid sequence at least 80%, preferably at least 85%, at least 90%, at least 95%, at least 98% or at least 99% identical to that of a reference CDR.

“Surface plasmon resonance”, as used herein, refers to an optical phenomenon that allows for the analysis of specific binding interactions in real-time, for example through detection of alterations in protein concentrations within a biosensor matrix, such as by using a BIAcore system (Pharmacia Biosensor AB, Uppsala, Sweden and Piscataway, N.J.). For further descriptions, see Jonsson, U., et al. (1993) Ann. Biol. Clin. 51:19-26; Jonsson, U., et al., (1991) Biotechniques 11:620-627; Johnsson, B., et al., (1995) J. Mol. Recognit. 8:125-131; and Johnnson, B., et al., (1991) Anal. Biochem. 198:268-277.

“Therapeutically effective amount”, as used herein, is meant an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

“Transformation”, as used herein, refers to any process by which exogenous DNA is introduced into a host cell. Transformation may occur under natural or artificial conditions using various methods well known in the art. Transformation may rely on any known method for the insertion of foreign nucleic acid sequences into a prokaryotic or eukaryotic host cell. In some embodiments, a particular transformation methodology is selected based on the host cell being transformed and may include, but is not limited to, viral infection, electroporation, mating, lipofection. In some embodiments, a “transformed” cell is stably transformed in that the inserted DNA is capable of replication either as an autonomously replicating plasmid or as part of the host chromosome. In some embodiments, a transformed cell transiently expresses introduced nucleic acid for limited periods of time.

“Vector”, as used herein, refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.”

### DETAILED DESCRIPTION OF CERTAIN EMBODIMENTS

One of the biggest hurdles in designing effective injectable therapeutics is balancing the benefits of extending the pharmacokinetic AUC (area under the curve of a drug over time) of the therapeutic with the increased risk of off-target toxicities as it gets slowly cleared from the system. (Matthay, K. K. et al. (2007) J Clin Oncol 25, 1054-1060). Blood and marrow toxicities are among the most common toxicities, but these may be reversible. In contrast, extramedullary toxicities, such as renal and hepatic toxicities, can be slow to recover and potentially serious and/or lethal to a subject. If a therapeutic is too small (<70 kDa) and filtered through the renal glomeruli, either larger doses or extended dosing regimens are necessary to overcome the short serum half-life, which is associated with the accompanying shortcomings of excessive cost, logistics, and increased risk of organ toxicity. Chemotherapeutic drugs, such as cisplatin (^{˜}300 Da) or microtubule poisons, are examples where extramedullary toxicities (renal) encountered during dose escalation is prohibitive. (Pinzani, V. et al. (1994) Cancer Chemoth Pharm 35, 1-9). Others chemotherapeutics, such as cyclophosphamide, where extramedullary toxicity is reduced but not absent, prolonged exposure will cause severe myelosuppression, myelodysplasia or even leukemia. For a small therapeutic protein, even one that is target-specific and extremely potent such as blinatumomab (CD19×CD3 bispecific antibody, ^{˜}50 kDa), quantitative delivery into the tumor is suboptimal, even with continuous infusion. (Topp, M. S. et al. (2014) J Clin Oncol; Topp, M. S. et al. (2015) Lancet Oncol 16, 57-66). On the other hand, when a therapeutic is too large (e.g. IgM, >1000 kDa), it may take many days to clear from the blood compartment, with difficulty penetrating tumor tissues or filtering through the kidney. For therapeutics in between this range (e.g., IgG, ^{˜}150 kDa), metabolism occurs through the retinculoendothelial system or liver and half-lives range from 1-4 weeks, where they recirculate in the blood/marrow, typically achieving a therapeutic index (ratio of AUC of tumor to AUC of blood/marrow) of <5:1. Such a low ratio is a setup for myelotoxicity, lymphotoxicity and major organ toxicities. An alternative approach is compartmental therapies, where the therapeutic is not given intravenously, but instead directly into the disease compartment (e.g., CSF or peritoneal cavity) to maximize drug level and efficacy. Parham, P. (2005) Nat Rev Immunol 5, 201-214; Kramer, K. et al. (2008) in ISPNO 2008; Kramer, K. et al. (2010) J Neuro-Oncol 97, 409-418). While this drug delivery strategy can be highly tumor-selective, its benefit is limited to those with localized disease in easily accessible body compartments. For human cancers where 90% of patients die from metastatic disease (Weigelt, B. et al. (2005) Nat Rev Cancer 5, 591-602) compartmental therapy is generally palliative but not curative.

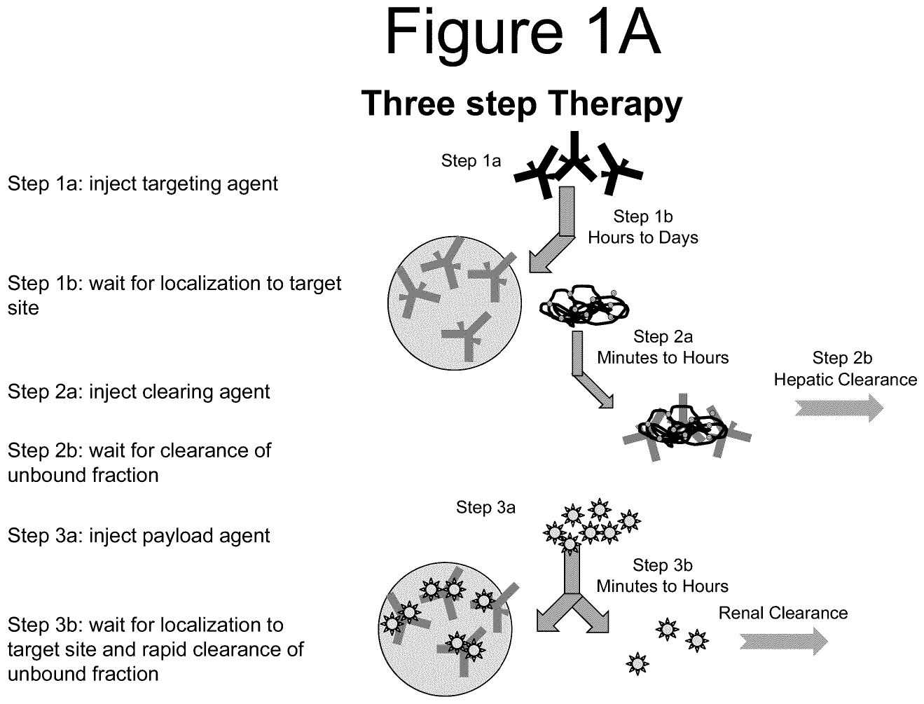

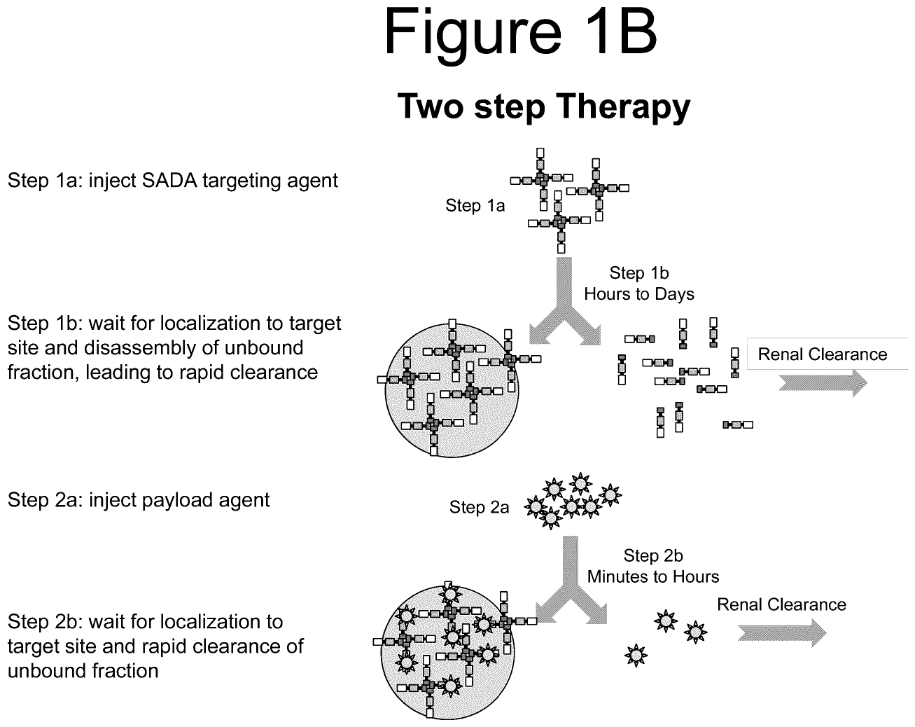

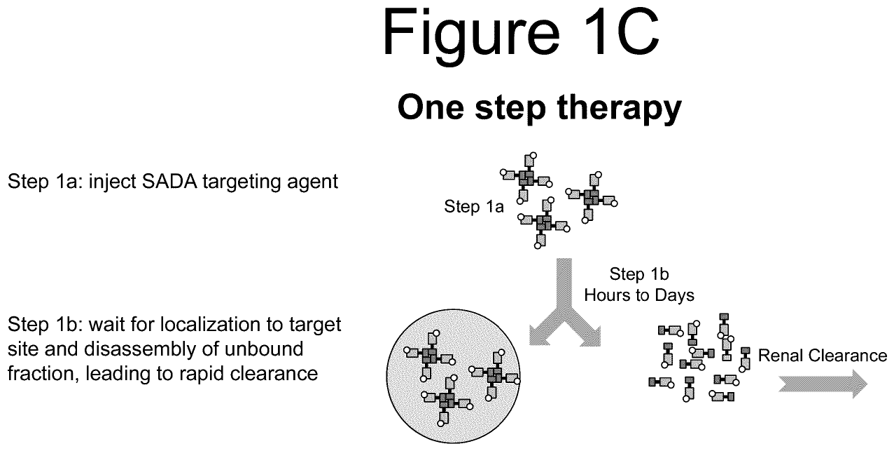

Many groups are now focusing on pretargeted therapies, where targeting and payload steps are separated into two steps. Various pretargeting (multistep) platforms have been successfully built to improve the therapeutic index, in some cases 10-100 fold. (Pagel, J. M. et al. (2003) Blood 101, 2340-2348; Carr, W. H. et al. (2005) J Immunol 175, 5222-5229; Thomas, R. et al. (2008) J Immunol 180, 6743-6750; Cheal, S. M. et al. (2014) Mol Cancer Ther 13, 1803-1812; Cheung, N. K. et al. (2004) J Nucl Med 45, 867-877). But in order not to delay the critical last payload step, the excess unbound antibody from the first step must be removed from the circulation, necessitating a clearing agent, and therefore creating a three-step procedure (FIG. 1A): 1) pretargeting antibody, 2) clearing agent, and 3) payload. Whereas a two-step approach (FIG. 1b) in drug delivery is already laborious; a multistep (23) approach increases complexity substantially, a setup for reducing compliance. An equally important consideration is the immunogenicity of these antibody constructs (e.g., streptavidin), which prevents repeat dosing in patients. Furthermore, some designs (e.g., streptavidin) have created unwanted off-target retention in critical organs, such as the kidneys, reducing their clinical utility.

Thus, there is an on-going need for agents that have effective kinetic and/or pharmacological properties with reduced or without associated toxicities.

### SADA Domains

The present disclosure encompasses a recognition that SADA domains can impart certain desirable functional characteristics to a conjugate. For example, the present disclosure provides an insight that SADA domains can be designed and/or tailored to achieve environmentally-dependent multimerization with beneficial kinetic, thermodynamic, and/or pharmacologic properties. For example, it is recognized that SADA domains may be part of a conjugate that permits effective delivery of a payload to a target site of interest while minimizing risk of off-target interactions.

The present disclosure also encompasses the recognition that most multimerization domains cannot be used for building a SADA domain. The present disclosure describes a number of beneficial characteristics and/or properties that may be used to select for an effective SADA domain. Example 11 describes a number of exemplary characteristics for selecting and/or designing an effective SADA domain. In some embodiments, a SADA domain may be selected for and/or designed to have certain beneficial properties. For example, in some embodiments, a SADA domain maintain a stable self-assembled multimeric state in vitro, to allow for manufacturability, but disassemble in vivo in a predictable way, such as, for example, to allow an initial prolonged serum half-life, followed by rapid clearance to reduce unwanted serum exposure. Additionally, a self-assembled multiunit SADA conjugate complex must be of sufficient size to ensure exceeding of the renal clearance threshold (^{˜}70 kDa), while falling below this cutoff when disassembled into monomeric subunits. Further beneficial properties of a SADA domain can include being non-immunogenic (e.g., of human origin), being of sufficient solubility and/or not being prone to aggregation or denaturation/instability during GMP manufacture.

Numerous multimerization domains would not meet the criteria of an effective SADA domain. For example, the most common multimerization domain, the human Fc domain derived from immunoglobulin IgG, would not qualify due to its covalent homodimerization with irreversible self-assembly. As a covalent dimer, it does not break into subunits in the serum for renal clearance. Even for IgG₄-Fc, which undergoes Fab exchange, the stable format is still an intact IgG₄ and not two Fab-Fc half molecules. Another example is streptavidin, which has been used previously to tetramerize single-chain fragments (scFv) for pre-targeted radioimmunotherapy (PRIT). Steptavidin was a clinical failure because of its high immunogenicity and intrinsic affinity for kidney tissues. (Pagel, J. M. et al. (2003) Blood 101, 2340-2348; Carr, W. H. et al. (2005) J Immunol 175, 5222-5229; Cheung, N. K. et al. (2004) J Nucl Med 45, 867-877; Parham, P. et al. (2011) J Immunol 187, 11-19; Zhang, M. L. et al. (2003) Proc. Natl. Acad. Sci. U.S.A. 100, 1891-1895; Oei, A. L. et al. (2008) Int J Cancer 123, 1848-1853). Other domains have not been successful partly due of their complexity, their size, or their instability during expression or purification, leading to difficulties during manufacturing and downstream processing.

The present disclosure encompasses the recognition that a SADA conjugate may have properties that permit a single-step (FIG. 1C) or two-step (FIG. 1B) targeting strategy. Further, it is recognized that these properties may improve antibody delivery, payload delivery, and their therapeutic indices for a targeted therapy (e.g., PRIT). As a proof of concept, we describe here design of a SADA domain derived from human p53, p63 and p73, and apply this to a Pretargeted Radio-Immuno-Therapy system (SADA-PRIT) as well as a cytokine therapy system (SADA-Cytokine). This modular self-clearing platform can be adapted to nearly any type of drug delivery: radioisotopes, cytokines, cytotoxic agents, protein toxins, peptides and nanoparticles, etc. It can also be used for trapping or sequestration of circulating ligands or receptors (e.g. drugs, toxins, venoms, growth factors, etc.) for hepatic or renal clearance, engaging immune cells to target cells (e.g. T-cell engagement, NK-cell engagement, etc.), or simply blocking receptor-ligand interactions.

The present disclosure encompasses the recognition that by modulating the self-association affinity of a SADA domain, including a combination of more than one independent SADA domain, one can regulate how quickly the multimeric complex disassembles into renally clearable subunits, therefore substantially influencing the pharmacokinetics of the therapeutic. In some embodiments, self-association affinity of a SADA domain allows for preferential self-assembly into a multimeric state at relatively high concentrations in vitro (>100 nM) but to prefer a disassembled lower order multimeric state (e.g., a monomeric state) at lower concentrations, which can allow for rapid renal clearance. The rate of disassembly of a SADA domain may be engineered to achieve a serum half-life that maximizes therapeutic index. In addition, the disassembly tendency (dissociation constant) of a SADA domain can be engineered to increase with decreasing pH or increasing temperature, whereby the multimeric forms will disassemble into monomeric units to enhance renal clearance. Therapeutics which benefit from extended half-lives can use more strongly associating domains in order to form larger complexes, while those that need a relatively short half-lives can use weaker associating domains. In some embodiments, a SADA domain is fused to a binding domain, wherein the binding domain binds a target in vivo, such that whenever target is present at sufficient concentration or density, this binding is strengthened by a multivalent avidity or cooperative binding to the target.

In some embodiments, by combining SADA domain, such as a tetramerizing SADA domain (e.g., p53, p63, p73, hnRNPC, SNAP-23, Stefin B, KCNQ4, CBFA2T1) with a dimerization domain such as a strong antiparallel dimerization domain (e.g., HNF1α) (Ahmed, M. et al. (2015) Oncolmmunology 4, e989776) or a strong antiparallel dimerization domain or trap (e.g., IL15Rα) (Chirifu, M. et al. (2007) _{Nat Immunol} 8, 1001-1007), a higher order multimerization platform can be built where the disassembly is sequential, from octamer to tetramer to dimer.

The present disclosure encompasses a recognition that association and disassociation rates of a SADA domain polypeptide can affect the pharmacokinetic properties of SADA conjugates (e.g., antibody-based SADA conjugates, SADA-Cytokine conjugates). In some embodiments, SADA domains are human derived multimerization domains that are sufficiently stable enough to multimerize tethered protein units in a non-covalent manner. In some embodiments, the present disclosure recognizes that it may be desirable to select a SADA domain that lacks unpaired cysteine residues. In some embodiments, it is recognized that it is beneficial to minimize exposed hydrophobic surfaces present in a SADA domain.

### Exemplary SADA Domains

In some embodiments, a SADA domain is composed of a multimerization domains which are each composed of helical bundles that associate in a parallel or anti-parallel orientation. In some embodiments, a SADA domain is selected from the group of one of the following human proteins: p53, p63, p73, heterogeneous nuclear Ribonucleoprotein (hnRNPC) C, or N-terminal domain of Synaptosomal-associated protein 23 (SNAP-23), Stefin B (Cystatin B), Potassium voltage-gated channel subfamily KQT member 4 (KCNQ4), Cyclin-D-related protein (CBFA2T1), or variants or fragments thereof. Provided below are polypeptide and nucleic acid sequences for exemplary SADA domains.

Human p53 tetramerization domain amino acid
sequence (321-359)
SEQ ID NO: 1
KPLDGEYFTLQIRGRERFEMFRELNEALELKDAQAGKEP
Human p53 tetramerization domain nucleotide
sequence
SEQ ID NO: 2
AAACCTCTGGATGGCGAGTACTTTACCCTGCAGATTAGAGGCCGCGAACG
ATTCGAGATGTTTCGCGAACTGAATGAGGCCCTGGAACTGAAGGATGCTC
AGGCAGGCAAGGAGCCA
Human p63 tetramerization domain amino acid
sequence (396-450)
SEQ ID NO: 3
RSPDDELLYLPVRGRETYEMLLKIKESLELMQYLPQHTIETYRQQQQQQH
QHLLQKQ
Human p63 tetramerization domain nucleotide
sequence
SEQ ID NO: 4
AGATCCCCCGACGATGAGCTGCTGTACCTGCCTGTGAGGGGCCGGGAGAC
CTATGAAATGCTGCTGAAGATCAAAGAGAGCCTGGAACTGATGCAGTACC
TGCCACAGCACACCATTGAAACATATAGGCAACAACAGCAGCAGCAGCAT
CAGCATCTGCTGCAGAAGCAG
Human p73 tetramerization domain amino acid
sequence (348-399)
SEQ ID NO: 5
RHGDEDTYYLQVRGRENFEILMKLKESLELMELVPQPLVDSYRQQQQLLQ
RP
Human p73 tetramerization domain nucleotide
sequence
SEQ ID NO: 6
AGGCACGGCGACGAAGATACCTACTATCTGCAGGTGAGGGGACGGGAGAA
CTTCGAAATCCTGATGAAGCTGAAAGAGTCCCTGGAACTGATGGAGCTGG
TGCCCCAGCCTCTGGTCGACAGCTACAGACAGCAGCAGCAGCTGCTGCAG
AGGCCA
Human HNRNPC tetramerization domain amino
acid sequence (194-220)
SEQ ID NO: 7
QAIKKELTQIKQKVDSLLENLEKIEKE
Human HNRNPC tetramerization domain nucleotide
sequence
SEQ ID NO: 8
CAAGCTATAAAGAAGGAACTCACCCAGATTAAGCAAAAGGTTGACTCACT
GTTGGAAAATCTTGAGAAAATAGAAAAGGAA
Human SNAP-23 tetramerization domain amino acid
sequence (23-76)
SEQ ID NO: 9
STRRILGLAIESQDAGIKTITMLDEQKEQLNRIEEGLDQINKDMRETEKT
LTEL
Human SNAP-23 tetramerization domain nucleotide
sequence
SEQ ID NO: 10
TCTACCCGCAGGATCTTGGGACTTGCTATAGAGTCACAGGACGCCGGAAT
AAAAACTATCACTATGCTTGATGAACAGAAGGAACAACTGAATCGGATTG
AGGAAGGACTGGACCAGATTAACAAGGACATGCGAGAGACCGAAAAAACA
CTCACTGAGTTG
Human Stefin B tetramerization domain amino acid
sequence (2-98)
SEQ ID NO: 11
MCGAPSATQPATAETQHIADQVRSQLEEKENKKFPVFKAVSFKSQVVAGT
NYFIKVHVGDEDFVHLRVFQSLPHENKPLTLSNYQTNKAKHDELTYF
Human Stefin B tetramerization domain nucleotide
sequence
SEQ ID NO: 12
ATGTGCGGGGCGCCCTCCGCCACGCAGCCGGCCACCGCCGAGACCCAGCA
CATCGCCGACCAGGTGAGGTCCCAGCTTGAAGAGAAAGAAAACAAGAAGT
TCCCTGTGTTTAAGGCCGTGTCATTCAAGAGCCAGGTGGTCGCGGGGACA
AACTACTTCATCAAGGTGCACGTCGGCGACGAGGACTTCGTACACCTGCG
AGTGTTCCAATCTCTCCCTCATGAAAACAAGCCCTTGACCTTATCTAACT
ACCAGACCAACAAAGCCAAGCATGATGAGCTGACCTATTTC
KCNQ4 tetramerization domain amino acid sequence
(611-640)
SEQ ID NO: 13
DEISMMGRVVKVEKQVQSIEHKLDLLLGFY
KCNQ4 tetramerization domain nucleotide sequence
SEQ ID NO: 14
GATGAAATCAGCATGATGGGACGCGTGGTCAAGGTGGAGAAGCAGGTGCA
GTCCATCGAGCACAAGCTGGACCTGCTGTTGGGCTTCTAT
CBFA2T1 tetramerization domain amino acid sequence
(462-521)
SEQ ID NO: 15
TVAEAKRQAAEDALAVINQQEDSSESCWNCGRKASETCSGCNTARYCGSF
CQHKDWEKHH
CBFA2T1 tetramerization domain nucleotide sequence
SEQ ID NO: 16
ACGGTCGCCGAGGCCAAACGGCAGGCGGCGGAGGACGCACTGGCAGTTAT
CAATCAGCAGGAGGATTCAAGCGAGAGTTGCTGGAATTGTGGCCGTAAAG
CGAGTGAAACCTGCAGTGGCTGTAACACAGCCCGATACTGTGGCTCATTT
TGCCAGCACAAAGACTGGGAGAAGCACCAT

In some embodiments, a SADA polypeptide is or comprises a tetramerization domain of p53, p63, p73, hnRNPC, SNAP-23, Stefin B, KCNQ4, or CBFA2T1. In some embodiments, a SADA polypeptide is or comprises a sequence that is at least 75%, 80%, 85%, 90%, 91%, 92%, 93%, 94%, 95%, 96%, 97%, 98%, 99% or 100% identical to a sequence as set forth in any one of SEQ ID NOs: 1, 3, 5, 7, 9, 11, 13, and 15. In some embodiments, a SADA polypeptide is or comprises a sequence that is at least 75%, 80%, 85%, 90%, 91%, 92%, 93%, 94%, 95%, 96%, 97%, 98%, or 99% identical to a sequence as set forth in any one of SEQ ID NOs: 1, 3, 5, 7, 11, and 13, and wherein the underlined amino acid residues in these sequences above are conserved.

SADA conjugates and Uses

The present disclosure encompasses a recognition that SADA domains can impart certain desirable functional characteristics to a conjugate. For example, the present disclosure provides an insight that SADA domains can be designed and/or tailored to achieve environmentally-dependent multimerization with beneficial kinetic, thermodynamic, and/or pharmacologic properties. For example, it is recognized that SADA domains may be part of a conjugate that permit effective delivery of a payload to a target site of interest while minimizing risk of off-target interactions.

Among other things, the present disclosure provides various conjugates comprising a SADA domain linked to one or more binding domains. In some embodiments, such conjugates are characterized in that they multimerize to form a complex of a desired size under relevant conditions (e.g., in a solution in which the conjugate is present above a threshold concentration or pH and/or when present at a target site characterized by a relevant level or density of receptors for the payload), and disassemble to a smaller form under other conditions (e.g., absent the relevant environmental multimerization trigger).

The present disclosure provides, among other things, an appreciation that a SADA conjugate may have improved characteristics compared to a conjugate without a SADA domain. In some embodiments, a SADA conjugate includes a binding domain. In some embodiments, improved characteristics include that a multimeric conjugate has increased avidity/binding to a target, increased specificity for target cells or tissues, and/or extended initial serum half-life. In some embodiments, improved characteristics include that through dissociation to smaller states (e.g. dimeric or monomeric) exhibit reduced non-specific binding, decreased toxicity, and/or improved renal clearance.

In some embodiments, a SADA conjugate comprises (i) a self-assembly disassembly (SADA) polypeptide having an amino acid sequence that shows at least 75% identity with that of a human homo-multimerizing polypeptide and is characterized by one or more multimerization dissociation constants (K_{D}); and (ii) at least a first binding domain that binds to a first target and is covalently linked to the SADA polypeptide. In some embodiments, a SADA conjugate is constructed and arranged so that it adopts a first multimerization state and one or more higher-order multimerization states. In some embodiments, a first multimerization state is less than about ^{˜}70 kDa in size. In some embodiments, a first multimerization state is an unmultimerized state (e.g., a monomer or a dimer). In some embodiments, a first multimerization state is a monomer. In some embodiments, a first multimerization state is a dimer. In some embodiments, a first multimerization state is a multimerized state (e.g., a trimer or a tetramer). In some embodiments, a higher-order multimerization states is a homo-tetramer or higher-order homo-multimer greater than 150 kDa in size. In some embodiments, a higher-order homo-multimerized conjugate is stable in aqueous solution when the conjugate is present at a concentration above the SADA polypeptide K_{D}. In some embodiments, a SADA conjugate transitions from a higher-order multimerization state(s) to a first multimerization state under physiological conditions when the concentration of the conjugate is below the SADA polypeptide K_{D}.

In some embodiments, a SADA polypeptide is covalently linked to a binding domain via a linker. Any suitable linker known in the art can be used. In some embodiments, a SADA polypeptide is linked to a binding domain via a polypeptide linker. In some embodiments, a polypeptide linker is a Gly-Ser linker. In some embodiments, a polypeptide linker is or comprises a sequence of (GGGGS)n, where n represents the number of repeating GGGGS units and is 1, 2, 3, 4, 5, 6, 7, 8, 9, 10, 11, 12, 13, 14, 15, 16, 17, 18, 19, 20, 25, 30 or more. In some embodiments, a binding domain is directly fused to a SADA polypeptide.

The present disclosure provides SADA conjugates as described herein that may be used in a method of treatment of the human or animal body, or in a method of diagnosis. In some embodiments, a SADA conjugate has a binding domain that can bind to a moiety associated with a target, such as target cells and/or tissues. In some embodiments a target cell is a tumor cell. In some embodiments, a SADA conjugate is capable of selectively binding a tumor that expresses moiety for which a binding domain has affinity. In some embodiments, a SADA conjugate may be suitable for therapeutic treatment of patients.

In some embodiments, as will be understood in the art, a SADA conjugate may be utilized without further modification. In some embodiments, a SADA conjugate may be incorporated into a composition or formulation. In some embodiments, a SADA conjugate comprises a binding domain that non-covalently binds to a therapeutic payload. In some embodiments, they may be chemically associated or linked (e.g., covalently linked) with one or more other agents or entities, e.g., with a therapeutic payload.

In some embodiments, a SADA conjugate may be used for targeted therapy and/or diagnostics. The present disclosure encompasses the recognition that a SADA conjugate may have properties that permit a single-step (FIG. 1C) or two-step (FIG. 1B) targeting strategy. Further, it is recognized that these properties may improve antibody delivery, payload delivery, and their therapeutic indices for a targeted therapy (e.g., PRIT). As a proof of concept, we describe here design of a SADA domain derived from human p53, p63 and p73, and apply this to a Pretargeted Radio-Immuno-Therapy system (SADA-PRIT) as well as a cytokine therapy system (SADA-Cytokine). This modular self-clearing platform can be adapted to nearly any type of drug delivery: radioisotopes, cytokines, cytotoxic agents, protein toxins, peptides and nanoparticles, etc. It can also be used for trapping or sequestration of circulating ligands or receptors (e.g. drugs, toxins, venoms, growth factors, etc.) for hepatic or renal clearance, engaging immune cells to target cells (e.g. T-cell engagement, NK-cell engagement, etc.), or simply blocking receptor-ligand interactions.

In some embodiments, a SADA-PRIT delivery system comprises: a multiunit antibody of (1) non-immunogenic human or humanized components, (2) sufficient initial self-assembled molecular size above the renal threshold to allow for continual blood circulation (e.g., range 12-96 hours) and quantitative uptake into tumors, (3) an inherent ability to disassemble into small units below the renal threshold, such that any remaining unbound protein will be excreted through the kidney (e.g. range 12-96 hours) without the requirement for any clearing agent, and thereby permitting (4) a final payload to be carried by a ligand small enough to efficiently penetrate tissues and bind with high affinity to the pretargeted antibody, while also allowing for any unbound payload to be excreted through the kidney, within minutes to hours after administration. Because multimeric self-assembly is in part a concentration dependent phenomenon, this system takes advantage of the fact that the SADA multimers will have an increased local concentration at their target sites (such as a tumor) where the multimer is stabilized by multivalent binding that favors self-assembly, while simultaneously having a decreased local concentration at non-target sites (e.g. blood) that favors disassembly followed by rapid renal clearance.

In some embodiments, a SADA conjugate (e.g., SADA-Cytokine or SADA-BiDE), a binding domain (e.g. antibody, cytokine, enzyme, fluorophore, small molecule inhibitor, etc.) can be covalently attached to a SADA polypeptide and be selectively delivered to the target. In some embodiments, a SADA conjugate can further comprise a payload. In some embodiments, a SADA conjugate may be covalently or non-covalently associated with a payload. In some embodiments, the payload may be or comprise a therapeutic agent payload (e.g., a toxic payload). In some embodiments the payload may be or comprise a detection agent payload. Without wishing to be bound by theory, it is envisions that selective delivery of a SADA conjugate and/or a SADA conjugate with a payload, may be due, at least in part, by virtue of the increased substrate avidity through multiunit assembly or enhanced endocytosis, allowing for maximal effect at the target sites (tumor, effector cells, etc.) while minimizing off target side effects due to the rapid clearance from non-targeted tissues.

In some embodiments, a SADA conjugate comprises a SADA domain and a binding domain that can bind to and sequester one or more target moieties or entities (e.g., a SADA-Trap conjugate). In some embodiments of the SADA platform soluble proteins or peptides (e.g. tumor factors, growth factors, inhibitory proteins, activation molecules, venoms, toxins, etc.), haptens, or chemicals can be sequestered by a SADA-Trap, and renally cleared. In a fully self-assembled state, the multimerized SADA-Trap can bind and capture relatively small soluble targets (<50 kDa) (in the blood, CSF, peritoneum, other body fluids or compartments, etc.) more effectively than classic Fab-based traps, by virtue of its enhanced avidity and its initial long serum half-life. After circulating for a specified period of time, the SADA-Trap will be disassembled into Trap:Target monomers and rapidly cleared renally. Similarly, when targeted to large soluble targets (>60 kDa), the SADA-Trap can bind and inhibit their function by blocking their active sites, or enhancing their metabolism by the liver.

In some embodiments, a SADA conjugate comprises a SADA domain and a binding domain that can bind to one or more targets that are associated with a white blood cell (e.g., a SADA-BiWE conjugate). In some embodiment of the SADA platform, a white blood cell engaging bispecific (BiWE), can be multimerized by the SADA domain (SADA-BiWE) to more effectively activate white blood cells against an antigen of interest. As opposed to classic bispecific engagers, such as blinatumomab, allowing for multivalent binding allows the targeted white blood cell to recognize low-density targets (such as low frequency peptide-HLA complexes) or classically difficult targets with low affinity antibodies (such as carbohydrate antigens). Furthermore, unlike IgG based bispecifics, the SADA domain allows for rapid clearance of unbound SADA-BiWE, limiting their off-target exposure. Additionally, their increased avidity should allow for better retention on both target and effector cell populations, providing a long period of activity without needing an excess of circulating mAb.

### Conjugate Production

In some embodiments, conjugates comprising a SADA-domain as described herein may be produced from nucleic acid molecules using molecular biological methods known to the art. Nucleic acid molecules are inserted into a vector that is able to express the fusion proteins in when introduced into an appropriate host cell. Appropriate host cells include, but are not limited to, bacterial, yeast, insect, and mammalian cells. Any of the methods known to one skilled in the art for the insertion of DNA fragments into a vector may be used to construct expression vectors encoding the fusion proteins of the present invention under control of transcriptional/translational control signals. These methods may include in vitro recombinant DNA and synthetic techniques and in vivo recombination (See Sambrook et al. Molecular Cloning, A Laboratory Manual, Cold Spring Harbor Laboratory; Current Protocols in Molecular Biology, Eds. Ausubel, et al, Greene Publ. Assoc., Wiley-Interscience, NY).

Expression of nucleic acid molecules in accordance with the present invention may be regulated by a second nucleic acid sequence so that the molecule is expressed in a host transformed with the recombinant DNA molecule. For example, expression of the nucleic acid molecules of the invention may be controlled by a promoter and/or enhancer element that are known in the art.

Nucleic acid constructs include sequences that encode SADA conjugates that include a SADA domain and a binding domain. In some embodiments, a binding domain of a SADA conjugate is an antibody or antibody component. Typically, such antibody components will be generated from V_{H} and/or V_{L} regions. After identification and selection of antibodies or antibody components exhibiting desired binding and/or functional properties, variable regions of each antibody are isolated, amplified, cloned and sequenced. Modifications may be made to the V_{H} and V_{L} nucleotide sequences, including additions of nucleotide sequences encoding amino acids and/or carrying restriction sites, deletions of nucleotide sequences encoding amino acids, or substitutions of nucleotide sequences encoding amino acids. The antibodies and/or antibody components may be generated from human, humanized or chimeric antibodies.

Nucleic acid constructs of the present invention are inserted into an expression vector or viral vector by methods known to the art, and nucleic acid molecules are operatively linked to an expression control sequence.

Where appropriate, nucleic acid sequences that encode humanized antibodies and multi-specific binding agents as described herein may be modified to include codons that are optimized for expression in a particular cell type or organism (e.g., see U.S. Pat. Nos. 5,670,356 and 5,874,304). Codon optimized sequences are synthetic sequences, and preferably encode the identical polypeptide (or a biologically active fragment of a full length polypeptide which has substantially the same activity as the full length polypeptide) encoded by the non-codon optimized parent polynucleotide. In some embodiments, the coding region of the genetic material encoding antibody components, in whole or in part, may include an altered sequence to optimize codon usage for a particular cell type (e.g., a eukaryotic or prokaryotic cell). For example, the coding sequence for a humanized heavy (or light) chain variable region as described herein may be optimized for expression in a bacterial cells. Alternatively, the coding sequence may be optimized for expression in a mammalian cell (e.g., a CHO). Such a sequence may be described as a codon-optimized sequence.

An expression vector containing a nucleic acid molecule is transformed into a suitable host cell to allow for production of the protein encoded by the nucleic acid constructs. Exemplary host cells include prokaryotes (e.g., E. coli) and eukaryotes (e.g., a COS or CHO cell). Host cells transformed with an expression vector are grown under conditions permitting production of a SADA conjugate of the present invention followed by recovery of the SADA conjugate.

SADA conjugates of the present disclosure may be purified by any technique, which allows for the subsequent formation of a stable antibody or binding agent molecule. For example, not wishing to be bound by theory, SADA conjugates may be recovered from cells either as soluble polypeptides or as inclusion bodies, from which they may be extracted quantitatively by 8M guanidinium hydrochloride and dialysis. In order to further purify SADA conjugates of the present invention, conventional ion exchange chromatography, hydrophobic interaction chromatography, reverse phase chromatography or gel filtration may be used. SADA conjugates of the present invention may also be recovered from conditioned media following secretion from eukaryotic or prokaryotic cells.

A variety of technologies for conjugating agents, or components thereof, with other moieties or entities are well known in the art and may be utilized in accordance with the practice of the present disclosure. To give but one example, radioactively-labeled SADA conjugates may be produced according to well-known technologies in the art.

For instance, in some embodiments, SADA conjugates can be iodinated by contact with sodium and/or potassium iodide and a chemical oxidizing agent such as sodium hypochlorite, or an enzymatic oxidizing agent, such as lactoperoxidase. In some embodiments, SADA conjugates may be labeled with technetium-99m by ligand exchange process, for example, by reducing pertechnate with stannous solution, chelating the reduced technetium onto a Sephadex column and applying the antibody to this column. In some embodiments, provided SADA conjugates are labeled using direct labeling techniques, e.g., by incubating pertechnate, a reducing agent such as SNCl2, a buffer solution such as sodium-potassium phthalate solution, and the antibody. Intermediary functional groups which are often used to bind radioisotopes which exist as metallic ions to antibody are diethylenetriaminepentaacetic acid (DTPA), or ethylene diaminetetracetic acid (EDTA), or 1,4,7,10-tetraazacyclododecane-1,4,7,10-tetraacetic acid (DOTA), or p-aminobenzyl-DOTA (Bn-DOTA). Radioactive isotopes may be detected by, for example, dosimetry.

### Administration

The present disclosure provides methods of administering an effective amount of a conjugate comprising a SADA domain as described herein (e.g., antibody-based SADA conjugates, SADA-Cytokine conjugates) to a subject in need of treatment.

To give but a few examples, in some embodiments, a SADA conjugate as described herein is administered under conditions and for a period of time (e.g., according to a dosing regimen) sufficient for it to saturate a target or target cells (e.g., tumor cells). In some embodiments, unbound SADA conjugate clears from the blood stream after administration; in some such embodiments, such removal occurs (e.g., is permitted to occur) prior to administration of another agent.

In some particular embodiments, a SADA conjugate as described herein is administered in combination with another agent that targets Bn-DOTA. In some such embodiments, the another agent carries a payload. In some embodiments, the payload may be or comprise a therapeutic agent payload (e.g., a toxic payload). In some embodiments the payload may be or comprise a detection agent payload.

In some particular embodiments, a SADA domain as described herein (e.g., antibody-based SADA conjugates, SADA-Cytokine conjugates) as described herein is administered so that tumor cells are saturated, and subsequently a second agent, that targets Bn-DOTA (and may carry a payload) is administered. Optionally, at least one third agent that targets Bn-DOTA (e.g., and may carry a different payload) may be administered.

In some embodiments, additional agents are administered a period of time after administration of a SADA conjugate described herein, which period of time may be sufficient to permit clearance of unbound therapeutic agent. In some embodiments, additional agents are administered without further administration of the therapeutic agent. For example, in some embodiments, a SADA conjugate as described herein is administered according to a regimen that includes at least one cycle of: (i) administration of the SADA conjugate (optionally so that relevant tumor cells are saturated); (ii) administration of a second and, optionally at least one third agent (e.g., that targets Bn-DOTA, and may optionally carry a payload); (iii) optional additional administration of the second and/or third agents, without additional administration of the SADA conjugate. In some embodiments, a therapeutic regimen may comprise multiple such cycles; in some embodiments, a regimen may comprise 1, 2, 3, 4, 5, 6, 7, 8, 9, 10 or more cycles.

In some embodiments, a therapeutic regimen comprises only a single cycle that includes administration of a SADA conjugate; in some embodiments such a therapeutic regimen may comprise one or more cycles that include steps (ii) and, optionally, (iii) but do not include additional administrations of the SADA conjugate.

Those of ordinary skill in the art, reading the present disclosure, will readily appreciate that therapy with a SADA conjugate described herein (e.g., antibody-based SADA conjugates, SADA-Cytokine conjugates), may in certain embodiments be combined with other therapies, and particularly including other anti-tumor therapies. In some embodiments, such other anti-tumor therapies may be or comprise, for example administration of one or more chemotherapeutic agents, immunomodulatory agents, radiation therapy, high-frequency ultrasound therapy, surgery, etc.

In some embodiments, relative timing of administration of a SADA conjugate described herein (e.g., antibody-based SADA conjugates, SADA-Cytokine conjugates) and another therapy with which it is combined may be selected to optimize effect.

SADA conjugates as described herein may be administered through various methods known in the art for the therapeutic and/or diagnostic delivery of agents. For example, proteins or nucleic acids can be used for the therapeutic delivery of a SADA or a nucleic acid encoding a SADA conjugate of the present disclosure, e.g., cellular transfection, gene therapy, direct administration with a delivery vehicle or pharmaceutically acceptable carrier, indirect delivery by providing recombinant cells comprising a nucleic acid encoding a SADA conjugate of the present disclosure. In some embodiments, administration of a SADA conjugate induces killing of or inhibits growth of target cells in a subject.

Various delivery systems are known and can be used to administer a SADA conjugate of the present disclosure, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Routes of administration can be enteral or parenteral and include, but are not limited to, intravenous, subcutaneous, intramuscular, parenteral, transdermal, or transmucosal (e.g., oral or nasal). In some embodiments, SADA conjugates of the present disclosure are administered intravenously. In some embodiments, SADA conjugates of the present disclosure are administered subcutaneously. In some embodiments, SADA conjugates of the present disclosure are administered together with other biologically active agents.

In some embodiments, prior administration of a SADA conjugate as described herein permits combination therapy in which the agent with which the SADA conjugate is combined shows a broader therapeutic index than it does when administered alone (i.e., without the prior administration of a therapeutic agent as described herein). In some embodiments, such a broader therapeutic index is at least a log fold improved.

### Formulation

The present disclosure further provides compositions comprising SADA conjugates of the present disclosure and a pharmaceutically acceptable carrier or excipient. The composition, if desired, can also contain one or more additional therapeutic and/or diagnostic agents.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

Formulations of the pharmaceutical compositions described herein may be prepared by any method known or hereafter developed in the art of pharmacology. In general, such preparatory methods include the step of bringing the active ingredient into association with a diluent or another excipient and/or one or more other accessory ingredients, and then, if necessary and/or desirable, shaping and/or packaging the product into a desired single- or multi-dose unit.

A pharmaceutical composition in accordance with the present invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient that would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100% (w/w) active ingredient.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by the United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

General considerations in the formulation and/or manufacture of pharmaceutical agents may be found, for example, in Remington: The Science and Practice of Pharmacy 21st ed., Uppincott Williams & Wilkins, 2005 (incorporated herein by reference).

The present disclosure further provides a pharmaceutical pack or kit comprising one or more containers filled with at least one SADA conjugate as described herein. Kits may be used in any applicable method, including, for example, therapeutically or diagnostically. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

Identification and/or Characterization of SADA Conjugates

In some embodiments, a SADA-conjugate may be identified or characterized by a method comprising steps of (i) providing a conjugate comprising a self-assembly disassembly (SADA) polypeptide and a binding domain and (ii) determining a threshold condition (e.g., concentration, pH/pOH, oxidation/reduction state) wherein the conjugate substantially adopts a multimeric form greater than about ^{˜}70 kDa. Any methods known in the art can be used to assess the multimeric form of an antibody agent, include chromatographic methods. In some embodiments, the step of providing comprises providing a conjugate in which the SADA polypeptide is a test polypeptide and the step of determining comprises identifying the multimerization domain as useful in the conjugate if the critical multimerization concentration is within a range of about 100 nM to 1 mM. In some embodiments, the step of providing comprises providing a plurality of conjugates, and the step of determining comprises determining the threshold for each of the conjugates. In some embodiments, each conjugate in the plurality comprises the same binding domain but differs in the SADA polypeptide.

In some embodiments, a SADA-conjugate may be identified or characterized by a method comprising steps of (i) providing a conjugate comprising a self-assembly disassembly (SADA) polypeptide and a binding domain, (ii) administering the composition to a subject and (iii) determining the affinity of the conjugate for a target. Any methods known in the art for determining the affinity of a conjugate for a target may be used in the art. In some embodiments, affinity may be assessed as binding affinity. In some embodiments, affinity by be assessed by localization, using any techniques known in the art to visualize localization.

In some embodiments, a SADA-conjugate may be identified or characterized by a method that includes analysis of one or more conjugates in a plurality of conjugates. In some embodiments, a SADA-conjugate may be identified or characterized by a method comprising steps of (i) providing composition comprising a plurality of conjugates, each comprising a SADA polypeptide and a binding domain, (ii) administering the composition to a subject and (iii) determining the affinity of one or more of the conjugates for a target. In some embodiments, a step of determining comprises determining the affinity for a target for each of the conjugates. In some embodiments, a method includes a step of determining the rate of clearance of one or more conjugate from blood. In some embodiments, a method includes a step of determining the rate of clearance of a conjugate from blood for each of a plurality of conjugates. In some embodiments, a plurality of conjugates includes SADA conjugates that comprise the same binding domain but differ in the SADA polypeptide.

In some embodiments, a SADA-conjugate may be identified or characterized as preferred relative to another conjugate in a plurality of conjugates when the preferred conjugate shows increased avidity for a target and/or when the preferred conjugate is more rapidly cleared from the blood.

In some embodiments, a SADA-conjugate may be identified or characterized by a method that includes steps of (i) providing a composition comprising a SADA conjugate, and (ii) formulating the conjugate with a pharmaceutically acceptable carrier or excipient to produce a composition in which the conjugate is present at a concentration sufficient for at least 90% of the conjugate to adopt the higher-order multimerized state. In some embodiments, a conjugate in the composition is at a concentration of 50 nM, 100 nM, 500 nM, 1 μM, 10 μM, 50 μM, 100 μM, 200 μM, 300 μM, 400 μM, 500 μM, or 1 mM.

### EXEMPLARY EMBODIMENTS

Exemplary embodiment 1. A polypeptide conjugate comprising: a self-assembly disassembly (SADA) polypeptide having an amino acid sequence that shows at least 75% identity with that of a human homo-multimerizing polypeptide and being characterized by one or more multimerization dissociation constants (K_{D}); and at least a first binding domain that binds to a first target and is covalently linked to the SADA polypeptide,
the conjugate being constructed and arranged so that it adopts a first multimerization state and one or more higher-order multimerization states, where:
   the first multimerization state is less than about ^{˜}70 kDa in size,
   at least one of the higher-order multimerization states is a homo-tetramer or higher-order homo-multimer greater than 150 kDa in size,
   where the higher-order homo-multimerized conjugate is stable in aqueous solution when the conjugate is present at a concentration above the SADA polypeptide K_{D}, and
   the conjugate transitions from the higher-order multimerization state(s) to the first multimerization state under physiological conditions when the concentration of the conjugate is below the SADA polypeptide K_{D}.

Exemplary embodiment 2. The conjugate of exemplary embodiment 1, where the higher-order homo-multimerized conjugate is stable for a period of at least 24 hr at 37° C. in an aqueous buffer with a pH of about 7.

Exemplary embodiment 3. The conjugate of exemplary embodiment 2 or 3, where the higher-order homo-multimerized conjugate is stable for a period of at least 48 hours, 72 hours, 1 week, 2 weeks, 1 month, 2 months, 3 months, or more.

Exemplary embodiment 4. The conjugate of any one of exemplary embodiments 1-3, where the higher-order homo-multimerized conjugate is stable over 3 or more freeze-thaw cycles.

Exemplary embodiment 5. The conjugate of any one of exemplary embodiments 1-4, where the transition of the conjugate from the higher-order multimerization state to the first multimerization state is characterized by a K_{off} within a range of 1×10⁻⁶ to 1×10⁻⁴ (s⁻¹).

Exemplary embodiment 6. The conjugate of any one of exemplary embodiments 1-5, where the SADA polypeptide has a total buried surface area of 900 A2 to 4000 A2.

Exemplary embodiment 7. The conjugate of any one of exemplary embodiments 1-6, where the SADA polypeptide lacks unpaired cysteine residues.

Exemplary embodiment 8. The conjugate of any one of exemplary embodiments 1-7, where the SADA polypeptide comprises a tetramerization, pentamerization or hexamerization domain.

Exemplary embodiment 9. The conjugate of any one of exemplary embodiments 1-8, where the SADA polypeptide is or comprises a tetramerization domain of any one of p53, p63, p73, hnRNPC, SNAP-23, Stefin B, KCNQ4, and CBFA2T1.

Exemplary embodiment 10. The conjugate of any one of exemplary embodiments 1-8, where the SADA polypeptide is or comprises a tetramerization domain of p53.

Exemplary embodiment 11. The conjugate of any one of exemplary embodiments 1-8, where the SADA polypeptide is or comprises a tetramerization domain of p63.

Exemplary embodiment 12. The conjugate of any one of exemplary embodiments 1-8, where the SADA polypeptide is or comprises a tetramerization domain of p73.

Exemplary embodiment 13. The conjugate of any one of exemplary embodiments 1-8, where the SADA polypeptide is or comprises a tetramerization domain of hnRNPC.

Exemplary embodiment 14. The conjugate of any one of exemplary embodiments 1-8, where the SADA polypeptide is or comprises a tetramerization domain of SNAP-23.

Exemplary embodiment 15. The conjugate of any one of exemplary embodiments 1-8, where the SADA polypeptide is or comprises a tetramerization domain of Stefin B.

Exemplary embodiment 16. The conjugate of any one of exemplary embodiments 1-8, where the SADA polypeptide is or comprises a tetramerization domain of KCNQ4.

Exemplary embodiment 17. The conjugate of any one of exemplary embodiments 1-8, where the SADA polypeptide is or comprises a tetramerization domain of CBFA2T1.

Exemplary embodiment 18. The conjugate of any one of exemplary embodiments 1-9, where the SADA polypeptide is or comprises a sequence that is at least 90%, at least 95%, at least 96%, at least 97%, at least 98%, or at least 99% identical to a sequence as set forth in any one of SEQ ID NOs: 1, 3, 5, 7, 9, 11, 13, and 15.

Exemplary embodiment 19. The conjugate of any one of exemplary embodiments 1-18, where the first target is an in situ target.

Exemplary embodiment 20. The conjugate of exemplary embodiment 19, where the first target is an in situ target that is or comprises an entity selected from the group consisting of: a cell-surface moiety, a cytokine, a receptor ligand, a peptide, a hormone, a metabolite, and a hapten.

Exemplary embodiment 21. The conjugate of any one of exemplary embodiments 1-18, where the first target is a payload target.

Exemplary embodiment 22. The conjugate of exemplary embodiment 21, where the first target is a therapeutic payload.

Exemplary embodiment 23. The conjugate of exemplary embodiment 21, where the first target is a diagnostic payload.

Exemplary embodiment 24. The conjugate of any one of exemplary embodiments 21-23, where the payload target is a drug, a polypeptide (such as a toxin, enzyme, cytokine, chemokine, receptor, or biologic), a chemical probe (such as a fluorescent dye or biotin tag), a radioactive isotope, or a nanoparticle.

Exemplary embodiment 25. The conjugate of any one of exemplary embodiments 1-24, further comprising a second binding domain that binds to a second target, which is different from the first target.

Exemplary embodiment 26. The conjugate of exemplary embodiment 25, where the conjugate comprises at least two binding domains and wherein the conjugate in the second multimerization state is at least octavalent.

Exemplary embodiment 27. The conjugate of exemplary embodiment 25 or 26, where the second target is an in situ target.

Exemplary embodiment 28. The conjugate of exemplary embodiment 27, where the second target is an in situ target that is or comprises an entity selected from the group consisting of: a cell-surface moiety, a cytokine, a receptor ligand, a peptide, a hormone, a metabolite, and a hapten.

Exemplary embodiment 29. The conjugate of exemplary embodiment 25 or 26, where the second target is a payload target.

Exemplary embodiment 30. The conjugate of exemplary embodiment 29, where the second target is a therapeutic payload.

Exemplary embodiment 31. The conjugate of exemplary embodiment 29, where the second target is a diagnostic payload.

Exemplary embodiment 32. The conjugate of any one of exemplary embodiments 29-31, where the payload target is a drug, a polypeptide (such as a toxin, enzyme, cytokine, chemokine, receptor, or biologic), a chemical probe (such as a fluorescent dye or biotin tag), a radioactive isotope, or a nanoparticle.

Exemplary embodiment 33. The conjugate of of any one of exemplary embodiments 1-24, where the first target is a cell surface moiety.

Exemplary embodiment 34. The conjugate of exemplary embodiment 25 or 26, where the second target is a cell surface moiety.

Exemplary embodiment 35. The conjugate of exemplary embodiment 33 or 34, where the cell surface moiety is specifically expressed or enriched on a subset of cells in an organism.

Exemplary embodiment 36. The conjugate of exemplary embodiment 35, where the cell surface moiety is specifically expressed or enriched on tumor cells.

Exemplary embodiment 37. The conjugate of any one of exemplary embodiments 34-36, where the cell surface moiety is a cell surface receptor.

Exemplary embodiment 38. The conjugate of any one of exemplary embodiments 1-24, where the first binding domain is or comprises a ligand for a cell surface receptor.

Exemplary embodiment 39. The conjugate of any one of exemplary embodiments 25-36, where the first and/or second binding domain is or comprises a ligand for a cell surface receptor.

Exemplary embodiment 40. The conjugate of any one of exemplary embodiments 1-24, where the first binding domain is or comprises a cytokine receptor binding domain.

Exemplary embodiment 41. The conjugate of any one of exemplary embodiments 25-36, where the first and/or second binding domain is or comprises a cytokine receptor binding domain.

Exemplary embodiment 42. The conjugate of exemplary embodiment 40 or 41, where the conjugate is further complexed with a soluble cytokine polypeptide.

Exemplary embodiment 43. The conjugate of exemplary embodiment 42, where the cytokine receptor is IL15Rα and the soluble cytokine polypeptide is IL15.

Exemplary embodiment 44. The conjugate of any one of exemplary embodiments 1-24, where the first binding domain is or comprises an antibody, antibody component, or antigen-binding antibody fragment specific for a cell surface target.

Exemplary embodiment 45. The conjugate of any one of exemplary embodiments 25-36, where the first and/or second binding domain is or comprises an antibody, antibody component, or antigen-binding antibody fragment specific for a cell surface target.

Exemplary embodiment 46. The conjugate of exemplary embodiment 44 or 45, where the first and/or second binding domain is an antibody component.

Exemplary embodiment 47. The conjugate of exemplary embodiment 44 or 45, where the first and/or second binding domain is an antigen-binding antibody fragment.

Exemplary embodiment 48. The conjugate of exemplary embodiment 44 or 45, where the first and/or second binding domain is an scFv.

Exemplary embodiment 49. The conjugate of any one of exemplary embodiments 45-48, where the first binding domain is an anti-GD2, anti-Globo H, anti-GPA33, anti-PSMA, anti-polysialic acid, anti-Lew, anti-L1CAM, anti-HER2, anti-B7H3, anti-CD33, anti-peptide/MHC, anti-glypican3, or anti-GD3 binding domain.

Exemplary embodiment 50. The conjugate of exemplary embodiment 49, where the first binding domain is an anti-GD2 antibody, antibody component, or antigen-binding antibody fragment.

Exemplary embodiment 51. The conjugate of exemplary embodiment 49, where the first binding domain is an anti-GD2 scFv.

Exemplary embodiment 52. The conjugate of exemplary embodiment 49, where the first binding domain is an anti-HER2 antibody, antibody component, or antigen-binding antibody fragment.

Exemplary embodiment 53. The conjugate of exemplary embodiment 49, where the first binding domain is an anti-HER2 scFv.

Exemplary embodiment 54. The conjugate of any one of exemplary embodiments 1-36, where the SADA polypeptide is or comprises a sequence as set forth in any one of SEQ ID NOs: 1, 3, 5, 7, 9, 11, 13, and 15.

Exemplary embodiment 55. The conjugate of any one of exemplary embodiments 1-36, where the conjugate comprises a polypeptide sequence that is at least 80% identical to a sequence as set forth in any one of SEQ ID NOs: 17, 19, 21, 23, 25, 27, 29, 31, 33, 35, 37, 39, 41, 43, 45, 47, 49, 51, 53, 55, 57, 59, 61, 63, 67, 69, 71, 73, 75, 77, 79, 81, 83, 85, 87, 89, 91, 93, 95, and 97.

Exemplary embodiment 56. The conjugate of any one of exemplary embodiments 1-36, where the conjugate comprises a polypeptide sequence that is at least 90% identical to a sequence as set forth in any one of SEQ ID NOs: 17, 19, 21, 23, 25, 27, 29, 31, 33, 35, 37, 39, 41, 43, 45, 47, 49, 51, 53, 55, 57, 59, 61, 63, 67, 69, 71, 73, 75, 77, 79, 81, 83, 85, 87, 89, 91, 93, 95, and 97.

Exemplary embodiment 57. The conjugate of any one of exemplary embodiments 1-36, where the conjugate comprises a polypeptide sequence that is at least 95% identical to a sequence as set forth in any one of SEQ ID NOs: 17, 19, 21, 23, 25, 27, 29, 31, 33, 35, 37, 39, 41, 43, 45, 47, 49, 51, 53, 55, 57, 59, 61, 63, 67, 69, 71, 73, 75, 77, 79, 81, 83, 85, 87, 89, 91, 93, 95, and 97.

Exemplary embodiment 58. The conjugate of any one of exemplary embodiments 1-36, where the conjugate comprises a polypeptide sequence that is 98% identical to a sequence as set forth in any one of SEQ ID NOs: 17, 19, 21, 23, 25, 27, 29, 31, 33, 35, 37, 39, 41, 43, 45, 47, 49, 51, 53, 55, 57, 59, 61, 63, 67, 69, 71, 73, 75, 77, 79, 81, 83, 85, 87, 89, 91, 93, 95, and 97.

Exemplary embodiment 59. The conjugate of any one of exemplary embodiments 1-58, further characterized in that the binding domain binds a target at an in vivo site, where the target is present at sufficient density such that the conjugate is substantially in the higher-order multimerization state at the site.

Exemplary embodiment 60. The conjugate of any one of exemplary embodiments 1-58, further characterized in that the binding domain binds a target, where the target is present at sufficient concentration such that higher order multimerization state of the SADA polypeptide is stabilized.

Exemplary embodiment 61. The conjugate of any one of exemplary embodiments 1-60, further comprising a dimerization domain or a second SADA domain.

Exemplary embodiment 62. The conjugate of any one of exemplary embodiments 1-61, where the conjugate can exist in one or more additional multimeric states.

Exemplary embodiment 63. The conjugate of exemplary embodiment 61, where the conjugate comprises a second SADA domain and can exist in one or more additional multimeric states.

Exemplary embodiment 64. The conjugate of exemplary embodiment 61, where the conjugate comprises a second SADA domain and can exist in two or more additional multimeric states.

Exemplary embodiment 65. The conjugate of any one of exemplary embodiments 1-64, where the conjugate is substantially not immunogenic in a human subject.

Exemplary embodiment 66. The conjugate of any one of exemplary embodiments 1-65, where the first binding domain is or comprises an antibody component.

Exemplary embodiment 67. The conjugate of any one of exemplary embodiments 1-66, where the first binding domain is or comprises a scFv.

Exemplary embodiment 68. The conjugate of exemplary embodiment 66 or 67, where the conjugate further comprises a second binding domain, wherein the second binding domain is or comprises an antibody component.

Exemplary embodiment 69. The conjugate of exemplary embodiment 68, where the second binding domain is or comprises a scFv.

Exemplary embodiment 70. The conjugate of exemplary embodiment 68 or 69, where the first and second binding domains are part of a bispecific antibody agent.

Exemplary embodiment 71. The conjugate of exemplary embodiment 70, where the bispecific antibody agent comprises a first binding domain that binds a tumor target and a second binding domain that binds a metal-Bn-DOTA.

Exemplary embodiment 72. The conjugate of exemplary embodiment 71, where the bispecific antibody agent comprises a first binding domain that binds a tumor target and a second binding domain that binds an immune-cell activating receptor.

Exemplary embodiment 73. The conjugate of exemplary embodiment 71 or 72, where the first binding domain that binds a tumor target is an anti-GD2, anti-Globo H, anti-GPA33, anti-PSMA, anti-polysialic acid, anti-Lew, anti-L1CAM, anti-HER2, anti-B7H3, anti-CD33, anti-peptide/MHC, anti-glypican3, or anti-GD3 binding domain.

Exemplary embodiment 74. The conjugate of exemplary embodiment 73, where the first binding domain is an anti-GD2 scFv.

Exemplary embodiment 75. The conjugate of exemplary embodiment 73, where the first binding domain is an anti-HER2 scFv.

Exemplary embodiment 76. A nucleic acid sequence encoding a conjugate of any one of exemplary embodiments 1-75.

Exemplary embodiment 77. The nucleic acid sequence of exemplary embodiment 76, where the nucleic acid comprises a sequence that is at least 75%, 80%, 85%, 90%, 91%, 92%, 93%, 94%, 95%, 96%, 97%, 98%, or 99% identical to a sequence as set forth in any one of SEQ ID NOs: 2, 4, 6, 8, 10, 12, 14 and 16.

Exemplary embodiment 78. The nucleic acid sequence of exemplary embodiment 76, where the nucleic acid comprises a sequence as set forth in any one of SEQ ID NOs: 2, 4, 6, 8, 10, 12, 14 and 16.

Exemplary embodiment 79. The nucleic acid sequence of any one of exemplary embodiments 76-78, comprising a sequence that is at least 75%, 80%, 85%, 90%, 91%, 92%, 93%, 94%, 95%, 96%, 97%, 98%, or 99% identical to a sequence as set forth in any one of SEQ ID NOs: 18, 20, 22, 24, 26, 28, 30, 32, 34, 36, 38, 40, 42, 44, 46, 48, 50, 52, 54, 56, 58, 60, 62, 64, 68, 70, 72, 74, 76, 78, 80, 82, 84, 86, 88, 90, 92, 94, 96, and 98.

Exemplary embodiment 80. The nucleic acid sequence of any one of exemplary embodiments 76-78, comprising a sequence as set forth in any one of SEQ ID NOs: 18, 20, 22, 24, 26, 28, 30, 32, 34, 36, 38, 40, 42, 44, 46, 48, 50, 52, 54, 56, 58, 60, 62, 64, 68, 70, 72, 74, 76, 78, 80, 82, 84, 86, 88, 90, 92, 94, 96, and 98.

Exemplary embodiment 81. A vector comprising the nucleic acid sequence of any one of exemplary embodiments 76-80.

Exemplary embodiment 82. A host cell comprising the vector of exemplary embodiment 81.

Exemplary embodiment 83. The host cell of exemplary embodiment 82, where the host cell is selected from the group consisting of a bacterial, yeast, insect or mammalian cell.

Exemplary embodiment 84. The host cell of exemplary embodiment 83, where the host cell is selected from the group consisting of E. coli, Pichia pastoris, Sf9, COS, HEK293 and a CHO cell.

Exemplary embodiment 85. A composition comprising the conjugate of any one of exemplary embodiments 1-75.

Exemplary embodiment 86. The composition of exemplary embodiment 85, where the composition is formulated for injection so that stable binding between the conjugate and its target is detectable at its target tissue for a period of time at least 24 hours long, and wherein the conjugate is substantially undetectable in at least one non-target tissue within 72 hours post-injection without any extraneous drug or clearing agent.

Exemplary embodiment 87. The composition of exemplary embodiment 86, wherein the non-target tissue is selected from the group consisting of blood, gastrointestinal tissue, lymphoid tissue, nervous system tissue, renal tissue, hepatic tissue, and combinations thereof.

Exemplary embodiment 88. The composition of exemplary embodiment 86, where the non-target tissue is or comprises blood.

Exemplary embodiment 89. The composition of any one of exemplary embodiments 86-88, where the target tissue is or comprises a tumor tissue.

Exemplary embodiment 90. A composition comprising an isolated nucleic acid sequence of any one of exemplary embodiments 76-80.

Exemplary embodiment 91. A method comprising steps of providing a liquid composition comprising the conjugate of any one of exemplary embodiments 1-75 in the higher-order multimeric state; and administering the composition to a subject.

Exemplary embodiment 92. The method of exemplary embodiment 91, where the step of administering comprises delivering so that conjugate that is not bound to the target tissue disassembles into the first multimerization state or a monomeric state, whereas conjugate that is bound to the target is substantially in the higher-order multimeric state.

Exemplary embodiment 93. The method of exemplary embodiment 91 or 92, where the extent of the conjugate in the higher-order multimeric state may be or is assessed by measuring the retention of the conjugate at a target site.

Exemplary embodiment 94. The method of exemplary embodiment 91 or 92, where the extent of conjugate in the first multimerization state or monomeric state may be or is assessed by measuring the amount of conjugate in the blood of a subject.

Exemplary embodiment 95. The method of exemplary embodiment 91 or 92, where the extent of conjugate in the first multimerization state or monomeric state may be or is assessed by direct radiolabeling.

Exemplary embodiment 96. The method of exemplary embodiment 91 or 92, where the extent of conjugate in the first multimerization state or monomeric state may be or is assessed by measuring the rate of clearance of the conjugate into the urine.

Exemplary embodiment 97. The method of any one of exemplary embodiments 91-96, where the step of administering is to a subject suffering from or susceptible to cancer.

Exemplary embodiment 98. The method of exemplary embodiment 97, where the cancer is selected from a multiple myeloma, leukemia, acute leukemia, acute lymphoblastic leukemia (ALL), B-cell, T-cell or FAB ALL, acute myeloid leukemia (AML), chromic myelocytic leukemia (CML), chronic lymphocytic leukemia (CLL), hairy cell leukemia, myelodysplastic syndrome (MDS), a lymphoma, Hodgkin's disease, a malignant lymphoma, non-hodgkin's lymphoma, Burkitt's lymphoma, multiple myeloma, Kaposi's sarcoma, solid tumor, colorectal cancer, renal cancer, pancreatic cancer, prostate cancer, nasopharyngeal cancer, malignant histiocytosis, adenocarcinoma, sarcoma, hemangioma, sarcoma, cerebral tumor, bone tumor, breast cancer, squamous cell carcinoma, stomach cancer, melanoma and mesothelioma.

Exemplary embodiment 99. Use of a conjugate of any one of exemplary embodiments 1-75 in treating cancer.

Exemplary embodiment 100. A method comprising steps of: providing a liquid composition comprising the conjugate of any one of exemplary embodiments 71-75; and administering the composition to a subject that is suffering from cancer.

Exemplary embodiment 101. A method of treating or diagnosing cancer in a subject, the method comprising steps of: providing a liquid composition comprising the conjugate of any one of exemplary embodiments 71-75 in a concentration sufficient that greater than 90% of the conjugate is in the higher-order multimerization state; and administering the composition to a subject that is suffering from or susceptible to cancer.

Exemplary embodiment 102. The method of exemplary embodiment 101, where the concentration of conjugate is within a range of 50 nM to 1 mM.

Exemplary embodiment 103. The method of exemplary embodiment 101, where the concentration of conjugate is within a range of 100 nM to 10 μM.

Exemplary embodiment 104. The method of exemplary embodiment 101, where the concentration of conjugate is within a range of 100 nM to 100 μM.

Exemplary embodiment 105. The method of exemplary embodiment 101, where the concentration of conjugate is within a range of 500 nM to 500 μM.

Exemplary embodiment 106. The method of exemplary embodiment 101, where the concentration of conjugate is within a range of 1 μM to 1 mM.

Exemplary embodiment 107. The method of any one of exemplary embodiments 100-106, where the cancer is selected from a multiple myeloma, leukemia, acute leukemia, acute lymphoblastic leukemia (ALL), B-cell, T-cell or FAB ALL, acute myeloid leukemia (AML), chromic myelocytic leukemia (CML), chronic lymphocytic leukemia (CLL), hairy cell leukemia, myelodysplastic syndrome (MDS), a lymphoma, Hodgkin's disease, a malignant lymphoma, non-hodgkin's lymphoma, Burkitt's lymphoma, multiple myeloma, Kaposi's sarcoma, solid tumor, colorectal cancer, renal cancer, pancreatic cancer, prostate cancer, nasopharyngeal cancer, malignant histiocytosis, adenocarcinoma, sarcoma, hemangioma, sarcoma, cerebral tumor, bone tumor, breast cancer, squamous cell carcinoma, stomach cancer, melanoma and mesothelioma.

Exemplary embodiment 108. A method of pre-targeted radio immunotherapy, the method comprising steps of: providing a liquid composition comprising the conjugate of any one of exemplary embodiments 71-75 in the higher order multimeric form; administering the composition to a subject that is suffering from or susceptible to cancer; and subsequently administering a radiolabeled Bn-DOTA to the subject.

Exemplary embodiment 109. The method of exemplary embodiment 108, wherein the method does not include the administration of a clearing agent.

Exemplary embodiment 110. A method of pre-targeted radio immunotherapy, the method comprising steps of: providing a liquid composition comprising the conjugate of any one of exemplary embodiments 71-75 in a concentration of at least 50 nM, 100 nM, 500 nM, 1 μM, 10 μM, 50 μM, 100 μM, 200 μM, 300 μM, 400 μM, 500 μM, or 1 mM; administering the composition to a subject that is suffering from or susceptible to cancer.

Exemplary embodiment 111. The method of exemplary embodiment 110, where the concentration of conjugate is within a range of 50 nM to 1 mM.

Exemplary embodiment 112. The method of exemplary embodiment 110, where the concentration of conjugate is within a range of 100 nM to 10 μM.

Exemplary embodiment 113. The method of exemplary embodiment 110, where the concentration of conjugate is within a range of 100 nM to 100 μM.

Exemplary embodiment 114. The method of exemplary embodiment 110, where the concentration of conjugate is within a range of 500 nM to 500 μM.

Exemplary embodiment 115. The method of exemplary embodiment 110, where the concentration of conjugate is within a range of 1 μM to 1 mM.

Exemplary embodiment 116. The method of any one of exemplary embodiments 110-115, where conjugate in the higher order multimeric form.

Exemplary embodiment 117. The method of any one of exemplary embodiments 110-116, where a radiolabeled agent comprising a Bn-DOTA is covalently attached to the conjugate.

Exemplary embodiment 118. The method of any one of exemplary embodiments 110-116, where a radiolabeled Bn-DOTA is non-covalently complexed with the conjugate.

Exemplary embodiment 119. The method of any one of exemplary embodiments 110-118, where the method does not include the administration of a clearing agent.

Exemplary embodiment 120. A method comprising steps of: providing a liquid composition comprising the conjugate of any one of exemplary embodiments 1-75, where at least 90% of the conjugate in the composition is in the higher order multimeric form; and administering the composition to a subject from whom a target entity is to be removed, wherein the conjugate is capable of binding the target entity.

Exemplary embodiment 121. A method of identifying or characterizing a conjugate, the method comprising steps of: providing a conjugate comprising a self-assembly disassembly (SADA) polypeptide and a binding domain; determining a threshold condition (concentration, pH/pOH, oxidation/reduction state) wherein the conjugate substantially adopts a multimeric form greater than about ^{˜}70 kDa.

Exemplary embodiment 122. The method of exemplary embodiment 121, where the step of providing comprises providing a conjugate in which the SADA polypeptide is a test polypeptide and the step of determining comprises identifying the multimerization domain as useful in the conjugate if the critical multimerization concentration is within a range of about 100 nM to 1 mM.

Exemplary embodiment 123. The method of exemplary embodiment 121 or 122, where the step of providing comprises providing a plurality of conjugates, and the step of determining comprises determining the threshold for each of the conjugates.

Exemplary embodiment 124. The method of any one of exemplary embodiments 121-123, where each conjugate in the plurality comprises the same binding domain but differs in the SADA polypeptide.

Exemplary embodiment 125. The method of any one of exemplary embodiments 121-124, where the SADA polypeptide is or comprises a tetramerization domain of any one of p53, p63, p73, hnRNPC, SNAP-23, Stefin B, KCNQ4, and CBFA2T1.

Exemplary embodiment 126. A method of identifying or characterizing a conjugate, the method comprising steps of: providing a conjugate comprising a self-assembly disassembly (SADA) polypeptide and a binding domain; administering the composition to a subject; and determining the affinity of the conjugate for a target.

Exemplary embodiment 127. The method of exemplary embodiment 126 where the step of providing comprises providing a plurality of conjugates, and the step of determining comprises determining the affinity for a target for each of the conjugates.

Exemplary embodiment 128. The method of exemplary embodiment 126 or 127, further comprising s step of determining the rate of clearance of the conjugate from blood.

Exemplary embodiment 129. The method of exemplary embodiment 128, where the step of determining the rate of clearance of the conjugate from blood is for each of the conjugates.

Exemplary embodiment 130. The method of any one of exemplary embodiments 126-129, where each conjugate in the plurality comprises the same binding domain but differs in the SADA polypeptide.

Exemplary embodiment 131. The method of any one of exemplary embodiments 126-130, further comprising a step of identifying one or more conjugates in the plurality as preferred relative to another conjugate in the plurality when the preferred conjugate shows increased avidity for a target and/or when the preferred conjugate is more rapidly cleared from the blood.

Exemplary embodiment 132. A method of producing a composition, the method comprising steps of: providing a composition comprising the conjugate of any one of exemplary embodiments 71-75; formulating the conjugate with a pharmaceutically acceptable carrier or excipient to produce a composition in which the conjugate is present at a concentration sufficient for at least 90% of the conjugate to adopt the higher-order multimerized state.

Exemplary embodiment 133. The method of exemplary embodiment 132, where the concentration of conjugate is within a range of 50 nM to 1 mM.

Exemplary embodiment 134. The method of exemplary embodiment 132, where the concentration of conjugate is within a range of 100 nM to 10 μM.

Exemplary embodiment 135. The method of exemplary embodiment 132, where the concentration of conjugate is within a range of 100 nM to 100 μM.

Exemplary embodiment 136. The method of exemplary embodiment 132, where the concentration of conjugate is within a range of 500 nM to 500 μM.

Exemplary embodiment 136. The method of exemplary embodiment 132, where the concentration of conjugate is within a range of 1 μM to 1 mM.

Other features of the invention will become apparent in the course of the following descriptions of exemplary embodiments, which are given for illustration of the invention and are not intended to be limiting thereof.

### EXEMPLIFICATION

### Example 1—Production of an Exemplary Conjugate with a SADA Domain

This example demonstrates the production of exemplary SADA conjugates with a first binding domain that binds a payload (e.g., a molecular payload), a second domain that binds a cellular target (e.g., a cell surface target) and a SADA domain. Specifically, this example describes the production of exemplary bispecific antibody-based conjugates comprising a tandem-scFv bispecific antibody with two different scFv's linked by a G₄S linker and followed by a tetrameric SADA tag. Three constructs were produced (P53-BIDE, P63-BIDE, P73-BIDE), each comprising a first scFv with specificity for tumor cells (a humanized anti-GD2 scFv) and a second scFv with specificity for a metal-chelate of Benzyl-1,4,7,10-tetraazacyclododecane-1,4,7,10-tetraacetic acid, [metal]-Bn-DOTA, which recognizes Bn-DOTA when chelating metal ions such as Lu-177, Y-86, Y-90, In-111, etc. The constructs, P53-BiDE and P53-BiDE(noHIS) (which lacks a terminal HIS tag) included a SADA domain that is derived from the human p53 tetramerization domain. The construct, P63-BiDE, included a SADA domain that is derived from the human p63 tetramerization domain. The construct, P73-BiDE included a SADA domain that is derived from the human p73 tetramerization domain. The amino acid sequences and the cDNA nucleotide sequences of these constructs are shown below.

P53-BIDE(noHIS) polypeptide (hu3F8-scFv, huC825-scFv,
huP53-tet, GS linker, (IgG3 spacer))
SEQ ID NO: 17
EIVMTQTPATLSVSAGERVTITCKASQSVSNDVTWYQQKPGQAPRLLIYSASNRYSGVPARFS
GSGYGTEFTFTISSVQSEDFAVYFCQQDYSSFGCGTKLEIKRGGGGSGGGGSGGGGSQVQLVE
SGPGVVQPGRSLRISCAVSGFSVTNYGVHWVRQPPGKCLEWLGVIWAGGITNYNSAFMSRLTI
SKDNSKNTVYLQMNSLRAEDTAMYYCASRGGHYGYALDYWGQGTLVTVSSGGGGSGGGGSGGG
GSGGGGSHVQLVESGGGLVQPGGSLRLSCAASGFSLTDYGVHWVRQAPGKGLEWLGVIWSGGG
TAYNTALISRFTISRDNSKNTLYLQMNSLRAEDTAVYYCARRGSYPYNYFDAWGCGTLVTVSS
GGGGSGGGGSGGGGSQAVVTQEPSLTVSPGGTVTLTCGSSTGAVTASNYANWVQQKPGQCPRG
LIGGHNNRPPGVPARFSGSLLGGKAALTLLGAQPEDEAEYYCALWYSDHWVIGGGTKLTVLG
(TPLGDTTHT)SGKPLDGEYFTLQIRGRERFEMFRELNEALELKDAQAGKEPGGSGGA
P53-BIDE(noHIS) cDNA (hu3F8-scFv, huC825-scFv, huP53-tet,
GS linker, (IgG3 spacer))
SEQ ID NO: 18
GAAATCGTCATGACTCAGACTCCCGCAACCCTGTCAGTGTCCGCTGGGGAACGTGTCACTATT
ACCTGCAAGGCATCTCAGAGCGTGAGCAACGACGTGACCTGGTATCAGCAGAAGCCTGGCCAG
GCTCCACGACTGCTGATCTATTCCGCAAGCAATCGCTACTCCGGAGTGCCCGCACGATTCTCT
GGAAGTGGGTACGGTACCGAGTTCACTTTTACCATTTCCAGCGTGCAGAGCGAAGACTTCGCT
GTCTATTTTTGCCAGCAGGATTACTCTAGTTTTGGCTGTGGAACAAAGCTGGAGATCAAAAGG
GGAGGAGGAGGTTCTGGCGGAGGAGGTAGTGGCGGAGGGGGTTCACAGGTGCAGCTGGTCGAA
TCTGGGCCAGGCGTGGTCCAGCCAGGACGTTCCCTGAGGATTAGCTGCGCCGTGAGCGGGTTC
TCTGTCACAAACTACGGAGTGCACTGGGTCCGTCAGCCACCTGGCAAATGTCTGGAGTGGCTG
GGAGTGATCTGGGCAGGAGGAATCACTAACTACAACTCTGCTTTTATGAGTCGCCTGACCATC
TCAAAGGACAACTCCAAAAATACAGTGTACCTGCAGATGAATTCACTGCGGGCAGAAGATACC
GCCATGTACTATTGCGCCTCCAGGGGGGGTCATTACGGCTATGCCCTGGACTATTGGGGCCAG
GGAACACTGGTGACTGTCTCATCCGGAGGAGGAGGATCCGGAGGAGGAGGTAGCGGCGGAGGG
GGTTCTGGCGGAGGGGGTAGTCACGTGCAGCTGGTCGAGTCCGGAGGAGGGCTGGTGCAGCCT
GGTGGCAGCCTGCGACTGTCTTGTGCCGCTAGTGGCTTCTCACTGACAGATTACGGCGTGCAT
TGGGTCCGACAGGCTCCAGGGAAGGGTCTGGAATGGCTGGGAGTGATTTGGTCTGGAGGGGGT
ACAGCTTATAACACTGCACTGATCAGTCGGTTCACTATCAGTAGAGACAACTCAAAGAACACC
CTGTACCTGCAGATGAACTCTCTGCGGGCCGAGGATACCGCTGTGTACTATTGCGCTAGGCGG
GGCAGTTACCCTTATAATTACTTTGACGCATGGGGCTGTGGAACCCTGGTGACAGTCAGCTCT
GGCGGAGGGGGTTCAGGCGGCGGCGGTTCCGGCGGAGGAGGTAGCCAGGCCGTGGTCACTCAG
GAGCCTTCCCTGACCGTGAGCCCAGGAGGAACAGTCACTCTGACCTGCGGGAGTTCAACCGGT
GCCGTGACAGCCTCCAACTACGCTAATTGGGTCCAGCAGAAGCCCGGGCAGTGTCCTAGAGGT
CTGATCGGGGGTCACAACAATCGTCCACCCGGAGTGCCAGCCAGGTTCTCAGGCTCCCTGCTG
GGCGGAAAAGCAGCACTGACTCTGCTGGGCGCTCAGCCAGAGGACGAAGCAGAGTACTATTGC
GCCCTGTGGTATTCTGATCACTGGGTCATCGGGGGTGGCACTAAGCTGACCGTGCTGGGC(AC
ACCCCTGGGAGACACCACACATACT)AGTGGCAAACCTCTGGATGGAGAGTACTTTACCCTGC
AGATTAGAGGCCGCGAACGATTCGAGATGTTTCGCGAACTGAATGAGGCCCTGGAACTGAAGG
ATGCTCAGGCAGGCAAGGAACCAGGCGGTAGCGGCGGCGCA
P53-BIDE polypeptide (hu3F8-scFv, huC825-scFv, huP53-tet,
GS linker, (IgG3 spacer))
SEQ ID NO: 19
EIVMTQTPATLSVSAGERVTITCKASQSVSNDVTWYQQKPGQAPRLLIYSASNRYSGVPARFS
GSGYGTEFTFTISSVQSEDFAVYFCQQDYSSFGCGTKLEIKRGGGGSGGGGSGGGGSGGGGSG
GGGSGGGGSQVQLVESGPGVVQPGRSLRISCAVSGFSVTNYGVHWVRQPPGKCLEWLGVIWAG
GITNYNSAFMSRLTISKDNSKNTVYLQMNSLRAEDTAMYYCASRGGHYGYALDYWGQGTLVTV
SSGGGGSGGGGSGGGGSGGGGSHVQLVESGGGLVQPGGSLRLSCAASGFSLTDYGVHWVRQAP
GKGLEWLGVIWSGGGTAYNTALISRFTISRDNSKNTLYLQMNSLRAEDTAVYYCARRGSYPYN
YFDAWGCGTLVTVSSGGGGSGGGGSGGGGSQAVVTQEPSLTVSPGGTVTLTCGSSTGAVTASN
YANWVQQKPGQCPRGLIGGHNNRPPGVPARFSGSLLGGKAALTLLGAQPEDEAEYYCALWYSD
HWVIGGGTKLTVLG(TPLGDTTHT)SGKPLDGEYFTLQIRGRERFEMFRELNEALELKDAQAG
KEPGGSGGAPHHHHHH
P53-BIDE cDNA (hu3F8-scFv, huC825-scFv, huP53-tet, GS linker,
(IgG3 spacer))
SEQ ID NO: 20
GAGATCGTGATGACCCAGACACCCGCAACACTGAGCGTGTCTGCCGGCGAAAGGGTCACTATT
ACCTGCAAGGCCAGTCAGTCAGTGTCCAACGACGTGACTTGGTACCAGCAGAAACCAGGCCAG
GCTCCCCGGCTGCTGATCTACAGCGCATCTAATAGATATAGCGGAGTGCCTGCTCGCTTCAGT
GGTTCAGGCTATGGAACTGAGTTCACCTTCACCATTTCCAGCGTGCAGTCCGAAGACTTCGCA
GTGTACTTTTGCCAGCAGGATTATTCTAGTTTTGGGTGTGGTACAAAGCTGGAGATCAAAAGG
GGAGGAGGAGGTAGTGGCGGAGGAGGTTCAGGCGGAGGGGGTAGCGGCGGAGGGGGTTCTGGC
GGCGGCGGTAGTGGCGGCGGAGGTAGCCAGGTGCAGCTGGTCGAATCCGGCCCTGGAGTGGTC
CAGCCAGGCAGGTCTCTGCGGATCAGTTGCGCCGTGTCCGGATTCAGCGTCACCAACTACGGA
GTGCACTGGGTCAGACAGCCACCTGGCAAGTGTCTGGAGTGGCTGGGAGTGATCTGGGCAGGA
GGAATCACAAACTACAACTCAGCTTTTATGTCCCGCCTGACTATTAGCAAGGACAACTCTAAA
AATACCGTGTATCTGCAGATGAATTCTCTGCGAGCCGAAGATACCGCTATGTACTATTGTGCA
TCCCGTGGGGGTCATTACGGCTATGCCCTGGATTATTGGGGGCAGGGTACCCTGGTGACAGTC
TCATCCGGCGGAGGGGGATCCGGAGGAGGAGGTAGCGGCGGAGGGGGTTCTGGCGGAGGGGGT
AGTCACGTGCAGCTGGTCGAGTCCGGAGGAGGGCTGGTGCAGCCTGGTGGCAGCCTGCGACTG
TCTTGTGCCGCTAGTGGCTTCTCACTGACAGATTACGGCGTGCATTGGGTCCGACAGGCTCCA
GGGAAGGGTCTGGAATGGCTGGGAGTGATTTGGTCTGGAGGGGGTACAGCTTATAACACTGCA
CTGATCAGTCGGTTCACTATCAGTAGAGACAACTCAAAGAACACCCTGTACCTGCAGATGAAC
TCTCTGCGGGCCGAGGATACCGCTGTGTACTATTGCGCTAGGCGGGGCAGTTACCCTTATAAT
TACTTTGACGCATGGGGCTGTGGAACCCTGGTGACAGTCAGCTCTGGCGGAGGGGGTTCAGGC
GGCGGCGGTTCCGGCGGAGGAGGTAGCCAGGCCGTGGTCACTCAGGAGCCTTCCCTGACCGTG
AGCCCAGGAGGAACAGTCACTCTGACCTGCGGGAGTTCAACCGGTGCCGTGACAGCCTCCAAC
TACGCTAATTGGGTCCAGCAGAAGCCCGGGCAGTGTCCTAGAGGTCTGATCGGGGGTCACAAC
AAPCGTCCACCCGGAGTGCCAGCCAGGTTCTCAGGCTCCCTGCTGGGCGGAAAAGCAGCACTG
ACTCTGCTGGGCGCTCAGCCAGAGGACGAAGCAGAGTACTATTGCGCCCTGTGGTATTCTGAT
CACTGGGTCATCGGGGGTGGCACTAAGCTGACCGTGCTGGGC(ACACCCCTGGGAGACACCAC
ACATACT)AGTGGGAAACCTCTGGATGGCGAGTACTTTACCCTGCAGATTAGAGGCCGCGAAC
GATTCGAGATGTTTCGCGAACTGAATGAGGCCCTGGAACTGAAGGATGCTCAGGCAGGCAAGG
AGCCAGGAGGGTCAGGAGGAGCACCGCACCATCATCATCACCAT
P63-BIDE polypeptide (hu3F8-scFv, huC825-scFv, huP63-tet,
GS linker, (IgG3 spacer))
SEQ ID NO: 21
EIVMTQTPATLSVSAGERVTITCKASQSVSNDVTWYQQKPGQAPRLLIYSASNRYSGVPARFS
GSGYGTEFTFTISSVQSEDFAVYFCQQDYSSFGCGTKLEIKRGGGGSGGGGSGGGGSGGGGSG
GGGSGGGGSQVQLVESGPGVVQPGRSLRISCAVSGFSVTNYGVHWVRQPPGKCLEWLGVIWAG
GITNYNSAFMSRLTISKDNSKNTVYLQMNSLRAEDTAMYYCASRGGHYGYALDYWGQGTLVTV
SSGGGGSGGGGSGGGGSGGGGSHVQLVESGGGLVQPGGSLRLSCAASGFSLTDYGVHWVRQAP
GKGLEWLGVIWSGGGTAYNTALISRFTISRDNSKNTLYLQMNSLRAEDTAVYYCARRGSYPYN
YFDAWGCGTLVTVSSGGGGSGGGGSGGGGSQAVVTQEPSLTVSPGGTVTLTCGSSTGAVTASN
YANWVQQKPGQCPRGLIGGHNNRPPGVPARFSGSLLGGKAALTLLGAQPEDEAEYYCALWYSD
HWVIGGGTKLTVLG(TPLGDTTHT)SGRSPDDELLYLPVRGRETYEMLLKIKESLELMQYLPQ
HTIETYRQQQQQQHQHLLQKQGGSGGAPHHHHHH
P63-BIDE cDNA (hu3F8-scFv, huC825-scFv, huP63-tet, GS linker,
(IgG3 spacer))
SEQ ID NO: 22
GAGATCGTGATGACCCAGACACCCGCAACACTGAGCGTGTCTGCCGGCGAAAGGGTCACTATT
ACCTGCAAGGCCAGTCAGTCAGTGTCCAACGACGTGACTTGGTACCAGCAGAAACCAGGCCAG
GCTCCCCGGCTGCTGATCTACAGCGCATCTAATAGATATAGCGGAGTGCCTGCTCGCTTCAGT
GGTTCAGGCTATGGAACTGAGTTCACCTTCACCATTTCCAGCGTGCAGTCCGAAGACTTCGCA
GTGTACTTTTGCCAGCAGGATTATTCTAGTTTTGGGTGTGGTACAAAGCTGGAGATCAAAAGG
GGAGGAGGAGGTAGTGGCGGAGGAGGTTCAGGCGGAGGGGGTAGCGGCGGAGGGGGTTCTGGC
GGCGGCGGTAGTGGCGGCGGAGGTAGCCAGGTGCAGCTGGTCGAATCCGGCCCTGGAGTGGTC
CAGCCAGGCAGGTCTCTGCGGATCAGTTGCGCCGTGTCCGGATTCAGCGTCACCAACTACGGA
GTGCACTGGGTCAGACAGCCACCTGGCAAGTGTCTGGAGTGGCTGGGAGTGATCTGGGCAGGA
GGAATCACAAACTACAACTCAGCTTTTATGTCCCGCCTGACTATTAGCAAGGACAACTCTAAA
AATACCGTGTATCTGCAGATGAATTCTCTGCGAGCCGAAGATACCGCTATGTACTATTGTGCA
TCCCGTGGGGGTCATTACGGCTATGCCCTGGATTATTGGGGGCAGGGTACCCTGGTGACAGTC
TCATCCGGAGGAGGAGGATCCGGAGGAGGAGGTAGCGGCGGAGGGGGTTCTGGCGGAGGGGGT
AGTCACGTGCAGCTGGTCGAGTCCGGAGGAGGGCTGGTGCAGCCTGGTGGCAGCCTGCGACTG
TCTTGTGCCGCTAGTGGCTTCTCACTGACAGATTACGGCGTGCATTGGGTCCGACAGGCTCCA
GGGAAGGGTCTGGAATGGCTGGGAGTGATTTGGTCTGGAGGGGGTACAGCTTATAACACTGCA
CTGATCAGTCGGTTCACTATCAGTAGAGACAACTCAAAGAACACCCTGTACCTGCAGATGAAC
TCTCTGCGGGCCGAGGATACCGCTGTGTACTATTGCGCTAGGCGGGGCAGTTACCCTTATAAT
TACTTTGACGCATGGGGCTGTGGAACCCTGGTGACAGTCAGCTCTGGCGGAGGGGGTTCAGGC
GGCGGCGGTTCCGGCGGAGGAGGTAGCCAGGCCGTGGTCACTCAGGAGCCTTCCCTGACCGTG
AGCCCAGGAGGAACAGTCACTCTGACCTGCGGGAGTTCAACCGGTGCCGTGACAGCCTCCAAC
TACGCTAATTGGGTCCAGCAGAAGCCCGGGCAGTGTCCTAGAGGTCTGATCGGGGGTCACAAC
AAPCGTCCACCCGGAGTGCCAGCCAGGTTCTCAGGCTCCCTGCTGGGCGGAAAAGCAGCACTG
ACTCTGCTGGGCGCTCAGCCAGAGGACGAAGCAGAGTACTATTGCGCCCTGTGGTATTCTGAT
CACTGGGTCATCGGGGGTGGCACTAAGCTGACCGTGCTGGGC(ACACCCCTGGGAGACACCAC
ACATACT)AGTGGGAGATCCCCCGACGATGAGCTGCTGTACCTGCCTGTGAGGGGCCGGGAGA
CCTATGAAATGCTGCTGAAGATCAAAGAGAGCCTGGAACTGATGCAGTACCTGCCACAGCACA
CCATTGAAACATATAGGCAACAACAGCAGCAGCAGCATCAGCATCTGCTGCAGAAGCAGGGAG
GGTCAGGAGGAGCACCGCACCATCATCATCACCAT
P73-BIDE polypeptide (hu3F8-scFv, huC825-scFv, huP73-tet,
GS linker, (IgG3 spacer))
SEQ ID NO: 23
EIVMTQTPATLSVSAGERVTITCKASQSVSNDVTWYQQKPGQAPRLLIYSASNRYSGVPARFS
GSGYGTEFTFTISSVQSEDFAVYFCQQDYSSFGCGTKLEIKRGGGGSGGGGSGGGGSGGGGSG
GGGSGGGGSQVQLVESGPGVVQPGRSLRISCAVSGFSVTNYGVHWVRQPPGKCLEWLGVIWAG
GITNYNSAFMSRLTISKDNSKNTVYLQMNSLRAEDTAMYYCASRGGHYGYALDYWGQGTLVTV
SSGGGGSGGGGSGGGGSGGGGSHVQLVESGGGLVQPGGSLRLSCAASGFSLTDYGVHWVRQAP
GKGLEWLGVIWSGGGTAYNTALISRFTISRDNSKNTLYLQMNSLRAEDTAVYYCARRGSYPYN
YFDAWGCGTLVTVSSGGGGSGGGGSGGGGSQAVVTQEPSLTVSPGGTVTLTCGSSTGAVTASN
YANWVQQKPGQCPRGLIGGHNNRPPGVPARFSGSLLGGKAALTLLGAQPEDEAEYYCALWYSD
HWVIGGGTKLTVLG(TPLGDTTHT)SGRHGDEDTYYLQVRGRENFEILMKLKESLELMELVPQ
PLVDSYRQQQQLLQRPGGSGGAPHHHHHH
P73-BIDE cDNA (hu3F8-scFv, huC825-scFv, huP73-tet, GS linker,
(IgG3 spacer))
SEQ ID NO: 24
GAGATCGTGATGACCCAGACACCCGCAACACTGAGCGTGTCTGCCGGCGAAAGGGTCACTATT
ACCTGCAAGGCCAGTCAGTCAGTGTCCAACGACGTGACTTGGTACCAGCAGAAACCAGGCCAG
GCTCCCCGGCTGCTGATCTACAGCGCATCTAATAGATATAGCGGAGTGCCTGCTCGCTTCAGT
GGTTCAGGCTATGGAACTGAGTTCACCTTCACCATTTCCAGCGTGCAGTCCGAAGACTTCGCA
GTGTACTTTTGCCAGCAGGATTATTCTAGTTTTGGGTGTGGTACAAAGCTGGAGATCAAAAGG
GGAGGAGGAGGTAGTGGCGGAGGAGGTTCAGGCGGAGGGGGTAGCGGCGGAGGGGGTTCTGGC
GGCGGCGGTAGTGGCGGCGGAGGTAGCCAGGTGCAGCTGGTCGAATCCGGCCCTGGAGTGGTC
CAGCCAGGCAGGTCTCTGCGGATCAGTTGCGCCGTGTCCGGATTCAGCGTCACCAACTACGGA
GTGCACTGGGTCAGACAGCCACCTGGCAAGTGTCTGGAGTGGCTGGGAGTGATCTGGGCAGGA
GGAATCACAAACTACAACTCAGCTTTTATGTCCCGCCTGACTATTAGCAAGGACAACTCTAAA
AATACCGTGTATCTGCAGATGAATTCTCTGCGAGCCGAAGATACCGCTATGTACTATTGTGCA
TCCCGTGGGGGTCATTACGGCTATGCCCTGGATTATTGGGGGCAGGGTACCCTGGTGACAGTC
TCATCCGGAGGAGGAGGATCCGGAGGAGGAGGTAGCGGCGGAGGGGGTTCTGGCGGAGGGGGT
AGTCACGTGCAGCTGGTCGAGTCCGGAGGAGGGCTGGTGCAGCCTGGTGGCAGCCTGCGACTG
TCTTGTGCCGCTAGTGGCTTCTCACTGACAGATTACGGCGTGCATTGGGTCCGACAGGCTCCA
GGGAAGGGTCTGGAATGGCTGGGAGTGATTTGGTCTGGAGGGGGTACAGCTTATAACACTGCA
CTGATCAGTCGGTTCACTATCAGTAGAGACAACTCAAAGAACACCCTGTACCTGCAGATGAAC
TCTCTGCGGGCCGAGGATACCGCTGTGTACTATTGCGCTAGGCGGGGCAGTTACCCTTATAAT
TACTTTGACGCATGGGGCTGTGGAACCCTGGTGACAGTCAGCTCTGGCGGAGGGGGTTCAGGC
GGCGGCGGTTCCGGCGGAGGAGGTAGCCAGGCCGTGGTCACTCAGGAGCCTTCCCTGACCGTG
AGCCCAGGAGGAACAGTCACTCTGACCTGCGGGAGTTCAACCGGTGCCGTGACAGCCTCCAAC
TACGCTAATTGGGTCCAGCAGAAGCCCGGGCAGTGTCCTAGAGGTCTGATCGGGGGTCACAAC
AAPCGTCCACCCGGAGTGCCAGCCAGGTTCTCAGGCTCCCTGCTGGGCGGAAAAGCAGCACTG
ACTCTGCTGGGCGCTCAGCCAGAGGACGAAGCAGAGTACTATTGCGCCCTGTGGTATTCTGAT
CACTGGGTCATCGGGGGTGGCACTAAGCTGACCGTGCTGGGC(ACACCCCTGGGAGACACCAC
ACATACT)AGTGGGAGGCACGGCGACGAAGATACCTACTATCTGCAGGTGAGGGGACGGGAGA
ACTTCGAAATCCTGATGAAGCTGAAAGAGTCCCTGGAACTGATGGAGCTGGTGCCCCAGCCTC
TGGTCGACAGCTACAGACAGCAGCAGCAGCTGCTGCAGAGGCCAGGAGGGTCAGGAGGAGCAC
CGCACCATCATCATCACCAT
P53-BIDE(SL) polypeptide (hu3F8-scFv, huC825scFv, huP53-tet,
GS linker, (IgG3 spacer))
SEQ ID NO: 25
EIVMTQTPATLSVSAGERVTITCKASQSVSNDVTWYQQKPGQAPRLLIYSASNRYSGVPARFS
GSGYGTEFTFTISSVQSEDFAVYFCQQDYSSFGCGTKLEIKRGGGGSGGGGSGGGGSQVQLVE
SGPGVVQPGRSLRISCAVSGFSVTNYGVHWVRQPPGKCLEWLGVIWAGGITNYNSAFMSRLTI
SKDNSKNTVYLQMNSLRAEDTAMYYCASRGGHYGYALDYWGQGTLVTVSSGGGGSGGGGSGGG
GSGGGGSHVQLVESGGGLVQPGGSLRLSCAASGFSLTDYGVHWVRQAPGKGLEWLGVIWSGGG
TAYNTALISRFTISRDNSKNTLYLQMNSLRAEDTAVYYCARRGSYPYNYFDAWGCGTLVTVSS
GGGGSGGGGSGGGGSQAVVTQEPSLTVSPGGTVTLTCGSSTGAVTASNYANWVQQKPGQCPRG
LIGGHNNRPPGVPARFSGSLLGGKAALTLLGAQPEDEAEYYCALWYSDHWVIGGGTKLTVLG
(TPLGDYYHY)SGKPLDGEYFTLQIRGRERFEMFRELNEALELKDAQAGKEPGGSGGAPHHHH
HH
P53-BIDE(SL) cDNA (hu3F8-scFv, huC825-scFv, huP53-tet,
GS linker, (IgG3 spacer))
SEQ ID NO: 26
GAAATCGTCATGACTCAGACTCCCGCAACCCTGTCAGTGTCCGCTGGGGAACGTGTCACTATT
ACCTGCAAGGCATCTCAGAGCGTGAGCAACGACGTGACCTGGTATCAGCAGAAGCCTGGCCAG
GCTCCACGACTGCTGATCTATTCCGCAAGCAATCGCTACTCCGGAGTGCCCGCACGATTCTCT
GGAAGTGGGTACGGTACCGAGTTCACTTTTACCATTTCCAGCGTGCAGAGCGAAGACTTCGCT
GTCTATTTTTGCCAGCAGGATTACTCTAGTTTTGGCTGTGGAACAAAGCTGGAGATCAAAAGG
GGAGGAGGAGGTTCTGGCGGAGGAGGTAGTGGCGGAGGGGGTTCACAGGTGCAGCTGGTCGAA
TCTGGGCCAGGCGTGGTCCAGCCAGGACGTTCCCTGAGGATTAGCTGCGCCGTGAGCGGGTTC
TCTGTCACAAACTACGGAGTGCACTGGGTCCGTCAGCCACCTGGCAAATGTCTGGAGTGGCTG
GGAGTGATCTGGGCAGGAGGAATCACTAACTACAACTCTGCTTTTATGAGTCGCCTGACCATC
TCAAAGGACAACTCCAAAAATACAGTGTACCTGCAGATGAATTCACTGCGGGCAGAAGATACC
GCCATGTACTATTGCGCCTCCAGGGGGGGTCATTACGGCTATGCCCTGGACTATTGGGGCCAG
GGAACACTGGTGACTGTCTCATCCGGAGGAGGAGGATCCGGAGGAGGAGGTAGCGGCGGAGGG
GGTTCTGGCGGAGGGGGTAGTCACGTGCAGCTGGTCGAGTCCGGAGGAGGGCTGGTGCAGCCT
GGTGGCAGCCTGCGACTGTCTTGTGCCGCTAGTGGCTTCTCACTGACAGATTACGGCGTGCAT
TGGGTCCGACAGGCTCCAGGGAAGGGTCTGGAATGGCTGGGAGTGATTTGGTCTGGAGGGGGT
ACAGCTTATAACACTGCACTGATCAGTCGGTTCACTATCAGTAGAGACAACTCAAAGAACACC
CTGTACCTGCAGATGAACTCTCTGCGGGCCGAGGATACCGCTGTGTACTATTGCGCTAGGCGG
GGCAGTTACCCTTATAATTACTTTGACGCATGGGGCTGTGGAACCCTGGTGACAGTCAGCTCT
GGCGGAGGGGGTTCAGGCGGCGGCGGTTCCGGCGGAGGAGGTAGCCAGGCCGTGGTCACTCAG
GAGCCTTCCCTGACCGTGAGCCCAGGAGGAACAGTCACTCTGACCTGCGGGAGTTCAACCGGT
GCCGTGACAGCCTCCAACTACGCTAATTGGGTCCAGCAGAAGCCCGGGCAGTGTCCTAGAGGT
CTGATCGGGGGTCACAACAATCGTCCACCCGGAGTGCCAGCCAGGTTCTCAGGCTCCCTGCTG
GGCGGAAAAGCAGCACTGACTCTGCTGGGCGCTCAGCCAGAGGACGAAGCAGAGTACTATTGC
GCCCTGTGGTATTCTGATCACTGGGTCATCGGGGGTGGCACTAAGCTGACCGTGCTGGGC(AC
ACCCCTGGGAGACACCACACATACT)AGTGGGAAACCTCTGGATGGCGAGTACTTTACCCTGC
AGATTAGAGGCCGCGAACGATTCGAGATGTTTCGCGAACTGAATGAGGCCCTGGAACTGAAGG
ATGCTCAGGCAGGCAAGGAGCCAGGAGGGTCAGGAGGAGCACCGCACCATCATCATCACCAT
P63-BIDE(SL) polypeptide (hu3F8-scFv, huC825-scFv, huP63-tet,
GS linker, (IgG3 spacer))
SEQ ID NO: 27
EIVMTQTPATLSVSAGERVTITCKASQSVSNDVTWYQQKPGQAPRLLIYSASNRYSGVPARFS
GSGYGTEFTFTISSVQSEDFAVYFCQQDYSSFGCGTKLEIKRGGGGSGGGGSGGGGSQVQLVE
SGPGVVQPGRSLRISCAVSGFSVTNYGVHWVRQPPGKCLEWLGVIWAGGITNYNSAFMSRLTI
SKDNSKNTVYLQMNSLRAEDTAMYYCASRGGHYGYALDYWGQGTLVTVSSGGGGSGGGGSGGG
GSGGGGSHVQLVESGGGLVQPGGSLRLSCAASGFSLTDYGVHWVRQAPGKGLEWLGVIWSGGG
TAYNTALISRFTISRDNSKNTLYLQMNSLRAEDTAVYYCARRGSYPYNYFDAWGCGTLVTVSS
GGGGSGGGGSGGGGSQAVVTQEPSLTVSPGGTVTLTCGSSTGAVTASNYANWVQQKPGQCPRG
LIGGHNNRPPGVPARFSGSLLGGKAALTLLGAQPEDEAEYYCALWYSDHWVIGGGTKLTVLG
(TPLGDTTHT)SGRSPDDELLYLPVRGRETYEMLLKIKESLELMQYLPQHTIETYRQQQQQQH
QHLLQKQGGSGGAPHHHHHH
P63-BIDE(SL) cDNA (hu3F8-scFv, huC825-scFv, huP63-tet,
GS linker, (IgG3 spacer))
SEQ ID NO: 28
GAAATCGTCATGACTCAGACTCCCGCAACCCTGTCAGTGTCCGCTGGGGAACGTGTCACTATT
ACCTGCAAGGCATCTCAGAGCGTGAGCAACGACGTGACCTGGTATCAGCAGAAGCCTGGCCAG
GCTCCACGACTGCTGATCTATTCCGCAAGCAATCGCTACTCCGGAGTGCCCGCACGATTCTCT
GGAAGTGGGTACGGTACCGAGTTCACTTTTACCATTTCCAGCGTGCAGAGCGAAGACTTCGCT
GTCTATTTTTGCCAGCAGGATTACTCTAGTTTTGGCTGTGGAACAAAGCTGGAGATCAAAAGG
GGAGGAGGAGGTTCTGGCGGAGGAGGTAGTGGCGGAGGGGGTTCACAGGTGCAGCTGGTCGAA
TCTGGGCCAGGCGTGGTCCAGCCAGGACGTTCCCTGAGGATTAGCTGCGCCGTGAGCGGGTTC
TCTGTCACAAACTACGGAGTGCACTGGGTCCGTCAGCCACCTGGCAAATGTCTGGAGTGGCTG
GGAGTGATCTGGGCAGGAGGAATCACTAACTACAACTCTGCTTTTATGAGTCGCCTGACCATC
TCAAAGGACAACTCCAAAAATACAGTGTACCTGCAGATGAATTCACTGCGGGCAGAAGATACC
GCCATGTACTATTGCGCCTCCAGGGGGGGTCATTACGGCTATGCCCTGGACTATTGGGGCCAG
GGAACACTGGTGACTGTCTCATCCGGAGGAGGAGGATCCGGAGGAGGAGGTAGCGGCGGAGGG
GGTTCTGGCGGAGGGGGTAGTCACGTGCAGCTGGTCGAGTCCGGAGGAGGGCTGGTGCAGCCT
GGTGGCAGCCTGCGACTGTCTTGTGCCGCTAGTGGCTTCTCACTGACAGATTACGGCGTGCAT
TGGGTCCGACAGGCTCCAGGGAAGGGTCTGGAATGGCTGGGAGTGATTTGGTCTGGAGGGGGT
ACAGCTTATAACACTGCACTGATCAGTCGGTTCACTATCAGTAGAGACAACTCAAAGAACACC
CTGTACCTGCAGATGAACTCTCTGCGGGCCGAGGATACCGCTGTGTACTATTGCGCTAGGCGG
GGCAGTTACCCTTATAATTACTTTGACGCATGGGGCTGTGGAACCCTGGTGACAGTCAGCTCT
GGCGGAGGGGGTTCAGGCGGCGGCGGTTCCGGCGGAGGAGGTAGCCAGGCCGTGGTCACTCAG
GAGCCTTCCCTGACCGTGAGCCCAGGAGGAACAGTCACTCTGACCTGCGGGAGTTCAACCGGT
GCCGTGACAGCCTCCAACTACGCTAATTGGGTCCAGCAGAAGCCCGGGCAGTGTCCTAGAGGT
CTGATCGGGGGTCACAACAATCGTCCACCCGGAGTGCCAGCCAGGTTCTCAGGCTCCCTGCTG
GGCGGAAAAGCAGCACTGACTCTGCTGGGCGCTCAGCCAGAGGACGAAGCAGAGTACTATTGC
GCCCTGTGGTATTCTGATCACTGGGTCATCGGGGGTGGCACTAAGCTGACCGTGCTGGGC(AC
ACCCCYGGGAGACACCACACAYACY)AGYGGGAGATCCCCCGACGATGAGCTGCTGTACCTGC
CTGTGAGGGGCCGGGAGACCTATGAAATGCTGCTGAAGATCAAAGAGAGCCTGGAACTGATGC
AGTACCTGCCACAGCACACCATTGAAACATATAGGCAACAACAGCAGCAGCAGCATCAGCATC
TGCTGCAGAAGCAGGGAGGGTCAGGAGGAGCACCGCACCATCATCATCACCATT
P73-BIDE(SL) polypeptide (hu3F8-scFv, huC825scFv, huP73-tet,
GS linker, (IgG3 spacer))
SEQ ID NO: 29
EIVMTQTPATLSVSAGERVTITCKASQSVSNDVTWYQQKPGQAPRLLIYSASNRYSGVPARFS
GSGYGTEFTFTISSVQSEDFAVYFCQQDYSSFGCGTKLEIKRGGGGSGGGGSGGGGSQVQLVE
SGPGVVQPGRSLRISCAVSGFSVTNYGVHWVRQPPGKCLEWLGVIWAGGITNYNSAFMSRLTI
SKDNSKNTVYLQMNSLRAEDTAMYYCASRGGHYGYALDYWGQGTLVTVSSGGGGSGGGGSGGG
GSGGGGSHVQLVESGGGLVQPGGSLRLSCAASGFSLTDYGVHWVRQAPGKGLEWLGVIWSGGG
TAYNTALISRFTISRDNSKNTLYLQMNSLRAEDTAVYYCARRGSYPYNYFDAWGCGTLVTVSS
GGGGSGGGGSGGGGSQAVVTQEPSLTVSPGGTVTLTCGSSTGAVTASNYANWVQQKPGQCPRG
LIGGHNNRPPGVPARFSGSLLGGKAALTLLGAQPEDEAEYYCALWYSDHWVIGGGTKLTVLG
(TTEGDTTHT)SGRHGDEDTYYLQVRGRENFEILMKLKESLELMELVPQPLVDSYRQQQQLLQ
RPGGSGGAPHHHHHH
P73-BIDE(SL) cDNA (hu3F8-scFv, huC825-scFv, huP73-tet,
GS linker, (IgG3 spacer))
SEQ ID NO: 30
GAAATCGTCATGACTCAGACTCCCGCAACCCTGTCAGTGTCCGCTGGGGAACGTGTCACTATT
ACCTGCAAGGCATCTCAGAGCGTGAGCAACGACGTGACCTGGTATCAGCAGAAGCCTGGCCAG
GCTCCACGACTGCTGATCTATTCCGCAAGCAATCGCTACTCCGGAGTGCCCGCACGATTCTCT
GGAAGTGGGTACGGTACCGAGTTCACTTTTACCATTTCCAGCGTGCAGAGCGAAGACTTCGCT
GTCTATTTTTGCCAGCAGGATTACTCTAGTTTTGGCTGTGGAACAAAGCTGGAGATCAAAAGG
GGAGGAGGAGGTTCTGGCGGAGGAGGTAGTGGCGGAGGGGGTTCACAGGTGCAGCTGGTCGAA
TCTGGGCCAGGCGTGGTCCAGCCAGGACGTTCCCTGAGGATTAGCTGCGCCGTGAGCGGGTTC
TCTGTCACAAACTACGGAGTGCACTGGGTCCGTCAGCCACCTGGCAAATGTCTGGAGTGGCTG
GGAGTGATCTGGGCAGGAGGAATCACTAACTACAACTCTGCTTTTATGAGTCGCCTGACCATC
TCAAAGGACAACTCCAAAAATACAGTGTACCTGCAGATGAATTCACTGCGGGCAGAAGATACC
GCCATGTACTATTGCGCCTCCAGGGGGGGTCATTACGGCTATGCCCTGGACTATTGGGGCCAG
GGAACACTGGTGACTGTCTCATCCGGAGGAGGAGGATCCGGAGGAGGAGGTAGCGGCGGAGGG
GGTTCTGGCGGAGGGGGTAGTCACGTGCAGCTGGTCGAGTCCGGAGGAGGGCTGGTGCAGCCT
GGTGGCAGCCTGCGACTGTCTTGTGCCGCTAGTGGCTTCTCACTGACAGATTACGGCGTGCAT
TGGGTCCGACAGGCTCCAGGGAAGGGTCTGGAATGGCTGGGAGTGATTTGGTCTGGAGGGGGT
ACAGCTTATAACACTGCACTGATCAGTCGGTTCACTATCAGTAGAGACAACTCAAAGAACACC
CTGTACCTGCAGATGAACTCTCTGCGGGCCGAGGATACCGCTGTGTACTATTGCGCTAGGCGG
GGCAGTTACCCTTATAATTACTTTGACGCATGGGGCTGTGGAACCCTGGTGACAGTCAGCTCT
GGCGGAGGGGGTTCAGGCGGCGGCGGTTCCGGCGGAGGAGGTAGCCAGGCCGTGGTCACTCAG
GAGCCTTCCCTGACCGTGAGCCCAGGAGGAACAGTCACTCTGACCTGCGGGAGTTCAACCGGT
GCCGTGACAGCCTCCAACTACGCTAATTGGGTCCAGCAGAAGCCCGGGCAGTGTCCTAGAGGT
CTGATCGGGGGTCACAACAATCGTCCACCCGGAGTGCCAGCCAGGTTCTCAGGCTCCCTGCTG
GGCGGAAAAGCAGCACTGACTCTGCTGGGCGCTCAGCCAGAGGACGAAGCAGAGTACTATTGC
GCCCTGTGGTATTCTGATCACTGGGTCATCGGGGGTGGCACTAAGCTGACCGTGCTGGGC(AC
ACCCCTGGGAGACACCACACATACT)AGTGGGAGGCACGGCGACGAAGATACCTACTATCTGC
AGGTGAGGGGACGGGAGAACTTCGAAATCCTGATGAAGCTGAAAGAGTCCCTGGAACTGATGG
AGCTGGTGCCCCAGCCTCTGGTCGACAGCTACAGACAGCAGCAGCAGCTGCTGCAGAGGCCAG
GAGGGTCAGGAGGAGCACCGCACCATCATCATCACCAT
P53-BIDE(LL) polypeptide (hu3F8-scFv, huC825-scFv, huP53-tet,
GS linker, (IgG3 spacer))
SEQ ID NO: 31
EIVMTQTPATLSVSAGERVTITCKASQSVSNDVTWYQQKPGQAPRLLIYSASNRYSGVPARFS
GSGYGTEFTFTISSVQSEDFAVYFCQQDYSSFGCGTKLEIKRGGGGSGGGGSGGGGSGGGGSG
GGGSGGGGSQVQLVESGPGVVQPGRSLRISCAVSGFSVTNYGVHWVRQPPGKCLEWLGVIWAG
GITNYNSAFMSRLTISKDNSKNTVYLQMNSLRAEDTAMYYCASRGGHYGYALDYWGQGTLVTV
SSGGGGSGGGGSGGGGSGGGGSHVQLVESGGGLVQPGGSLRLSCAASGFSLTDYGVHWVRQAP
GKGLEWLGVIWSGGGTAYNTALISRFTISRDNSKNTLYLQMNSLRAEDTAVYYCARRGSYPYN
YFDAWGCGTLVTVSSGGGGSGGGGSGGGGSGGGGSGGGGSGGGGSQAVVTQEPSLTVSPGGTV
TLTCGSSTGAVTASNYANWVQQKPGQCPRGLIGGHNNRPPGVPARFSGSLLGGKAALTLLGAQ
PEDEAEYYCALWYSDHWVIGGGTKLTVLG(TPLGDTTHT)SGKPLDGEYFTLQIRGRERFEMF
RELNEALELKDAQAGKEPGGSGGAPHHHHHH
P53-BIDE(LL) cDNA (hu3F8-scFv, huC825-scFv, huP53-tet,
GS linker, (IgG3 spacer))
SEQ ID NO: 32
GAGATCGTGATGACCCAGACACCCGCAACACTGAGCGTGTCTGCCGGCGAAAGGGTCACTATT
ACCTGCAAGGCCAGTCAGTCAGTGTCCAACGACGTGACTTGGTACCAGCAGAAACCAGGCCAG
GCTCCCCGGCTGCTGATCTACAGCGCATCTAATAGATATAGCGGAGTGCCTGCTCGCTTCAGT
GGTTCAGGCTATGGAACTGAGTTCACCTTCACCATTTCCAGCGTGCAGTCCGAAGACTTCGCA
GTGTACTTTTGCCAGCAGGATTATTCTAGTTTTGGGTGTGGTACAAAGCTGGAGATCAAAAGG
GGAGGAGGAGGTAGTGGCGGAGGAGGTTCAGGCGGAGGGGGTAGCGGCGGAGGGGGTTCTGGC
GGCGGCGGTAGTGGCGGCGGAGGTAGCCAGGTGCAGCTGGTCGAATCCGGCCCTGGAGTGGTC
CAGCCAGGCAGGTCTCTGCGGATCAGTTGCGCCGTGTCCGGATTCAGCGTCACCAACTACGGA
GTGCACTGGGTCAGACAGCCACCTGGCAAGTGTCTGGAGTGGCTGGGAGTGATCTGGGCAGGA
GGAATCACAAACTACAACTCAGCTTTTATGTCCCGCCTGACTATTAGCAAGGACAACTCTAAA
AATACCGTGTATCTGCAGATGAATTCTCTGCGAGCCGAAGATACCGCTATGTACTATTGTGCA
TCCCGTGGGGGTCATTACGGCTATGCCCTGGATTATTGGGGGCAGGGTACCCTGGTGACAGTC
TCATCCGGCGGAGGGGGATCCGGCGGCGGAGGATCTGGCGGAGGTGGAAGTGGGGGAGGCGGA
TCTCATGTGCAGCTGGTGGAAAGCGGAGGCGGCCTGGTGCAGCCTGGGGGATCTCTGAGACTG
TCTTGTGCCGCCAGCGGCTTCTCCCTGACCGATTATGGCGTGCACTGGGTGCGACAGGCCCCT
GGCAAAGGACTGGAATGGCTGGGAGTGATTTGGAGTGGCGGAGGCACCGCCTACAACACCGCC
CTGATCTCCCGGTTCACCATCAGCCGGGACAACTCCAAGAACACCCTGTACCTGCAGATGAAC
TCCCTGCGGGCCGAGGACACCGCTGTGTACTACTGCGCCAGACGGGGCTCCTACCCCTACAAC
TACTTCGACGCTTGGGGCTGCGGCACCCTCGTGACAGTGTCTAGCGGAGGGGGAGGTTCTGGG
GGCGGAGGTTCAGGTGGTGGTGGTTCCGGGGGTGGTGGCTCTGGTGGCGGTGGTTCTGGCGGT
GGCGGATCTCAGGCTGTCGTGACCCAGGAACCCAGCCTGACTGTGTCTCCTGGCGGAACCGTG
ACCCTGACCTGCGGATCTTCTACCGGCGCTGTGACCGCCAGCAACTACGCCAATTGGGTGCAG
CAGAAACCTGGACAGTGCCCTAGAGGCCTGATCGGCGGCCACAACAACAGACCTCCAGGCGTG
CCAGCCCGGTTCTCTGGATCTCTGCTGGGCGGAAAGGCCGCTCTGACACTGCTGGGTGCTCAG
CCTGAGGACGAGGCCGAGTACTACTGTGCCCTGTGGTACTCCGACCACTGGGTCATCGGAGGC
GGGACCAAGCTGACCGTGCTGGGA(ACACCCCTGGGAGACACCACACATACT)AGTGGGAAAC
CTCTGGATGGCGAGTACTTTACCCTGCAGATTAGAGGCCGCGAACGATTCGAGATGTTTCGCG
AACTGAATGAGGCCCTGGAACTGAAGGATGCTCAGGCAGGCAAGGAGCCAGGAGGGTCAGGAG
GAGCACCGCACCATCATCATCACCAT
P63-BIDE(LL) polypeptide (hu3F8-scFv, huC825-scFv, huP63-tet,
GS linker, (IgG3 spacer))
SEQ ID NO: 33
EIVMTQTPATLSVSAGERVTITCKASQSVSNDVTWYQQKPGQAPRLLIYSASNRYSGVPARFS
GSGYGTEFTFTISSVQSEDFAVYFCQQDYSSFGCGTKLEIKRGGGGSGGGGSGGGGSGGGGSG
GGGSGGGGSQVQLVESGPGVVQPGRSLRISCAVSGFSVTNYGVHWVRQPPGKCLEWLGVIWAG
GITNYNSAFMSRLTISKDNSKNTVYLQMNSLRAEDTAMYYCASRGGHYGYALDYWGQGTLVTV
SSGGGGSGGGGSGGGGSGGGGSHVQLVESGGGLVQPGGSLRLSCAASGFSLTDYGVHWVRQAP
GKGLEWLGVIWSGGGTAYNTALISRFTISRDNSKNTLYLQMNSLRAEDTAVYYCARRGSYPYN
YFDAWGCGTLVTVSSGGGGSGGGGSGGGGSGGGGSGGGGSGGGGSQAVVTQEPSLTVSPGGTV
TLTCGSSTGAVTASNYANWVQQKPGQCPRGLIGGHNNRPPGVPARFSGSLLGGKAALTLLGAQ
PEDEAEYYCALWYSDHWVIGGGTKLTVLG(TPLGDTTHT)SGRSPDDELLYLPVRGRETYEML
LKIKESLELMQYLPQHTIETYRQQQQQQHQHLLQKQGGSGGAPHHHHHH
P63-BIDE(LL) cDNA (hu3F8-scFv, huC825-scFv, huP63-tet,
GS linker, (IgG3 spacer))
SEQ ID NO: 34
GAGATCGTGATGACCCAGACACCCGCAACACTGAGCGTGTCTGCCGGCGAAAGGGTCACTATT
ACCTGCAAGGCCAGTCAGTCAGTGTCCAACGACGTGACTTGGTACCAGCAGAAACCAGGCCAG
GCTCCCCGGCTGCTGATCTACAGCGCATCTAATAGATATAGCGGAGTGCCTGCTCGCTTCAGT
GGTTCAGGCTATGGAACTGAGTTCACCTTCACCATTTCCAGCGTGCAGTCCGAAGACTTCGCA
GTGTACTTTTGCCAGCAGGATTATTCTAGTTTTGGGTGTGGTACAAAGCTGGAGATCAAAAGG
GGAGGAGGAGGTAGTGGCGGAGGAGGTTCAGGCGGAGGGGGTAGCGGCGGAGGGGGTTCTGGC
GGCGGCGGTAGTGGCGGCGGAGGTAGCCAGGTGCAGCTGGTCGAATCCGGCCCTGGAGTGGTC
CAGCCAGGCAGGTCTCTGCGGATCAGTTGCGCCGTGTCCGGATTCAGCGTCACCAACTACGGA
GTGCACTGGGTCAGACAGCCACCTGGCAAGTGTCTGGAGTGGCTGGGAGTGATCTGGGCAGGA
GGAATCACAAACTACAACTCAGCTTTTATGTCCCGCCTGACTATTAGCAAGGACAACTCTAAA
AATACCGTGTATCTGCAGATGAATTCTCTGCGAGCCGAAGATACCGCTATGTACTATTGTGCA
TCCCGTGGGGGTCATTACGGCTATGCCCTGGATTATTGGGGGCAGGGTACCCTGGTGACAGTC
TCATCCGGAGGAGGAGGATCCGGAGGAGGAGGTAGCGGCGGAGGGGGTTCTGGCGGAGGGGGT
AGTCATGTGCAGCTGGTGGAAAGCGGAGGCGGCCTGGTGCAGCCTGGGGGATCTCTGAGACTG
TCTTGTGCCGCCAGCGGCTTCTCCCTGACCGATTATGGCGTGCACTGGGTGCGACAGGCCCCT
GGCAAAGGACTGGAATGGCTGGGAGTGATTTGGAGTGGCGGAGGCACCGCCTACAACACCGCC
CTGATCTCCCGGTTCACCATCAGCCGGGACAACTCCAAGAACACCCTGTACCTGCAGATGAAC
TCCCTGCGGGCCGAGGACACCGCTGTGTACTACTGCGCCAGACGGGGCTCCTACCCCTACAAC
TACTTCGACGCTTGGGGCTGCGGCACCCTCGTGACAGTGTCTAGCGGAGGGGGAGGTTCTGGG
GGCGGAGGTTCAGGTGGTGGTGGTTCCGGGGGTGGTGGCTCTGGTGGCGGTGGTTCTGGCGGT
GGCGGATCTCAGGCTGTCGTGACCCAGGAACCCAGCCTGACTGTGTCTCCTGGCGGAACCGTG
ACCCTGACCTGCGGATCTTCTACCGGCGCTGTGACCGCCAGCAACTACGCCAATTGGGTGCAG
CAGAAACCTGGACAGTGCCCTAGAGGCCTGATCGGCGGCCACAACAACAGACCTCCAGGCGTG
CCAGCCCGGTTCTCTGGATCTCTGCTGGGCGGAAAGGCCGCTCTGACACTGCTGGGTGCTCAG
CCTGAGGACGAGGCCGAGTACTACTGTGCCCTGTGGTACTCCGACCACTGGGTCATCGGAGGC
GGGACCAAGCTGACCGTGCTGGGA(ACACCCCTGGGAGACACCACACATACT)AGTGGGAGAT
CCCCCGACGATGAGCTGCTGTACCTGCCTGTGAGGGGCCGGGAGACCTATGAAATGCTGCTGA
AGATCAAAGAGAGCCTGGAACTGATGCAGTACCTGCCACAGCACACCATTGAAACATATAGGC
AACAACAGCAGCAGCAGCATCAGCATCTGCTGCAGAAGCAGGGAGGGTCAGGAGGAGCACCGC
ACCATCATCATCACCAT
P73-BIDE(LL) polypeptide (hu3F8-scFv, huC825-scFv, huP73-tet,
GS linker, (IgG3 spacer))
SEQ ID NO: 35
EIVMTQTPATLSVSAGERVTITCKASQSVSNDVTWYQQKPGQAPRLLIYSASNRYSGVPARFS
GSGYGTEFTFTISSVQSEDFAVYFCQQDYSSFGCGTKLEIKRGGGGSGGGGSGGGGSGGGGSG
GGGSGGGGSQVQLVESGPGVVQPGRSLRISCAVSGFSVTNYGVHWVRQPPGKCLEWLGVIWAG
GITNYNSAFMSRLTISKDNSKNTVYLQMNSLRAEDTAMYYCASRGGHYGYALDYWGQGTLVTV
SSGGGGSGGGGSGGGGSGGGGSHVQLVESGGGLVQPGGSLRLSCAASGFSLTDYGVHWVRQAP
GKGLEWLGVIWSGGGTAYNTALISRFTISRDNSKNTLYLQMNSLRAEDTAVYYCARRGSYPYN
YFDAWGCGTLVTVSSGGGGSGGGGSGGGGSGGGGSGGGGSGGGGSQAVVTQEPSLTVSPGGTV
TLTCGSSTGAVTASNYANWVQQKPGQCPRGLIGGHNNRPPGVPARFSGSLLGGKAALTLLGAQ
PEDEAEYYCALWYSDHWVIGGGTKLTVLG(TPLGDTTHT)SGRHGDEDTYYLQVRGRENFEIL
MKLKESLELMELVPQPLVDSYRQQQQLLQRPGGSGGAPHHHHHH
P73-BIDE(LL) cDNA (hu3F8-scFv, huC825-scFv, huP73-tet,
GS linker, (IgG3 spacer))
SEQ ID NO: 36
GAGATCGTGATGACCCAGACACCCGCAACACTGAGCGTGTCTGCCGGCGAAAGGGTCACTATT
ACCTGCAAGGCCAGTCAGTCAGTGTCCAACGACGTGACTTGGTACCAGCAGAAACCAGGCCAG
GCTCCCCGGCTGCTGATCTACAGCGCATCTAATAGATATAGCGGAGTGCCTGCTCGCTTCAGT
GGTTCAGGCTATGGAACTGAGTTCACCTTCACCATTTCCAGCGTGCAGTCCGAAGACTTCGCA
GTGTACTTTTGCCAGCAGGATTATTCTAGTTTTGGGTGTGGTACAAAGCTGGAGATCAAAAGG
GGAGGAGGAGGTAGTGGCGGAGGAGGTTCAGGCGGAGGGGGTAGCGGCGGAGGGGGTTCTGGC
GGCGGCGGTAGTGGCGGCGGAGGTAGCCAGGTGCAGCTGGTCGAATCCGGCCCTGGAGTGGTC
CAGCCAGGCAGGTCTCTGCGGATCAGTTGCGCCGTGTCCGGATTCAGCGTCACCAACTACGGA
GTGCACTGGGTCAGACAGCCACCTGGCAAGTGTCTGGAGTGGCTGGGAGTGATCTGGGCAGGA
GGAATCACAAACTACAACTCAGCTTTTATGTCCCGCCTGACTATTAGCAAGGACAACTCTAAA
AATACCGTGTATCTGCAGATGAATTCTCTGCGAGCCGAAGATACCGCTATGTACTATTGTGCA
TCCCGTGGGGGTCATTACGGCTATGCCCTGGATTATTGGGGGCAGGGTACCCTGGTGACAGTC
TCATCCGGAGGAGGAGGATCCGGAGGAGGAGGTAGCGGCGGAGGGGGTTCTGGCGGAGGGGGT
AGTCATGTGCAGCTGGTGGAAAGCGGAGGCGGCCTGGTGCAGCCTGGGGGATCTCTGAGACTG
TCTTGTGCCGCCAGCGGCTTCTCCCTGACCGATTATGGCGTGCACTGGGTGCGACAGGCCCCT
GGCAAAGGACTGGAATGGCTGGGAGTGATTTGGAGTGGCGGAGGCACCGCCTACAACACCGCC
CTGATCTCCCGGTTCACCATCAGCCGGGACAACTCCAAGAACACCCTGTACCTGCAGATGAAC
TCCCTGCGGGCCGAGGACACCGCTGTGTACTACTGCGCCAGACGGGGCTCCTACCCCTACAAC
TACTTCGACGCTTGGGGCTGCGGCACCCTCGTGACAGTGTCTAGCGGAGGGGGAGGTTCTGGG
GGCGGAGGTTCAGGTGGTGGTGGTTCCGGGGGTGGTGGCTCTGGTGGCGGTGGTTCTGGCGGT
GGCGGATCTCAGGCTGTCGTGACCCAGGAACCCAGCCTGACTGTGTCTCCTGGCGGAACCGTG
ACCCTGACCTGCGGATCTTCTACCGGCGCTGTGACCGCCAGCAACTACGCCAATTGGGTGCAG
CAGAAACCTGGACAGTGCCCTAGAGGCCTGATCGGCGGCCACAACAACAGACCTCCAGGCGTG
CCAGCCCGGTTCTCTGGATCTCTGCTGGGCGGAAAGGCCGCTCTGACACTGCTGGGTGCTCAG
CCTGAGGACGAGGCCGAGTACTACTGTGCCCTGTGGTACTCCGACCACTGGGTCATCGGAGGC
GGGACCAAGCTGACCGTGCTGGGA(ACACCCCTGGGAGACACCACACATACT)AGTGGGAGGC
ACGGCGACGAAGATACCTACTATCTGCAGGTGAGGGGACGGGAGAACTTCGAAATCCTGATGA
AGCTGAAAGAGTCCCTGGAACTGATGGAGCTGGTGCCCCAGCCTCTGGTCGACAGCTACAGAC
AGCAGCAGCAGCTGCTGCAGAGGCCAGGAGGGTCAGGAGGAGCACCGCACCATCATCATCACC
AT
P53-mBIDE(noHIS) polypeptide (hu3F8-scFv, C825-scFv,
huP53-tet, GS linker, (IgG3 spacer))
SEQ ID NO: 37
EIVMTQTPATLSVSAGERVTITCKASQSVSNDVTWYQQKPGQAPRLLIYSASNRYSGVPARFS
GSGYGTEFTFTISSVQSEDFAVYFCQQDYSSFGCGTKLEIKRGGGGSGGGGSGGGGSQVQLVE
SGPGVVQPGRSLRISCAVSGFSVTNYGVHWVRQPPGKCLEWLGVIWAGGITNYNSAFMSRLTI
SKDNSKNTVYLQMNSLRAEDTAMYYCASRGGHYGYALDYWGQGTLVTVSSGGGGSGGGGSGGG
GSGGGGSHVKLQESGPGLVQPSQSLSLTCTVSGFSLTDYGVHWVRQSPGKGLEWLGVIWSGGG
TAYNTALISRLNIYRDNSKNQVFLEMNSLQAEDTAMYYCARRGSYPYNYFDAWGCGTTVTVSS
GGGGSGGGGSGGGGSQAVVIQESALTTPPGETVTLTCGSSTGAVTASNYANWVQEKPDHCFTG
LIGGHNNRPPGVPARFSGSLIGDKAALTIAGTQTEDEAIYFCALWYSDHWVIGGGTRLTVLG
(TPLGDTTHT)SGKPEDGEYFTLQIRGRERFEMFRELNEALELKDAQAGKEPGGSGGA
P53-mBIDE(noHIS) cDNA (hu3F8-scFv, C825-scFv, huP53-tet,
GS linker, (IgG3 spacer))
SEQ ID NO: 38
GAAATCGTCATGACTCAGACTCCCGCAACCCTGTCAGTGTCCGCTGGGGAACGTGTCACTATT
ACCTGCAAGGCATCTCAGAGCGTGAGCAACGACGTGACCTGGTATCAGCAGAAGCCTGGCCAG
GCTCCACGACTGCTGATCTATTCCGCAAGCAATCGCTACTCCGGAGTGCCCGCACGATTCTCT
GGAAGTGGGTACGGTACCGAGTTCACTTTTACCATTTCCAGCGTGCAGAGCGAAGACTTCGCT
GTCTATTTTTGCCAGCAGGATTACTCTAGTTTTGGCTGTGGAACAAAGCTGGAGATCAAAAGG
GGAGGAGGAGGTTCTGGCGGAGGAGGTAGTGGCGGAGGGGGTTCACAGGTGCAGCTGGTCGAA
TCTGGGCCAGGCGTGGTCCAGCCAGGACGTTCCCTGAGGATTAGCTGCGCCGTGAGCGGGTTC
TCTGTCACAAACTACGGAGTGCACTGGGTCCGTCAGCCACCTGGCAAATGTCTGGAGTGGCTG
GGAGTGATCTGGGCAGGAGGAATCACTAACTACAACTCTGCTTTTATGAGTCGCCTGACCATC
TCAAAGGACAACTCCAAAAATACAGTGTACCTGCAGATGAATTCACTGCGGGCAGAAGATACC
GCCATGTACTATTGCGCCTCCAGGGGGGGTCATTACGGCTATGCCCTGGACTATTGGGGCCAG
GGAACACTGGTGACTGTCTCATCCGGAGGAGGAGGATCCGGAGGAGGAGGTAGCGGCGGAGGG
GGTTCTGGCGGAGGGGGTAGTCACGTGAAGCTGCAGGAAAGCGGCCCTGGACTGGTGCAGCCT
TCCCAGTCTCTGTCCCTGACCTGCACCGTGTCCGGCTTCTCCCTGACCGATTACGGCGTGCAC
TGGGTGCGACAGTCTCCAGGCAAGGGCCTGGAATGGCTGGGAGTGATTTGGAGCGGTGGCGGA
ACCGCCTACAACACCGCCCTGATCTCCCGGCTGAACATCTACCGGGACAACTCCAAGAACCAG
GTGTTCCTGGAAATGAACTCCCTGCAGGCAGAGGACACCGCCATGTACTACTGCGCCAGACGG
GGCTCCTACCCCTACAACTACTTCGACGCTTGGGGCTGCGGCACCACCGTGACAGTGTCTAGC
GGAGGTGGTGGATCTGGGGGCGGAGGTAGCGGAGGGGGAGGTTCTCAGGCTGTCGTGATCCAG
GAATCTGCCCTGACCACCCCCCCTGGCGAGACAGTGACACTGACCTGCGGATCTTCCACCGGC
GCTGTGACCGCCTCCAACTACGCCAACTGGGTGCAGGAAAAGCCCGACCACTGCTTCACCGGC
CTGATCGGCGGCCACAACAACAGACCTCCAGGCGTGCCAGCCCGGTTCTCCGGCTCTCTGATC
GGAGATAAGGCCGCCCTGACAATCGCCGGCACCCAGACAGAGGACGAGGCTATCTACTTCTGC
GCCCTGTGGTACAGCGACCACTGGGTCATCGGCGGAGGCACCAGACTGACCGTGCTGGGA(AC
ACCCCTGGGAGACACCACACATACT)AGTGGCAAACCTCTGGATGGAGAGTACTTTACCCTGC
AGATTAGAGGCCGCGAACGATTCGAGATGTTTCGCGAACTGAATGAGGCCCTGGAACTGAAGG
ATGCTCAGGCAGGCAAGGAACCAGGCGGTAGCGGCGGCGCA
P53-mBIDE polypeptide (hu3F8-scFv, C825-scFv, huP53-tet,
GS linker, (IgG3 spacer))
SEQ ID NO: 39
EIVMTQTPATLSVSAGERVTITCKASQSVSNDVTWYQQKPGQAPRLLIYSASNRYSGVPARFS
GSGYGTEFTFTISSVQSEDFAVYFCQQDYSSFGCGTKLEIKRGGGGSGGGGSGGGGSGGGGSG
GGGSGGGGSQVQLVESGPGVVQPGRSLRISCAVSGFSVTNYGVHWVRQPPGKCLEWLGVIWAG
GITNYNSAFMSRLTISKDNSKNTVYLQMNSLRAEDTAMYYCASRGGHYGYALDYWGQGTLVTV
SSGGGGSGGGGSGGGGSGGGGSHVKLQESGPGLVQPSQSLSLTCTVSGFSLTDYGVHWVRQSP
GKGLEWLGVIWSGGGTAYNTALISRLNIYRDNSKNQVFLEMNSLQAEDTAMYYCARRGSYPYN
YFDAWGCGTTVTVSSGGGGSGGGGSGGGGSQAVVIQESALTTPPGETVTLTCGSSTGAVTASN
YANWVQEKPDHCFTGLIGGHNNRPPGVPARFSGSLIGDKAALTIAGTQTEDEAIYFCALWYSD
HWVIGGGTRLTVLG(TPLGDTTHT)SGKPLDGEYFTLQIRGRERFEMFRELNEALELKDAQAG
KEPGGSGGAPHHHHHH
P53-mBIDE cDNA (hu3F8-scFv, C825-scFv, huP53-tet, GS linker,
(IgG3 spacer))
SEQ ID NO: 40
GAGATCGTGATGACCCAGACACCCGCAACACTGAGCGTGTCTGCCGGCGAAAGGGTCACTATT
ACCTGCAAGGCCAGTCAGTCAGTGTCCAACGACGTGACTTGGTACCAGCAGAAACCAGGCCAG
GCTCCCCGGCTGCTGATCTACAGCGCATCTAATAGATATAGCGGAGTGCCTGCTCGCTTCAGT
GGTTCAGGCTATGGAACTGAGTTCACCTTCACCATTTCCAGCGTGCAGTCCGAAGACTTCGCA
GTGTACTTTTGCCAGCAGGATTATTCTAGTTTTGGGTGTGGTACAAAGCTGGAGATCAAAAGG
GGAGGAGGAGGTAGTGGCGGAGGAGGTTCAGGCGGAGGGGGTAGCGGCGGAGGGGGTTCTGGC
GGCGGCGGTAGTGGCGGCGGAGGTAGCCAGGTGCAGCTGGTCGAATCCGGCCCTGGAGTGGTC
CAGCCAGGCAGGTCTCTGCGGATCAGTTGCGCCGTGTCCGGATTCAGCGTCACCAACTACGGA
GTGCACTGGGTCAGACAGCCACCTGGCAAGTGTCTGGAGTGGCTGGGAGTGATCTGGGCAGGA
GGAATCACAAACTACAACTCAGCTTTTATGTCCCGCCTGACTATTAGCAAGGACAACTCTAAA
AATACCGTGTATCTGCAGATGAATTCTCTGCGAGCCGAAGATACCGCTATGTACTATTGTGCA
TCCCGTGGGGGTCATTACGGCTATGCCCTGGATTATTGGGGGCAGGGTACCCTGGTGACAGTC
TCATCCGGCGGAGGGGGATCCGGAGGAGGAGGTAGCGGCGGAGGGGGTTCTGGCGGAGGGGGT
AGTCACGTGAAGCTGCAGGAAAGCGGCCCTGGACTGGTGCAGCCTTCCCAGTCTCTGTCCCTG
ACCTGCACCGTGTCCGGCTTCTCCCTGACCGATTACGGCGTGCACTGGGTGCGACAGTCTCCA
GGCAAGGGCCTGGAATGGCTGGGAGTGATTTGGAGCGGTGGCGGAACCGCCTACAACACCGCC
CTGATCTCCCGGCTGAACATCTACCGGGACAACTCCAAGAACCAGGTGTTCCTGGAAATGAAC
TCCCTGCAGGCAGAGGACACCGCCATGTACTACTGCGCCAGACGGGGCTCCTACCCCTACAAC
TACTTCGACGCTTGGGGCTGCGGCACCACCGTGACAGTGTCTAGCGGAGGTGGTGGATCTGGG
GGCGGAGGTAGCGGAGGGGGAGGTTCTCAGGCTGTCGTGATCCAGGAATCTGCCCTGACCACC
CCCCCTGGCGAGACAGTGACACTGACCTGCGGATCTTCCACCGGCGCTGTGACCGCCTCCAAC
TACGCCAACTGGGTGCAGGAAAAGCCCGACCACTGCTTCACCGGCCTGATCGGCGGCCACAAC
AACAGACCTCCAGGCGTGCCAGCCCGGTTCTCCGGCTCTCTGATCGGAGATAAGGCCGCCCTG
ACAATCGCCGGCACCCAGACAGAGGACGAGGCTATCTACTTCTGCGCCCTGTGGTACAGCGAC
CACTGGGTCATCGGCGGAGGCACCAGACTGACCGTGCTGGGA(ACACCCCTGGGAGACACCAC
ACATACT)AGTGGGAAACCTCTGGATGGCGAGTACTTTACCCTGCAGATTAGAGGCCGCGAAC
GATTCGAGATGTTTCGCGAACTGAATGAGGCCCTGGAACTGAAGGATGCTCAGGCAGGCAAGG
AGCCAGGAGGGTCAGGAGGAGCACCGCACCATCATCATCACCAT
P63-mBIDE polypeptide (hu3F8-scFv, C825-scFv, huP63-tet,
GS linker, (IgG3 spacer))
SEQ ID NO: 41
EIVMTQTPATLSVSAGERVTITCKASQSVSNDVTWYQQKPGQAPRLLIYSASNRYSGVPARFS
GSGYGTEFTFTISSVQSEDFAVYFCQQDYSSFGCGTKLEIKRGGGGSGGGGSGGGGSGGGGSG
GGGSGGGGSQVQLVESGPGVVQPGRSLRISCAVSGFSVTNYGVHWVRQPPGKCLEWLGVIWAG
GITNYNSAFMSRLTISKDNSKNTVYLQMNSLRAEDTAMYYCASRGGHYGYALDYWGOGTLVTV
SSGGGGSGGGGSGGGGSGGGGSHVKLQESGPGLVQPSQSLSLTCTVSGFSLTDYGVHWVRQSP
GKGLEWLGVIWSGGGTAYNTALISRLNIYRDNSKNQVFLEMNSLQAEDTAMYYCARRGSYPYN
YFDAWGCGTTVTVSSGGGGSGGGGSGGGGSQAVVIQESALTTPPGETVTLTCGSSTGAVTASN
YANWVQEKPDHCFTGLIGGHNNRPPGVPARFSGSLIGDKAALTIAGTQTEDEAIYFCALWYSD
HWVIGGGTRLTVLG(TPLGDTTHT)SGRSPDDELLYLPVRGRETYEMLLKIKESLELMQYLPQ
HTIETYRQQQQQQHQHLLQKQGGSGGAPHHHHHH
P63-mBIDE cDNA (hu3F8-scFv, C825-scFv, huP63-tet, GS linker,
(IgG3 spacer))
SEQ ID NO: 42
GAGATCGTGATGACCCAGACACCCGCAACACTGAGCGTGTCTGCCGGCGAAAGGGTCACTATT
ACCTGCAAGGCCAGTCAGTCAGTGTCCAACGACGTGACTTGGTACCAGCAGAAACCAGGCCAG
GCTCCCCGGCTGCTGATCTACAGCGCATCTAATAGATATAGCGGAGTGCCTGCTCGCTTCAGT
GGTTCAGGCTATGGAACTGAGTTCACCTTCACCATTTCCAGCGTGCAGTCCGAAGACTTCGCA
GTGTACTTTTGCCAGCAGGATTATTCTAGTTTTGGGTGTGGTACAAAGCTGGAGATCAAAAGG
GGAGGAGGAGGTAGTGGCGGAGGAGGTTCAGGCGGAGGGGGTAGCGGCGGAGGGGGTTCTGGC
GGCGGCGGTAGTGGCGGCGGAGGTAGCCAGGTGCAGCTGGTCGAATCCGGCCCTGGAGTGGTC
CAGCCAGGCAGGTCTCTGCGGATCAGTTGCGCCGTGTCCGGATTCAGCGTCACCAACTACGGA
GTGCACTGGGTCAGACAGCCACCTGGCAAGTGTCTGGAGTGGCTGGGAGTGATCTGGGCAGGA
GGAATCACAAACTACAACTCAGCTTTTATGTCCCGCCTGACTATTAGCAAGGACAACTCTAAA
AATACCGTGTATCTGCAGATGAATTCTCTGCGAGCCGAAGATACCGCTATGTACTATTGTGCA
TCCCGTGGGGGTCATTACGGCTATGCCCTGGATTATTGGGGGCAGGGTACCCTGGTGACAGTC
TCATCCGGAGGAGGAGGATCCGGAGGAGGAGGTAGCGGCGGAGGGGGTTCTGGCGGAGGGGGT
AGTCACGTGAAGCTGCAGGAAAGCGGCCCTGGACTGGTGCAGCCTTCCCAGTCTCTGTCCCTG
ACCTGCACCGTGTCCGGCTTCTCCCTGACCGATTACGGCGTGCACTGGGTGCGACAGTCTCCA
GGCAAGGGCCTGGAATGGCTGGGAGTGATTTGGAGCGGTGGCGGAACCGCCTACAACACCGCC
CTGATCTCCCGGCTGAACATCTACCGGGACAACTCCAAGAACCAGGTGTTCCTGGAAATGAAC
TCCCTGCAGGCAGAGGACACCGCCATGTACTACTGCGCCAGACGGGGCTCCTACCCCTACAAC
TACTTCGACGCTTGGGGCTGCGGCACCACCGTGACAGTGTCTAGCGGAGGTGGTGGATCTGGG
GGCGGAGGTAGCGGAGGGGGAGGTTCTCAGGCTGTCGTGATCCAGGAATCTGCCCTGACCACC
CCCCCTGGCGAGACAGTGACACTGACCTGCGGATCTTCCACCGGCGCTGTGACCGCCTCCAAC
TACGCCAACTGGGTGCAGGAAAAGCCCGACCACTGCTTCACCGGCCTGATCGGCGGCCACAAC
AACAGACCTCCAGGCGTGCCAGCCCGGTTCTCCGGCTCTCTGATCGGAGATAAGGCCGCCCTG
ACAATCGCCGGCACCCAGACAGAGGACGAGGCTATCTACTTCTGCGCCCTGTGGTACAGCGAC
CACTGGGTCATCGGCGGAGGCACCAGACTGACCGTGCTGGGA(ACACCCCTGGGAGACACCAC
ACATACT)AGTGGGAGATCCCCCGACGATGAGCTGCTGTACCTGCCTGTGAGGGGCCGGGAGA
CCTATGAAATGCTGCTGAAGATCAAAGAGAGCCTGGAACTGATGCAGTACCTGCCACAGCACA
CCATTGAAACATATAGGCAACAACAGCAGCAGCAGCATCAGCATCTGCTGCAGAAGCAGGGAG
GGTCAGGAGGAGCACCGCACCATCATCATCACCATT
P73-mBIDE polypeptide (hu3F8-scFv, C825-scFv, huP73-tet,
GS linker, (IgG3 spacer))
SEQ ID NO: 43
EIVMTQTPATLSVSAGERVTITCKASQSVSNDVTWYQQKPGQAPRLLIYSASNRYSGVPARFS
GSGYGTEFTFTISSVQSEDFAVYFCQQDYSSFGCGTKLEIKRGGGGSGGGGSGGGGSGGGGSG
GGGSGGGGSQVQLVESGPGVVQPGRSLRISCAVSGFSVTNYGVHWVRQPPGKCLEWLGVIWAG
GITNYNSAFMSRLTISKDNSKNTVYLQMNSLRAEDTAMYYCASRGGHYGYALDYWGQGTLVTV
SSGGGGSGGGGSGGGGSGGGGSHVKLQESGPGLVQPSQSLSLTCTVSGFSLTDYGVHWVRQSP
GKGLEWLGVIWSGGGTAYNTALISRLNIYRDNSKNQVFLEMNSLQAEDTAMYYCARRGSYPYN
YFDAWGCGTTVTVSSGGGGSGGGGSGGGGSQAVVIQESALTTPPGETVTLTCGSSTGAVTASN
YANWVQEKPDHCFTGLIGGHNNRPPGVPARFSGSLIGDKAALTIAGTQTEDEAIYFCALWYSD
HWVIGGGTRLTVLG(TPLGDTTHT)SGRHGDEDTYYLQVRGRENFEILMKLKESLELMELVPQ
PLVDSYRQQQQLLQRPGGSGGAPHHHHHH
P73-mBIDE cDNA (hu3F8-scFv, C825-scFv, huP73-tet, GS linker,
(IgG3 spacer))
SEQ ID NO: 44
GAGATCGTGATGACCCAGACACCCGCAACACTGAGCGTGTCTGCCGGCGAAAGGGTCACTATT
ACCTGCAAGGCCAGTCAGTCAGTGTCCAACGACGTGACTTGGTACCAGCAGAAACCAGGCCAG
GCTCCCCGGCTGCTGATCTACAGCGCATCTAATAGATATAGCGGAGTGCCTGCTCGCTTCAGT
GGTTCAGGCTATGGAACTGAGTTCACCTTCACCATTTCCAGCGTGCAGTCCGAAGACTTCGCA
GTGTACTTTTGCCAGCAGGATTATTCTAGTTTTGGGTGTGGTACAAAGCTGGAGATCAAAAGG
GGAGGAGGAGGTAGTGGCGGAGGAGGTTCAGGCGGAGGGGGTAGCGGCGGAGGGGGTTCTGGC
GGCGGCGGTAGTGGCGGCGGAGGTAGCCAGGTGCAGCTGGTCGAATCCGGCCCTGGAGTGGTC
CAGCCAGGCAGGTCTCTGCGGATCAGTTGCGCCGTGTCCGGATTCAGCGTCACCAACTACGGA
GTGCACTGGGTCAGACAGCCACCTGGCAAGTGTCTGGAGTGGCTGGGAGTGATCTGGGCAGGA
GGAATCACAAACTACAACTCAGCTTTTATGTCCCGCCTGACTATTAGCAAGGACAACTCTAAA
AATACCGTGTATCTGCAGATGAATTCTCTGCGAGCCGAAGATACCGCTATGTACTATTGTGCA
TCCCGTGGGGGTCATTACGGCTATGCCCTGGATTATTGGGGGCAGGGTACCCTGGTGACAGTC
TCATCCGGAGGAGGAGGATCCGGAGGAGGAGGTAGCGGCGGAGGGGGTTCTGGCGGAGGGGGT
AGTCACGTGAAGCTGCAGGAAAGCGGCCCTGGACTGGTGCAGCCTTCCCAGTCTCTGTCCCTG
ACCTGCACCGTGTCCGGCTTCTCCCTGACCGATTACGGCGTGCACTGGGTGCGACAGTCTCCA
GGCAAGGGCCTGGAATGGCTGGGAGTGATTTGGAGCGGTGGCGGAACCGCCTACAACACCGCC
CTGATCTCCCGGCTGAACATCTACCGGGACAACTCCAAGAACCAGGTGTTCCTGGAAATGAAC
TCCCTGCAGGCAGAGGACACCGCCATGTACTACTGCGCCAGACGGGGCTCCTACCCCTACAAC
TACTTCGACGCTTGGGGCTGCGGCACCACCGTGACAGTGTCTAGCGGAGGTGGTGGATCTGGG
GGCGGAGGTAGCGGAGGGGGAGGTTCTCAGGCTGTCGTGATCCAGGAATCTGCCCTGACCACC
CCCCCTGGCGAGACAGTGACACTGACCTGCGGATCTTCCACCGGCGCTGTGACCGCCTCCAAC
TACGCCAACTGGGTGCAGGAAAAGCCCGACCACTGCTTCACCGGCCTGATCGGCGGCCACAAC
AACAGACCTCCAGGCGTGCCAGCCCGGTTCTCCGGCTCTCTGATCGGAGATAAGGCCGCCCTG
ACAATCGCCGGCACCCAGACAGAGGACGAGGCTATCTACTTCTGCGCCCTGTGGTACAGCGAC
CACTGGGTCATCGGCGGAGGCACCAGACTGACCGTGCTGGGA(ACACCCCTGGGAGACACCAC
ACATACT)AGTGGGAGGCACGGCGACGAAGATACCTACTATCTGCAGGTGAGGGGACGGGAGA
ACTTCGAAATCCTGATGAAGCTGAAAGAGTCCCTGGAACTGATGGAGCTGGTGCCCCAGCCTC
TGGTCGACAGCTACAGACAGCAGCAGCAGCTGCTGCAGAGGCCAGGAGGGTCAGGAGGAGCAC
CGCACCATCATCATCACCAT
P53-mBIDE(SL) polypeptide (hu3F8-scFv, C825-scFv, huP53-tet,
GS linker, (IgG3 spacer))
SEQ ID NO: 45
EIVMTQTPATLSVSAGERVTITCKASQSVSNDVTWYQQKPGQAPRLLIYSASNRYSGVPARFS
GSGYGTEFTFTISSVQSEDFAVYFCQQDYSSFGCGTKLEIKRGGGGSGGGGSGGGGSQVQLVE
SGPGVVQPGRSLRISCAVSGFSVTNYGVHWVRQPPGKCLEWLGVIWAGGITNYNSAFMSRLTI
SKDNSKNTVYLQMNSLRAEDTAMYYCASRGGHYGYALDYWGQGTLVTVSSGGGGSGGGGSGGG
GSGGGGSHVKLQESGPGLVOPSQSLSLTCTVSGFSLTDYGVHWVRQSPGKGLEWLGVIWSGGG
TAYNTALISRLNIYRDNSKNQVFLEMNSLQAEDTAMYYCARRGSYPYNYFDAWGCGTTVTFSS
GGGGSGGGGSGGGGSQAVVIQESALTTPPGETVTLTCGSSTGAVTASNYANWVQEKPDHCFTG
LIGGHNNRPPGVPARFSGSLIGDKAALTIAGTQTEDEAIYFCALWYSDHWVIGGGTRLTVLG
(TPLGDTTHT)SGKPLDGEYFTLQIRGRERFEMFRELNEALELKDAQAGKEPGGSGGAPHHHH
HH
P53-mBIDE(SL) cDNA (hu3F8-scFv, C825-scFv, huP53-tet,
GS linker, (IgG3 spacer))
SEQ ID NO: 46
GAAATCGTCATGACTCAGACTCCCGCAACCCTGTCAGTGTCCGCTGGGGAACGTGTCACTATT
ACCTGCAAGGCATCTCAGAGCGTGAGCAACGACGTGACCTGGTATCAGCAGAAGCCTGGCCAG
GCTCCACGACTGCTGATCTATTCCGCAAGCAATCGCTACTCCGGAGTGCCCGCACGATTCTCT
GGAAGTGGGTACGGTACCGAGTTCACTTTTACCATTTCCAGCGTGCAGAGCGAAGACTTCGCT
GTCTATTTTTGCCAGCAGGATTACTCTAGTTTTGGCTGTGGAACAAAGCTGGAGATCAAAAGG
GGAGGAGGAGGTTCTGGCGGAGGAGGTAGTGGCGGAGGGGGTTCACAGGTGCAGCTGGTCGAA
TCTGGGCCAGGCGTGGTCCAGCCAGGACGTTCCCTGAGGATTAGCTGCGCCGTGAGCGGGTTC
TCTGTCACAAACTACGGAGTGCACTGGGTCCGTCAGCCACCTGGCAAATGTCTGGAGTGGCTG
GGAGTGATCTGGGCAGGAGGAATCACTAACTACAACTCTGCTTTTATGAGTCGCCTGACCATC
TCAAAGGACAACTCCAAAAATACAGTGTACCTGCAGATGAATTCACTGCGGGCAGAAGATACC
GCCATGTACTATTGCGCCTCCAGGGGGGGTCATTACGGCTATGCCCTGGACTATTGGGGCCAG
GGAACACTGGTGACTGTCTCATCCGGAGGAGGAGGATCCGGAGGAGGAGGTAGCGGCGGAGGG
GGTTCTGGCGGAGGGGGTAGTCACGTGAAGCTGCAGGAAAGCGGCCCTGGACTGGTGCAGCCT
TCCCAGTCTCTGTCCCTGACCTGCACCGTGTCCGGCTTCTCCCTGACCGATTACGGCGTGCAC
TGGGTGCGACAGTCTCCAGGCAAGGGCCTGGAATGGCTGGGAGTGATTTGGAGCGGTGGCGGA
ACCGCCTACAACACCGCCCTGATCTCCCGGCTGAACATCTACCGGGACAACTCCAAGAACCAG
GTGTTCCTGGAAATGAACTCCCTGCAGGCAGAGGACACCGCCATGTACTACTGCGCCAGACGG
GGCTCCTACCCCTACAACTACTTCGACGCTTGGGGCTGCGGCACCACCGTGACAGTGTCTAGC
GGAGGTGGTGGATCTGGGGGCGGAGGTAGCGGAGGGGGAGGTTCTCAGGCTGTCGTGATCCAG
GAATCTGCCCTGACCACCCCCCCTGGCGAGACAGTGACACTGACCTGCGGATCTTCCACCGGC
GCTGTGACCGCCTCCAACTACGCCAACTGGGTGCAGGAAAAGCCCGACCACTGCTTCACCGGC
CTGATCGGCGGCCACAACAACAGACCTCCAGGCGTGCCAGCCCGGTTCTCCGGCTCTCTGATC
GGAGATAAGGCCGCCCTGACAATCGCCGGCACCCAGACAGAGGACGAGGCTATCTACTTCTGC
GCCCTGTGGTACAGCGACCACTGGGTCATCGGCGGAGGCACCAGACTGACCGTGCTGGGA(AC
ACCCCTGGGAGACACCACACATACT)AGTGGGAAACCTCTGGATGGCGAGTACTTTACCCTGC
AGATTAGAGGCCGCGAACGATTCGAGATGTTTCGCGAACTGAATGAGGCCCTGGAACTGAAGG
ATGCTCAGGCAGGCAAGGAGCCAGGAGGGTCAGGAGGAGCACCGCACCATCATCATCACCAT
P63-mBIDE(SL) polypeptide (hu3F8-scFv, C825-scFv, huP63-tet,
GS linker, (IgG3 spacer))
SEQ ID NO: 47
EIVMTQTPATLSVSAGERVTITCKASQSVSNDVTWYQQKPGQAPRLLIYSASNRYSGVPARFS
GSGYGTEFTFTISSVQSEDFAVYFCQQDYSSFGCGTKLEIKRGGGGSGGGGSGGGGSQVQLVE
SGPGVVQPGRSLRISCAVSGFSVTNYGVHWVRQPPGKCLEWLGVIWAGGITNYNSAFMSRLTI
SKDNSKNTVYLQMNSLRAEDTAMYYCASRGGHYGYALDYWGQGTLVTVSSGGGGSGGGGSGGG
GSGGGGSHVKLQESGPGLVQPSQSLSLTCTVSGFSLTDYGVHWVRQSPGKGLEWLGVIWSGGG
TAYNTALISRLNIYRDNSKNQVFLEMNSLQAEDTAMYYCARRGSYPYNYFDAWGCGTTVTVSS
GGGGSGGGGSGGGGSQAVVIQESALTTPPGETVTLTCGSSTGAVTASNYANWVQEKPDHCFTG
LIGGHNNRPPGVPARFSGSLIGDKAALTIAGTQTEDEAIYFCALWYSDHWVIGGGTRLTVLG
(TPLGDTTHT)SGRSPDDELLYLPVRGRETYEMLLKIKESLELMQYLPQHTIETYRQQQQQQH
QHLLQKQGGSGGAPHHHHHH
P63-mBIDE(SL) cDNA (hu3F8-scFv, C825-scFv, huP63-tet,
GS linker, (IgG3 spacer))
SEQ ID NO: 48
GAAATCGTCATGACTCAGACTCCCGCAACCCTGTCAGTGTCCGCTGGGGAACGTGTCACTATT
ACCTGCAAGGCATCTCAGAGCGTGAGCAACGACGTGACCTGGTATCAGCAGAAGCCTGGCCAG
GCTCCACGACTGCTGATCTATTCCGCAAGCAATCGCTACTCCGGAGTGCCCGCACGATTCTCT
GGAAGTGGGTACGGTACCGAGTTCACTTTTACCATTTCCAGCGTGCAGAGCGAAGACTTCGCT
GTCTATTTTTGCCAGCAGGATTACTCTAGTTTTGGCTGTGGAACAAAGCTGGAGATCAAAAGG
GGAGGAGGAGGTTCTGGCGGAGGAGGTAGTGGCGGAGGGGGTTCACAGGTGCAGCTGGTCGAA
TCTGGGCCAGGCGTGGTCCAGCCAGGACGTTCCCTGAGGATTAGCTGCGCCGTGAGCGGGTTC
TCTGTCACAAACTACGGAGTGCACTGGGTCCGTCAGCCACCTGGCAAATGTCTGGAGTGGCTG
GGAGTGATCTGGGCAGGAGGAATCACTAACTACAACTCTGCTTTTATGAGTCGCCTGACCATC
TCAAAGGACAACTCCAAAAATACAGTGTACCTGCAGATGAATTCACTGCGGGCAGAAGATACC
GCCATGTACTATTGCGCCTCCAGGGGGGGTCATTACGGCTATGCCCTGGACTATTGGGGCCAG
GGAACACTGGTGACTGTCTCATCCGGAGGAGGAGGATCCGGAGGAGGAGGTAGCGGCGGAGGG
GGTTCTGGCGGAGGGGGTAGTCACGTGAAGCTGCAGGAAAGCGGCCCTGGACTGGTGCAGCCT
TCCCAGTCTCTGTCCCTGACCTGCACCGTGTCCGGCTTCTCCCTGACCGATTACGGCGTGCAC
TGGGTGCGACAGTCTCCAGGCAAGGGCCTGGAATGGCTGGGAGTGATTTGGAGCGGTGGCGGA
ACCGCCTACAACACCGCCCTGATCTCCCGGCTGAACATCTACCGGGACAACTCCAAGAACCAG
GTGTTCCTGGAAATGAACTCCCTGCAGGCAGAGGACACCGCCATGTACTACTGCGCCAGACGG
GGCTCCTACCCCTACAACTACTTCGACGCTTGGGGCTGCGGCACCACCGTGACAGTGTCTAGC
GGAGGTGGTGGATCTGGGGGCGGAGGTAGCGGAGGGGGAGGTTCTCAGGCTGTCGTGATCCAG
GAATCTGCCCTGACCACCCCCCCTGGCGAGACAGTGACACTGACCTGCGGATCTTCCACCGGC
GCTGTGACCGCCTCCAACTACGCCAACTGGGTGCAGGAAAAGCCCGACCACTGCTTCACCGGC
CTGATCGGCGGCCACAACAACAGACCTCCAGGCGTGCCAGCCCGGTTCTCCGGCTCTCTGATC
GGAGATAAGGCCGCCCTGACAATCGCCGGCACCCAGACAGAGGACGAGGCTATCTACTTCTGC
GCCCTGTGGTACAGCGACCACTGGGTCATCGGCGGAGGCACCAGACTGACCGTGCTGGGA(AC
ACCCCTGGGAGACACCACACATACT)AGTGGGAGATCCCCCGACGATGAGCTGCTGTACCTGC
CTGTGAGGGGCCGGGAGACCTATGAAATGCTGCTGAAGATCAAAGAGAGCCTGGAACTGATGC
AGTACCTGCCACAGCACACCATTGAAACATATAGGCAACAACAGCAGCAGCAGCATCAGCATC
TGCTGCAGAAGCAGGGAGGGTCAGGAGGAGCACCGCACCATCATCATCACCATT
P73-mBIDE(SL) polypeptide (hu3F8-scFv, C825-scFv, huP73-tet,
GS linker, (IgG3 spacer))
SEQ ID NO: 49
EIVMTQTPATLSVSAGERVTITCKASQSVSNDVTWYQQKPGQAPRLLIYSASNRYSGVPARFS
GSGYGTEFTFTISSVQSEDFAVYFCQQDYSSFGCGTKLEIKRGGGGSGGGGSGGGGSQVQLVE
SGPGVVQPGRSLRISCAVSGFSVTNYGVHWVRQPPGKCLEWLGVIWAGGITNYNSAFMSRLTI
SKDNSKNTVYLQMNSLRAEDTAMYYCASRGGHYGYALDYWGQGTLVTVSSGGGGSGGGGSGGG
GSGGGGSHVKLQESGPGLVOPSQSLSLTCTVSGFSLTDYGVHWVRQSPGKGLEWLGVIWSGGG
TAYNTALISRLNIYRDNSKNQVFLEMNSLQAEDTAMYYCARRGSYPYNYFDAWGCGTTVTVSS
GGGGSGGGGSGGGGSQAVVIQESALTTPPGETVTLTCGSSTGAVTASNYANWVQEKPDHCFTG
LIGGHNNRPPGVPARFSGSLIGDKAALTIAGTQTEDEAIYFCALWYSDHWVIGGGTRLTVLG
(TPLGDTTHT)SGRHGDEDTYYLQVRGRENFEILMKLKESLELMELVPQPLVDSYRQQQQLLQ
RPGGSGGAPHHHHHH
P73-mBIDE(SL) cDNA (hu3F8-scFv, C825-scFv, huP73-tet,
GS linker, (IgG3 spacer))
SEQ ID NO: 50
GAAATCGTCATGACTCAGACTCCCGCAACCCTGTCAGTGTCCGCTGGGGAACGTGTCACTATT
ACCTGCAAGGCATCTCAGAGCGTGAGCAACGACGTGACCTGGTATCAGCAGAAGCCTGGCCAG
GCTCCACGACTGCTGATCTATTCCGCAAGCAATCGCTACTCCGGAGTGCCCGCACGATTCTCT
GGAAGTGGGTACGGTACCGAGTTCACTTTTACCATTTCCAGCGTGCAGAGCGAAGACTTCGCT
GTCTATTTTTGCCAGCAGGATTACTCTAGTTTTGGCTGTGGAACAAAGCTGGAGATCAAAAGG
GGAGGAGGAGGTTCTGGCGGAGGAGGTAGTGGCGGAGGGGGTTCACAGGTGCAGCTGGTCGAA
TCTGGGCCAGGCGTGGTCCAGCCAGGACGTTCCCTGAGGATTAGCTGCGCCGTGAGCGGGTTC
TCTGTCACAAACTACGGAGTGCACTGGGTCCGTCAGCCACCTGGCAAATGTCTGGAGTGGCTG
GGAGTGATCTGGGCAGGAGGAATCACTAACTACAACTCTGCTTTTATGAGTCGCCTGACCATC
TCAAAGGACAACTCCAAAAATACAGTGTACCTGCAGATGAATTCACTGCGGGCAGAAGATACC
GCCATGTACTATTGCGCCTCCAGGGGGGGTCATTACGGCTATGCCCTGGACTATTGGGGCCAG
GGAACACTGGTGACTGTCTCATCCGGAGGAGGAGGATCCGGAGGAGGAGGTAGCGGCGGAGGG
GGTTCTGGCGGAGGGGGTAGTCACGTGAAGCTGCAGGAAAGCGGCCCTGGACTGGTGCAGCCT
TCCCAGTCTCTGTCCCTGACCTGCACCGTGTCCGGCTTCTCCCTGACCGATTACGGCGTGCAC
TGGGTGCGACAGTCTCCAGGCAAGGGCCTGGAATGGCTGGGAGTGATTTGGAGCGGTGGCGGA
ACCGCCTACAACACCGCCCTGATCTCCCGGCTGAACATCTACCGGGACAACTCCAAGAACCAG
GTGTTCCTGGAAATGAACTCCCTGCAGGCAGAGGACACCGCCATGTACTACTGCGCCAGACGG
GGCTCCTACCCCTACAACTACTTCGACGCTTGGGGCTGCGGCACCACCGTGACAGTGTCTAGC
GGAGGTGGTGGATCTGGGGGCGGAGGTAGCGGAGGGGGAGGTTCTCAGGCTGTCGTGATCCAG
GAATCTGCCCTGACCACCCCCCCTGGCGAGACAGTGACACTGACCTGCGGATCTTCCACCGGC
GCTGTGACCGCCTCCAACTACGCCAACTGGGTGCAGGAAAAGCCCGACCACTGCTTCACCGGC
CTGATCGGCGGCCACAACAACAGACCTCCAGGCGTGCCAGCCCGGTTCTCCGGCTCTCTGATC
GGAGATAAGGCCGCCCTGACAATCGCCGGCACCCAGACAGAGGACGAGGCTATCTACTTCTGC
GCCCTGTGGTACAGCGACCACTGGGTCATCGGCGGAGGCACCAGACTGACCGTGCTGGGA(AC
ACCCCTGGGAGACACCACACATACT)AGTGGGAGGCACGGCGACGAAGATACCTACTATCTGC
AGGTGAGGGGACGGGAGAACTTCGAAATCCTGATGAAGCTGAAAGAGTCCCTGGAACTGATGG
AGCTGGTGCCCCAGCCTCTGGTCGACAGCTACAGACAGCAGCAGCAGCTGCTGCAGAGGCCAG
GAGGGTCAGGAGGAGCACCGCACCATCATCATCACCAT
P53-mBIDE(LL) polypeptide (hu3F8-scFv, C825-scFv, huP53-tet,
GS linker, (IgG3 spacer))
SEQ ID NO: 51
EIVMTQTPATLSVSAGERVTITCKASQSVSNDVTWYQQKPGQAPRLLIYSASNRYSGVPARFS
GSGYGTEFTFTISSVQSEDFAVYFCQQDYSSFGCGTKLEIKRGGGGSGGGGSGGGGSGGGGSG
GGGSGGGGSQVQLVESGPGVVQPGRSLRISCAVSGFSVTNYGVHWVRQPPGKCLEWLGVIWAG
GITNYNSAFMSRLTISKDNSKNTVYLQMNSLRAEDTAMYYCASRGGHYGYALDYWGQGTLVTV
SSGGGGSGGGGSGGGGSGGGGSHVKLQESGPGLVQPSQSLSLTCTVSGFSLTDYGVHWVRQSP
GKGLEWLGVIWSGGGTAYNTALISRLNIYRDNSKNQVFLEMNSLQAEDTAMYYCARRGSYPYN
YFDAWGCGTTVTVSSGGGGSGGGGSGGGGSGGGGSGGGGSGGGGSQAVVIQESALTTPPGETV
TLTCGSSTGAVTASNYANWVQEKPDHCFTGLIGGHNNRPPGVPARFSGSLIGDKAALTIAGTQ
TEDEAIYFCALWYSDHWVIGGGTRLTVLG(TPLGDTTHT)SGKPLDGEYFTLQIRGRERFEMF
RELNEALELKDAQAGKEPGGSGGAPHHHHHH
P53-mBIDE(LL) cDNA (hu3F8-scFv, C825-scFv, huP53-tet,
GS linker, (IgG3 spacer))
SEQ ID NO: 52
GAGATCGTGATGACCCAGACACCCGCAACACTGAGCGTGTCTGCCGGCGAAAGGGTCACTATT
ACCTGCAAGGCCAGTCAGTCAGTGTCCAACGACGTGACTTGGTACCAGCAGAAACCAGGCCAG
GCTCCCCGGCTGCTGATCTACAGCGCATCTAATAGATATAGCGGAGTGCCTGCTCGCTTCAGT
GGTTCAGGCTATGGAACTGAGTTCACCTTCACCATTTCCAGCGTGCAGTCCGAAGACTTCGCA
GTGTACTTTTGCCAGCAGGATTATTCTAGTTTTGGGTGTGGTACAAAGCTGGAGATCAAAAGG
GGAGGAGGAGGTAGTGGCGGAGGAGGTTCAGGCGGAGGGGGTAGCGGCGGAGGGGGTTCTGGC
GGCGGCGGTAGTGGCGGCGGAGGTAGCCAGGTGCAGCTGGTCGAATCCGGCCCTGGAGTGGTC
CAGCCAGGCAGGTCTCTGCGGATCAGTTGCGCCGTGTCCGGATTCAGCGTCACCAACTACGGA
GTGCACTGGGTCAGACAGCCACCTGGCAAGTGTCTGGAGTGGCTGGGAGTGATCTGGGCAGGA
GGAATCACAAACTACAACTCAGCTTTTATGTCCCGCCTGACTATTAGCAAGGACAACTCTAAA
AATACCGTGTATCTGCAGATGAATTCTCTGCGAGCCGAAGATACCGCTATGTACTATTGTGCA
TCCCGTGGGGGTCATTACGGCTATGCCCTGGATTATTGGGGGCAGGGTACCCTGGTGACAGTC
TCATCCGGCGGAGGGGGATCCGGCGGCGGAGGATCTGGCGGAGGTGGAAGTGGGGGAGGCGGA
TCTCACGTGAAGCTGCAGGAAAGCGGCCCTGGACTGGTGCAGCCTTCCCAGTCTCTGTCCCTG
ACCTGCACCGTGTCCGGCTTCTCCCTGACCGATTACGGCGTGCACTGGGTGCGACAGTCTCCA
GGCAAGGGCCTGGAATGGCTGGGAGTGATTTGGAGCGGTGGCGGAACCGCCTACAACACCGCC
CTGATCTCCCGGCTGAACATCTACCGGGACAACTCCAAGAACCAGGTGTTCCTGGAAATGAAC
TCCCTGCAGGCAGAGGACACCGCCATGTACTACTGCGCCAGACGGGGCTCCTACCCCTACAAC
TACTTCGACGCTTGGGGCTGCGGCACCACCGTGACAGTGTCTAGCGGAGGTGGTGGATCTGGG
GGCGGAGGTAGCGGAGGGGGAGGTTCTGGAGGTGGTGGATCTGGGGGCGGAGGTAGCGGAGGG
GGAGGTTCTCAGGCTGTCGTGATCCAGGAATCTGCCCTGACCACCCCCCCTGGCGAGACAGTG
ACACTGACCTGCGGATCTTCCACCGGCGCTGTGACCGCCTCCAACTACGCCAACTGGGTGCAG
GAAAAGCCCGACCACTGCTTCACCGGCCTGATCGGCGGCCACAACAACAGACCTCCAGGCGTG
CCAGCCCGGTTCTCCGGCTCTCTGATCGGAGATAAGGCCGCCCTGACAATCGCCGGCACCCAG
ACAGAGGACGAGGCTATCTACTTCTGCGCCCTGTGGTACAGCGACCACTGGGTCATCGGCGGA
GGCACCAGACTGACCGTGCTGGGA(ACACCCCTGGGAGACACCACACATACT)AGTGGGAAAC
CTCTGGATGGCGAGTACTTTACCCTGCAGATTAGAGGCCGCGAACGATTCGAGATGTTTCGCG
AACTGAATGAGGCCCTGGAACTGAAGGATGCTCAGGCAGGCAAGGAGCCAGGAGGGTCAGGAG
GAGCACCGCACCATCATCATCACCAT
P63-mBIDE(LL) polypeptide (hu3F8-scFv, C825-scFv, huP63-tet,
GS linker, (IgG3 spacer))
SEQ ID NO: 53
EIVMTQTPATLSVSAGERVTITCKASQSVSNDVTWYQQKPGQAPRLLIYSASNRYSGVPARFS
GSGYGTEFTFTISSVQSEDFAVYFCQQDYSSFGCGTKLEIKRGGGGSGGGGSGGGGSGGGGSG
GGGSGGGGSQVQLVESGPGVVQPGRSLRISCAVSGFSVTNYGVHWVRQPPGKCLEWLGVIWAG
GITNYNSAFMSRLTISKDNSKNTVYLQMNSLRAEDTAMYYCASRGGHYGYALDYWGQGTLVTV
SSGGGGSGGGGSGGGGSGGGGSHVKLQESGPGLVQPSQSLSLTCTVSGFSLTDYGVHWVRQSP
GKGLEWLGVIWSGGGTAYNTALISRLNIYRDNSKNQVFLEMNSLQAEDTAMYYCARRGSYPYN
YFDAWGCGTTVTVSSGGGGSGGGGSGGGGSGGGGSGGGGSGGGGSQAVVIQESALTTPPGETV
TLTCGSSTGAVTASNYANWVQEKPDHCFTGLIGGHNNRPPGVPARFSGSLIGDKAALTIAGTQ
TEDEAIYFCALWYSDHWVIGGGTRLTVLG(TPLGDTTHT)SGRSPDDELLYLPVRGRETYEML
LKIKESLELMQYLPQHTIETYRQQQQQQHQHLLQKQGGSGGAPHHHHHH
P63-mBIDE(LL) cDNA (hu3F8-scFv, C825-scFv, huP63-tet,
GS linker, (IgG3 spacer))
SEQ ID NO: 54
GAGATCGTGATGACCCAGACACCCGCAACACTGAGCGTGTCTGCCGGCGAAAGGGTCACTATT
ACCTGCAAGGCCAGTCAGTCAGTGTCCAACGACGTGACTTGGTACCAGCAGAAACCAGGCCAG
GCTCCCCGGCTGCTGATCTACAGCGCATCTAATAGATATAGCGGAGTGCCTGCTCGCTTCAGT
GGTTCAGGCTATGGAACTGAGTTCACCTTCACCATTTCCAGCGTGCAGTCCGAAGACTTCGCA
GTGTACTTTTGCCAGCAGGATTATTCTAGTTTTGGGTGTGGTACAAAGCTGGAGATCAAAAGG
GGAGGAGGAGGTAGTGGCGGAGGAGGTTCAGGCGGAGGGGGTAGCGGCGGAGGGGGTTCTGGC
GGCGGCGGTAGTGGCGGCGGAGGTAGCCAGGTGCAGCTGGTCGAATCCGGCCCTGGAGTGGTC
CAGCCAGGCAGGTCTCTGCGGATCAGTTGCGCCGTGTCCGGATTCAGCGTCACCAACTACGGA
GTGCACTGGGTCAGACAGCCACCTGGCAAGTGTCTGGAGTGGCTGGGAGTGATCTGGGCAGGA
GGAATCACAAACTACAACTCAGCTTTTATGTCCCGCCTGACTATTAGCAAGGACAACTCTAAA
AATACCGTGTATCTGCAGATGAATTCTCTGCGAGCCGAAGATACCGCTATGTACTATTGTGCA
TCCCGTGGGGGTCATTACGGCTATGCCCTGGATTATTGGGGGCAGGGTACCCTGGTGACAGTC
TCATCCGGAGGAGGAGGATCCGGAGGAGGAGGTAGCGGCGGAGGGGGTTCTGGCGGAGGGGGT
AGTCACGTGAAGCTGCAGGAAAGCGGCCCTGGACTGGTGCAGCCTTCCCAGTCTCTGTCCCTG
ACCTGCACCGTGTCCGGCTTCTCCCTGACCGATTACGGCGTGCACTGGGTGCGACAGTCTCCA
GGCAAGGGCCTGGAATGGCTGGGAGTGATTTGGAGCGGTGGCGGAACCGCCTACAACACCGCC
CTGATCTCCCGGCTGAACATCTACCGGGACAACTCCAAGAACCAGGTGTTCCTGGAAATGAAC
TCCCTGCAGGCAGAGGACACCGCCATGTACTACTGCGCCAGACGGGGCTCCTACCCCTACAAC
TACTTCGACGCTTGGGGCTGCGGCACCACCGTGACAGTGTCTAGCGGAGGTGGTGGATCTGGG
GGCGGAGGTAGCGGAGGGGGAGGTTCTGGAGGTGGTGGATCTGGGGGCGGAGGTAGCGGAGGG
GGAGGTTCTCAGGCTGTCGTGATCCAGGAATCTGCCCTGACCACCCCCCCTGGCGAGACAGTG
ACACTGACCTGCGGATCTTCCACCGGCGCTGTGACCGCCTCCAACTACGCCAACTGGGTGCAG
GAAAAGCCCGACCACTGCTTCACCGGCCTGATCGGCGGCCACAACAACAGACCTCCAGGCGTG
CCAGCCCGGTTCTCCGGCTCTCTGATCGGAGATAAGGCCGCCCTGACAATCGCCGGCACCCAG
ACAGAGGACGAGGCTATCTACTTCTGCGCCCTGTGGTACAGCGACCACTGGGTCATCGGCGGA
GGCACCAGACTGACCGTGCTGGGA(ACACCCCTGGGAGACACCACACATACT)AGTGGGAGAT
CCCCCGACGATGAGCTGCTGTACCTGCCTGTGAGGGGCCGGGAGACCTATGAAATGCTGCTGA
AGATCAAAGAGAGCCTGGAACTGATGCAGTACCTGCCACAGCACACCATTGAAACATATAGGC
AACAACAGCAGCAGCAGCATCAGCATCTGCTGCAGAAGCAGGGAGGGTCAGGAGGAGCACCGC
ACCATCATCATCACCAT
P73-mBIDE(LL) polypeptide (hu3F8-scFv, C825-scFv, huP73-tet,
GS linker, (IgG3 spacer))
SEQ ID NO: 55
EIVMTQTPATLSVSAGERVTITCKASQSVSNDVTWYQQKPGQAPRLLIYSASNRYSGVPARFS
GSGYGTEFTFTISSVQSEDFAVYFCQQDYSSFGCGTKLEIKRGGGGSGGGGSGGGGSGGGGSG
GGGSGGGGSQVQLVESGPGVVQPGRSLRISCAVSGFSVTNYGVHWVRQPPGKCLEWLGVIWAG
GITNYNSAFMSRLTISKDNSKNTVYLQMNSLRAEDTAMYYCASRGGHYGYALDYWGOGTLVTV
SSGGGGSGGGGSGGGGSGGGGSHVKLQESGPGLVQPSQSLSLTCTVSGFSLTDYGVHWVRQSP
GKGLEWLGVIWSGGGTAYNTALISRLNIYRDNSKNQVFLEMNSLQAEDTAMYYCARRGSYPYN
YFDAWGCGTTVTVSSGGGGSGGGGSGGGGSGGGGSGGGGSGGGGSQAVVIQESALTTPPGETV
TLTCGSSTGAVTASNYANWVQEKPDHCFTGLIGGHNNRPPGVPARFSGSLIGDKAALTIAGTQ
TEDEAIYFCALWYSDHWVIGGGTRLTVLG(TPLGDTTHT)SGRHGDEDTYYLQVRGRENFEIL
MKLKESLELMELVPQPLVDSYRQQQQLLQRPGGSGGAPHHHHHH
P73-mBIDE(LL) cDNA (hu3F8-scFv, C825-scFv, huP73-tet,
GS linker, (IgG3 spacer))
SEQ ID NO: 56
GAGATCGTGATGACCCAGACACCCGCAACACTGAGCGTGTCTGCCGGCGAAAGGGTCACTATT
ACCTGCAAGGCCAGTCAGTCAGTGTCCAACGACGTGACTTGGTACCAGCAGAAACCAGGCCAG
GCTCCCCGGCTGCTGATCTACAGCGCATCTAATAGATATAGCGGAGTGCCTGCTCGCTTCAGT
GGTTCAGGCTATGGAACTGAGTTCACCTTCACCATTTCCAGCGTGCAGTCCGAAGACTTCGCA
GTGTACTTTTGCCAGCAGGATTATTCTAGTTTTGGGTGTGGTACAAAGCTGGAGATCAAAAGG
GGAGGAGGAGGTAGTGGCGGAGGAGGTTCAGGCGGAGGGGGTAGCGGCGGAGGGGGTTCTGGC
GGCGGCGGTAGTGGCGGCGGAGGTAGCCAGGTGCAGCTGGTCGAATCCGGCCCTGGAGTGGTC
CAGCCAGGCAGGTCTCTGCGGATCAGTTGCGCCGTGTCCGGATTCAGCGTCACCAACTACGGA
GTGCACTGGGTCAGACAGCCACCTGGCAAGTGTCTGGAGTGGCTGGGAGTGATCTGGGCAGGA
GGAATCACAAACTACAACTCAGCTTTTATGTCCCGCCTGACTATTAGCAAGGACAACTCTAAA
AATACCGTGTATCTGCAGATGAATTCTCTGCGAGCCGAAGATACCGCTATGTACTATTGTGCA
TCCCGTGGGGGTCATTACGGCTATGCCCTGGATTATTGGGGGCAGGGTACCCTGGTGACAGTC
TCATCCGGAGGAGGAGGATCCGGAGGAGGAGGTAGCGGCGGAGGGGGTTCTGGCGGAGGGGGT
AGTCACGTGAAGCTGCAGGAAAGCGGCCCTGGACTGGTGCAGCCTTCCCAGTCTCTGTCCCTG
ACCTGCACCGTGTCCGGCTTCTCCCTGACCGATTACGGCGTGCACTGGGTGCGACAGTCTCCA
GGCAAGGGCCTGGAATGGCTGGGAGTGATTTGGAGCGGTGGCGGAACCGCCTACAACACCGCC
CTGATCTCCCGGCTGAACATCTACCGGGACAACTCCAAGAACCAGGTGTTCCTGGAAATGAAC
TCCCTGCAGGCAGAGGACACCGCCATGTACTACTGCGCCAGACGGGGCTCCTACCCCTACAAC
TACTTCGACGCTTGGGGCTGCGGCACCACCGTGACAGTGTCTAGCGGAGGTGGTGGATCTGGG
GGCGGAGGTAGCGGAGGGGGAGGTTCTGGAGGTGGTGGATCTGGGGGCGGAGGTAGCGGAGGG
GGAGGTTCTCAGGCTGTCGTGATCCAGGAATCTGCCCTGACCACCCCCCCTGGCGAGACAGTG
ACACTGACCTGCGGATCTTCCACCGGCGCTGTGACCGCCTCCAACTACGCCAACTGGGTGCAG
GAAAAGCCCGACCACTGCTTCACCGGCCTGATCGGCGGCCACAACAACAGACCTCCAGGCGTG
CCAGCCCGGTTCTCCGGCTCTCTGATCGGAGATAAGGCCGCCCTGACAATCGCCGGCACCCAG
ACAGAGGACGAGGCTATCTACTTCTGCGCCCTGTGGTACAGCGACCACTGGGTCATCGGCGGA
GGCACCAGACTGACCGTGCTGGGA(ACACCCCTGGGAGACACCACACATACT)AGTGGGAGGC
ACGGCGACGAAGATACCTACTATCTGCAGGTGAGGGGACGGGAGAACTTCGAAATCCTGATGA
AGCTGAAAGAGTCCCTGGAACTGATGGAGCTGGTGCCCCAGCCTCTGGTCGACAGCTACAGAC
AGCAGCAGCAGCTGCTGCAGAGGCCAGGAGGGTCAGGAGGAGCACCGCACCATCATCATCACC
AT

All constructs (including SADA-BiDEs) were cloned into standard IgG expression vectors using common molecular cloning techniques. Genes were either synthesized, PCR amplified or digested from other sources and ligated together using PCR or standard DNA ligases.

All constructs (including SADA-BiDEs) were expressed in either CHO-S, expiCHO or expi293 (HEK) suspension cell lines. Expression was either from a stable line (P53-BIDE(NOHIS)) or after transient expression (all others). P53-BIDE(NOHIS) was purified using one-step affinity purification using Protein-L resin (captoL). Briefly, supernatant from the host cells was harvested, filtered and run along the affinity column. The column was washed and bound protein was eluted by low pH elution. pH was neutralized and the buffer was dialyzed to a final storage buffer overnight. All other constructs followed the same basic protocol except used a nickel-NTA resin instead of protein, and elution was via high concentration imidazole instead of low pH.

It is envisioned that such an exemplary constructs (e.g., P53-BIDE(NOHIS), P53-BiDE, P63-BiDE, P73-BiDE) may be useful for pretargeted radioimmunotherapy (PRIT). Schematic diagrams for various 3-step, 2-step and 1-step PRIT methods are depicted in FIG. 1A-C, respectively.

### Example 2—Stability of an Exemplary Conjugate with a SADA Domain In Vitro

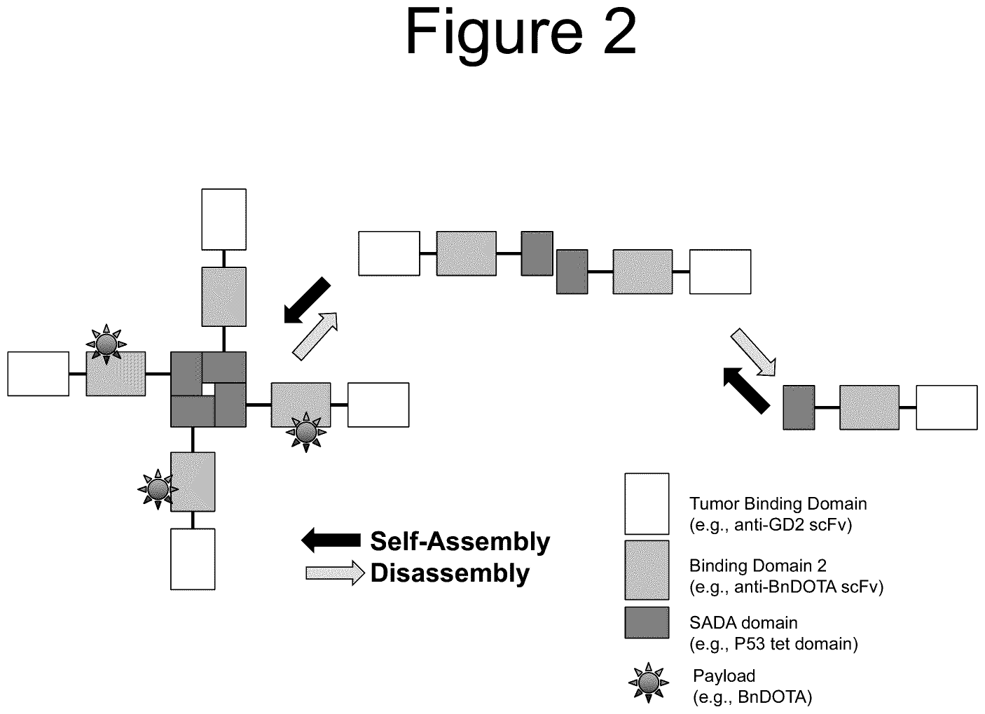

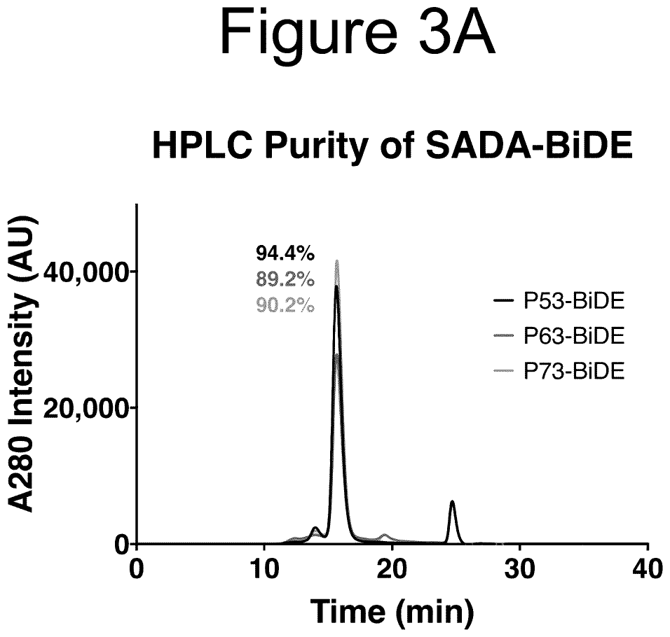

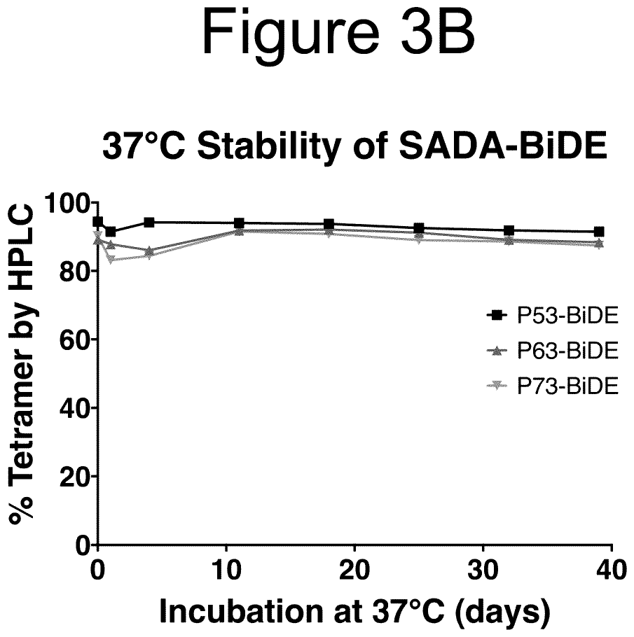

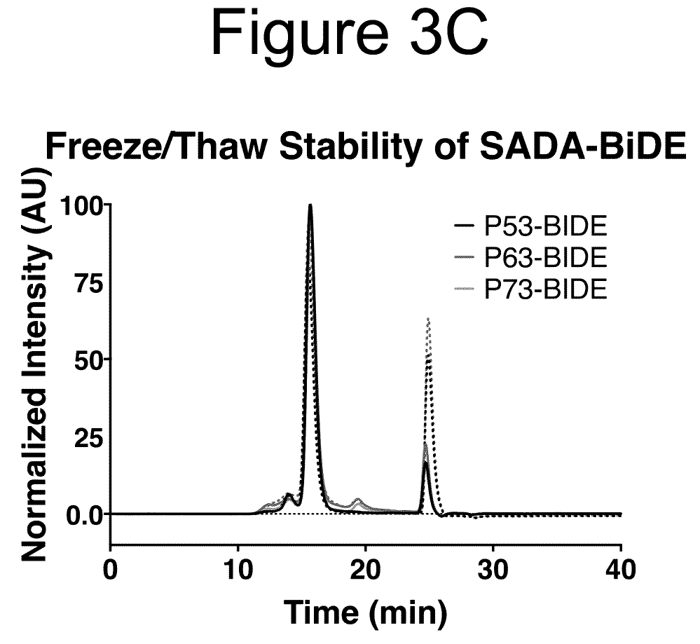

This Example demonstrates that an exemplary bispecific antibody-based conjugate with a SADA domain is highly stable in vitro. In particular, this Example describes biochemical purity analysis of a preparation of SADA conjugate as described in FIGS. 3A to 3C, P53-BIDE, P63-BiDE and P73-BiDE. Each SADA-BiDE self-assembles into a stable homo-tetramer through its SADA domain (i.e., p53, p63 or p73 tetramerization domains). Therefore, each can exist as an individual monomer (quarter), a dimer of monomers (half: dimer) or a dimer of dimers (full: tetramer). See schematic illustration of an exemplary SADA-BiDE conjugate in FIG. 2.

As shown in FIG. 3, P53-BIDE, P63-BiDE, and P73-BiDE show extremely high in vitro stability, comparable to that of an IgG. After single-step affinity purification, HPLC analysis of a preparation of all three SADA-BiDEs showed a major peak at ^{˜}16 min (^{˜}90%) with a calculated molecular weight of ^{˜}200 kDa (FIG. 3A). The expected and calculated size by HPLC standards, is ^{˜}200 kDa, similar to an IgG-scFv (Cheal, S. M. et al. (2014) Mol Cancer Ther 13, 1803-1812; Xu, H. et al. (2015) Cancer immunology research 3, 266-277). A small earlier peak (^{˜}14 min) denotes smaller aggregates of each SADA-BiDE (2-3 complexes) and a later peak (^{˜}25 min) is a non-specific peak from the storage buffer (sodium citrate). Therefore, P53-BiDE, P63-BiDE, and P73-BiDE exists in vitro predominantly as a tetramer.

Moreover, all SADA-BiDEs were found to be highly stable, as shown in FIG. 3B. Preparations of P53-BIDE, P63-BiDE, and P73-BiDE remained stable for over four weeks at 37° C., with purity of the tetramer remaining unchanged over time. Additionally, all SADA-BiDEs remained tetrameric and did not show any loss in concentration or formations of aggregates/monomers after multiple freeze/thaw cycles (5 cycles; −80° C. to 25° C.) (FIG. 3C). Thus HPLC analysis provided herein documents the high in vitro stability of an exemplary tetrameric bispecific antibody-based conjugate with a SADA domain, which suggests a strong potential for manufacturability of these multimeric conjugates.

Analysis of the in vitro and in vivo functional activities of P53-BIDE, P63-BiDE, P73-BiDE and P53-BiDE(noHIS) is provided in the examples that follow. These examples demonstrate the potential of bispecific antibody-based conjugates with a SADA domain as effective agents for PRIT.

### Example 3—Dissociation Kinetics of Exemplary SADA Conjugates In Vitro

This Example describes the dissociation kinetics of exemplary bispecific antibody-based conjugates with a SADA domain. In particular, this Example measures the rates of dissociation of exemplary p53, p63, and p73 SADA-BiDES. P53-BiDE, P63-BiDE and P73-BiDE, respectively, using fluorescence correlation spectroscopy (FCS). The samples were labeled with Cy3-labeled ¹⁷⁵Lu-Bn-DOTA and prepared at a concentration of 500 nM, then rapidly diluted to 0.5 nM and then fluctuations in fluorescent intensity were measure over the course of 2 hours. Measurements were taken with a Zeiss LSM 880 confocal microscope. Normalized autocorrelations functions G(T) were then plotted to determine the diffusion times for each SADA-BiDE over time. All samples were compared against a monomeric GD2-BiDE

To determine the dissociation rate k_{off}, the diffusion times were plotted as a function of time. A one-phase exponential decay curve fit model was utilized to determine k_{off} and half-life (R² of 0.69-0.72). The results indicated that the P63-BiDE had the slowest dissociation rate.

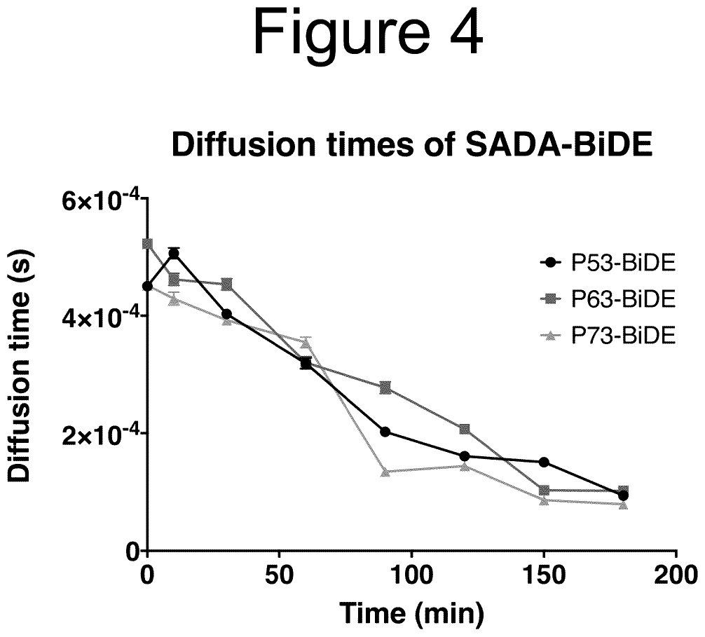

TABLE 3
Dissociation kinetics of SADA-BiDEs (See also, e.g., FIG. 4)

| | P53-BiDE | P63-BiDE | P73-BiDE |
|---|---|---|---|
| k_{off} (sec⁻¹) | 11.2 ± 1.4 × 10⁻⁵ | 6.3 ± 1.4 × 10⁻⁵ | 9.5 ± 1.3 × 10⁻⁵ |
| half-life | 104 | 185 | 122 |
| (min) | | | |

### Example 4—Target Binding Affinity Exemplary Bispecific Antibody-Based SADA Conjugates with a SADA Domain

This example documents the binding characteristics of an exemplary bispecific antibody-based conjugate with a SADA domain. In particular, this Example demonstrates that exemplary SADA-BiDE bispecific antibody-based conjugates with a SADA domain (P53-BIDE, P63-BiDE, P73-BiDE) effectively bind in vitro to their targets.

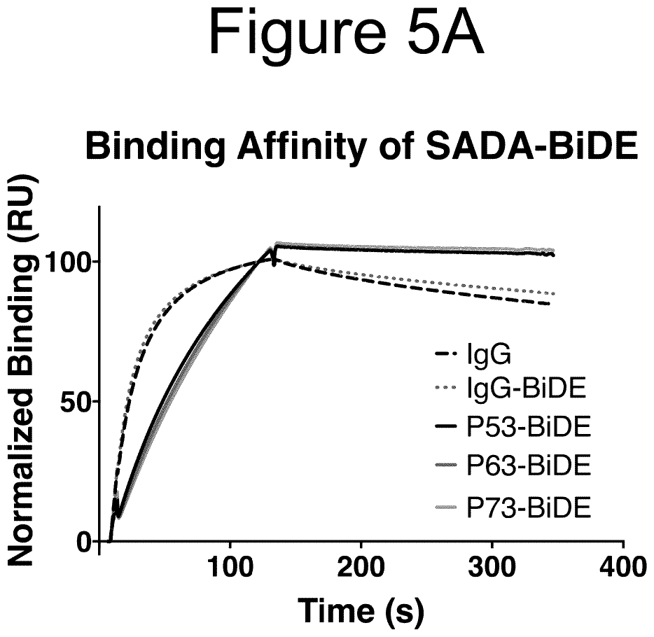

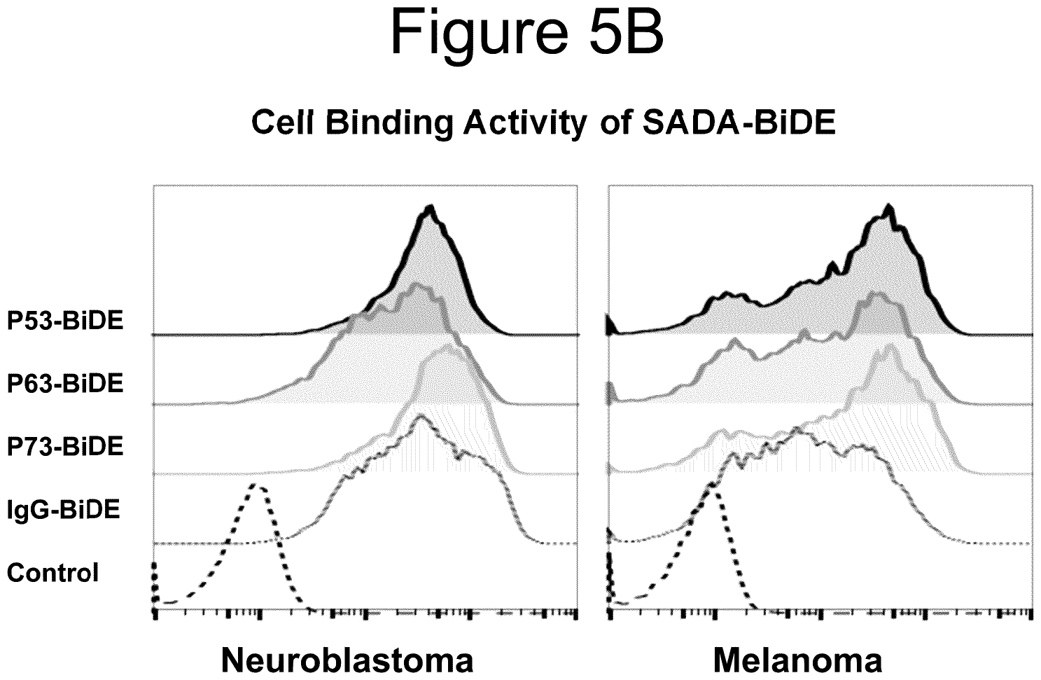

As shown in FIG. 5A, all three SADA-BiDEs exhibited improved binding to their tumor target (GD2), as measured by SPR, over both a standard IgG (hu3F8-IgG) (Cheung, N. K., et al. (2012) Oncolmmunology 1, 477-486) and an IgG-scFv (hu3F8-IgG-scFv) (Cheal, S. M. et al. (2014) Mol Cancer Ther 13, 1803-1812). Table 4 shows SPR calculated affinity data, and fold increase over IgG and IgG-BiDE constructs. Data was fitted using a two-state reaction model. Strikingly, the off rate kinetics (k_{off}) (FIG. 5A), which are thought to be critically important in determining the effectiveness of most receptor based therapeutics, had an improvement of 1e3-6e4 fold over hu3F8-IgG or IgG-BiDE, as well as a 3-10 fold improvement in K_{D} (Table 4). Without being bound to theory, it is envisioned that, in at least some embodiments, multimerization through a SADA domain may stabilize and/or otherwise provide useful attributes to an antibody agent.

TABLE 4
SPR affinity data of SADA-BiDEs (See also, e.g., FIG. 5A)

| | ka1 | kd1 | ka2 | kd2 | K_{D} | kd1 fold | KD fold |
|---|---|---|---|---|---|---|---|
| | (1/Ms) | (1/s) | (1/s) | (1/s) | (M) | over IgG | over IgG |
| IgG | 1.1E+06 | 1.2E+00 | 1.5E−01 | 7.0E−04 | 5.0E−09 | 1 | 1 |
| IgG- | 2.8E+06 | 3.0E+00 | 1.6E−01 | 6.1E−04 | 4.0E−09 | 0.4 | 1 |
| BiDE | | | | | | | |
| P53- | 3.7E+04 | 3.4E−04 | 7.5E−03 | 3.9E−04 | 4.6E−10 | 3691 | 11 |
| BiDE | | | | | | | |
| P63- | 3.1E+04 | 6.2E−05 | 4.9E−04 | 2.1E−03 | 1.6E−09 | 20129 | 3 |
| BiDE | | | | | | | |
| P73- | 2.6E+04 | 2.0E−05 | 5.0E−03 | 1.3E−03 | 1.5E−10 | 62807 | 32 |
| BiDE | | | | | | | |

Further, preparations of various SADA-BiDEs (P53-BIDE, P63-BIDE, P73-BIDE) exhibited robust binding to two different GD2(+) tumor lines, IMR32-Luc (Neuroblastoma) and M14-Luc (Melanoma). FIG. 5B depicts a FACS analysis using a fluorescently labeled ¹⁵Lu-Bn-DOTA conjugate, thus demonstrating that each SADA-BiDE can bind both to the GD2 on the cell surface in the context of two different tumor cell lines and also simultaneously bind a second antigen (Bn-DOTA), which is critical for PRIT.

### Example 5—Clearance of a Bispecific Antibody-Based Conjugate with a SADA Domain in Vivo

This Example demonstrates in vivo clearance of an exemplary bispecific antibody-based conjugate with a SADA domain. In particular, this Example demonstrates that an exemplary tetrameric bispecific antibody-based conjugate with a SADA domain (P53-BIDE(NOHIS)) is rapidly cleared, even without the use of a clearing agent (CA). Thus, in vivo, using nude mice, use of a SADA technology eliminates the need for a CA.

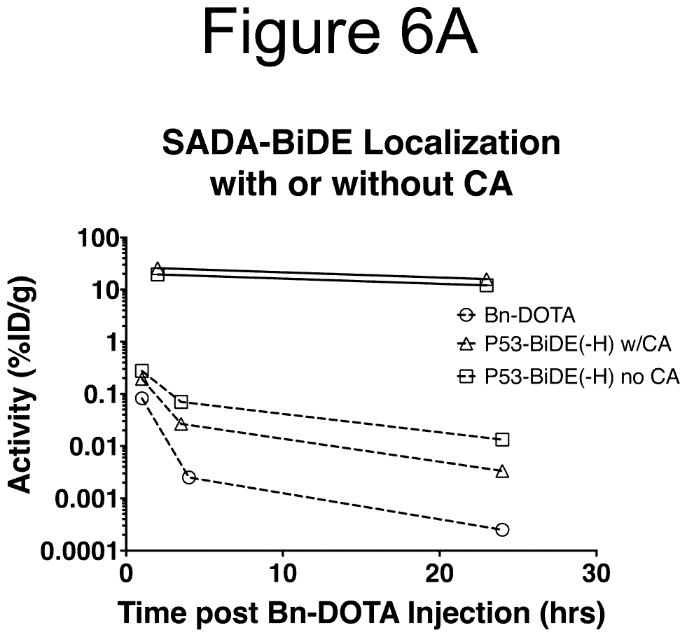

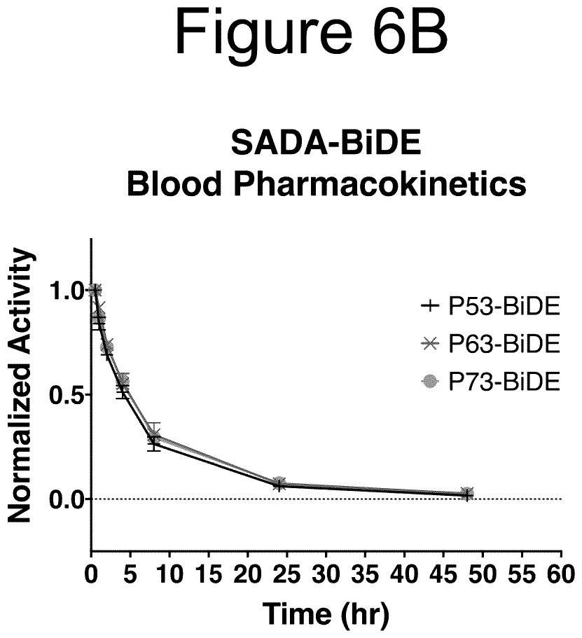

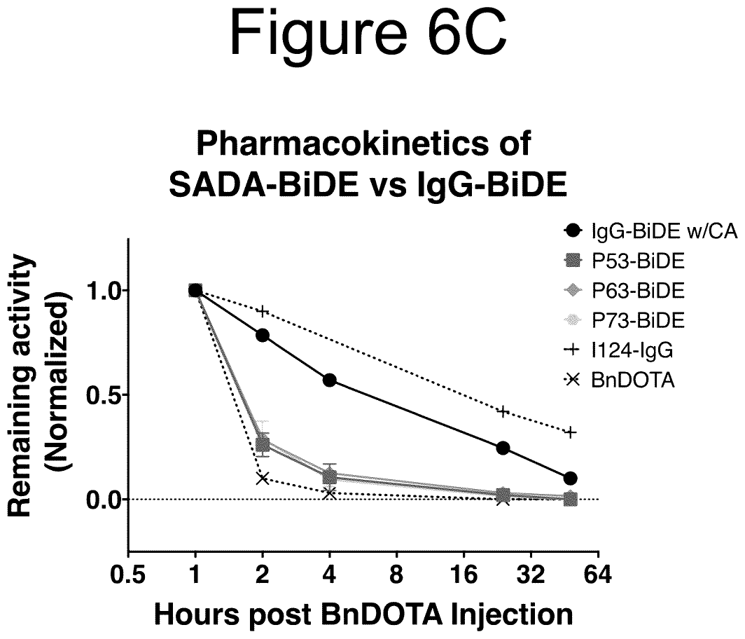

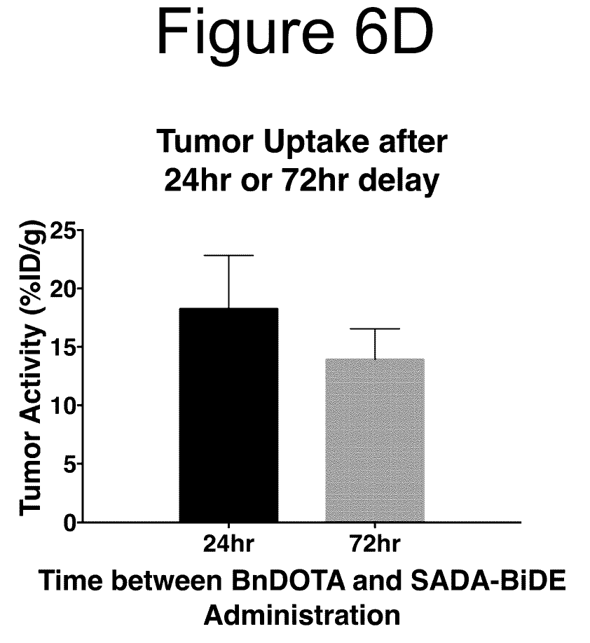

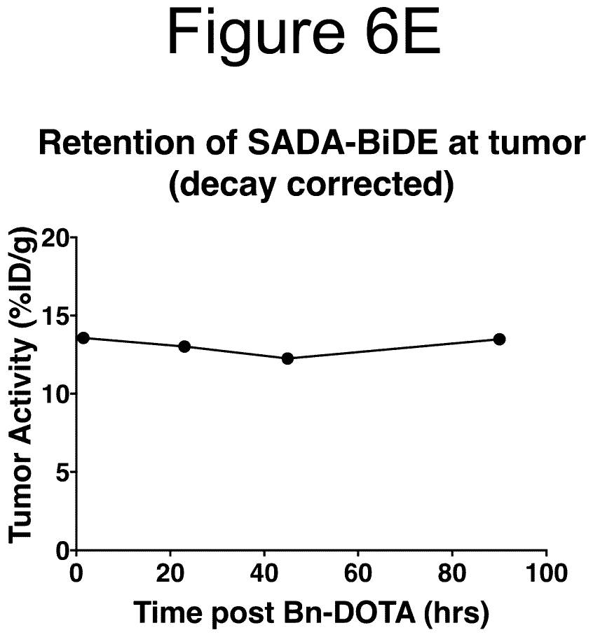

In PRIT, an IgG-BiDE-based therapeutic has significant serum levels during the first 72 hours, necessitating the use of CA (Cheal, S. M. et al. (2014) Mol Cancer Ther 13, 1803-1812). In contrast, as illustrated in FIG. 6A, an exemplary bispecific antibody-based conjugates with a SADA domain (P53-BIDE(NOHIS)) is almost completely cleared on its own between 24 and 72 hours after injection without any CA. Administration of a CA had minimal effect on the clearance of an exemplary bispecific antibody-based conjugates with a SADA domain (P53-BIDE(NOHIS)), with detectable blood levels nearly identical to Bn-DOTA single treatment, suggesting almost all SADA-BiDE has cleared from the body before payload administration. As illustrated in FIG. 6A, clearance of P53-BIDE(NOHIS), even when CA was provided within this same window, had only a minor effect, decreasing residual blood activity by a negligible amount. Importantly, addition of a CA did not alter tumor uptake significantly. This Example confirms, among other things, that an exemplary bispecific antibody-based conjugates with a SADA domain (P53-BIDE(NOHIS)) is rapidly cleared from the blood without the use of a CA. Further, these data support that P53-BIDE(NOHIS) is capable of achieving high therapeutic indices even without a CA (low off target activity, high on target activity).

In a tumor free mouse, over 99% of unbound injected Bn-DOTA typically clears from the murine serum within four hours, with the vast majority of it being excreted in the urine within the first 30 minutes. In contrast, previous studies have shown that between 3 to 5% of directly labeled IgG will remain in the blood 48 hours after injection. (Azzopardi, N. et al. (2011) Clin Cancer Res 17, 6329-6337). As illustrated in FIG. 6B, over a 48 hours period, nearly 0.01% ID/g of directly labeled ¹³¹I-SADA-BiDE activity remains in the, indicating that P53-BiDE, P63-BiDE and P73-BiDE can all but completely cleared from the blood within 48 hours, without clearing agent.

Each dataset was analyzed using a two-phase decay model and the calculated values are presented here along with the integration of the curves (AUC), see Table 5. Here P53-BIDE and P63-BIDE stand out again, although the values are quite close. P53-BIDE has a longer portion of its decay during the slow component, but has a lower slow half-life. P63-BIDE has a greater portion in the fast component, but a substantially longer slow-half-life.

TABLE 5
Calculated values based on 2-phase decay model for
P53-BIDE, P63-BIDE and P73-BIDE

| Normalized | P53-BIDE | P63-BIDE | P73-BIDE |
|---|---|---|---|
| Y0 | 1.50 | 1.11 | 1.48 |
| Plateau | 0.03 | 0.02 | 0.04 |
| PercentFast | 36.73 | 43.16 | 33.88 |
| KFast | 3.03 | 0.35 | 3.58 |
| KSlow | 0.17 | 0.11 | 0.16 |
| Half Life (Slow) | 4.15 | 6.42 | 4.43 |
| Half Life (Fast) | 0.23 | 1.99 | 0.19 |
| Tau (slow) | 5.98 | 9.26 | 6.40 |
| Tau (fast) | 0.33 | 2.87 | 0.28 |
| Rate constant ratio | 18.13 | 3.23 | 22.91 |
| Total Area (AUC) | 7.51 | 8.55 | 8.45 |
| Std. Error | 0.35 | 0.60 | 0.28 |
| 95% Confidence | 6.83 to | 7.37 to | 7.90 to |
| Interval | 8.19 | 9.73 | 8.99 |

In tumor bearing mice treated with either IgG-BiDE or SADA-BiDE (P53-BiDE, P63-BiDE, P73-BiDE), as shown in FIG. 6C, SADA-BiDE administration leads to minimal Bn-DOTA retention in the blood, as compared to the IgG-BiDE. Even while the IgG-BiDE received CA and the SADA-BiDE did not, the Bn-DOTA clears very rapidly, indicating very minimal SADA-BiDE remains in the blood 48 hours after pretargeting. This again highlights the exemplary pharmacokinetics of the SADA-BiDES for PRIT. Additionally it shows that the kinetics are similar between three different SADA domains in three different SADA-BiDE conjugates. Furthermore the representative overlays suggest that by the time of payload delivery SADA-BiDEs treated mice show a clearance of Bn-DOTA that almost exactly follows typical Bn-DOTA single administration, further proving that almost all SADA-BiDE has self cleared by this interval. By contrast, IgG-BiDE treated mice show a clearance curve similar to a directly labeled IgG, suggesting that while most excess IgG-BiDE has been removed from the serum via CA, the remaining amount binds the payload and clears slowly, exposing the blood to unwanted levels of payload activity.

Importantly, even though, as described in the previous examples, P53-BIDE(NOHIS), P53-BiDE, P63-BiDE and P73-BiDE is rapidly cleared from the serum, total tumor uptake of was not affected. With both 24 hours and 72 hours between P53-BIDE(NOHIS) and ⁷Lu-Bn-DOTA injections, significant activity (^{˜}15% ID/g) was measured at the tumor site (FIG. 6D)

Furthermore, SADA-BiDE P53-BIDE(NOHIS) is stably retained at the target site, even after 96 hours, as shown in FIG. 6E. This extended retention at the target contrasts the rapid clearance from all non-target tissues, such as the blood, displaying the exemplary in vivo activity of the SADA-BiDE.

These data demonstrate the surprising and contrasting in vivo behavior of exemplary SADA-based conjugates, P53-BiDE, P63-BiDE, P73-BiDE, which are rapidly cleared from blood and remains stably bound to a tumor site. Further, these data suggest, among other things, that there is substantial flexibility in the time interval between SADA-antibody conjugates and payload injections, which is an important consideration during clinical applications. Without wishing to be bound by theory, we propose that SADA-based conjugates have altered behavior based on target antigen density: in the presence of its cognate antigen, the self-assembled multimeric state demonstrates high avidity, thereby stabilizing its retention in the tumor site, while absence of the antigen (i.e. at off-target sites), the multimer disassembles into monomeric units which are then rapidly cleared renally.

### Example 6—Pharmacokinetics and Tissue Biodistribution of Exemplary Antibody-Based Sada Conjugates

This example describes the tissue biodistribution of exemplary bispecific antibody-based SADA conjugates. In particular, this Example demonstrates that exemplary bispecific antibody-based conjugates with three SADA domain (P53-BiDE, P63-BiD3, P73-BiDE) exhibit promising tissue biodistribution in vivo.

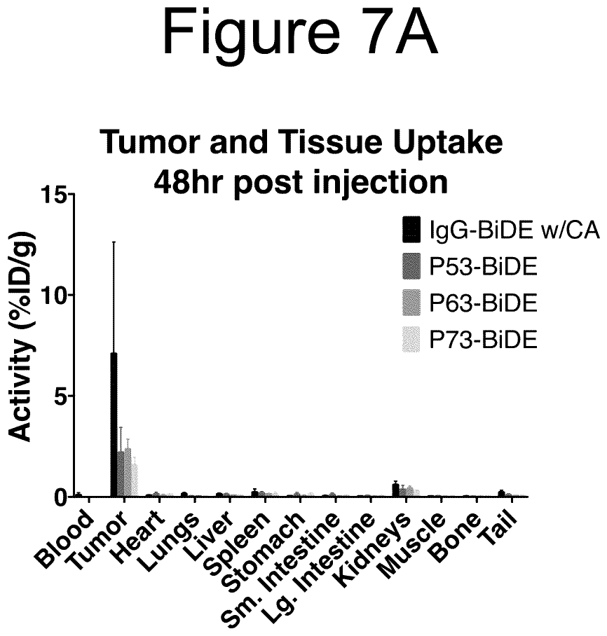

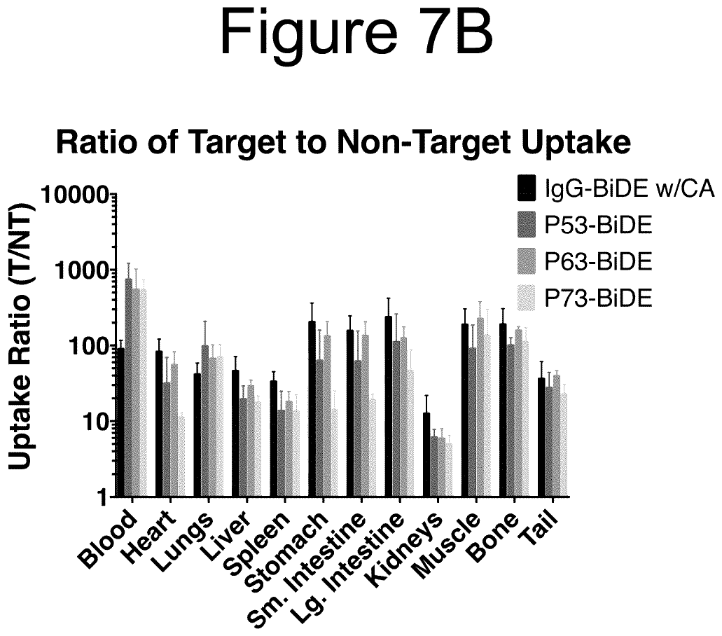

As illustrated in FIGS. 7A-7B and Tables 6a and 6b, all three SADA-BiDE conjugates have promising tissue biodistribution, even in comparison with a corresponding IgG-BiDE conjugate. Previously reported antibody-based therapeutics for PRIT, such as IgG-BiDE platforms (Cheal, S. M. et al. (2014) Mol Cancer Ther 13, 1803-1812), or biotin/streptavidin complexes (Cheung, N. K. et al. (2004) J Nucl Med 45, 867-877), are limited by biodistribution. For example, a clearing agent must be used with IgG-scFv platforms to remove excess unbound antibody. Streptavidin-based therapeutics, in addition issues related to immunogenicity of administering a bacterial protein, also have unwanted off-target effects resulting from the unusually high kidney uptake of these agents. In contrast, P53-BIDE, P63-BiDE and P73-BiDE had minimal kidney uptake, not significantly different from the uptake of Bn-DOTA alone (FIG. 7A and Table 6a). When compared to a IgG-BiDE platform, even with the additional benefit of clearing agents (CA), all three SADA-BiDEs were able to achieve remarkably low non-target uptake in nearly every tissue leading to very high therapeutic indices (FIG. 7B and Table 6b), despite no clearing agent being used. In particular, uptake was lower in the blood, spleen, liver and kidneys, all critically important tissues that are often damaged during conventional radioimmunotherapy.

TABLE 6a
Biodistribution (% ID/g uptake)
(See also, e.g., FIG. 7A)

| % ID/g uptake | IgG- | | | |
|---|---|---|---|---|
| per tissue | BiDE | P53- | P63- | P73- |
| (Lower is Better) | w/CA | BIDE | BIDE | BIDE |
| Blood | 0.099 | 0.003 | 0.006 | 0.003 |
| Tumor | 7.097 | 2.204 | 2.366 | 1.581 |
| Heart | 0.078 | 0.143 | 0.065 | 0.139 |
| Lungs | 0.156 | 0.036 | 0.042 | 0.024 |
| Liver | 0.143 | 0.122 | 0.081 | 0.089 |
| Spleen | 0.231 | 0.188 | 0.141 | 0.148 |
| Stomach | 0.043 | 0.130 | 0.042 | 0.142 |
| Sm. Intestine | 0.049 | 0.114 | 0.028 | 0.082 |
| Lg. Intestine | 0.031 | 0.051 | 0.025 | 0.052 |
| Kidneys | 0.602 | 0.369 | 0.422 | 0.321 |
| Muscle | 0.035 | 0.040 | 0.016 | 0.027 |
| Bone | 0.036 | 0.021 | 0.015 | 0.019 |
| Tail | 0.226 | 0.094 | 0.060 | 0.074 |

TABLE 6b
Biodistribution (Tumor:non-Tumor % ID/g ratio)
(See also, e.g., FIG. 7B)

| Tumor to Non- | IgG- | | | |
|---|---|---|---|---|
| Tumor Uptake Ratio | BiDE | P53- | P63- | P73- |
| (Higher is better) | w/CA | BIDE | BIDE | BIDE |
| Blood | 90 | 745 | 548 | 540 |
| Heart | 83 | 32 | 55 | 11 |
| Lungs | 42 | 98 | 67 | 70 |
| Liver | 46 | 20 | 29 | 18 |
| Spleen | 33 | 14 | 18 | 14 |
| Stomach | 205 | 63 | 133 | 14 |
| Sm. Intestine | 157 | 62 | 135 | 19 |
| Lg. Intestine | 237 | 112 | 125 | 46 |
| Kidneys | 13 | 6 | 6 | 5 |
| Muscle | 189 | 91 | 226 | 136 |
| Bone | 191 | 101 | 158 | 112 |
| Tail | 36 | 28 | 40 | 23 |

### Example 7—Complete Tumor Ablation with a Bispecific Antibody-Based Conjugate with a SADA Domain

This Example documents the in vivo efficacy of SADA-based antibody conjugates to mediate a reduction in tumor burden in mice. In particular, this Example demonstrates, among other things, that a two-step PRIT regimen using an exemplary tetrameric bispecific antibody-based conjugates with a SADA domain (P53-BIDE(NOHIS)) can relieve tumor burden, and even completely ablate tumors in vivo.

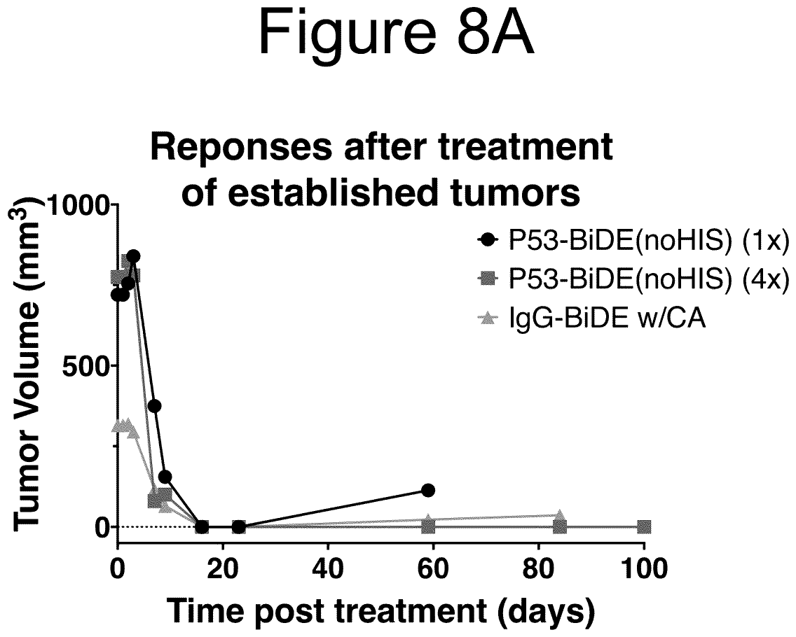

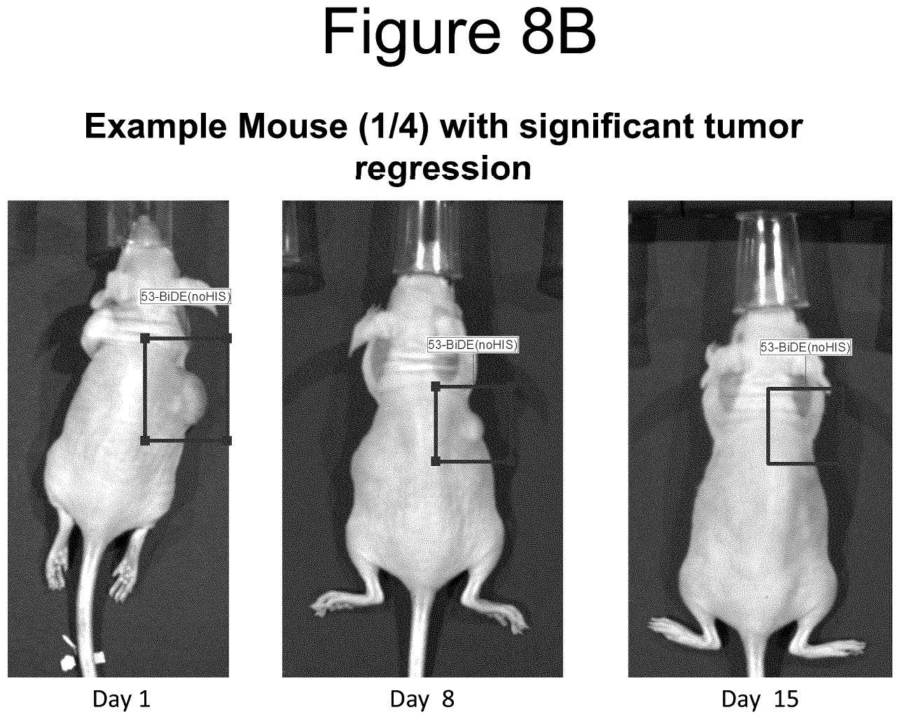

In mice with significant tumor burden (>500 mm³ tumor volumes) a single 250 μg (1.25 nmol) dose of P53-BIDE(NOHIS) was administered followed 24 hour later by administration of 2mCi of ¹⁷Lu-Bn-DOTA. As shown in FIGS. 8A and 8B, this two-step PRIT therapy with P53-BIDE(NOHIS) was able to completely ablate tumors in all four mice treated. Thus, two-step PRIT therapy using P53-BIDE(NOHIS), even with only 24 hours between administration of P53-BIDE(NOHIS) and ¹⁷⁷Lu-Bn-DOTA, and importantly without the use of a CA, is a highly effective tumor therapy. Furthermore, even administration of up to four doses of P53-BIDE(NOHIS), totaling 2 mCi of ¹⁷⁷Lu-Bn-DOTA, did not induce any clinical or histologic toxicity (data not shown). To date, no off-target toxicity was observed in any of the treated mice. This Example demonstrates, among other things, that two-step PRIT using a SADA-based antibody conjugate effectively reduces tumor burden in vivo and further suggests that such a therapy may be curative.

### Example 8—Production of Exemplary SADA-Cytokine Multimers

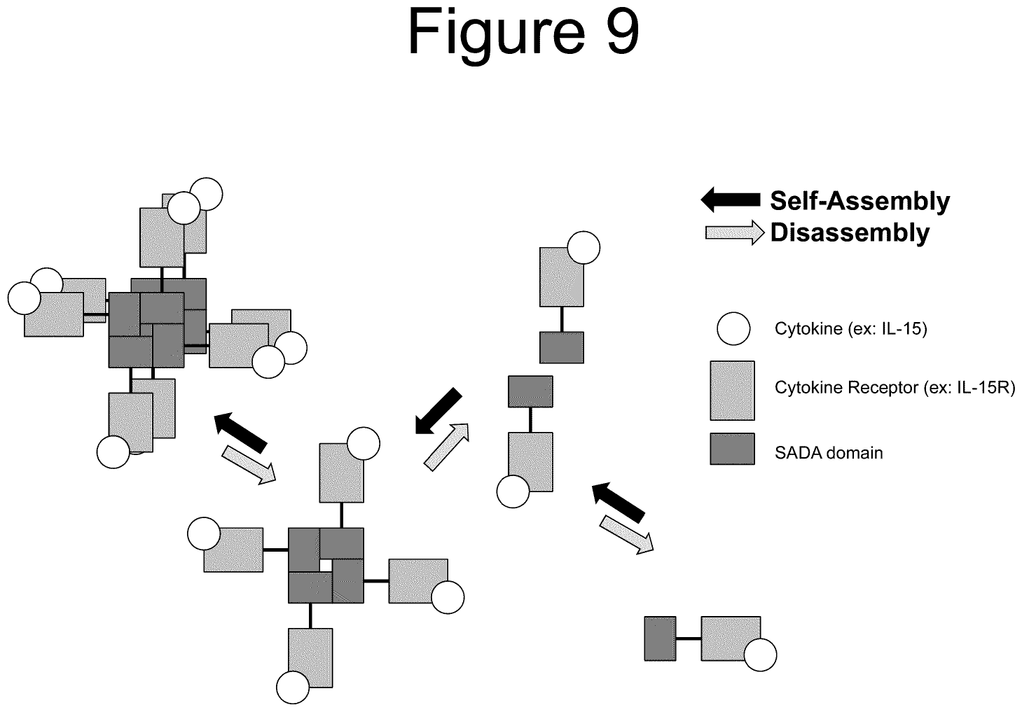

This example demonstrates the production of exemplary cytokine-based conjugates with SADA domains. Specifically, this example describes the production of SADA-Cytokine multimers using three different exemplary SADA domains: p53, p63 and p73, as illustrated in FIG. 9.

In addition to these three exemplary SADA domains and, as a proof of concept for using multiple different SADA domains, we used a cytokine complex that can dimerize with itself, thus creating an additional layer of self-assembly and disassembly, resulting in an octameric complex when fully assembly (FIG. 9). Without wishing to be bound by theory, it is envisioned that, in at least some embodiments, use of both tetramerization and a dimerizable cytokine will result in hierarchical self-assembly and disassembly resulting in four distinct dates for the construct: octamer (full), tetramer (half), dimer (quarter), and monomer (eighth). Specifically, in this example a IL15Rα/IL15 cytokine complex was used, each monomer containing both a covalently linked polypeptide (IL15Rα) and a soluble polypeptide (IL15) that attaches non-covalently with subnanomolar affinity. Since the IL15Rα self-dimerizes through its built-in anti-parallel sequence (Azzopardi, N. et al. (2011) Clin Cancer Res 17, 6329-6337), the full complex is made up of 8 pairs of IL15Rα/IL15. With a molecular size of ^{˜}200 kDa, the octamer exceeds the renal threshold, but the unbound dimer or monomer of IL15Rα/IL15 is small enough to be cleared through the kidneys after disassembly. A schematic is shown in FIG. 9.

Three different SADA-Cytokine multimers were produced: P53-Cytokine (IL15Rα, huP53-tet), P63-Cytokine (IL15Rα, huP63-tet), P73-Cytokine IL15Rα, huP73-tet), each of associates non-covalently with a corresponding soluble cytokine polypeptide (sIL15) at high affinity to form a SADA-Cytokine dimer, which then self-assembles into a SADA-cytokine octomer. The amino acid sequences and cDNA nucleotide sequences of P53-Cytokine, P63-Cytokine, P73-Cytokine and sIL15 are shown below.

P53-Cytokine polypeptide (IL15Rα, huP53-tet,
(IgG3 spacer))
SEQ ID NO: 57
ITCPPPMSVEHADIWVKSYSLYSRERYICNSGFKRKAGTSSLTECVLNK
ATNVAHWTTPSLKCIR(TPLGDTTHT)SGKPLDGEYFTLQIRGRERFEM
FRELNEALELKDAQAGKEPGGSGGAPHHHHHH
P53-Cytokine cDNA (IL15Rα, huP53-tet,
(IgG3 spacer)))
SEQ ID NO: 58
ATCACCTGTCCTCCACCCATGTCTGTGGAACACGCCGACATCTGGGTCA
AGTCCTACTCCCTGTACTCCAGAGAGCGGTACATCTGCAACTCCGGCTT
CAAGCGGAAGGCCGGCACCTCTAGCCTGACCGAGTGCGTGCTGAACAAG
GCCACCAACGTGGCCCACTGGACCACCCCATCCCTGAAGTGCATCAGAA
CACCCCTGGGTGACACCACACATACTAGTGGGAAACCTCTGGATGGCGA
GTACTTTACCCTGCAGATTAGAGGCCGCGAACGATTCGAGATGTTTCGC
GAACTGAATGAGGCCCTGGAACTGAAGGATGCTCAGGCAGGCAAGGAGC
CAGGAGGGTCAGGAGGAGCACCGCACCATCATCATCACCAT
P63-Cytokine polypeptide (IL15Rα, huP63-tet,
(IgG3 spacer)))
SEQ ID NO: 59
ITCPPPMSVEHADIWVKSYSLYSRERYICNSGFKRKAGTSSLTECVLNK
ATNVAHWTTPSLKCIR(TPLGDTTHT)SGRSPDDELLYLPVRGRETYEM
LLKIKESLELMQYLPQHTIETYRQQQQQQHQHLLQKQGGSGGAPHHHHH
H
P63-Cytokine cDNA (IL15Rα, huP63-tet,
(IgG3 spacer)))
SEQ ID NO: 60
ATCACCTGTCCTCCACCCATGTCTGTGGAACACGCCGACATCTGGGTCA
AGTCCTACTCCCTGTACTCCAGAGAGCGGTACATCTGCAACTCCGGCTT
CAAGCGGAAGGCCGGCACCTCTAGCCTGACCGAGTGCGTGCTGAACAAG
GCCACCAACGTGGCCCACTGGACCACCCCATCCCTGAAGTGCATCAGAA
CACCCCTGGGTGACACCACACATACTAGTGGGAGATCCCCCGACGATGA
GCTGCTGTACCTGCCTGTGAGGGGCCGGGAGACCTATGAAATGCTGCTG
AAGATCAAAGAGAGCCTGGAACTGATGCAGTACCTGCCACAGCACACCA
TTGAAACATATAGGCAACAACAGCAGCAGCAGCATCAGCATCTGCTGCA
GAAGCAGGGAGGGTCAGGAGGAGCACCGCACCATCATCATCACCAT
P73-Cytokine polypeptide (IL15Rα, huP73-tet,
(IgG3 spacer))
SEQ ID NO: 61
ITCPPPMSVEHADIWVKSYSLYSRERYICNSGFKRKAGTSSLTECVLNK
ATNVAHWTTPSLKCIR(TPLGDTTHT)SGRHGDEDTYYLQVRGRENFEI
LMKLKESLELMELVPQPLVDSYRQQQQLLQRPGGSGGAPHHHHHH
P73-Cytokine cDNA (IL15Rα, huP73-tet,
(IgG3 spacer))
SEQ ID NO: 62
ATCACCTGTCCTCCACCCATGTCTGTGGAACACGCCGACATCTGGGTCA
AGTCCTACTCCCTGTACTCCAGAGAGCGGTACATCTGCAACTCCGGCTT
CAAGCGGAAGGCCGGCACCTCTAGCCTGACCGAGTGCGTGCTGAACAAG
GCCACCAACGTGGCCCACTGGACCACCCCATCCCTGAAGTGCATCAGAA
CACCCCTGGGTGACACCACACATACTAGTGGGAGGCACGGCGACGAAGA
TACCTACTATCTGCAGGTGAGGGGACGGGAGAACTTCGAAATCCTGATG
AAGCTGAAAGAGTCCCTGGAACTGATGGAGCTGGTGCCCCAGCCTCTGG
TCGACAGCTACAGACAGCAGCAGCAGCTGCTGCAGAGGCCAGGAGGGTC
AGGAGGAGCACCGCACCATCATCATCACCAT
IL-15 polypeptide
SEQ ID NO: 63
NWVNVISDLKKIEDLIQSMHIDATLYTESDVHPSCKVTAMKCFLLELQV
ISLESGDASIHDTVENLIILANNSLSSNGNVTESGCKECEELEEKNIKE
FLQSFVHIVQMFINTS
IL-15 cDNA
SEQ ID NO: 64
ATGGGCTGGTCCTGCATCATCCTGTTTCTGGTGGCTACCGCCACCGGCA
ACTGGGTCAACGTGATCTCCGACCTGAAGAAGATCGAGGACCTGATCCA
GTCCATGCACATCGACGCCACCCTGTACACCGAGTCCGACGTGCACCCC
TCCTGCAAAGTGACCGCCATGAAGTGCTTTCTGCTGGAACTGCAAGTGA
TCTCCCTGGAATCCGGCGACGCCTCCATCCACGACACCGTGGAAAATCT
GATCATCCTGGCCAACAACTCCCTGTCCTCCAACGGCAACGTGACCGAG
AGCGGCTGCAAAGAGTGCGAGGAACTGGAAGAGAAGAACATCAAAGAGT
TTCTGCAGTCCTTCGTGCACATCGTGCAGATGTTCATCAACACCAGC

### Example 9—Stability of Exemplary SADA-Cytokine Multimers

This Example demonstrates the stability of exemplary SADA-Cytokine multimers. In particular, this Example describes biochemical purity analysis of preparations of three different exemplary SADA-Cytokine multimers (P53-Cytokine, P63-Cytokine and P73-Cytokine), each of which employs a different SADA domain.

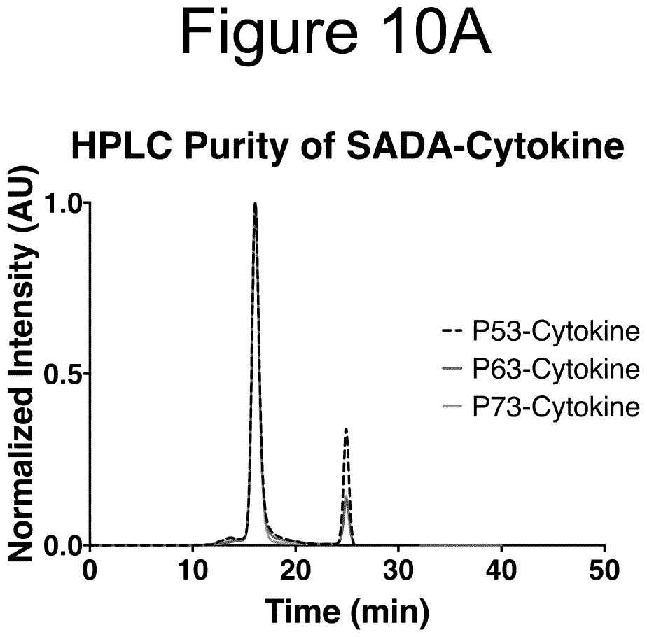

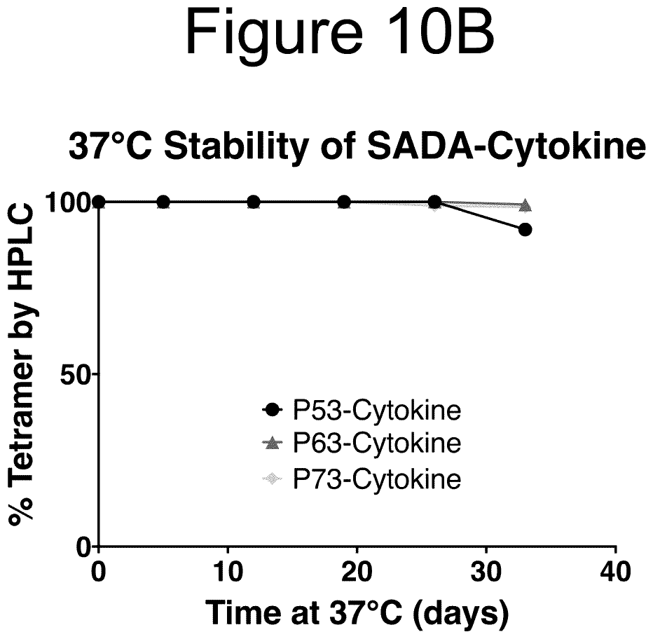

As illustrated in FIG. 10, each of the SADA-Cytokine multimers tested showed high in vitro stability. Preparations of P53-Cytokine, P63-Cytokine and P73-Cytokine were each able to form highly stable multimers of consistent size, as shown in HPLC chromatograms depicted in FIG. 10A, which have a major peak that corresponded with purity above 98%. Further, each of the constructs maintained their self-assembled multimeric state for over 30 days at 37° C. (FIG. 10B). Thus HPLC analysis provided herein demonstrates, among other things, the high in vitro stability of different SADA-Cytokine multimers that employ different SADA domains. These data demonstrate, among other things, the high stability of SADA-Cytokine complexes in vitro, and further suggests a strong potential for manufacturability.

### Example 10—In Vitro Cell Toxicity/Activity of Exemplary SADA-Cytokine Multimers

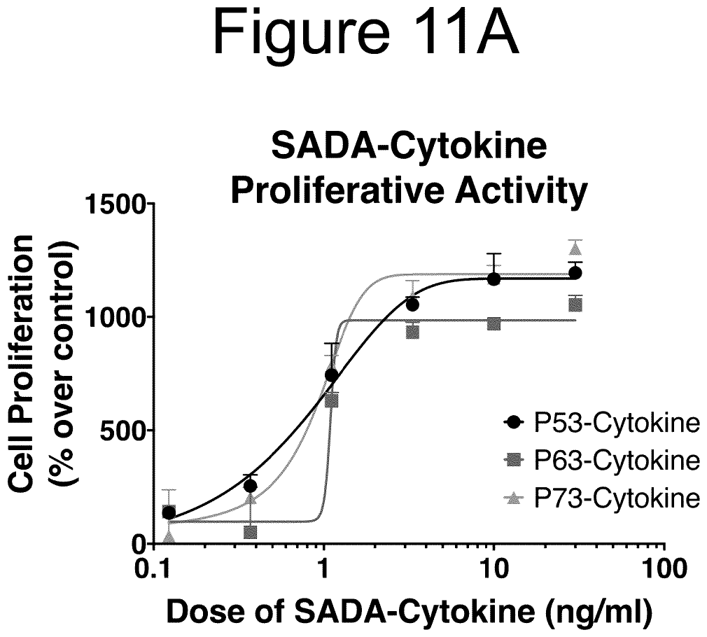

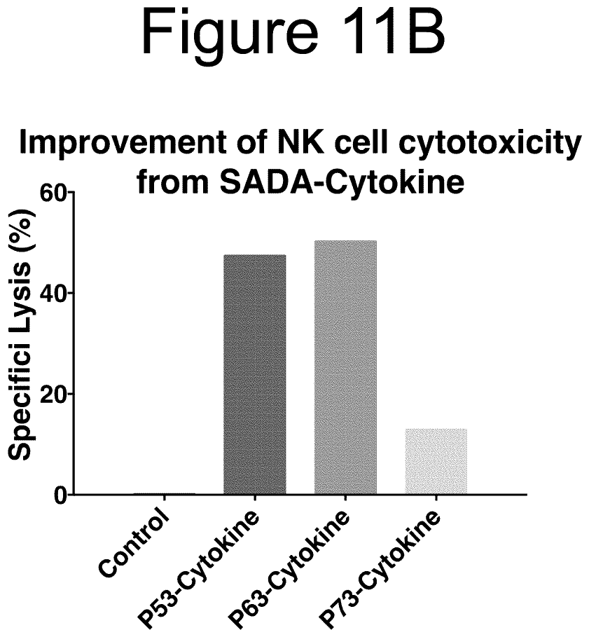

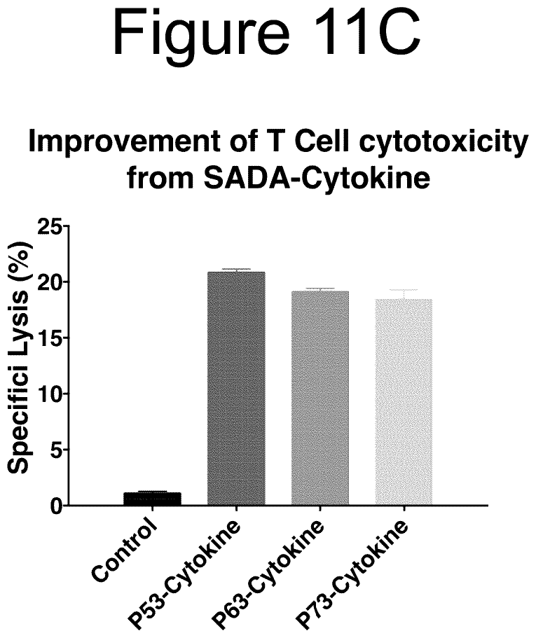

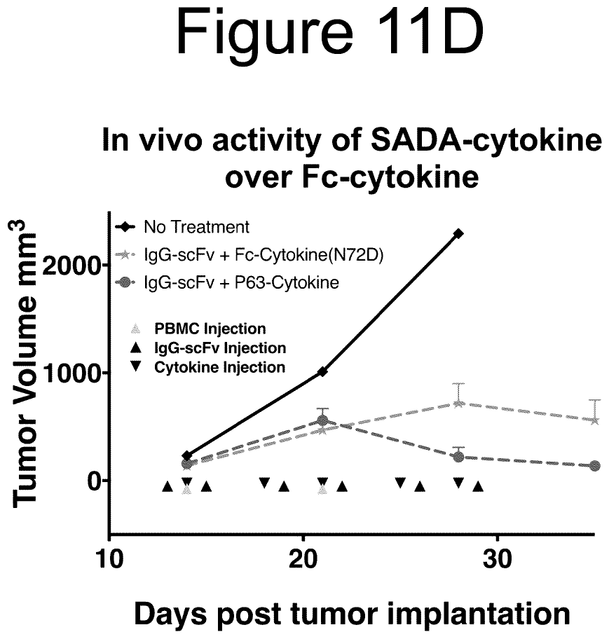

This example documents the in vitro activity of exemplary SADA-cytokine multimers. In particular, this Example demonstrates that preparations of three different exemplary SADA-cytokine multimers each have robust in vitro activity. Specifically, P53-Cytokine, P63-Cytokine and P73-Cytokine each exhibited strong IL15 signaling activity in vitro. As shown in FIG. 11A, P53-Cytokine, P63-Cytokine and P73-Cytokine each lead to robust proliferation of TIB214, an IL15 sensitive cell line relative to untreated control cells. Additionally, each complex could prime effector immune cells to kill more strongly. Human NK cells were incubated in 1 nM concentrations of P53-Cytokine, P63-Cytokine or P73-Cytokine for three days. As shown in FIG. 11B, each SADA-Cytokine multimeric complex increased antibody-independent cytotoxic response against a GD2(+) neuroblastoma cell line. Further, when incubated with human T cells for three days, each SADA-cytokine multimeric complex strongly increased IgG-scFv dependent killing of tumor cells (FIG. 11C) (Xu, H. et al. (2015) Cancer immunology research 3, 266-277). Importantly, these complexes showed improved functional activity over Fc dimerized versions (Liu et al. 2016 JBC, http://www.jbc.org/content/291/46/238691in vivo, as shown in FIG. 11D, suggesting their self-assembled multimeric state improved their activity through 2+ multimeric binding.

Without being bound to theory, it is envisioned that, in at least some embodiments, hierarchical multimerization or increased valency of constructs may improve binding activity, functional activity, increased stability and/or otherwise provide useful attributes to an therapeutic polypeptide.

### Example 11—Structural Analysis of SADA Domains

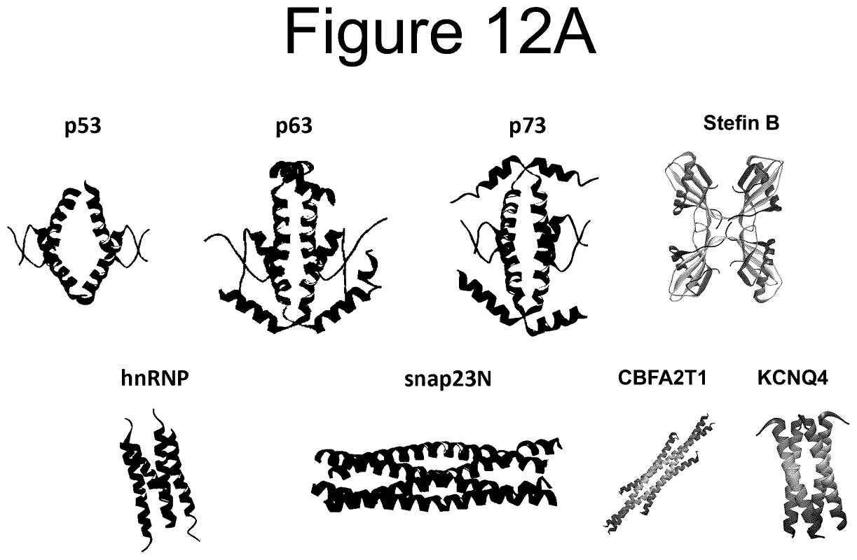

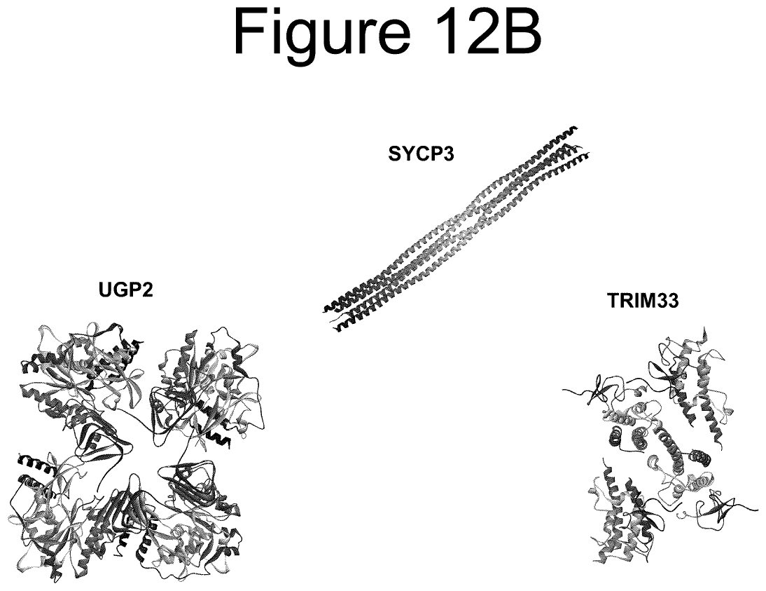

This example documents the characteristics of polypeptides for use as a SADA domain. Association and disassociation rates of a SADA domain polypeptide will affect the pharmacokinetic properties of SADA conjugates (e.g., antibody-based SADA conjugates, SADA-Cytokine conjugates). SADA domains are human derived multimerization domains that are sufficiently stable enough to multimerize tethered protein units in a non-covalent manner. In some embodiments, a SADA domain is composed of a multimerization domains from one of following human proteins: p53, p63, p73, heterogeneous nuclear Ribonucleoprotein C (hnRNPC), or N-terminal domain of Synaptosomal-associated protein 23 (SNAP-23), Stefin B (Cystatin B), Potassium voltage-gated channel subfamily KQT member 4 (KCNQ4), Cyclin-D-related protein (CBFA2T1), which are each composed of helical bundles that associate in a parallel or anti-parallel orientation (Table 7 and FIGS. 12A and 12B). Moreover, in some embodiments, a SADA domain lacks unpaired cysteine residues and/or large exposed hydrophobic surfaces, which without being bound by theory, are suggested to lead to aggregation. Each of the SADA domains described in Table 7a (i.e., p53, p63, p73, hnRNPC, SNAP-23, Stefin B, KCNQ4, and CBFA2T1) are absent of unpaired cysteine residues and large exposed hydrophobic surfaces.

TABLE 7a
Structural properties of SADA domains from analysis of crystal structures

| | | | | Buried | No. H | | | |
|---|---|---|---|---|---|---|---|---|
| | | | | SA | bonds | Buried SA | No. H bonds | Total |
| | | | | (dimer: | (dimer: | (monomer: | (monomer: | buried |
| | | MW of | | dimer) | dimer) | monomer) | monomer) | surface |
| Protein Complex | Conformation | monomer | PDB ID | (Å2) | (Å2) | (Å2) | (Å2) | area (Å2) |
| Tetramerization | Anti-parallel | 3.8 kDa | 2J0Z | 242 | 3 | 478 | 20 | 1199 |
| domain of p53 | homotetramer | | | | | | | |
| (residues 321-359) | | | | | | | | |
| Tetramerization | Anti-parallel | 6.1 kDa | 2WQI | 1066 | 32 | 617 | 24 | 2301 |
| domain of p73 | homotetramer | | | | | | | |
| (residues 348-399) | | | | | | | | |
| Tetramerization | Anti-parallel | 7.3 kDa | 4A9Z | 1188 | 33 | 646 | 32 | 2480 |
| domain of p63 | homotetramer | | | | | | | |
| (residues 396-450) | | | | | | | | |
| Oligomerization | Anti-parallel | 3.3 kDa | 1TXP | 630 | 3 | 172 | 4 | 973 |
| domain of hnRNP | homotetramer | | | | | | | |
| (residues 194-220) | | | | | | | | |
| Oligomerization | Parallel | 6.2 kDa | 1NHL | 957 | 16 | 465 | 9 | 1887 |
| domain of SNAP-23 | homotetramer | | | | | | | |
| (residues 23-76) | | | | | | | | |
| Oligomerization | domain swapped | 11.1 kDa | 2OCT | 1520 | 70 | 1028 | 51 | 3576 |
| domain of Stefin B | homotetramer | | | | | | | |
| (residues 2-98) | | | | | | | | |
| Oligomerization | parallel | 3.5 kDa | 2OVC | 628 | 10 | 314 | 5 | 1256 |
| domain of KCNQ4 | homotetramer | | | | | | | |
| (residues 611-640) | | | | | | | | |
| Oligomerization | anti-parallel | 7.5 kDa | 4JOL | 1207 | 18 | 514 | 15 | 2235 |
| domain of | homotetramer | | | | | | | |
| CBFA2T1 | | | | | | | | |
| (residues 462-521) | | | | | | | | |

TABLE 7b
Structural properties of potential SADA domains from analysis of
crystal structures

| | | | | | No. H | | No. H | Total |
|---|---|---|---|---|---|---|---|---|
| | | | | | bonds | Buried SA | bonds | buried |
| | | | | | (dimer: | (monomer: | (monomer: | surface |
| | | MW of | PDB | Protein | dimer) | monomer) | monomer) | area |
| | Conformation | monomer | ID | Complex | (Å2) | (Å2) | (Å2) | (Å2) |
| Oligomerization | anti-parallel | 17.2 kDa | 4CPC | 3209 | 62 | 1052 | 23 | 5313 |
| domain of SYCP3, | homotetramer | | | | | | | |
| (residues 81-221) | | | | | | | | |
| Oligomerizaiton | large paralllel | 54.3 kDa | 4R7P | 177 | 7 | 64 | 2 | 305 |
| domain of UGP2 | homotetramer | | | | | | | |
| (residues 24-508) | | | | | | | | |
| Oligomerization | anti-parallel | 11.0 kDa | 3U5O | 469 | 17 | 96 | 4 | 661 |
| domain of | homotetramer | | | | | | | |
| TRIM33 (residues | | | | | | | | |
| 958-1055) | | | | | | | | |

In some embodiments, a SADA domain is able to associate to form homo-tetramers, and further that can dissociate into dimers and monomers. The association and disassociation rates of a p53 tetramerization domain, was measured to have a dissociation constant (K_{D}, which is equal to k_{off}/kₒₙ) at 37° C. for tetramers dissociating into dimers of 150 nM (half-life of 2.5 minutes), and a dissociation constant of dimers into monomers of 1 nM (half-life of 13 min), based on fluorescence correlation spectroscopy (Matthay, K. K. et al. (2007) J Clin Oncol 25, 1054-1060). However accurate measurements of the association and disassociation rates of the other homo-tetramerization domains listed in Table 7a (i.e., p63, p73, hnRNPC, SNAP-23, Stefin B, KCNQ4, and CBFA2T1) have not been previously been reported. Since the crystal structures of each of the SADA domains listed in Table 7a (i.e., the tetramerization domains of p53, p63, p73, hnRNPC, SNAP-23, Stefin B, KCNQ4, and CBFA2T1) are known, the crystal structures were analyzed to determine the relative dissociation constants based on buried surface area of the complexes. Without wishing to be bound by theory, it has been suggested that the buried surface area of protein:protein complexes significantly correlate inversely to the log of the measured dissociation constants (Pinzani, V. et al. (1994) Cancer Chemoth Pharm 35, 1-9). Based on these observations, the crystal structures of the tetramerization domains of p53, p63, p73, hnRNPC, SNAP-23, Stefin B, KCNQ4, and CBFA2T1 were analyzed for buried surface area at the dimer:dimer and monomer:monomer interfaces, number of interface hydrogen bonds and the total buried surface area (Table 7a). The calculations were made using Biovia Discovery Studio (Dassault Systemes, San Diego CA). Based on these calculations, we extrapolated that the tetramerization domains of p63, p73, SNAP-23, Stefin B, and CBFA2T1 (957-1520 Å² of buried surface area of the dimer:dimer interfaces) will have a smaller dissociation constant in the tetramer-to-dimer transition than hnRNPC (630 Å²), KCNQ4 (628 Å²) or p53 (242 Å²). Additionally, the dimer-to-monomer dissociations constants of p53, p63, p73, SNAP-23, Stefin B, KCNQ4, and CBFA2T1 (314-1028 Å² of buried surface area of monomer:monomer interface) will be significantly lower than hnRNPC (172 Å²). Based on the total buried surface area, p63, p73, SNAP-23, Stefin B, and CBFA2T1 SADA domains (1887-3576 Å²) will have smaller overall observed dissociation constants (tetramer-to-monomer) than p53 (1199 Å²), hnRNPC (973 Å²), KCNQ4 (1256 Å²).

Additionally, three other potential SADA domains were analyzed (Table 7b) synaptonemal complex protein (SYCP3), UDP-glucose pyrophosphorylase (UGP2), and E3 ubiquitin-protein ligase (TRIM33). Based on these calculated buried surface area measurements, we extrapolate that UGP2 and TRIM33 would diassociate too quickly not bind to the target sufficiently. Furthermore the buried surface area measurements of SYCP3 suggest it would diassociate too slowly and provide unwanted exposure to normal tissues.

Based on these calculated buried surface area measurements and the expected relative dissociation constants, a SADA domain can be selected for the specific type of application. In some applications a rapid clearance rate may be desirable (e.g., SADA-PRIT), and so a SADA domain that has a faster dissociation/disassembly rate (e.g., p53, hnRNPC, KCNQ4) may be preferred. In some applications a longer serum half-life may be desired (e.g., certain SADA-Cytokine, SADA-BiDE, or SADA-BiWE applications), and so a SADA domain that has a slower dissociation/disassembly rate (e.g., p63, p73, SNAP-23, Stefin B, or CBFA2T1) may be chosen. It is also envisioned that a SADA domain can be engineered (e.g., introduce amino acid mutations or post-translational modifications) to increase or decrease the dissociation constants for the different applications. A SADA domain can also be selected for having parallel (SNAP-23 or KCNQ4), anti-parallel orientation (p53, p63, p73, hnRNPC, or CBFA2T1) or domain swapped oriengation (Stefin B), which without being bound by theory, is suggested to affect the ability of the tethered therapeutic protein to cooperatively bind its target. Thus, it is contemplated by the present invention to alter or tune various elements of a SADA domain to optimize biochemical and/or functional properties of a multimeric protein therapeutic to for each specific application.

### Example 12—Exemplary Tumor Binding Conjugates with SADA Domains

This example describes binding of tumor-targeted SADA conjugates to tumor antigens. Specifically, this example shows in vitro activity of an exemplary bispecific antibody based conjugate against the HER2 antigen using a P53 SADA domain, e.g., a HER2 P53-BiDE. This example confirms that SADA conjugates can be used to target different antigens (e.g., different tumor antigens) and different cell types (e.g. different tumor types). Provided below are polypeptide sequences and nucleotide sequences for various exemplary HER2-targeted SADA conjugates.

HER2 (HL DS) P53 BiDE (LL) polypeptide (hu4D5-scFv,
huC825-scFv, huP53-tet, GS linker, (IgG3 spacer))
SEQ ID NO: 65
EVQLVESGGGLVQPGGSLRLSCAASGFNIKDTYIHWVRQAPGKCLEWVARIYPTNG
YTRYADSVKGRFTISADTSKNTAYLQMNSLRAEDTAVYYCSRWGGDGFYAMDYW
GQGTLVTVSSGGGGSGGGGSGGGGSGGGGSGGGGSGGGGSDIQMTQSPSSLSASVG
DRVTITCRASQDVNTAVAWYQQKPGKAPKLLIYSASFLYSGVPSRFSGSRSGTDFTLT
ISSLQPEDFATYYCQQHYTTPPTFGQCTKVEIKRGGGGSGGGGSGGGGSGGGGSHVQ
LVESGGGLVQPGGSLRLSCAASGFSLTDYGVHWVRQAPGKGLEWLGVIWSGGGTAYNTA
LISRFTISRDNSKNTLYLQMNSLRAEDTAVYYCARRGSYPYNYFDAWGCGTLVTVSSGGGGS
GGGGSGGGGSGGGGSGGGGSGGGGSQAVVTQEPSLTVSPGGTVTLTCGSSTGAVTASNY
ANWVQQKPGQCPRGLIGGHNNRPPGVPARFSGSLLGGKAALTLLGAQPEDEAEYYCALW
YSDHWVIGGGTKLTVLG(TPLGDTTHT)SGKPLDGEYFTLQIRGRERFEMFRELNEA
LELKDAQAGKEPGGSGGAPHHHHHH
HER2 (HL DS) P53 BiDE (LL) cDNA (hu4D5-scFv, huC825-scFv, huP53-tet,
GS linker, (IgG3 spacer))
SEQ ID NO: 66
GAAGTGCAGCTGGTCGAATCCGGGGGGGGCCTGGTGCAGCCTGGAGGGTCACTGAG
ACTGTCCTGTGCCGCATCTGGGTTCAATATCAAGGACACCTACATCCACTGGGTGCGG
CAGGCACCTGGCAAGtGtCTGGAGTGGGTGGCAAGGATCTATCCAACCAACGGCTACA
CACGGTATGCCGACTCCGTGAAGGGCCGGTTCACCATCTCCGCCGATACCTCTAAGAA
CACAGCCTACCTGCAGATGAATTCTCTGAGGGCCGAGGATACAGCCGTGTACTATTGC
AGCCGCTGGGGAGGCGACGGCTTCTACGCTATGGACTATTGGGGCCAGGGCACCCTG
GTGACAGTGAGCTCTGGCGGCGGCGGATCCGGAGGAGGAGGCAGCGGCGGAGGA
GGCTCCGGAGGAGGCGGCTCTGGCGGCGGCGGCAGCGGCGGCGGCGGCTCCGAC
ATCCAGATGACCCAGTCCCCATCTAGCCTGAGCGCCTCCGTGGGCGACAGGGTGACC
ATCACATGCCGCGCCAGCCAGGATGTGAATACAGCCGTGGCCTGGTACCAGCAGAAG
CCAGGCAAGGCCCCCAAGCTGCTGATCTACTCTGCCAGCTTCCTGTATAGCGGAGTGC
CATCCCGGTTTTCCGGCAGCCGGAGCGGCACCGACTTCACCCTGACAATCTCCTCTCT
GCAGCCTGAGGATTTTGCCACATACTATTGTCAGCAGCACTATACCACACCCCCTACAT
TCGGACAGTGTACAAAGGTCGAGATCAAACGCGGCGGAGGGGGATCCGGCGGCGGA
GGATCTGGCGGAGGTGGAAGTGGGGGAGGCGGATCTCATGTGCAGCTGGTGGAAA
GCGGAGGCGGCCTGGTGCAGCCTGGGGGATCTCTGAGACTGTCTTGTGCCGCCAGC
GGCTTCTCCCTGACCGATTATGGCGTGCACTGGGTGCGACAGGCCCCTGGCAAAGGA
CTGGAATGGCTGGGAGTGATTTGGAGTGGCGGAGGCACCGCCTACAACACCGCCCTG
ATCTCCCGGTTCACCATCAGCCGGGACAACTCCAAGAACACCCTGTACCTGCAGATGA
ACTCCCTGCGGGCCGAGGACACCGCTGTGTACTACTGCGCCAGACGGGGCTCCTACC
CCTACAACTACTTCGACGCTTGGGGCTGCGGCACCCTCGTGACAGTGTCTAGCGGAG
GGGGAGGTTCTGGGGGCGGAGGTTCAGGTGGTGGTGGTTCCGGGGGTGGTGGCTCT
GGTGGCGGTGGTTCTGGCGGTGGCGGATCTCAGGCTGTCGTGACCCAGGAACCCAG
CCTGACTGTGTCTCCTGGCGGAACCGTGACCCTGACCTGCGGATCTTCTACCGGCGC
TGTGACCGCCAGCAACTACGCCAATTGGGTGCAGCAGAAACCTGGACAGTGCCCTAG
AGGCCTGATCGGCGGCCACAACAACAGACCTCCAGGCGTGCCAGCCCGGTTCTCTGG
ATCTCTGCTGGGCGGAAAGGCCGCTCTGACACTGCTGGGTGCTCAGCCTGAGGACGA
GGCCGAGTACTACTGTGCCCTGTGGTACTCCGACCACTGGGTCATCGGAGGCGGGAC
CAAGCTGACCGTGCTGGGA(ACACCCCTGGGAGACACCACACATACT)AGTGGGAA
ACCTCTGGATGGCGAGTACTTTACCCTGCAGATTAGAGGCCGCGAACGATT
CGAGATGTTTCGCGAACTGAATGAGGCCCTGGAACTGAAGGATGCTCAGGC
AGGCAAGGAGCCAGGAGGGTCAGGAGGAGCACCGCACCATCATCATCACCAT
HER2 (HL) P53 BiDE (LL) polypeptide (hu4D5-scFv, huC825-scFv,
huP53-tet, GS linker, (IgG3 spacer))
SEQ ID NO: 67
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSGGGGSGGGGSGGGGSGGGGSGGGGSGGGGSDIQMTQSPSSLSASVG
DRVTITCRASQDVNTAVAWYQQKPGKAPKLLIYSASFLYSGVPSRFSGSRSGTDFTLT
ISSLQPEDFATYYCQQHYTTPPTFGQGTKVEIKRGGGGSGGGGSGGGGSGGGGSHVQ
LVESGGGLVQPGGSLRLSCAASGFSLTDYGVHWVRQAPGKGLEWLGVIWSGGGTAYNTA
LISRFTISRDNSKNTLYLQMNSLRAEDTAVYYCARRGSYPYNYFDAWGCGTLVTVSSGGGGS
GGGGSGGGGSGGGGSGGGGSGGGGSQAVVTQEPSLTVSPGGTVTLTCGSSTGAVTASNY
ANWVQQKPGQCPRGLIGGHNNRPPGVPARFSGSLLGGKAALTLLGAQPEDEAEYYCALW
YSDHWVIGGGTKLTVLG(TPLGDTTHT)SGKPLDGEYFTLQIRGRERFEMFRELNEA
LELKDAQAGKEPGGSGGAPHHHHHH
HER2 (HL) P53 BiDE (LL) cDNA (hu4D5-scFv, huC825-scFv, huP53-tet,
GS linker, (IgG3 spacer))
SEQ ID NO: 68
GAAGTGCAGCTGGTCGAATCCGGGGGGGGCCTGGTGCAGCCTGGAGGGTCACTGAG
ACTGTCCTGTGCCGCATCTGGGTTCAATATCAAGGACACCTACATCCACTGGGTGCGG
CAGGCACCTGGCAAGGGACTGGAGTGGGTGGCAAGGATCTATCCAACCAACGGCTAC
ACACGGTATGCCGACTCCGTGAAGGGCCGGTTCACCATCTCCGCCGATACCTCTAAGA
ACACAGCCTACCTGCAGATGAATTCTCTGAGGGCCGAGGATACAGCCGTGTACTATTG
CAGCCGCTGGGGAGGCGACGGCTTCTACGCTATGGACTATTGGGGCCAGGGCACCCT
GGTGACAGTGAGCTCTGGCGGCGGCGGATCCGGAGGAGGAGGCAGCGGCGGAGG
AGGCTCCGGAGGAGGCGGCTCTGGCGGCGGCGGCAGCGGCGGCGGCGGCTCCGA
CATCCAGATGACCCAGTCCCCATCTAGCCTGAGCGCCTCCGTGGGCGACAGGGTGAC
CATCACATGCCGCGCCAGCCAGGATGTGAATACAGCCGTGGCCTGGTACCAGCAGAA
GCCAGGCAAGGCCCCCAAGCTGCTGATCTACTCTGCCAGCTTCCTGTATAGCGGAGTG
CCATCCCGGTTTTCCGGCAGCCGGAGCGGCACCGACTTCACCCTGACAATCTCCTCTC
TGCAGCCTGAGGATTTTGCCACATAGTATTGTCAGCAGCACTATACCACACCCCCTACA
TTCGGACAGGGGACAAAGGTCGAGATCAAACGCGGCGGAGGGGGATCCGGCGGCG
GAGGATCTGGCGGAGGTGGAAGTGGGGGAGGCGGATCTCATGTGCAGCTGGTGG
AAAGCGGAGGCGGCCTGGTGCAGCCTGGGGGATCTCTGAGACTGTCTTGTGCCGCCA
GCGGCTTCTCCCTGACCGATTATGGCGTGCACTGGGTGCGACAGGCCCCTGGCAAAG
GACTGGAATGGCTGGGAGTGATTTGGAGTGGCGGAGGCACCGCCTACAACACCGCCC
TGATCTCCCGGTTCACCATCAGCCGGGACAACTCCAAGAACACCCTGTACCTGCAGAT
GAACTCCCTGCGGGCCGAGGACACCGCTGTGTACTACTGCGCCAGACGGGGCTCCTA
CCCCTACAACTACTTCGACGCTTGGGGCTGCGGCACCCTCGTGACAGTGTCTAGCGG
AGGGGGAGGTTCTGGGGGCGGAGGTTCAGGTGGTGGTGGTTCCGGGGGTGGTGGCT
CTGGTGGCGGTGGTTCTGGCGGTGGCGGATCTCAGGCTGTCGTGACCCAGGAACCCA
GCCTGACTGTGTCTCCTGGCGGAACCGTGACCCTGACCTGCGGATCTTCTACCGGCG
CTGTGACCGCCAGCAACTACGCCAATTGGGTGCAGCAGAAACCTGGACAGTGCCCTA
GAGGCCTGATCGGCGGCCACAACAACAGACCTCCAGGCGTGCCAGCCCGGTTCTCTG
GATCTCTGCTGGGCGGAAAGGCCGCTCTGACACTGCTGGGTGCTCAGCCTGAGGACG
AGGCCGAGTACTACTGTGCCCTGTGGTACTCCGACCACTGGGTCATCGGAGGCGGGA
CCAAGCTGACCGTGCTGGGA(ACACCCCTGGGAGACACCACACATACT)AGTGGGA
AACCTCTGGATGGCGAGTACTTTACCCTGCAGATTAGAGGCCGCGAACGAT
TCGAGATGTTTCGCGAACTGAATGAGGCCCTGGAACTGAAGGATGCTCAGG
CAGGCAAGGAGCCAGGAGGGTCAGGAGGAGCACCGCACCATCATCATCACCAT
HER2 (LHDS) P53 BiDE (LL) polypeptide (hu4D5-scFv,
huC825-scFv, huP53-tet, GS linker, (IgG3 spacer))
SEQ ID NO: 69
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQCTKVEIKRGGGGSGG
GGSGGGGSGGGGSGGGGSGGGGSEVQLVESGGGLVQPGGSLRLSCAASGFNIKDTY
IHWVRQAPGKCLEWVARIYPTNGYTRYADSVKGRFTISADTSKNTAYLQMNSLRAE
DTAVYYCSRWGGDGFYAMDYWGQGTLVTVSSGGGGSGGGGSGGGGSGGGGSHVQ
LVESGGGLVQPGGSLRLSCAASGFSLTDYGVHWVRQAPGKGLEWLGVIWSGGGTAYNTA
LISRFTISRDNSKNTLYLQMNSLRAEDTAVYYCARRGSYPYNYFDAWGCGTLVTVSSGGGGS
GGGGSGGGGSGGGGSGGGGSGGGGSQAVVTQEPSLTVSPGGTVTLTCGSSTGAVTASNY
ANWVQQKPGQCPRGLIGGHNNRPPGVPARFSGSLLGGKAALTLLGAQPEDEAEYYCALW
YSDHWVIGGGTKLTVLG(TPLGDTTHT)SGKPLDGEYFTLQIRGRERFEMFRELNEA
LELKDAQAGKEPGGSGGAPHHHHHH
HER2 (LHDS) P53 BiDE (LL) cDNA (hu4D5-scFv, huC825-scFv,
huP53-tet, GS linker, (IgG3 spacer))
SEQ ID NO: 70
GATATTCAGATGACTCAGTCCCCTAGTTCACTGTCTGCCTCAGTCGGAGATCGGGTCA
CTATCACTTGTCGGGCTTCTCAGGATGTGAACACCGCCGTGGCCTGGTACCAGCAGAA
GCCAGGCAAGGCCCCCAAGCTGCTGATCTACTCTGCCAGCTTCCTGTATTCCGGAGTG
CCATCTCGGTTTTCCGGCAGCCGGAGCGGCACCGACTTCACCCTGACAATCAGCTCC
CTGCAGCCTGAGGATTTTGCCACATACTATTGCCAGCAGCACTATACCACACCCCCTAC
CTTCGGCCAGtGCACAAAGGTGGAGATCAAGAGGGGAGGAGGAGGATCCGGAGGAG
GAGGCAGCGGAGGCGGCGGCTCCGGCGGCGGCGGCTCTGGCGGCGGCGGCAGC
GGAGGAGGCGGCTCCGAGGTGCAGCTGGTGGAGTCCGGCGGCGGCCTGGTGCAGC
CCGGCGGCAGCCTGCGGCTGTCCTGTGCCGCCTCTGGCTTTAACATCAAGGACACCT
ACATCCACTGGGTGAGGCAGGCACCTGGCAAGtGCCTGGAGTGGGTGGCAAGGATCT
ATCCAACCAATGGCTACACAAGATATGCCGACTCCGTGAAGGGCCGCTTTACCATCAG
CGCCGATACCTCCAAGAACACAGCCTACCTGCAGATGAATTCTCTGCGGGCCGAGGAT
ACAGCCGTGTACTATTGCTCCAGATGGGGCGGCGACGGCTTCTATGCTATGGACTATT
GGGGGCAGGGAACTCTGGTCACTGTCTCCTCTGGCGGAGGGGGATCCGGCGGCGG
AGGATCTGGCGGAGGTGGAAGTGGGGGAGGCGGATCTCATGTGCAGCTGGTGGAA
AGCGGAGGCGGCCTGGTGCAGCCTGGGGGATCTCTGAGACTGTCTTGTGCCGCCAG
CGGCTTCTCCCTGACCGATTATGGCGTGCACTGGGTGCGACAGGCCCCTGGCAAAGG
ACTGGAATGGCTGGGAGTGATTTGGAGTGGCGGAGGCACCGCCTACAACACCGCCCT
GATCTCCCGGTTCACCATCAGCCGGGACAACTCCAAGAACACCCTGTACCTGCAGATG
AACTCCCTGCGGGCCGAGGACACCGCTGTGTACTACTGCGCCAGACGGGGCTCCTAC
CCCTACAACTACTTCGACGCTTGGGGCTGCGGCACCCTCGTGACAGTGTCTAGCGGA
GGGGGAGGTTCTGGGGGCGGAGGTTCAGGTGGTGGTGGTTCCGGGGGTGGTGGCTC
TGGTGGCGGTGGTTCTGGCGGTGGCGGATCTCAGGCTGTCGTGACCCAGGAACCCAG
CCTGACTGTGTCTCCTGGCGGAACCGTGACCCTGACCTGCGGATCTTCTACCGGCGC
TGTGACCGCCAGCAACTACGCCAATTGGGTGCAGCAGAAACCTGGACAGTGCCCTAG
AGGCCTGATCGGCGGCCACAACAACAGACCTCCAGGCGTGCCAGCCCGGTTCTCTGG
ATCTCTGCTGGGCGGAAAGGCCGCTCTGACACTGCTGGGTGCTCAGCCTGAGGACGA
GGCCGAGTACTACTGTGCCCTGTGGTACTCCGACCACTGGGTCATCGGAGGCGGGAC
CAAGCTGACCGTGCTGGGA(ACACCCCTGGGAGACACCACACATACT)AGTGGGAA
ACCTCTGGATGGCGAGTACTTTACCCTGCAGATTAGAGGCCGCGAACGATT
CGAGATGTTTCGCGAACTGAATGAGGCCCTGGAACTGAAGGATGCTCAGGC
AGGCAAGGAGCCAGGAGGGTCAGGAGGAGCACCGCACCATCATCATCACCAT
HER2 (LH) P53 BiDE (LL) polypeptide (hu4D5-scFv, huC825-scFv,
huP53-tet, GS linker, (IgG3 spacer))
SEQ ID NO: 71
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRGGGGSGG
GGSGGGGSGGGGSGGGGSGGGGSEVQLVESGGGLVQPGGSLRLSCAASGFNIKDTY
IHWVRQAPGKGLEWVARIYPTNGYTRYADSVKGRFTISADTSKNTAYLQMNSLRAE
DTAVYYCSRWGGDGFYAMDYWGQGTLVTVSSGGGGSGGGGSGGGGSGGGGSHVQ
LVESGGGLVQPGGSLRLSCAASGFSLTDYGVHWVRQAPGKGLEWLGVIWSGGGTAYNTA
LISRFTISRDNSKNTLYLQMNSLRAEDTAVYYCARRGSYPYNYFDAWGCGTLVTVSSGGGGS
GGGGSGGGGSGGGGSGGGGSGGGGSQAVVTQEPSLTVSPGGTVTLTCGSSTGAVTASNY
ANWVQQKPGQCPRGLIGGHNNRPPGVPARFSGSLLGGKAALTLLGAQPEDEAEYYCALW
YSDHWVIGGGTKLTVLG(TPLGDTTHT)SGKPLDGEYFTLQIRGRERFEMFRELNEA
LELKDAQAGKEPGGSGGAPHHHHHH
HER2 (LH) P53 BiDE (LL) cDNA (hu4D5-scFv, huC825-scFv,
huP53-tet, GS linker, (IgG3 spacer))
SEQ ID NO: 72
GATATTCAGATGACTCAGTCCCCTAGTTCACTGTCTGCCTCAGTCGGAGATCGGGTCA
CTATCACTTGTCGGGCTTCTCAGGATGTGAACACCGCCGTGGCCTGGTACCAGCAGAA
GCCAGGCAAGGCCCCCAAGCTGCTGATCTACTCTGCCAGCTTCCTGTATTCCGGAGTG
CCATCTCGGTTTTCCGGCAGCCGGAGCGGCACCGACTTCACCCTGACAATCAGCTCC
CTGCAGCCTGAGGATTTTGCCACATACTATTGCCAGCAGCACTATACCACACCCCCTAC
CTTCGGCCAGGGCACAAAGGTGGAGATCAAGAGGGGAGGAGGAGGATCCGGAGGA
GGAGGCAGCGGAGGCGGCGGCTCCGGCGGCGGCGGCTCTGGCGGCGGCGGCAG
CGGAGGAGGCGGCTCCGAGGTGCAGCTGGTGGAGTCCGGCGGCGGCCTGGTGCAG
CCCGGCGGCAGCCTGCGGCTGTCCTGTGCCGCCTCTGGCTTTAACATCAAGGACACC
TACATCCACTGGGTGAGGCAGGCACCTGGCAAGGGCCTGGAGTGGGTGGCAAGGATC
TATCCAACCAATGGCTACACAAGATATGCCGACTCCGTGAAGGGCCGCTTTACCATCA
GCGCCGATACCTCCAAGAACACAGCCTACCTGCAGATGAATTCTCTGCGGGCCGAGG
ATACAGCCGTGTACTATTGCTCCAGATGGGGCGGCGACGGCTTCTATGCTATGGACTA
TTGGGGGCAGGGAACTCTGGTCACTGTCTCCTCTGGCGGAGGGGGATCCGGCGGCG
GAGGATCTGGCGGAGGTGGAAGTGGGGGAGGCGGATCTCATGTGCAGCTGGTGG
AAAGCGGAGGCGGCCTGGTGCAGCCTGGGGGATCTCTGAGACTGTCTTGTGCCGCCA
GCGGCTTCTCCCTGACCGATTATGGCGTGCACTGGGTGCGACAGGCCCCTGGCAAAG
GACTGGAATGGCTGGGAGTGATTTGGAGTGGCGGAGGCACCGCCTACAACACCGCCC
TGATCTCCCGGTTCACCATCAGCCGGGACAACTCCAAGAACACCCTGTACCTGCAGAT
GAACTCCCTGCGGGCCGAGGACACCGCTGTGTACTACTGCGCCAGACGGGGCTCCTA
CCCCTACAACTACTTCGACGCTTGGGGCTGCGGCACCCTCGTGACAGTGTCTAGCGG
AGGGGGAGGTTCTGGGGGCGGAGGTTCAGGTGGTGGTGGTTCCGGGGGTGGTGGCT
CTGGTGGCGGTGGTTCTGGCGGTGGCGGATCTCAGGCTGTCGTGACCCAGGAACCCA
GCCTGACTGTGTCTCCTGGCGGAACCGTGACCCTGACCTGCGGATCTTCTACCGGCG
CTGTGACCGCCAGCAACTACGCCAATTGGGTGCAGCAGAAACCTGGACAGTGCCCTA
GAGGCCTGATCGGCGGCCACAACAACAGACCTCCAGGCGTGCCAGCCCGGTTCTCTG
GATCTCTGCTGGGCGGAAAGGCCGCTCTGACACTGCTGGGTGCTCAGCCTGAGGACG
AGGCCGAGTACTACTGTGCCCTGTGGTACTCCGACCACTGGGTCATCGGAGGCGGGA
CCAAGCTGACCGTGCTGGGA(ACACCCCTGGGAGACACCACACATACT)AGTGGGA
AACCTCTGGATGGCGAGTACTTTACCCTGCAGATTAGAGGCCGCGAACGAT
TCGAGATGTTTCGCGAACTGAATGAGGCCCTGGAACTGAAGGATGCTCAGG
CAGGCAAGGAGCCAGGAGGGTCAGGAGGAGCACCGCACCATCATCATCACCAT
HER2 (HL DS) P63 BiDE (LL) polypeptide (hu4D5-scFv,
huC825-scFv, huP63-tet, GS linker, (IgG3 spacer))
SEQ ID NO: 73
EVQLVESGGGLVQPGGSLRLSCAASGFNIKDTYIHWVRQAPGKCLEWVARIYPTNG
YTRYADSVKGRFTISADTSKNTAYLQMNSLRAEDTAVYYCSRWGGDGFYAMDYW
GQGTLVTVSSGGGGSGGGGSGGGGSGGGGSGGGGSGGGGSDIQMTQSPSSLSASVG
DRVTITCRASQDVNTAVAWYQQKPGKAPKLLIYSASFLYSGVPSRFSGSRSGTDFTLT
ISSLQPEDFATYYCQQHYTTPPTFGQCTKVEIKRGGGGSGGGGSGGGGSGGGGSHVQ
LVESGGGLVQPGGSLRLSCAASGFSLTDYGVHWVRQAPGKGLEWLGVIWSGGGTAYNTA
LISRFTISRDNSKNTLYLQMNSLRAEDTAVYYCARRGSYPYNYFDAWGCGTLVTVSSGGGGS
GGGGSGGGGSGGGGSGGGGSGGGGSQAVVTQEPSLTVSPGGTVTLTCGSSTGAVTASNY
ANWVQQKPGQCPRGLIGGHNNRPPGVPARFSGSLLGGKAALTLLGAQPEDEAEYYCALW
YSDHWVIGGGTKLTVLG(TPLGDTTHT)SGRSPDDELLYLPVRGRETYEMLLKIKESL
ELMQYLPQHTIETYRQQQQQQHQHLLQKQGGSGGAPHHHHHH
HER2 (HL DS) P63 BiDE (LL) cDNA (hu4D5-scFv, huC825-scFv,
huP63-tet, GS linker, (IgG3 spacer))
SEQ ID NO: 74
GAAGTGCAGCTGGTCGAATCCGGGGGGGGCCTGGTGCAGCCTGGAGGGTCACTGAG
ACTGTCCTGTGCCGCATCTGGGTTCAATATCAAGGACACCTACATCCACTGGGTGCGG
CAGGCACCTGGCAAGtGtCTGGAGTGGGTGGCAAGGATCTATCCAACCAACGGCTACA
CACGGTATGCCGACTCCGTGAAGGGCCGGTTCACCATCTCCGCCGATACCTCTAAGAA
CACAGCCTACCTGCAGATGAATTCTCTGAGGGCCGAGGATACAGCCGTGTACTATTGC
AGCCGCTGGGGAGGCGACGGCTTCTACGCTATGGACTATTGGGGCCAGGGCACCCTG
GTGACAGTGAGCTCTGGCGGCGGCGGATCCGGAGGAGGAGGCAGCGGCGGAGGA
GGCTCCGGAGGAGGCGGCTCTGGCGGCGGCGGCAGCGGCGGCGGCGGCTCCGAC
ATCCAGATGACCCAGTCCCCATCTAGCCTGAGCGCCTCCGTGGGCGACAGGGTGACC
ATCACATGCCGCGCCAGCCAGGATGTGAATACAGCCGTGGCCTGGTACCAGCAGAAG
CCAGGCAAGGCCCCCAAGCTGCTGATCTACTCTGCCAGCTTCCTGTATAGCGGAGTGC
CATCCCGGTTTTCCGGCAGCCGGAGCGGCACCGACTTCACCCTGACAATCTCCTCTCT
GCAGCCTGAGGATTTTGCCACATACTATTGTCAGCAGCACTATACCACACCCCCTACAT
TCGGACAGTGTACAAAGGTCGAGATCAAACGCGGCGGAGGGGGATCCGGCGGCGGA
GGATCTGGCGGAGGTGGAAGTGGGGGAGGCGGATCTCATGTGCAGCTGGTGGAAA
GCGGAGGCGGCCTGGTGCAGCCTGGGGGATCTCTGAGACTGTCTTGTGCCGCCAGC
GGCTTCTCCCTGACCGATTATGGCGTGCACTGGGTGCGACAGGCCCCTGGCAAAGGA
CTGGAATGGCTGGGAGTGATTTGGAGTGGCGGAGGCACCGCCTACAACACCGCCCTG
ATCTCCCGGTTCACCATCAGCCGGGACAACTCCAAGAACACCCTGTACCTGCAGATGA
ACTCCCTGCGGGCCGAGGACACCGCTGTGTACTACTGCGCCAGACGGGGCTCCTACC
CCTACAACTACTTCGACGCTTGGGGCTGCGGCACCCTCGTGACAGTGTCTAGCGGAG
GGGGAGGTTCTGGGGGCGGAGGTTCAGGTGGTGGTGGTTCCGGGGGTGGTGGCTCT
GGTGGCGGTGGTTCTGGCGGTGGCGGATCTCAGGCTGTCGTGACCCAGGAACCCAG
CCTGACTGTGTCTCCTGGCGGAACCGTGACCCTGACCTGCGGATCTTCTACCGGCGC
TGTGACCGCCAGCAACTACGCCAATTGGGTGCAGCAGAAACCTGGACAGTGCCCTAG
AGGCCTGATCGGCGGCCACAACAACAGACCTCCAGGCGTGCCAGCCCGGTTCTCTGG
ATCTCTGCTGGGCGGAAAGGCCGCTCTGACACTGCTGGGTGCTCAGCCTGAGGACGA
GGCCGAGTACTACTGTGCCCTGTGGTACTCCGACCACTGGGTCATCGGAGGCGGGAC
CAAGCTGACCGTGCTGGGA(ACACCCCTGGGAGACACCACACATACT)AGTGGGAG
ATCCCCCGACGATGAGCTGCTGTACCTGCCTGTGAGGGGCCGGGAGACCTA
TGAAATGCTGCTGAAGATCAAAGAGAGCCTGGAACTGATGCAGTACCTGCC
ACAGCACACCATTGAAACATATAGGCAACAACAGCAGCAGCAGCATCAGCA
TCTGCTGCAGAAGCAGGGAGGGTCAGGAGGAGCACCGCACCATCATCATCACCAT
HER2 (HL) P63 BiDE (LL) polypeptide (hu4D5-scFv,
huC825-scFv, huP63-tet, GS linker, (IgG3 spacer))
SEQ ID NO: 75
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSGGGGSGGGGSGGGGSGGGGSGGGGSGGGGSDIQMTQSPSSLSASVG
DRVTITCRASQDVNTAVAWYQQKPGKAPKLLIYSASFLYSGVPSRFSGSRSGTDFTLT
ISSLQPEDFATYYCQQHYTTPPTFGQGTKVEIKRGGGGSGGGGSGGGGSGGGGSHVQ
LVESGGGLVQPGGSLRLSCAASGFSLTDYGVHWVRQAPGKGLEWLGVIWSGGGTAYNTA
LISRFTISRDNSKNTLYLQMNSLRAEDTAVYYCARRGSYPYNYFDAWGCGTLVTVSSGGGGS
GGGGSGGGGSGGGGSGGGGSGGGGSQAVVTQEPSLTVSPGGTVTLTCGSSTGAVTASNY
ANWVQQKPGQCPRGLIGGHNNRPPGVPARFSGSLLGGKAALTLLGAQPEDEAEYYCALW
YSDHWVIGGGTKLTVLG(TPLGDTTHT)SGRSPDDELLYLPVRGRETYEMLLKIKESL
ELMQYLPQHTIETYRQQQQQQHQHLLQKQGGSGGAPHHHHHH
HER2 (HL) P63 BiDE (LL) cDNA (hu4D5-scFv, huC825-scFv,
huP63-tet, GS linker, (IgG3 spacer))
SEQ ID NO: 76
GAAGTGCAGCTGGTCGAATCCGGGGGGGGCCTGGTGCAGCCTGGAGGGTCACTGAG
ACTGTCCTGTGCCGCATCTGGGTTCAATATCAAGGACACCTACATCCACTGGGTGCGG
CAGGCACCTGGCAAGGGACTGGAGTGGGTGGCAAGGATCTATCCAACCAACGGCTAC
ACACGGTATGCCGACTCCGTGAAGGGCCGGTTCACCATCTCCGCCGATACCTCTAAGA
ACACAGCCTACCTGCAGATGAATTCTCTGAGGGCCGAGGATACAGCCGTGTACTATTG
CAGCCGCTGGGGAGGCGACGGCTTCTACGCTATGGACTATTGGGGCCAGGGCACCCT
GGTGACAGTGAGCTCTGGCGGCGGCGGATCCGGAGGAGGAGGCAGCGGCGGAGG
AGGCTCCGGAGGAGGCGGCTCTGGCGGCGGCGGCAGCGGCGGCGGCGGCTCCGA
CATCCAGATGACCCAGTCCCCATCTAGCCTGAGCGCCTCCGTGGGCGACAGGGTGAC
CATCACATGCCGCGCCAGCCAGGATGTGAATACAGCCGTGGCCTGGTACCAGCAGAA
GCCAGGCAAGGCCCCCAAGCTGCTGATCTACTCTGCCAGCTTCCTGTATAGCGGAGTG
CCATCCCGGTTTTCCGGCAGCCGGAGCGGCACCGACTTCACCCTGACAATCTCCTCTC
TGCAGCCTGAGGATTTTGCCACATAGTATTGTCAGCAGCACTATACCACACCCCCTACA
TTCGGACAGGGGACAAAGGTCGAGATCAAACGCGGCGGAGGGGGATCCGGCGGCG
GAGGATCTGGCGGAGGTGGAAGTGGGGGAGGCGGATCTCATGTGCAGCTGGTGG
AAAGCGGAGGCGGCCTGGTGCAGCCTGGGGGATCTCTGAGACTGTCTTGTGCCGCCA
GCGGCTTCTCCCTGACCGATTATGGCGTGCACTGGGTGCGACAGGCCCCTGGCAAAG
GACTGGAATGGCTGGGAGTGATTTGGAGTGGCGGAGGCACCGCCTACAACACCGCCC
TGATCTCCCGGTTCACCATCAGCCGGGACAACTCCAAGAACACCCTGTACCTGCAGAT
GAACTCCCTGCGGGCCGAGGACACCGCTGTGTACTACTGCGCCAGACGGGGCTCCTA
CCCCTACAACTACTTCGACGCTTGGGGCTGCGGCACCCTCGTGACAGTGTCTAGCGG
AGGGGGAGGTTCTGGGGGCGGAGGTTCAGGTGGTGGTGGTTCCGGGGGTGGTGGCT
CTGGTGGCGGTGGTTCTGGCGGTGGCGGATCTCAGGCTGTCGTGACCCAGGAACCCA
GCCTGACTGTGTCTCCTGGCGGAACCGTGACCCTGACCTGCGGATCTTCTACCGGCG
CTGTGACCGCCAGCAACTACGCCAATTGGGTGCAGCAGAAACCTGGACAGTGCCCTA
GAGGCCTGATCGGCGGCCACAACAACAGACCTCCAGGCGTGCCAGCCCGGTTCTCTG
GATCTCTGCTGGGCGGAAAGGCCGCTCTGACACTGCTGGGTGCTCAGCCTGAGGACG
AGGCCGAGTACTACTGTGCCCTGTGGTACTCCGACCACTGGGTCATCGGAGGCGGGA
CCAAGCTGACCGTGCTGGGA(ACACCCCTGGGAGACACCACACATACT)AGTGGGA
GATCCCCCGACGATGAGCTGCTGTACCTGCCTGTGAGGGGCCGGGAGACCT
ATGAAATGCTGCTGAAGATCAAAGAGAGCCTGGAACTGATGCAGTACCTGC
CACAGCACACCATTGAAACATATAGGCAACAACAGCAGCAGCAGCATCAGC
ATCTGCTGCAGAAGCAGGGAGGGTCAGGAGGAGCACCGCACCATCATCATCAC
CAT
HER2 (LH DS) P63 BiDE (LL) polypeptide (hu4D5-scFv,
huC825-scFv, huP63-tet, GS linker, (IgG3 spacer))
SEQ ID NO: 77
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQCTKVEIKRGGGGSGG
GGSGGGGSGGGGSGGGGSGGGGSEVQLVESGGGLVQPGGSLRLSCAASGFNIKDTY
IHWVRQAPGKCLEWVARIYPTNGYTRYADSVKGRFTISADTSKNTAYLQMNSLRAE
DTAVYYCSRWGGDGFYAMDYWGQGTLVTVSSGGGGSGGGGSGGGGSGGGGSHVQ
LVESGGGLVQPGGSLRLSCAASGFSLTDYGVHWVRQAPGKGLEWLGVIWSGGGTAYNTA
LISRFTISRDNSKNTLYLQMNSLRAEDTAVYYCARRGSYPYNYFDAWGCGTLVTVSSGGGGS
GGGGSGGGGSGGGGSGGGGSGGGGSQAVVTQEPSLTVSPGGTVTLTCGSSTGAVTASNY
ANWVQQKPGQCPRGLIGGHNNRPPGVPARFSGSLLGGKAALTLLGAQPEDEAEYYCALW
YSDHWVIGGGTKLTVLG(TPLGDTTHT)SGRSPDDELLYLPVRGRETYEMLLKIKESL
ELMQYLPQHTIETYRQQQQQQHQHLLQKQGGSGGAPHHHHHH
HER2 (LHDS) P63 BiDE(LL) cDNA (hu4D5-scFv, huC825-scFv,
huP63-tet, GS linker, (IgG3 spacer))
SEQ ID NO: 78
CTATCACTTGTCGGGCTTCTCAGGATGTGAACACCGCCGTGGCCTGGTACCAGCAGAA
GCCAGGCAAGGCCCCCAAGCTGCTGATCTACTCTGCCAGCTTCCTGTATTCCGGAGTG
CCATCTCGGTTTTCCGGCAGCCGGAGCGGCACCGACTTCACCCTGACAATCAGCTCC
CTGCAGCCTGAGGATTTTGCCACATACTATTGCCAGCAGCACTATACCACACCCCCTAC
CTTCGGCCAGtGCACAAAGGTGGAGATCAAGAGGGGAGGAGGAGGATCCGGAGGAG
GAGGCAGCGGAGGCGGCGGCTCCGGCGGCGGCGGCTCTGGCGGCGGCGGCAGC
GGAGGAGGCGGCTCCGAGGTGCAGCTGGTGGAGTCCGGCGGCGGCCTGGTGCAGC
CCGGCGGCAGCCTGCGGCTGTCCTGTGCCGCCTCTGGCTTTAACATCAAGGACACCT
ACATCCACTGGGTGAGGCAGGCACCTGGCAAGtGCCTGGAGTGGGTGGCAAGGATCT
ATCCAACCAATGGCTACACAAGATATGCCGACTCCGTGAAGGGCCGCTTTACCATCAG
CGCCGATACCTCCAAGAACACAGCCTACCTGCAGATGAATTCTCTGCGGGCCGAGGAT
ACAGCCGTGTACTATTGCTCCAGATGGGGCGGCGACGGCTTCTATGCTATGGACTATT
GGGGGCAGGGAACTCTGGTCACTGTCTCCTCTGGCGGAGGGGGATCCGGCGGCGG
AGGATCTGGCGGAGGTGGAAGTGGGGGAGGCGGATCTCATGTGCAGCTGGTGGAA
AGCGGAGGCGGCCTGGTGCAGCCTGGGGGATCTCTGAGACTGTCTTGTGCCGCCAG
CGGCTTCTCCCTGACCGATTATGGCGTGCACTGGGTGCGACAGGCCCCTGGCAAAGG
ACTGGAATGGCTGGGAGTGATTTGGAGTGGCGGAGGCACCGCCTACAACACCGCCCT
GATCTCCCGGTTCACCATCAGCCGGGACAACTCCAAGAACACCCTGTACCTGCAGATG
AACTCCCTGCGGGCCGAGGACACCGCTGTGTACTACTGCGCCAGACGGGGCTCCTAC
CCCTACAACTACTTCGACGCTTGGGGCTGCGGCACCCTCGTGACAGTGTCTAGCGGA
GGGGGAGGTTCTGGGGGCGGAGGTTCAGGTGGTGGTGGTTCCGGGGGTGGTGGCTC
TGGTGGCGGTGGTTCTGGCGGTGGCGGATCTCAGGCTGTCGTGACCCAGGAACCCAG
CCTGACTGTGTCTCCTGGCGGAACCGTGACCCTGACCTGCGGATCTTCTACCGGCGC
TGTGACCGCCAGCAACTACGCCAATTGGGTGCAGCAGAAACCTGGACAGTGCCCTAG
AGGCCTGATCGGCGGCCACAACAACAGACCTCCAGGCGTGCCAGCCCGGTTCTCTGG
ATCTCTGCTGGGCGGAAAGGCCGCTCTGACACTGCTGGGTGCTCAGCCTGAGGACGA
GGCCGAGTACTACTGTGCCCTGTGGTACTCCGACCACTGGGTCATCGGAGGCGGGAC
CAAGCTGACCGTGCTGGGA(ACACCCCTGGGAGACACCACACATACT)AGTGGGAG
ATCCCCCGACGATGAGCTGCTGTACCTGCCTGTGAGGGGCCGGGAGACCTA
TGAAATGCTGCTGAAGATCAAAGAGAGCCTGGAACTGATGCAGTACCTGCC
ACAGCACACCATTGAAACATATAGGCAACAACAGCAGCAGCAGCATCAGCA
TCTGCTGCAGAAGCAGGGAGGGTCAGGAGGAGCACCGCACCATCATCATCACCAT
HER2 (LH) P63 BiDE(LL) polypeptide (hu4D5-scFv, huC825-scFv,
huP63-tet, GS linker, (IgG3 spacer))
SEQ ID NO: 79
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRGGGGSGG
GGSGGGGSGGGGSGGGGSGGGGSEVQLVESGGGLVQPGGSLRLSCAASGFNIKDTY
IHWVRQAPGKGLEWVARIYPTNGYTRYADSVKGRFTISADTSKNTAYLQMNSLRAE
DTAVYYCSRWGGDGFYAMDYWGQGTLVTVSSGGGGSGGGGSGGGGSGGGGSHVQ
LVESGGGLVQPGGSLRLSCAASGFSLTDYGVHWVRQAPGKGLEWLGVIWSGGGTAYNTA
LISRFTISRDNSKNTLYLQMNSLRAEDTAVYYCARRGSYPYNYFDAWGCGTLVTVSSGGGGS
GGGGSGGGGSGGGGSGGGGSGGGGSQAVVTQEPSLTVSPGGTVTLTCGSSTGAVTASNY
ANWVQQKPGQCPRGLIGGHNNRPPGVPARFSGSLLGGKAALTLLGAQPEDEAEYYCALW
YSDHWVIGGGTKLTVLG(TPLGDTTHT)SGRSPDDELLYLPVRGRETYEMLLKIKESL
ELMQYLPQHTIETYRQQQQQQHQHLLQKQGGSGGAPHHHHHH
HER2 (LH) P63 BiDE(LL) cDNA (hu4D5-scFv, huC825-scFv,
huP63-tet, GS linker, (IgG3 spacer))
SEQ ID NO: 80
GATATTCAGATGACTCAGTCCCCTAGTTCACTGTCTGCCTCAGTCGGAGATCGGGTCA
CTATCACTTGTCGGGCTTCTCAGGATGTGAACACCGCCGTGGCCTGGTACCAGCAGAA
GCCAGGCAAGGCCCCCAAGCTGCTGATCTACTCTGCCAGCTTCCTGTATTCCGGAGTG
CCATCTCGGTTTTCCGGCAGCCGGAGCGGCACCGACTTCACCCTGACAATCAGCTCC
CTGCAGCCTGAGGATTTTGCCACATACTATTGCCAGCAGCACTATACCACACCCCCTAC
CTTCGGCCAGGGCACAAAGGTGGAGATCAAGAGGGGAGGAGGAGGATCCGGAGGA
GGAGGCAGCGGAGGCGGCGGCTCCGGCGGCGGCGGCTCTGGCGGCGGCGGCAG
CGGAGGAGGCGGCTCCGAGGTGCAGCTGGTGGAGTCCGGCGGCGGCCTGGTGCAG
CCCGGCGGCAGCCTGCGGCTGTCCTGTGCCGCCTCTGGCTTTAACATCAAGGACACC
TACATCCACTGGGTGAGGCAGGCACCTGGCAAGGGCCTGGAGTGGGTGGCAAGGATC
TATCCAACCAATGGCTACACAAGATATGCCGACTCCGTGAAGGGCCGCTTTACCATCA
GCGCCGATACCTCCAAGAACACAGCCTACCTGCAGATGAATTCTCTGCGGGCCGAGG
ATACAGCCGTGTACTATTGCTCCAGATGGGGCGGCGACGGCTTCTATGCTATGGACTA
TTGGGGGCAGGGAACTCTGGTCACTGTCTCCTCTGGCGGAGGGGGATCCGGCGGCG
GAGGATCTGGCGGAGGTGGAAGTGGGGGAGGCGGATCTCATGTGCAGCTGGTGG
AAAGCGGAGGCGGCCTGGTGCAGCCTGGGGGATCTCTGAGACTGTCTTGTGCCGCCA
GCGGCTTCTCCCTGACCGATTATGGCGTGCACTGGGTGCGACAGGCCCCTGGCAAAG
GACTGGAATGGCTGGGAGTGATTTGGAGTGGCGGAGGCACCGCCTACAACACCGCCC
TGATCTCCCGGTTCACCATCAGCCGGGACAACTCCAAGAACACCCTGTACCTGCAGAT
GAACTCCCTGCGGGCCGAGGACACCGCTGTGTACTACTGCGCCAGACGGGGCTCCTA
CCCCTACAACTACTTCGACGCTTGGGGCTGCGGCACCCTCGTGACAGTGTCTAGCGG
AGGGGGAGGTTCTGGGGGCGGAGGTTCAGGTGGTGGTGGTTCCGGGGGTGGTGGCT
CTGGTGGCGGTGGTTCTGGCGGTGGCGGATCTCAGGCTGTCGTGACCCAGGAACCCA
GCCTGACTGTGTCTCCTGGCGGAACCGTGACCCTGACCTGCGGATCTTCTACCGGCG
CTGTGACCGCCAGCAACTACGCCAATTGGGTGCAGCAGAAACCTGGACAGTGCCCTA
GAGGCCTGATCGGCGGCCACAACAACAGACCTCCAGGCGTGCCAGCCCGGTTCTCTG
GATCTCTGCTGGGCGGAAAGGCCGCTCTGACACTGCTGGGTGCTCAGCCTGAGGACG
AGGCCGAGTACTACTGTGCCCTGTGGTACTCCGACCACTGGGTCATCGGAGGCGGGA
CCAAGCTGACCGTGCTGGGA(ACACCCCTGGGAGACACCACACATACT)AGTGGGA
GATCCCCCGACGATGAGCTGCTGTACCTGCCTGTGAGGGGCCGGGAGACCT
ATGAAATGCTGCTGAAGATCAAAGAGAGCCTGGAACTGATGCAGTACCTGC
CACAGCACACCATTGAAACATATAGGCAACAACAGCAGCAGCAGCATCAGC
ATCTGCTGCAGAAGCAGGGAGGGTCAGGAGGAGCACCGCACCATCATCATCAC
CAT
HER2 (HL DS) P73 BiDE(LL) polypeptide (hu4D5-scFv,
huC825-scFv, huP73-tet, GS linker, (IgG3 spacer))
SEQ ID NO: 81
EVQLVESGGGLVQPGGSLRLSCAASGFNIKDTYIHWVRQAPGKCLEWVARIYPTNG
YTRYADSVKGRFTISADTSKNTAYLQMNSLRAEDTAVYYCSRWGGDGFYAMDYW
GQGTLVTVSSGGGGSGGGGSGGGGSGGGGSGGGGSGGGGSDIQMTQSPSSLSASVG
DRVTITCRASQDVNTAVAWYQQKPGKAPKLLIYSASFLYSGVPSRFSGSRSGTDFTLT
ISSLQPEDFATYYCQQHYTTPPTFGQCTKVEIKRGGGGSGGGGSGGGGSGGGGSHVQ
LVESGGGLVQPGGSLRLSCAASGFSLTDYGVHWVRQAPGKGLEWLGVIWSGGGTAYNTA
LISRFTISRDNSKNTLYLQMNSLRAEDTAVYYCARRGSYPYNYFDAWGCGTLVTVSSGGGGS
GGGGSGGGGSGGGGSGGGGSGGGGSQAVVTQEPSLTVSPGGTVTLTCGSSTGAVTASNY
ANWVQQKPGQCPRGLIGGHNNRPPGVPARFSGSLLGGKAALTLLGAQPEDEAEYYCALW
YSDHWVIGGGTKLTVLG(TPLGDTTHT)SGRHGDEDTYYLQVRGRENFEILMKLKES
LELMELVPQPLVDSYRQQQQLLQRPGGSGGAPHHHHHH
HER2 (HL DS) P73 BiDE(LL) cDNA (hu4D5-scFv, huC825-scFv,
huP73-tet, GS linker, (IgG3 spacer))
SEQ ID NO: 82
GAAGTGCAGCTGGTCGAATCCGGGGGGGGCCTGGTGCAGCCTGGAGGGTCACTGAG
ACTGTCCTGTGCCGCATCTGGGTTCAATATCAAGGACACCTACATCCACTGGGTGCGG
CAGGCACCTGGCAAGtGtCTGGAGTGGGTGGCAAGGATCTATCCAACCAACGGCTACA
CACGGTATGCCGACTCCGTGAAGGGCCGGTTCACCATCTCCGCCGATACCTCTAAGAA
CACAGCCTACCTGCAGATGAATTCTCTGAGGGCCGAGGATACAGCCGTGTACTATTGC
AGCCGCTGGGGAGGCGACGGCTTCTACGCTATGGACTATTGGGGCCAGGGCACCCTG
GTGACAGTGAGCTCTGGCGGCGGCGGATCCGGAGGAGGAGGCAGCGGCGGAGGA
GGCTCCGGAGGAGGCGGCTCTGGCGGCGGCGGCAGCGGCGGCGGCGGCTCCGAC
ATCCAGATGACCCAGTCCCCATCTAGCCTGAGCGCCTCCGTGGGCGACAGGGTGACC
ATCACATGCCGCGCCAGCCAGGATGTGAATACAGCCGTGGCCTGGTACCAGCAGAAG
CCAGGCAAGGCCCCCAAGCTGCTGATCTACTCTGCCAGCTTCCTGTATAGCGGAGTGC
CATCCCGGTTTTCCGGCAGCCGGAGCGGCACCGACTTCACCCTGACAATCTCCTCTCT
GCAGCCTGAGGATTTTGCCACATACTATTGTCAGCAGCACTATACCACACCCCCTACAT
TCGGACAGTGTACAAAGGTCGAGATCAAACGCGGCGGAGGGGGATCCGGCGGCGGA
GGATCTGGCGGAGGTGGAAGTGGGGGAGGCGGATCTCATGTGCAGCTGGTGGAAA
GCGGAGGCGGCCTGGTGCAGCCTGGGGGATCTCTGAGACTGTCTTGTGCCGCCAGC
GGCTTCTCCCTGACCGATTATGGCGTGCACTGGGTGCGACAGGCCCCTGGCAAAGGA
CTGGAATGGCTGGGAGTGATTTGGAGTGGCGGAGGCACCGCCTACAACACCGCCCTG
ATCTCCCGGTTCACCATCAGCCGGGACAACTCCAAGAACACCCTGTACCTGCAGATGA
ACTCCCTGCGGGCCGAGGACACCGCTGTGTACTACTGCGCCAGACGGGGCTCCTACC
CCTACAACTACTTCGACGCTTGGGGCTGCGGCACCCTCGTGACAGTGTCTAGCGGAG
GGGGAGGTTCTGGGGGCGGAGGTTCAGGTGGTGGTGGTTCCGGGGGTGGTGGCTCT
GGTGGCGGTGGTTCTGGCGGTGGCGGATCTCAGGCTGTCGTGACCCAGGAACCCAG
CCTGACTGTGTCTCCTGGCGGAACCGTGACCCTGACCTGCGGATCTTCTACCGGCGC
TGTGACCGCCAGCAACTACGCCAATTGGGTGCAGCAGAAACCTGGACAGTGCCCTAG
AGGCCTGATCGGCGGCCACAACAACAGACCTCCAGGCGTGCCAGCCCGGTTCTCTGG
ATCTCTGCTGGGCGGAAAGGCCGCTCTGACACTGCTGGGTGCTCAGCCTGAGGACGA
GGCCGAGTACTACTGTGCCCTGTGGTACTCCGACCACTGGGTCATCGGAGGCGGGAC
CAAGCTGACCGTGCTGGGA(ACACCCCTGGGAGACACCACACATACT)AGTGGGAG
GCACGGCGACGAAGATACCTACTATCTGCAGGTGAGGGGACGGGAGAACTT
CGAAATCCTGATGAAGCTGAAAGAGTCCCTGGAACTGATGGAGCTGGTGCC
CCAGCCTCTGGTCGACAGCTACAGACAGCAGCAGCAGCTGCTGCAGAGGCC
AGGAGGGTCAGGAGGAGCACCGCACCATCATCATCACCAT
HER2 (HL) P73 BiDE(LL) polypeptide (hu4D5-scFv, huC825-scFv,
huP73-tet, GS linker, (IgG3 spacer))
SEQ ID NO: 83
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSGGGGSGGGGSGGGGSGGGGSGGGGSGGGGSDIQMTQSPSSLSASVG
DRVTITCRASQDVNTAVAWYQQKPGKAPKLLIYSASFLYSGVPSRFSGSRSGTDFTLT
ISSLQPEDFATYYCQQHYTTPPTFGQGTKVEIKRGGGGSGGGGSGGGGSGGGGSHVQ
LVESGGGLVQPGGSLRLSCAASGFSLTDYGVHWVRQAPGKGLEWLGVIWSGGGTAYNTA
LISRFTISRDNSKNTLYLQMNSLRAEDTAVYYCARRGSYPYNYFDAWGCGTLVTVSSGGGGS
GGGGSGGGGSGGGGSGGGGSGGGGSQAVVTQEPSLTVSPGGTVTLTCGSSTGAVTASNY
ANWVQQKPGQCPRGLIGGHNNRPPGVPARFSGSLLGGKAALTLLGAQPEDEAEYYCALW
YSDHWVIGGGTKLTVLG(TPLGDTTHT)SGRHGDEDTYYLQVRGRENFEILMKLKES
LELMELVPQPLVDSYRQQQQLLQRPGGSGGAPHHHHHH
HER2 (HL) P73 BiDE(LL) cDNA (hu4D5-scFv, huC825-scFv,
huP73-tet, GS linker, (IgG3 spacer))
SEQ ID NO: 84
GAAGTGCAGCTGGTCGAATCCGGGGGGGGCCTGGTGCAGCCTGGAGGGTCACTGAG
ACTGTCCTGTGCCGCATCTGGGTTCAATATCAAGGACACCTACATCCACTGGGTGCGG
CAGGCACCTGGCAAGGGACTGGAGTGGGTGGCAAGGATCTATCCAACCAACGGCTAC
ACACGGTATGCCGACTCCGTGAAGGGCCGGTTCACCATCTCCGCCGATACCTCTAAGA
ACACAGCCTACCTGCAGATGAATTCTCTGAGGGCCGAGGATACAGCCGTGTACTATTG
CAGCCGCTGGGGAGGCGACGGCTTCTACGCTATGGACTATTGGGGCCAGGGCACCCT
GGTGACAGTGAGCTCTGGCGGCGGCGGATCCGGAGGAGGAGGCAGCGGCGGAGG
AGGCTCCGGAGGAGGCGGCTCTGGCGGCGGCGGCAGCGGCGGCGGCGGCTCCGA
CATCCAGATGACCCAGTCCCCATCTAGCCTGAGCGCCTCCGTGGGCGACAGGGTGAC
CATCACATGCCGCGCCAGCCAGGATGTGAATACAGCCGTGGCCTGGTACCAGCAGAA
GCCAGGCAAGGCCCCCAAGCTGCTGATCTACTCTGCCAGCTTCCTGTATAGCGGAGTG
CCATCCCGGTTTTCCGGCAGCCGGAGCGGCACCGACTTCACCCTGACAATCTCCTCTC
TGCAGCCTGAGGATTTTGCCACATACTATTGTCAGCAGCACTATACCACACCCCCTACA
TTCGGACAGGGGACAAAGGTCGAGATCAAACGCGGCGGAGGGGGATCCGGCGGCG
GAGGATCTGGCGGAGGTGGAAGTGGGGGAGGCGGATCTCATGTGCAGCTGGTGG
AAAGCGGAGGCGGCCTGGTGCAGCCTGGGGGATCTCTGAGACTGTCTTGTGCCGCCA
GCGGCTTCTCCCTGACCGATTATGGCGTGCACTGGGTGCGACAGGCCCCTGGCAAAG
GACTGGAATGGCTGGGAGTGATTTGGAGTGGCGGAGGCACCGCCTACAACACCGCCC
TGATCTCCCGGTTCACCATCAGCCGGGACAACTCCAAGAACACCCTGTACCTGCAGAT
GAACTCCCTGCGGGCCGAGGACACCGCTGTGTACTACTGCGCCAGACGGGGCTCCTA
CCCCTACAACTACTTCGACGCTTGGGGCTGCGGCACCCTCGTGACAGTGTCTAGCGG
AGGGGGAGGTTCTGGGGGCGGAGGTTCAGGTGGTGGTGGTTCCGGGGGTGGTGGCT
CTGGTGGCGGTGGTTCTGGCGGTGGCGGATCTCAGGCTGTCGTGACCCAGGAACCCA
GCCTGACTGTGTCTCCTGGCGGAACCGTGACCCTGACCTGCGGATCTTCTACCGGCG
CTGTGACCGCCAGCAACTACGCCAATTGGGTGCAGCAGAAACCTGGACAGTGCCCTA
GAGGCCTGATCGGCGGCCACAACAACAGACCTCCAGGCGTGCCAGCCCGGTTCTCTG
GATCTCTGCTGGGCGGAAAGGCCGCTCTGACACTGCTGGGTGCTCAGCCTGAGGACG
AGGCCGAGTACTACTGTGCCCTGTGGTACTCCGACCACTGGGTCATCGGAGGCGGGA
CCAAGCTGACCGTGCTGGGA(ACACCCCTGGGAGACACCACACATACT)AGTGGGA
GGCACGGCGACGAAGATACCTACTATCTGCAGGTGAGGGGACGGGAGAACT
TCGAAATCCTGATGAAGCTGAAAGAGTCCCTGGAACTGATGGAGCTGGTGC
CCCAGCCTCTGGTCGACAGCTACAGACAGCAGCAGCAGCTGCTGCAGAGGC
CAGGAGGGTCAGGAGGAGCACCGCACCATCATCATCACCAT
HER2 (LH DS) P73 BiDE(LL) polypeptide (hu4D5-scFv,
huC825-scFv, huP73-tet, GS linker, (IgG3 spacer))
SEQ ID NO: 85
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQCTKVEIKRGGGGSGG
GGSGGGGSGGGGSGGGGSGGGGSEVQLVESGGGLVQPGGSLRLSCAASGFNIKDTY
IHWVRQAPGKCLEWVARIYPTNGYTRYADSVKGRFTISADTSKNTAYLQMNSLRAE
DTAVYYCSRWGGDGFYAMDYWGQGTLVTVSSGGGGSGGGGSGGGGSGGGGSHVQ
LVESGGGLVQPGGSLRLSCAASGFSLTDYGVHWVRQAPGKGLEWLGVIWSGGGTAYNTA
LISRFTISRDNSKNTLYLQMNSLRAEDTAVYYCARRGSYPYNYFDAWGCGTLVTVSSGGGGS
GGGGSGGGGSGGGGSGGGGSGGGGSQAVVTQEPSLTVSPGGTVTLTCGSSTGAVTASNY
ANWVQQKPGQCPRGLIGGHNNRPPGVPARFSGSLLGGKAALTLLGAQPEDEAEYYCALW
YSDHWVIGGGTKLTVLG(TPLGDTTHT)SGRHGDEDTYYLQVRGRENFEILMKLKES
LELMELVPQPLVDSYRQQQQLLQRPGGSGGAPHHHHHH
HER2 (LHDS) P73 BiDE(LL) cDNA (hu4D5-scFv, huC825-scFv,
huP73-tet, GS linker, (IgG3 spacer))
SEQ ID NO: 86
GATATTCAGATGACTCAGTCCCCTAGTTCACTGTCTGCCTCAGTCGGAGATCGGGTCA
CTATCACTTGTCGGGCTTCTCAGGATGTGAACACCGCCGTGGCCTGGTACCAGCAGAA
GCCAGGCAAGGCCCCCAAGCTGCTGATCTACTCTGCCAGCTTCCTGTATTCCGGAGTG
CCATCTCGGTTTTCCGGCAGCCGGAGCGGCACCGACTTCACCCTGACAATCAGCTCC
CTGCAGCCTGAGGATTTTGCCACATACTATTGCCAGCAGCACTATACCACACCCCCTAC
CTTCGGCCAGtGCACAAAGGTGGAGATCAAGAGGGGAGGAGGAGGATCCGGAGGAG
GAGGCAGCGGAGGCGGCGGCTCCGGCGGCGGCGGCTCTGGCGGCGGCGGCAGC
GGAGGAGGCGGCTCCGAGGTGCAGCTGGTGGAGTCCGGCGGCGGCCTGGTGCAGC
CCGGCGGCAGCCTGCGGCTGTCCTGTGCCGCCTCTGGCTTTAACATCAAGGACACCT
ACATCCACTGGGTGAGGCAGGCACCTGGCAAGtGCCTGGAGTGGGTGGCAAGGATCT
ATCCAACCAATGGCTACACAAGATATGCCGACTCCGTGAAGGGCCGCTTTACCATCAG
CGCCGATACCTCCAAGAACACAGCCTACCTGCAGATGAATTCTCTGCGGGCCGAGGAT
ACAGCCGTGTACTATTGCTCCAGATGGGGCGGCGACGGCTTCTATGCTATGGACTATT
GGGGGCAGGGAACTCTGGTCACTGTCTCCTCTGGCGGAGGGGGATCCGGCGGCGG
AGGATGTGGCGGAGGTGGAAGTGGGGGAGGCGGATCTCATGTGCAGCTGGTGGAA
AGCGGAGGCGGCCTGGTGCAGCCTGGGGGATCTCTGAGACTGTCTTGTGCCGCCAG
CGGCTTCTCCCTGACCGATTATGGCGTGCACTGGGTGCGACAGGCCCCTGGCAAAGG
ACTGGAATGGCTGGGAGTGATTTGGAGTGGCGGAGGCACCGCCTACAACACCGCCCT
GATCTCCCGGTTCACCATCAGCCGGGACAACTCCAAGAACACCCTGTACCTGCAGATG
AACTCCCTGCGGGCCGAGGACACCGCTGTGTACTACTGCGCCAGACGGGGCTCCTAC
CCCTACAACTACTTCGACGCTTGGGGCTGCGGCACCCTCGTGACAGTGTCTAGCGGA
GGGGGAGGTTCTGGGGGCGGAGGTTCAGGTGGTGGTGGTTCCGGGGGTGGTGGCTC
TGGTGGCGGTGGTTCTGGCGGTGGCGGATCTCAGGCTGTCGTGACCCAGGAACCCAG
CCTGACTGTGTCTCCTGGCGGAACCGTGACCCTGACCTGCGGATCTTCTACCGGCGC
TGTGACCGCCAGCAACTACGCCAATTGGGTGCAGCAGAAACCTGGACAGTGCCCTAG
AGGCCTGATCGGCGGCCACAACAACAGACCTCCAGGCGTGCCAGCCCGGTTCTCTGG
ATCTCTGCTGGGCGGAAAGGCCGCTCTGACACTGCTGGGTGCTCAGCCTGAGGACGA
GGCCGAGTACTACTGTGCCCTGTGGTACTCCGACCACTGGGTCATCGGAGGCGGGAC
CAAGCTGACCGTGCTGGGA(ACACCCCTGGGAGACACCACACATACT)AGTGGGAG
GCACGGCGACGAAGATACCTACTATCTGCAGGTGAGGGGACGGGAGAACTT
CGAAATCCTGATGAAGCTGAAAGAGTCCCTGGAACTGATGGAGCTGGTGCC
CCAGCCTCTGGTCGACAGCTACAGACAGCAGCAGCAGCTGCTGCAGAGGCC
AGGAGGGTCAGGAGGAGCACCGCACCATCATCATCACCAT
HER2 (LH) P73 BiDE(LL) polypeptide (hu4D5-scFv, huC825-
scFv, huP73-tet, GS linker, (IgG3 spacer))
SEQ ID NO: 87
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRGGGGSGG
GGSGGGGSGGGGSGGGGSGGGGSEVQLVESGGGLVQPGGSLRLSCAASGFNIKDTY
IHWVRQAPGKGLEWVARIYPTNGYTRYADSVKGRFTISADTSKNTAYLQMNSLRAE
DTAVYYCSRWGGDGFYAMDYWGQGTLVTVSSGGGGSGGGGSGGGGSGGGGSHVQ
LVESGGGLVQPGGSLRLSCAASGFSLTDYGVHWVRQAPGKGLEWLGVIWSGGGTAYNTA
LISRFTISRDNSKNTLYLQMNSLRAEDTAVYYCARRGSYPYNYFDAWGCGTLVTVSSGGGGS
GGGGSGGGGSGGGGSGGGGSGGGGSQAVVTQEPSLTVSPGGTVTLTCGSSTGAVTASNY
ANWVQQKPGQCPRGLIGGHNNRPPGVPARFSGSLLGGKAALTLLGAQPEDEAEYYCALW
YSDHWVIGGGTKLTVLG(TPLGDTTHT)SGRHGDEDTYYLQVRGRENFEILMKLKES
LELMELVPQPLVDSYRQQQQLLQRPGGSGGAPHHHHHH
HER2 (LH) P73 BiDE(LL) cDNA (hu4D5-scFv, huC825-scFv,
huP73-tet, GS linker, (IgG3 spacer))
SEQ ID NO: 88
GATATTCAGATGACTCAGTCCCCTAGTTCACTGTCTGCCTCAGTCGGAGATCGGGTCA
CTATCACTTGTCGGGCTTCTCAGGATGTGAACACCGCCGTGGCCTGGTACCAGCAGAA
GCCAGGCAAGGCCCCCAAGCTGCTGATCTACTCTGCCAGCTTCCTGTATTCCGGAGTG
CCATCTCGGTTTTCCGGCAGCCGGAGCGGCACCGACTTCACCCTGACAATCAGCTCC
CTGCAGCCTGAGGATTTTGCCACATACTATTGCCAGCAGCACTATACCACACCCCCTAC
CTTCGGCCAGGGCACAAAGGTGGAGATCAAGAGGGGAGGAGGAGGATCCGGAGGA
GGAGGCAGCGGAGGCGGCGGCTCCGGCGGCGGCGGCTCTGGCGGCGGCGGCAG
CGGAGGAGGCGGCTCCGAGGTGCAGCTGGTGGAGTCCGGCGGCGGCCTGGTGCAG
CCCGGCGGCAGCCTGCGGCTGTCCTGTGCCGCCTCTGGCTTTAACATCAAGGACACC
TACATCCACTGGGTGAGGCAGGCACCTGGCAAGGGCCTGGAGTGGGTGGCAAGGATC
TATCCAACCAATGGCTACACAAGATATGCCGACTCCGTGAAGGGCCGCTTTACCATCA
GCGCCGATACCTCCAAGAACACAGCCTACCTGCAGATGAATTCTCTGCGGGCCGAGG
ATACAGCCGTGTACTATTGCTCCAGATGGGGCGGCGACGGCTTCTATGCTATGGACTA
TTGGGGGCAGGGAACTCTGGTCACTGTCTCCTCTGGCGGAGGGGGATCCGGCGGCG
GAGGATCTGGCGGAGGTGGAAGTGGGGGAGGCGGATCTCATGTGCAGCTGGTGG
AAAGCGGAGGCGGCCTGGTGCAGCCTGGGGGATCTCTGAGACTGTCTTGTGCCGCCA
GCGGCTTCTCCCTGACCGATTATGGCGTGCACTGGGTGCGACAGGCCCCTGGCAAAG
GACTGGAATGGCTGGGAGTGATTTGGAGTGGCGGAGGCACCGCCTACAACACCGCCC
TGATCTCCCGGTTCACCATCAGCCGGGACAACTCCAAGAACACCCTGTACCTGCAGAT
GAACTCCCTGCGGGCCGAGGACACCGCTGTGTACTACTGCGCCAGACGGGGCTCCTA
CCCCTACAACTACTTCGACGCTTGGGGCTGCGGCACCCTCGTGACAGTGTCTAGCGG
AGGGGGAGGTTCTGGGGGCGGAGGTTCAGGTGGTGGTGGTTCCGGGGGTGGTGGCT
CTGGTGGCGGTGGTTCTGGCGGTGGCGGATCTCAGGCTGTCGTGACCCAGGAACCCA
GCCTGACTGTGTCTCCTGGCGGAACCGTGACCCTGACCTGCGGATCTTCTACCGGCG
CTGTGACCGCCAGCAACTACGCCAATTGGGTGCAGCAGAAACCTGGACAGTGCCCTA
GAGGCCTGATCGGCGGCCACAACAACAGACCTCCAGGCGTGCCAGCCCGGTTCTCTG
GATCTCTGCTGGGCGGAAAGGCCGCTCTGACACTGCTGGGTGCTCAGCCTGAGGACG
AGGCCGAGTACTACTGTGCCCTGTGGTACTCCGACCACTGGGTCATCGGAGGCGGGA
CCAAGCTGACCGTGCTGGGA(ACACCCCTGGGAGACACCACACATACT)AGTGGGA
GGCACGGCGACGAAGATACCTACTATCTGCAGGTGAGGGGACGGGAGAACT
TCGAAATCCTGATGAAGCTGAAAGAGTCCCTGGAACTGATGGAGCTGGTGC
CCCAGCCTCTGGTCGACAGCTACAGACAGCAGCAGCAGCTGCTGCAGAGGC
CAGGAGGGTCAGGAGGAGCACCGCACCATCATCATCACCAT
HER2 (HL DS) HNRNPC BiDE(LL) polypeptide (hu4D5-scFv,
huC825-scFv, huHNRNPC-tet, GS linker, (IgG3 spacer))
SEQ ID NO: 89
EVQLVESGGGLVQPGGSLRLSCAASGFNIKDTYIHWVRQAPGKCLEWVARIYPTNG
YTRYADSVKGRFTISADTSKNTAYLQMNSLRAEDTAVYYCSRWGGDGFYAMDYW
GQGTLVTVSSGGGGSGGGGSGGGGSGGGGSGGGGSGGGGSDIQMTQSPSSLSASVG
DRVTITCRASQDVNTAVAWYQQKPGKAPKLLIYSASFLYSGVPSRFSGSRSGTDFTLT
ISSLQPEDFATYYCQQHYTTPPTFGQCTKVEIKRGGGGSGGGGSGGGGSGGGGSHVQ
LVESGGGLVQPGGSLRLSCAASGFSLTDYGVHWVRQAPGKGLEWLGVIWSGGGTAYNTA
LISRFTISRDNSKNTLYLQMNSLRAEDTAVYYCARRGSYPYNYFDAWGCGTLVTVSSGGGGS
GGGGSGGGGSGGGGSGGGGSGGGGSQAVVTQEPSLTVSPGGTVTLTCGSSTGAVTASNY
ANWVQQKPGQCPRGLIGGHNNRPPGVPARFSGSLLGGKAALTLLGAQPEDEAEYYCALW
YSDHWVIGGGTKLTVLG(TPLGDTTHT)SGQAIKKELTQIKQKVDSLLENLEKIEKEG
GSGGAPHHHHHH
HER2 (HL DS) HNRNPC BiDE(LL) cDNA (hu4D5-scFv,
huC825-scFv, huHNRNPC-tet, GS linker, (IgG3 spacer))
SEQ ID NO: 90
GAAGTGCAGCTGGTCGAATCCGGGGGGGGCCTGGTGCAGCCTGGAGGGTCACTGAG
ACTGTCCTGTGCCGCATCTGGGTTCAATATCAAGGACACCTACATCCACTGGGTGCGG
CAGGCACCTGGCAAGtGtCTGGAGTGGGTGGCAAGGATCTATCCAACCAACGGCTACA
CACGGTATGCCGACTCCGTGAAGGGCCGGTTCACCATCTCCGCCGATACCTCTAAGAA
CACAGCCTACCTGCAGATGAATTCTCTGAGGGCCGAGGATACAGCCGTGTACTATTGC
AGCCGCTGGGGAGGCGACGGCTTCTACGCTATGGACTATTGGGGCCAGGGCACCCTG
GTGACAGTGAGCTCTGGCGGCGGCGGATCCGGAGGAGGAGGCAGCGGCGGAGGA
GGCTCCGGAGGAGGCGGCTCTGGCGGCGGCGGCAGCGGCGGCGGCGGCTCCGAC
ATCCAGATGACCCAGTCCCCATCTAGCCTGAGCGCCTCCGTGGGCGACAGGGTGACC
ATCACATGCCGCGCCAGCCAGGATGTGAATACAGCCGTGGCCTGGTACCAGCAGAAG
CCAGGCAAGGCCCCCAAGCTGCTGATCTACTCTGCCAGCTTCCTGTATAGCGGAGTGC
CATCCCGGTTTTCCGGCAGCCGGAGCGGCACCGACTTCACCCTGACAATCTCCTCTCT
GCAGCCTGAGGATTTTGCCACATACTATTGTCAGCAGCACTATACCACACCCCCTACAT
TCGGACAGTGTACAAAGGTCGAGATCAAACGCGGCGGAGGGGGATCCGGCGGCGGA
GGATCTGGCGGAGGTGGAAGTGGGGGAGGCGGATCTCATGTGCAGCTGGTGGAAA
GCGGAGGCGGCCTGGTGCAGCCTGGGGGATCTCTGAGACTGTCTTGTGCCGCCAGC
GGCTTCTCCCTGACCGATTATGGCGTGCACTGGGTGCGACAGGCCCCTGGCAAAGGA
CTGGAATGGCTGGGAGTGATTTGGAGTGGCGGAGGCACCGCCTACAACACCGCCCTG
ATCTCCCGGTTCACCATCAGCCGGGACAACTCCAAGAACACCCTGTACCTGCAGATGA
ACTCCCTGCGGGCCGAGGACACCGCTGTGTACTACTGCGCCAGACGGGGCTCCTACC
CCTACAACTACTTCGACGCTTGGGGCTGCGGCACCCTCGTGACAGTGTCTAGCGGAG
GGGGAGGTTCTGGGGGCGGAGGTTCAGGTGGTGGTGGTTCCGGGGGTGGTGGCTCT
GGTGGCGGTGGTTCTGGCGGTGGCGGATCTCAGGCTGTCGTGACCCAGGAACCCAG
CCTGACTGTGTCTCCTGGCGGAACCGTGACCCTGACCTGCGGATCTTCTACCGGCGC
TGTGACCGCCAGCAACTACGCCAATTGGGTGCAGCAGAAACCTGGACAGTGCCCTAG
AGGCCTGATCGGCGGCCACAACAACAGACCTCCAGGCGTGCCAGCCCGGTTCTCTGG
ATCTCTGCTGGGCGGAAAGGCCGCTCTGACACTGCTGGGTGCTCAGCCTGAGGACGA
GGCCGAGTACTACTGTGCCCTGTGGTACTCCGACCACTGGGTCATCGGAGGCGGGAC
CAAGCTGACCGTGCTGGGA(ACACCCCTGGGAGACACCACACATACT)AGTGGGCA
GGCCATCAAGAAGGAGCTGACCCAGATCAAGCAGAAGGTGGACAGCCTGCT
GGAGAACCTGGAGAAGATCGAGAAGGAGGGAGGGTCAGGAGGAGCACCGCA
CCATCATCATCACCAT
HER2 (HL) HNRNPC BiDE(LL) polypeptide (hu4D5-scFv,
huC825-scFv, huHNRNPC-tet, GS linker, (IgG3 spacer))
SEQ ID NO: 91
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSGGGGSGGGGSGGGGSGGGGSGGGGSGGGGSDIQMTQSPSSLSASVG
DRVTITCRASQDVNTAVAWYQQKPGKAPKLLIYSASFLYSGVPSRFSGSRSGTDFTLT
ISSLQPEDFATYYCQQHYTTPPTFGQGTKVEIKRGGGGSGGGGSGGGGSGGGGSHVQ
LVESGGGLVQPGGSLRLSCAASGFSLTDYGVHWVRQAPGKGLEWLGVIWSGGGTAYNTA
LISRFTISRDNSKNTLYLQMNSLRAEDTAVYYCARRGSYPYNYFDAWGCGTLVTVSSGGGGS
GGGGSGGGGSGGGGSGGGGSGGGGSQAVVTQEPSLTVSPGGTVTLTCGSSTGAVTASNY
ANWVQQKPGQCPRGLIGGHNNRPPGVPARFSGSLLGGKAALTLLGAQPEDEAEYYCALW
YSDHWVIGGGTKLTVLG(TPLGDTTHT)SGQAIKKELTQIKQKVDSLLENLEKIEKEG
GSGGAPHHHHHH
HER2 (HL) HNRNPC BiDE(LL) cDNA (hu4D5-scFv,
huC825-scFv, huHNRNPC-tet, GS linker, (IgG3 spacer))
SEQ ID NO: 92
GAAGTGCAGCTGGTCGAATCCGGGGGGGGCCTGGTGCAGCCTGGAGGGTCACTGAG
ACTGTCCTGTGCCGCATCTGGGTTCAATATCAAGGACACCTACATCCACTGGGTGCGG
CAGGCACCTGGCAAGGGACTGGAGTGGGTGGCAAGGATCTATCCAACCAACGGCTAC
ACACGGTATGCCGACTCCGTGAAGGGCCGGTTCACCATCTCCGCCGATACCTCTAAGA
ACACAGCCTACCTGCAGATGAATTCTCTGAGGGCCGAGGATACAGCCGTGTACTATTG
CAGCCGCTGGGGAGGCGACGGCTTCTACGCTATGGACTATTGGGGCCAGGGCACCCT
GGTGACAGTGAGCTCTGGCGGCGGCGGATCCGGAGGAGGAGGCAGCGGCGGAGG
AGGCTCCGGAGGAGGCGGCTCTGGCGGCGGCGGCAGCGGCGGCGGCGGCTCCGA
CATCCAGATGACCCAGTCCCCATCTAGCCTGAGCGCCTCCGTGGGCGACAGGGTGAC
CATCACATGCCGCGCCAGCCAGGATGTGAATACAGCCGTGGCCTGGTACCAGCAGAA
GCCAGGCAAGGCCCCCAAGCTGCTGATCTACTCTGCCAGCTTCCTGTATAGCGGAGTG
CCATCCCGGTTTTCCGGCAGCCGGAGCGGCACCGACTTCACCCTGACAATCTCCTCTC
TGCAGCCTGAGGATTTTGCCACATAGTATTGTCAGCAGCACTATACCACACCCCCTACA
TTCGGACAGGGGACAAAGGTCGAGATCAAACGCGGCGGAGGGGGATCCGGCGGCG
GAGGATCTGGCGGAGGTGGAAGTGGGGGAGGCGGATCTCATGTGCAGCTGGTGG
AAAGCGGAGGCGGCCTGGTGCAGCCTGGGGGATCTCTGAGACTGTCTTGTGCCGCCA
GCGGCTTCTCCCTGACCGATTATGGCGTGCACTGGGTGCGACAGGCCCCTGGCAAAG
GACTGGAATGGCTGGGAGTGATTTGGAGTGGCGGAGGCACCGCCTACAACACCGCCC
TGATCTCCCGGTTCACCATCAGCCGGGACAACTCCAAGAACACCCTGTACCTGCAGAT
GAACTCCCTGCGGGCCGAGGACACCGCTGTGTACTACTGCGCCAGACGGGGCTCCTA
CCCCTACAACTACTTCGACGCTTGGGGCTGCGGCACCCTCGTGACAGTGTCTAGCGG
AGGGGGAGGTTCTGGGGGCGGAGGTTCAGGTGGTGGTGGTTCCGGGGGTGGTGGCT
CTGGTGGCGGTGGTTCTGGCGGTGGCGGATCTCAGGCTGTCGTGACCCAGGAACCCA
GCCTGACTGTGTCTCCTGGCGGAACCGTGACCCTGACCTGCGGATCTTCTACCGGCG
CTGTGACCGCCAGCAACTACGCCAATTGGGTGCAGCAGAAACCTGGACAGTGCCCTA
GAGGCCTGATCGGCGGCCACAACAACAGACCTCCAGGCGTGCCAGCCCGGTTCTCTG
GATCTCTGCTGGGCGGAAAGGCCGCTCTGACACTGCTGGGTGCTCAGCCTGAGGACG
AGGCCGAGTACTACTGTGCCCTGTGGTACTCCGACCACTGGGTCATCGGAGGCGGGA
CCAAGCTGACCGTGCTGGGA(ACACCCCTGGGAGACACCACACATACT)
AGTGGGCAGGCCATCAAGAAGGAGCTGACCCAGATCAAGCAGAAGGTGGAC
AGCCTGCTGGAGAACCTGGAGAAGATCGAGAAGGAGGGAGGGTCAGGAGGA
GCACCGCACCATCATCATCACCAT
HER2 (LH DS) HNRNPC BiDE(LL) polypeptide (hu4D5-scFv,
huC825-scFv, huHNRNPC-tet, GS linker, (IgG3 spacer))
SEQ ID NO: 93
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQCTKVEIKRGGGGSGG
GGSGGGGSGGGGSGGGGSGGGGSEVQLVESGGGLVQPGGSLRLSCAASGFNIKDTY
IHWVRQAPGKCLEWVARIYPTNGYTRYADSVKGRFTISADTSKNTAYLQMNSLRAE
DTAVYYCSRWGGDGFYAMDYWGQGTLVTVSSGGGGSGGGGSGGGGSGGGGSHVQ
LVESGGGLVQPGGSLRLSCAASGFSLTDYGVHWVRQAPGKGLEWLGVIWSGGGTAYNTA
LISRFTISRDNSKNTLYLQMNSLRAEDTAVYYCARRGSYPYNYFDAWGCGTLVTVSSGGGGS
GGGGSGGGGSGGGGSGGGGSGGGGSQAVVTQEPSLTVSPGGTVTLTCGSSTGAVTASNY
ANWVQQKPGQCPRGLIGGHNNRPPGVPARFSGSLLGGKAALTLLGAQPEDEAEYYCALW
YSDHWVIGGGTKLTVLG(TPLGDTTHT)SGQAIKKELTQIKQKVDSLLENLEKIEKEG
GSGGAPHHHHHH
HER2 (LHDS) HNRNPC BiDE(LL) cDNA (hu4D5-scFv,
huC825-scFv, huHNRNPC-tet, GS linker, (IgG3 spacer))
SEQ ID NO: 94
GATATTCAGATGACTCAGTCCCCTAGTTCACTGTCTGCCTCAGTCGGAGATCGGGTCA
CTATCACTTGTCGGGCTTCTCAGGATGTGAACACCGCCGTGGCCTGGTACCAGCAGAA
GCCAGGCAAGGCCCCCAAGCTGCTGATCTACTCTGCCAGCTTCCTGTATTCCGGAGTG
CCATCTCGGTTTTCCGGCAGCCGGAGCGGCACCGACTTCACCCTGACAATCAGCTCC
CTGCAGCCTGAGGATTTTGCCACATACTATTGCCAGCAGCACTATACCACACCCCCTAC
CTTCGGCCAGtGCACAAAGGTGGAGATCAAGAGGGGAGGAGGAGGATCCGGAGGAG
GAGGCAGCGGAGGCGGCGGCTCCGGCGGCGGCGGCTCTGGCGGCGGCGGCAGC
GGAGGAGGCGGCTCCGAGGTGCAGCTGGTGGAGTCCGGCGGCGGCCTGGTGCAGC
CCGGCGGCAGCCTGCGGCTGTCCTGTGCCGCCTCTGGCTTTAACATCAAGGACACCT
ACATCCACTGGGTGAGGCAGGCACCTGGCAAGtGCCTGGAGTGGGTGGCAAGGATCT
ATCCAACCAATGGCTACACAAGATATGCCGACTCCGTGAAGGGCCGCTTTACCATCAG
CGCCGATACCTCCAAGAACACAGCCTACCTGCAGATGAATTCTCTGCGGGCCGAGGAT
ACAGCCGTGTACTATTGCTCCAGATGGGGCGGCGACGGCTTCTATGCTATGGACTATT
GGGGGCAGGGAACTCTGGTCACTGTCTCCTCTGGCGGAGGGGGATCCGGCGGCGG
AGGATCTGGCGGAGGTGGAAGTGGGGGAGGCGGATCTCATGTGCAGCTGGTGGAA
AGCGGAGGCGGCCTGGTGCAGCCTGGGGGATCTCTGAGACTGTCTTGTGCCGCCAG
CGGCTTCTCCCTGACCGATTATGGCGTGCACTGGGTGCGACAGGCCCCTGGCAAAGG
ACTGGAATGGCTGGGAGTGATTTGGAGTGGCGGAGGCACCGCCTACAACACCGCCCT
GATCTCCCGGTTCACCATCAGCCGGGACAACTCCAAGAACACCCTGTACCTGCAGATG
AACTCCCTGCGGGCCGAGGACACCGCTGTGTACTACTGCGCCAGACGGGGCTCCTAC
CCCTACAACTACTTCGACGCTTGGGGCTGCGGCACCCTCGTGACAGTGTCTAGCGGA
GGGGGAGGTTCTGGGGGCGGAGGTTCAGGTGGTGGTGGTTCCGGGGGTGGTGGCTC
TGGTGGCGGTGGTTCTGGCGGTGGCGGATCTCAGGCTGTCGTGACCCAGGAACCCAG
CCTGACTGTGTCTCCTGGCGGAACCGTGACCCTGACCTGCGGATCTTCTACCGGCGC
TGTGACCGCCAGCAACTACGCCAATTGGGTGCAGCAGAAACCTGGACAGTGCCCTAG
AGGCCTGATCGGCGGCCACAACAACAGACCTCCAGGCGTGCCAGCCCGGTTCTCTGG
ATCTCTGCTGGGCGGAAAGGCCGCTCTGACACTGCTGGGTGCTCAGCCTGAGGACGA
GGCCGAGTACTACTGTGCCCTGTGGTACTCCGACCACTGGGTCATCGGAGGCGGGAC
CAAGCTGACCGTGCTGGGA(ACACCCCTGGGAGACACCACACATACT)AGTGGGCA
GGCCATCAAGAAGGAGCTGACCCAGATCAAGCAGAAGGTGGACAGCCTGCT
GGAGAACCTGGAGAAGATCGAGAAGGAGGGAGGGTCAGGAGGAGCACCGCA
CCATCATCATCACCAT
HER2 (LH) HNRNPC BiDE(LL) polypeptide (hu4D5-scFv,
huC825-scFv, huHNRNPC-tet, GS linker, (IgG3 spacer))
SEQ ID NO: 95
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRGGGGSGG
GGSGGGGSGGGGSGGGGSGGGGSEVQLVESGGGLVQPGGSLRLSCAASGFNIKDTY
IHWVRQAPGKGLEWVARIYPTNGYTRYADSVKGRFTISADTSKNTAYLQMNSLRAE
DTAVYYCSRWGGDGFYAMDYWGQGTLVTVSSGGGGSGGGGSGGGGSGGGGSHVQ
LVESGGGLVQPGGSLRLSCAASGFSLTDYGVHWVRQAPGKGLEWLGVIWSGGGTAYNTA
LISRFTISRDNSKNTLYLQMNSLRAEDTAVYYCARRGSYPYNYFDAWGCGTLVTVSSGGGGS
GGGGSGGGGSGGGGSGGGGSGGGGSQAVVTQEPSLTVSPGGTVTLTCGSSTGAVTASNY
ANWVQQKPGQCPRGLIGGHNNRPPGVPARFSGSLLGGKAALTLLGAQPEDEAEYYCALW
YSDHWVIGGGTKLTVLG(TPLGDTTHT)SGQAIKKELTQIKQKVDSLLENLEKIEKEG
GSGGAPHHHHHH
HER2 (LH) HNRNPC BiDE(LL) cDNA (hu4D5-scFv,
huC825-scFv, huHNRNPC-tet, GS linker, (IgG3 spacer))
SEQ ID NO: 96
GATATTCAGATGACTCAGTCCCCTAGTTCACTGTCTGCCTCAGTCGGAGATCGGGTCA
CTATCACTTGTCGGGCTTCTCAGGATGTGAACACCGCCGTGGCCTGGTACCAGCAGAA
GCCAGGCAAGGCCCCCAAGCTGCTGATCTACTCTGCCAGCTTCCTGTATTCCGGAGTG
CCATCTCGGTTTTCCGGCAGCCGGAGCGGCACCGACTTCACCCTGACAATCAGCTCC
CTGCAGCCTGAGGATTTTGCCACATACTATTGCCAGCAGCACTATACCACACCCCCTAC
CTTCGGCCAGGGCACAAAGGTGGAGATCAAGAGGGGAGGAGGAGGATCCGGAGGA
GGAGGCAGCGGAGGCGGCGGCTCCGGCGGCGGCGGCTCTGGCGGCGGCGGCAG
CGGAGGAGGCGGCTCCGAGGTGCAGCTGGTGGAGTCCGGCGGCGGCCTGGTGCAG
CCCGGCGGCAGCCTGCGGCTGTCCTGTGCCGCCTCTGGCTTTAACATCAAGGACACC
TACATCCACTGGGTGAGGCAGGCACCTGGCAAGGGCCTGGAGTGGGTGGCAAGGATC
TATCCAACCAATGGCTACACAAGATATGCCGACTCCGTGAAGGGCCGCTTTACCATCA
GCGCCGATACCTCCAAGAACACAGCCTACCTGCAGATGAATTCTCTGCGGGCCGAGG
ATACAGCCGTGTACTATTGCTCCAGATGGGGCGGCGACGGCTTCTATGCTATGGACTA
TTGGGGGCAGGGAACTCTGGTCACTGTCTCCTCTGGCGGAGGGGGATCCGGCGGCG
GAGGATCTGGCGGAGGTGGAAGTGGGGGAGGCGGATCTCATGTGCAGCTGGTGG
AAAGCGGAGGCGGCCTGGTGCAGCCTGGGGGATCTCTGAGACTGTCTTGTGCCGCCA
GCGGCTTCTCCCTGACCGATTATGGCGTGCACTGGGTGCGACAGGCCCCTGGCAAAG
GACTGGAATGGCTGGGAGTGATTTGGAGTGGCGGAGGCACCGCCTACAACACCGCCC
TGATCTCCCGGTTCACCATCAGCCGGGACAACTCCAAGAACACCCTGTACCTGCAGAT
GAACTCCCTGCGGGCCGAGGACACCGCTGTGTACTACTGCGCCAGACGGGGCTCCTA
CCCCTACAACTACTTCGACGCTTGGGGCTGCGGCACCCTCGTGACAGTGTCTAGCGG
AGGGGGAGGTTCTGGGGGCGGAGGTTCAGGTGGTGGTGGTTCCGGGGGTGGTGGCT
CTGGTGGCGGTGGTTCTGGCGGTGGCGGATCTCAGGCTGTCGTGACCCAGGAACCCA
GCCTGACTGTGTCTCCTGGCGGAACCGTGACCCTGACCTGCGGATCTTCTACCGGCG
CTGTGACCGCCAGCAACTACGCCAATTGGGTGCAGCAGAAACCTGGACAGTGCCCTA
GAGGCCTGATCGGCGGCCACAACAACAGACCTCCAGGCGTGCCAGCCCGGTTCTCTG
GATCTCTGCTGGGCGGAAAGGCCGCTCTGACACTGCTGGGTGCTCAGCCTGAGGACG
AGGCCGAGTACTACTGTGCCCTGTGGTACTCCGACCACTGGGTCATCGGAGGCGGGA
CCAAGCTGACCGTGCTGGGA(ACACCCCTGGGAGACACCACACATACT)AGTGGGC
AGGCCATCAAGAAGGAGCTGACCCAGATCAAGCAGAAGGTGGACAGCCTGC
TGGAGAACCTGGAGAAGATCGAGAAGGAGGGAGGGTCAGGAGGAGCACCGC
ACCATCATCATCACCAT

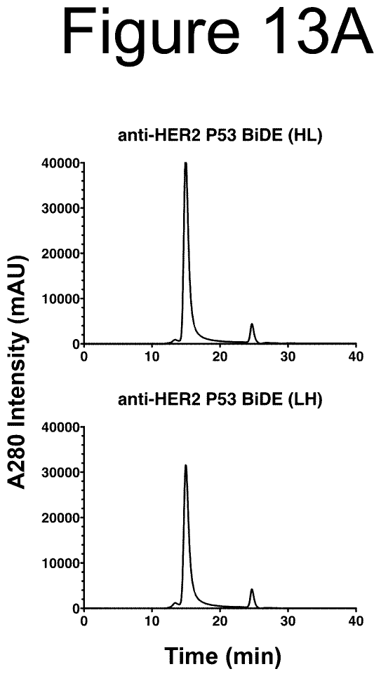

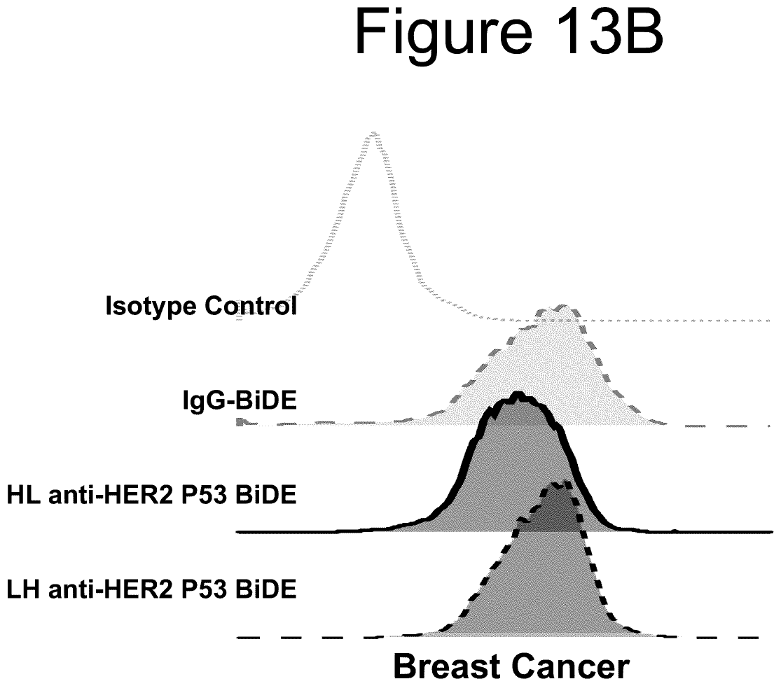

Exemplary anti-HER2 SADA-BiDE constructs of the present example exhibit tetrameric self-assembly, similar to SADA-BiDEs described above. Specifically, FIG. 13A shows SEC-HPLC chromatograms of two different scFv variants of anti-HER2 P53-BiDE constructs with an anti-HER2 scFv in a HL orientation in the upper graph and with an anti-HER2 scFv in a LH orientation in the lower graph. As shown, anti-HER2 P53-BiDE proteins are exceptionally pure after single-step affinity purification and retains a size of ^{˜}200 kDa (^{˜}16 min), which corresponds to the tetramerized form.

Moreover, exemplary anti-HER2 SADA-BiDE constructs have comparable binding characteristics to other SADA-BiDEs. FIG. 13B depicts the results of a FACS analysis on a HER2(+) cell line HCC1954 (breast cancer) using a fluorescently labeled ¹⁷⁵Lu-Bn-DOTA conjugate for detection. HER2/BnDOTA binding capacity of these exemplary anti-HER2 BiDEs (Black solid and dashed, filled) is comparable to that of IgG-BiDE (grey dashed, filled) suggesting strong tumor antigen and payload binding.

Accordingly, this example confirms, that pairing of various targeting and/or antigen binding portions with a SADA domains retains binding and other beneficial characteristics of SADA constructs. These data support that SADA constructs with various targeting domains can be useful.

### Example 13—Exemplary Conjugate with a hnRNPC SADA Domain

This example confirms that a HNRNPC tetramerization domain can act as a SADA domain and self-assemble to form tetrameric proteins. Specifically, this example shows in vitro analyses of an exemplary bispecific antibody based conjugate with a HNRNPC SADA domain, a HNRNPC-BiDE. Provided below are an exemplary polypeptide sequence (SEQ ID NO: 97) and corresponding nucleotide sequence (SEQ ID NO: 98) for an exemplary HNRNPC-BiDE construct.

GD2 HNRNPC BiDE(LL) polypeptide (hu3F8-scFv,
huC825-scFv, huHNRNPC-tet, GS linker,
(IgG3 spacer))
SEQ ID NO: 97
EIVMTQTPATLSVSAGERVTITCKASQSVSNDVTWYQQKPGQAPRLLIY
SASNRYSGVPARFSGSGYGTEFTFTISSVQSEDFAVYFCQQDYSSFGCG
TKLEIKRGGGGSGGGGSGGGGSGGGGSGGGGSGGGGSQVQLVESGPGVV
QPGRSLRISCAVSGFSVTNYGVHWVRQPPGKCLEWLGVIWAGGITNYNS
AFMSRLTISKDNSKNTVYLQMNSLRAEDTAMYYCASRGGHYGYALDYWG
QGTLVTVSSGGGGSGGGGSGGGGSGGGGSHVQLVESGGGLVQPGGSLRL
SCAASGFSLTDYGVHWVRQAPGKGLEWLGVIWSGGGTAYNTALISRFTI
SRDNSKNTLYLQMNSLRAEDTAVYYCARRGSYPYNYFDAWGCGTLVTVS
SGGGGSGGGGSGGGGSGGGGSGGGGSGGGGSQAVVTQEPSLTVSPGGTV
TLTCGSSTGAVTASNYANWVQQKPGQCPRGLIGGHNNRPPGVPARFSGS
LLGGKAALTLLGAQPEDEAEYYCALWYSDHWVIGGGTKLTVLG(TPLGD
TTHT)SGQAIKKELTQIKQKVDSLLENLEKIEKEGGSGGAPHHHHHH
GD2 HNRNPC BiDE(LL) cDNA (hu3F8-scFv,
huC825-scFv, huHNRNPC-tet, GS linker,
(IgG3 spacer))
SEQ ID NO: 98
GAGATCGTGATGACCCAGACACCCGCAACACTGAGCGTGTCTGCCGGCG
AAAGGGTCACTATTACCTGCAAGGCCAGTCAGTCAGTGTCCAACGACGT
GACTTGGTACCAGCAGAAACCAGGCCAGGCTCCCCGGCTGCTGATCTAC
AGCGCATCTAATAGATATAGCGGAGTGCCTGCTCGCTTCAGTGGTTCAG
GCTATGGAACTGAGTTCACCTTCACCATTTCCAGCGTGCAGTCCGAAGA
CTTCGCAGTGTACTTTTGCCAGCAGGATTATTCTAGTTTTGGGTGTGGT
ACAAAGCTGGAGATCAAAAGGGGAGGAGGAGGTAGTGGCGGAGGAGGTT
CAGGCGGAGGGGGTAGCGGCGGAGGGGGTTCTGGCGGCGGCGGTAGTGG
CGGCGGAGGTAGCCAGGTGCAGCTGGTCGAATCCGGCCCTGGAGTGGTC
CAGCCAGGCAGGTCTCTGCGGATCAGTTGCGCCGTGTCCGGATTCAGCG
TCACCAACTACGGAGTGCACTGGGTCAGACAGCCACCTGGCAAGTGTCT
GGAGTGGCTGGGAGTGATCTGGGCAGGAGGAATCACAAACTACAACTCA
GCTTTTATGTCCCGCCTGACTATTAGCAAGGACAACTCTAAAAATACCG
TGTATCTGCAGATGAATTCTCTGCGAGCCGAAGATACCGCTATGTACTA
TTGTGCATCCCGTGGGGGTCATTACGGCTATGCCCTGGATTATTGGGGG
CAGGGTACCCTGGTGACAGTCTCATCCGGCGGAGGGGGATCCGGCGGCG
GAGGATCTGGCGGAGGTGGAAGTGGGGGAGGCGGATCTCATGTGCAGCT
GGTGGAAAGCGGAGGCGGCCTGGTGCAGCCTGGGGGATCTCTGAGACTG
TCTTGTGCCGCCAGCGGCTTCTCCCTGACCGATTATGGCGTGCACTGGG
TGCGACAGGCCCCTGGCAAAGGACTGGAATGGCTGGGAGTGATTTGGAG
TGGCGGAGGCACCGCCTACAACACCGCCCTGATCTCCCGGTTCACCATC
AGCCGGGACAACTCCAAGAACACCCTGTACCTGCAGATGAACTCCCTGC
GGGCCGAGGACACCGCTGTGTACTACTGCGCCAGACGGGGCTCCTACCC
CTACAACTACTTCGACGCTTGGGGCTGCGGCACCCTCGTGACAGTGTCT
AGCGGAGGGGGAGGTTCTGGGGGCGGAGGTTCAGGTGGTGGTGGTTCCG
GGGGTGGTGGCTCTGGTGGCGGTGGTTCTGGCGGTGGCGGATCTCAGGC
TGTCGTGACCCAGGAACCCAGCCTGACTGTGTCTCCTGGCGGAACCGTG
ACCCTGACCTGCGGATCTTCTACCGGCGCTGTGACCGCCAGCAACTACG
CCAATTGGGTGCAGCAGAAACCTGGACAGTGCCCTAGAGGCCTGATCGG
CGGCCACAACAACAGACCTCCAGGCGTGCCAGCCCGGTTCTCTGGATCT
CTGCTGGGCGGAAAGGCCGCTCTGACACTGCTGGGTGCTCAGCCTGAGG
ACGAGGCCGAGTACTACTGTGCCCTGTGGTACTCCGACCACTGGGTCAT
CGGAGGCGGGACCAAGCTGACCGTGCTGGGA(ACACCCCTGGGAGACAC
CACACATACT)AGTGGGCAGGCCATCAAGAAGGAGCTGACCCAGATCAA
GCAGAAGGTGGACAGCCTGCTGGAGAACCTGGAGAAGATCGAGAAGGAG
GGAGGGTCAGGAGGAGCACCGCACCATCATCATCACCAT

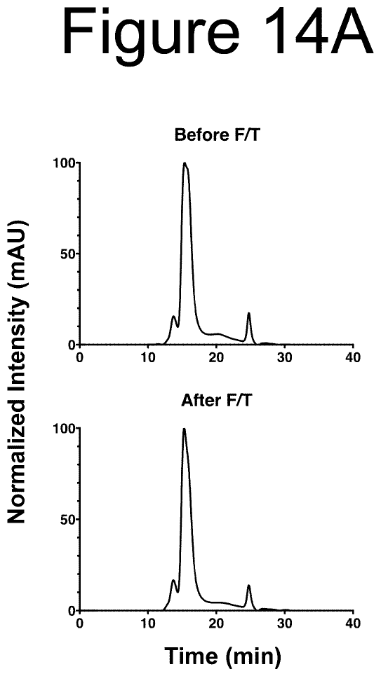

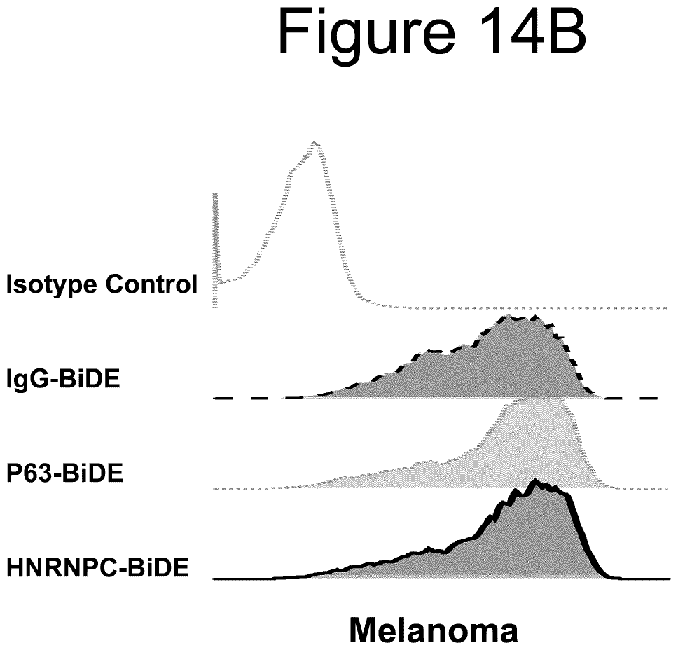

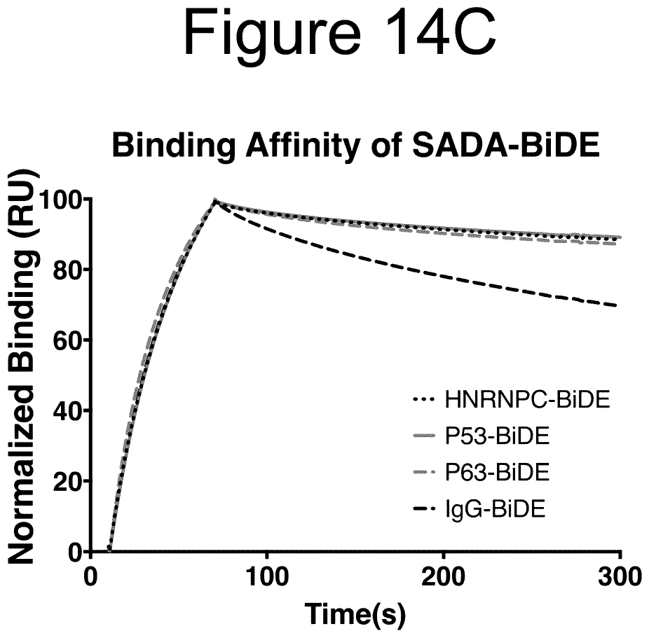

An exemplary HNRNPC-BiDE exhibits tetrameric self-assembly, similar to SADA-BiDEs described above. As shown in FIG. 14A, an exemplary HNRNPC-BiDE polypeptide construct forms a stable tetrameric multimer has shown by SEC-HPLC chromatogram. Single-step affinity purification of an exemplary HNRNPC-BiDE polypeptide and SEC-HPLC analysis shows a tetrameric multimer at the expected size of ^{˜}200 kDa (^{˜}16 min, upper graph), and this purity is maintained after five repeated freeze and thaw cycles (^{˜}16 min, lower graph). Thus, an exemplary HNRNPC-BiDE polypeptide shows high stability and a propensity to not form higher order aggregates. FIG. 14B shows the results of a FACS analysis on a GD2(+) cell line M14-Luc (Melanoma) using a fluorescently labeled ¹⁷⁵Lu-Bn-DOTA conjugate for detection. GD2/BnDOTA binding capacity of an exemplary HNRNPC-BiDE (Solid Black, filled) is compared against an IgG-BiDE (Dashed black, filled) a P63-BiDE (dotted grey, filled) or an isotype control (dashed grey, empty). An exemplary HNRNPC-BIDE shows identical binding to other anti-GD2 BiDEs, suggesting strong tumor antigen and payload binding, as expected from its multimeric state. FIG. 14C depicts normalized binding kinetics of the HNRNPC-BiDE (dotted black) against a GD2 tumor antigen using SPR, compared with the P53—(solid grey), P63—(dashed grey), or IgG-BiDEs (dashed black). Each construct was run as a concentration series across a streptavidin chip coated with biotin-GD2. The highest concentrations of each were then plotted together on a normalized Y-axis to better show the differences in k_{off}. Data was fitted using a two-state reaction model. HNRNPC-BiDE shows a greatly improved k_{off} rate compared with the IgG-BiDE, similar to the P53- and P63-BiDEs. These binding kinetics (Table 8) are evidence of tetrameric antigen binding.

TABLE 8
Association and dissociation kinetics of HNRNPC-BiDE

| | ka1 (1/Ms) | kd1 (1/s) | ka2 (1/s) | kd2 (1/s) | K_{D} (M) |
|---|---|---|---|---|---|
| HNRNPC- | 6.77E+05 | 6.87E−02 | 1.12E−01 | 1.37E−03 | 1.22E−09 |

BiDE

Accordingly, this example confirms, that hnRNPC functions as a SADA domain. These data confirms that different, unrelated polypeptides having characteristics of a SADA domain as described herein have similar in vitro characteristics and can confer beneficial properties to a SADA construct.

Having thus described at least several aspects and embodiments of this invention, it is to be appreciated that various alterations, modifications, and improvements will readily be apparent to those skilled in the art. Such alterations, modifications, and improvements are intended to be part of this disclosure, and are intended to be within the spirit and scope of the invention. Accordingly, the foregoing description and drawing are by way of example only and the invention is described in further detail by the claims that follow.

### EQUIVALENTS

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. The publications, websites and other reference materials referenced herein to describe the background of the invention and to provide additional detail regarding its practice are hereby incorporated by reference.

1. A nucleic acid sequence encoding a polypeptide conjugate comprising:
a self-assembly disassembly (SADA) polypeptide having an amino acid sequence that shows at least 75% identity with that of a human homo-multimerizing polypeptide and being characterized by one or more multimerization dissociation constants (K_{D}); and
at least a first binding domain that binds to a first target and is covalently linked to the SADA polypeptide,
the conjugate being constructed and arranged so that it adopts a first multimerization state and one or more higher-order multimerization states, wherein:
the first multimerization state is less than about ^{˜}70 kDa in size,
at least one of the higher-order multimerization states is a homo-tetramer or higher-order homo-multimer greater than 150 kDa in size,
wherein the higher-order homo-multimerized conjugate is stable in aqueous solution when the conjugate is present at a concentration above the SADA polypeptide K_{D}, and
the conjugate transitions from the higher-order multimerization state(s) to the first multimerization state under physiological conditions when the concentration of the conjugate is below the SADA polypeptide K_{D}, and optionally
wherein the SADA polypeptide:
(i) has a total buried surface area of 900 A2 to 4000 A2; and/or
(ii) lacks unpaired cysteine residues.

2. The nucleic acid sequence of claim 1, wherein the higher-order homo-multimerized conjugate is stable:
(i) for a period of at least 24 hr at 37° C. in an aqueous buffer with a pH of 6.0-8.0;
(ii) for a period of at least 24 hr at 37° C. in an aqueous buffer with a pH of 6.5-7.5;
(iii) for a period of at least 24 hr at 37° C. in an aqueous buffer with a pH of 6.8-7.2
(iv) for a period of at least 48 hours, 72 hours, 1 week, 2 weeks, 1 month, 2 months, 3 months, or more; and/or
(v) over 3 or more freeze-thaw cycles.

3. The nucleic acid sequence of claim 1, wherein the transition of the conjugate from the higher-order multimerization state to the first multimerization state is characterized by a K_{off} within a range of 1×10⁻⁶ to 1×10⁻⁴ (s⁻¹).

4. The nucleic acid sequence of claim 1, wherein the SADA polypeptide comprises a tetramerization, pentamerization or hexamerization domain.

5. The nucleic acid sequence of claim 4, wherein the SADA polypeptide comprises a tetramerization domain of any one of p53, p63, p73, hnRNPC, SNAP-23, Stefin B, KCNQ4, and CBFA2T1.

6. The nucleic acid sequence of claim 5, wherein the SADA polypeptide comprises:
(i) a sequence that is at least 90% identical to a sequence as set forth in any one of SEQ ID NOs: 1, 3, 5, 7, 9, 11, 13, and 15;
(ii) a sequence that is at least 95% identical to a sequence as set forth in any one of SEQ ID NOs: 1, 3, 5, 7, 9, 11, 13, and 15; and/or
(iii) a sequence as set forth in any one of SEQ ID NOs: 1, 3, 5, 7, 9, 11, 13, and 15.

7. The nucleic acid sequence of claim 1, wherein the first target is selected from the group consisting of an in situ target and a payload target, optionally wherein
the in situ target is or comprises an entity selected from the group consisting of: a cell-surface moiety, a cytokine, a receptor ligand, a peptide, a hormone, a metabolite, and a hapten; or
the payload target is a therapeutic payload or a diagnostic payload selected from the group consisting of a drug, a polypeptide (such as a toxin, enzyme, cytokine, chemokine, receptor, or biologic), a chemical probe (such as a fluorescent dye or biotin tag), a radioactive isotope, or a nanoparticle.

8. The nucleic acid sequence of claim 1, wherein the polypeptide conjugate further comprises a second binding domain that binds to a second target, which is different from the first target.

9. The nucleic acid sequence of claim 8, wherein the conjugate
comprises at least two binding domains and wherein the conjugate in the second multimerization state is at least octavalent;
can exist in one or more additional multimeric states; or
is substantially not immunogenic in a human subject.

10. The nucleic acid sequence of claim 8, wherein the second target is selected from the group consisting of an in situ target and a payload target, optionally wherein
the in situ target is or comprises an entity selected from the group consisting of: a cell-surface moiety, a cytokine, a receptor ligand, a peptide, a hormone, a metabolite, and a hapten; or
the payload target is a therapeutic payload or a diagnostic payload selected from the group consisting of a drug, a polypeptide (such as a toxin, enzyme, cytokine, chemokine, receptor, or biologic), a chemical probe (such as a fluorescent dye or biotin tag), a radioactive isotope, or a nanoparticle.

11. The nucleic acid sequence of claim 8, wherein the second target is a cell surface moiety, optionally wherein the cell surface moiety is a cell surface receptor or wherein the cell surface moiety is specifically expressed or enriched on a subset of cells in an organism, or on tumor cells.

12. The nucleic acid sequence of claim 1, wherein the polypeptide conjugate further comprises a dimerization domain or a second SADA domain.

13. The nucleic acid sequence of claim 1, wherein the first binding domain or the second binding domain is or comprises a ligand for a cell surface receptor, a cytokine receptor binding domain, or an antibody, antibody component, or antigen-binding antibody fragment specific for a cell surface target.

14. The nucleic acid sequence of claim 13,
wherein the conjugate is further complexed with a soluble cytokine polypeptide, optionally wherein the cytokine receptor is IL15Rα and the soluble cytokine polypeptide is IL15; or
wherein the first binding domain is an anti-GD2, anti-Globo H, anti-GPA33, anti-PSMA, anti-polysialic acid, anti-Lew^{Y}, anti-L1CAM, anti-HER2, anti-B7H3, anti-CD33, anti-peptide/MHC, anti-glypican3, or anti-GD3 binding domain; or
wherein the first and second binding domains are part of a bispecific antibody agent, optionally wherein bispecific antibody agent comprises a first binding domain that binds a tumor target and a second binding domain that binds a metal-Bn-DOTA or an immune-cell activating receptor.

15. The nucleic acid sequence of claim 14, wherein the bispecific antibody agent comprises a first binding domain that binds a tumor target and a second binding domain that binds a metal-Bn-DOTA.

16. The nucleic acid sequence of claim 15, wherein the nucleic acid comprises a sequence that encodes a conjugate comprising an amino acid sequence selected from the group consisting of SEQ ID Nos: 17, 19, 21, 23, 25, 27, 29, 31, 33, 35, 37, 39, 41, 43, 45, 47, 49, 51, 53, 55, 65, 67, 69, 71, 73, 75, 77, 79, 81, 83, 85, 87, 89, 91, 93, 95 and 97.

17. The nucleic acid sequence of claim 15, wherein the nucleic acid comprises a sequence selected from the group consisting of SEQ ID Nos: 18, 20, 22, 24, 26, 28, 30, 32, 34, 36, 38, 40, 42, 44, 46, 48, 50, 52, 54, 56, 66, 68, 70, 72, 74, 76, 78, 80, 82, 84, 86, 88, 90, 92, 94, 96 and 98.

18. A vector comprising the nucleic acid sequence of claim 1.

19. A host cell comprising the vector of claim 18, optionally wherein the host cell is selected from the group consisting of a bacterial, yeast, insect or mammalian cell.

20. A nucleic acid sequence encoding a polypeptide conjugate comprising a self-assembly disassembly (SADA) polypeptide having an amino acid sequence that is identical to a human homo-multimerizing polypeptide sequence comprising any one of SEQ ID NOs: 1, 3, 5, 7, 9, 11, 13, and 15 and having one or more multimerization dissociation constants (K_{D}); and
a bispecific antibody comprising a first scFv that binds a tumor target and a second scFv that binds a DOTA moiety comprising a radioactive payload, wherein (a) the first scFv is operably linked to the N-terminus of the second scFv, (b) the second scFv includes a V_{H}-CDR1 sequence comprising DYGVH (SEQ ID NO: 103), a V_{H}-CDR2 sequence comprising VIWSGGGTAYNTALIS (SEQ ID NO: 104), a V_{H}-CDR3 sequence comprising RGSYPYNYFDA (SEQ ID NO: 105), a VL-CDR1 sequence comprising GSSTGAVTASNYAN (SEQ ID NO: 106), a VL-CDR2 sequence comprising GHNNRPP (SEQ ID NO: 107), and a V_{H}-CDR3 sequence comprising ALWYSDHWV (SEQ ID NO: 108); and (c) the second scFv is operably linked to the N-terminus of the SADA polypeptide,
wherein the conjugate being constructed and arranged so that it adopts a first multimerization state and at least one additional multimerization state, wherein:
the first multimerization state is less than about ^{˜}70 kDa in size,
at least one additional multimerization state is a homo-tetramer or a homo-multimer greater than 150 kDa in size, and optionally
wherein the SADA polypeptide:
lacks unpaired cysteine residues.
